SEQUENCE LISTING (275 sequences)

SEQ ID NO: 1 - protein, 87 aa, Homo sapiens
RDMAEAHKEAMSRKLGQSESQGPPRAFAKVNSISPGSPSIAGLQVDDEIVEFGSVNTQNFQSLHNIGSVVQHSEGALAPTILLSVSM

SEQ ID NO: 2 - protein, 93 aa, Homo sapiens
LRKEPEIITVTLKKQNGMGLSIVAAKGAGQDKLGIYVKSVVKGGAADVDGRLAAGDQLLSVDGRSLVGLSQERAAELMTRTSSVVTLEVAKQG

SEQ ID NO: 3 - protein, 105 aa, Homo sapiens
LIRPSVISIIGLYKEKGKGLGFSIAGGRDCIRGQMGIFVKTIFPNGSAAEDGRLKEGDEILDVNGIPIKGLTFQEAIHTFKQIRSGLFVLTVRTKLVSPSLTNSS

SEQ ID NO: 4 - protein, 132 aa, Homo sapiens
GISSLGRKTPGPKDRIVMEVTLNKEPRVGLGIGACCLALENSPPGIYIHSLAPGSVAKMESNLSRGDQILEVNSVNVRHAALSKVHAILSKCPPGPVRLVIGRHPNPKVSEQEMDEVIARSTYQESKEANSS

SEQ ID NO: 5 - protein, 105 aa, Homo sapiens
QSENEEDVCFIVLNRKEGSGLGFSVAGGTDVEPKSITVHRVFSQGAASQEGTMNRGDFLLSVNGASLAGLAHGNVLKVLHQAQLHKDALVVIKKGMDQPRPSNSS

SEQ ID NO: 6 - protein, 101 aa, Homo sapiens
LGRSVAVHDALCVEVLKTSAGLGLSLDGGKSSVTGDGPLVIKRVYKGGAAEQAGIIEAGDEILAINGKPLVGLMHFDAWNIMKSVPEGPVQLLIRKHRNSS

SEQ ID NO: 7 - protein, 99 aa, Homo sapiens
REEGGMPQTVILPGPAAWGFRLSGGIDFNQPLVITRITPGSKAAAANLCPGDVILAIDGFGTESMTHADGQDRIKAAAHQLCLKIDRGETHLWSPHSIV

SEQ ID NO: 8 - protein, 85 aa, Homo sapiens
ILVEVQLSGGAPWGFTLKGGREHGEPLVITKIEEGSKAAAVDKLLAGDEIVGINDIGLSGFRQEAICLVKGSHKTLKLVVKRNSS

SEQ ID NO: 9 - protein, 104 aa, Homo sapiens
REKPLFTRDASQLKGTFLSTTLKKSNMGFGFTIIGGDEPDEFLQVKSVIPDGPAAQDGKMETGDVIVYINEVCVLGHTHADVVKLFQSVPIGQSVNLVLCRGYP

SEQ ID NO: 10 - protein, 91 aa, Homo sapiens
LSGATQAELMTLTIVKGAQGFGFTIADSPTGQRVKQILDIQGCPGLCEGDLIVEINQQNVQNLSHTEVVDILKDCPIGSETSLIIHRGGFF

SEQ ID NO: 11 - protein, 93 aa, Homo sapiens
HYKELDVHLRRMESGFGFRILGGDEPGQPILIGAVIAMGSADRDGRLHPGDELVYVDGIPVAGKTHRYVIDLMHHAARNGQVNLTVRRKVLCG

SEQ ID NO: 12 - protein, 106 aa, Homo sapiens
EGRGISSHSLQTSDAVIHRKENEGFGFVIISSLNRPESGSTITVPHKIGRIIDGSPADRCAKLKVGDRILAVNGQSIINMPHADIVKLIKDAGLSVTLRIIPQEEL

SEQ ID NO: 13 - protein, 98 aa, Homo sapiens
LSDYRQPQDFDYFTVDMEKGAKGFGFSIRGGREYKMDLYVLRLAEDGPAIRNGRMRVGDQIIEINGESTRDMTHARAIELIKSGGRRVRLLLKRGTGQ

SEQ ID NO: 14 - protein, 90 aa, Homo sapiens
HESVIGRNPEGQLGFELKGGAENGQFPYLGEVKPGKVAYESGSKLVSEELLLEVNETPVAGLTIRDVLAVIKHCKDPLRLKCVKQGGIHR

SEQ ID NO: 15 - protein, 106 aa, Homo sapiens
SVGHVRGPGPSVQHTTLNGDSLTSQLTLLGGNARGSFVHSVKPGSLAEKAGLREGHQLLLLEGCIRGERQSVPLDTCTKEEAHWTIQRCSGPVTLHYKVNHEGYRK

SEQ ID NO: 16 - protein, 105 aa, Homo sapiens
RRPARRILSQVTMLAFQGDALLEQISVIGGNLTGIFIHRVTPGSAADQMALRPGTQIVMVDYEASEPLFKAVLEDTTLEEAVGLLRRVDGFCCLSVKVNTDGYKR

SEQ ID NO: 17 - protein, 90 aa, Homo sapiens
TRVRLVQFQKNTDEPMGITLKMNELNHCIVARIMHGGMIHRQGTLHVGDEIREINGISVANQTVEQLQKMLREMRGSITFKIVPSYRTQS

SEQ ID NO: 18 - protein, 88 aa, Homo sapiens
LEQKAVLEQVQLDSPLGLEIHTTSNCQHFVSQVDTQVPTDSRLQIQPGDEVVQINEQVVVGWPRKNMVRELLREPAGLSLVLKKIPIP

SEQ ID NO: 19 - protein, 92 aa, Homo sapiens
QRKLVTVEKQDNETFGFEIQSYRPQNQNACSSEMFTLICKIQEDSPAHCAGLQAGDVLANINGVSTEGFTYKQVVDLIRSSGNLLTIETLNG

SEQ ID NO: 20 - protein, 109 aa, Homo sapiens
RCLIQTKGQRSMDGYPEQFCVRIEKNPGLGFSISGGISGQGNPFKPSDKGIFVTRVQPDGPASNLLQPGDKILQANGHSFVHMEHEKAVLLLKSFQNTVDLVIQRELTV

SEQ ID NO: 21 - protein, 101 aa, Homo sapiens
IQVNGTDADYEYEEITLERGNSGLGFSIAGGTDNPHIGDDSSIFITKIITGGAAAQDGRLRVNDCILQVNEVDVRDVTHSKAVEALKEAGSIVRLYVKRRN

SEQ ID NO: 22 - protein, 95 aa, Homo sapiens
IQLIKGPKGLGFSIAGGVGNQHIPGDNSIYVTKIIEGGAAHKDGKLQIGDKLLAVNNVCLEEVTHEEAVTALKNTSDFVYLKVAKPTSMYMNDGN

SEQ ID NO: 23 - protein, 85 aa, Homo sapiens
ILHRGSTGLGFNIVGGEDGEGIFISFILAGGPADLSGELRKGDRIISVNSVDLRAASHEQAAAALKNAGQAVTIVAQYRPEEYSR

SEQ ID NO: 24 - protein, 101 aa, Homo sapiens
ISYVNGTEIEYEFEEITLERGNSGLGFSIAGGTDNPHIGDDPGIFITKIIPGGAAAEDGRLRVNDCILRVNEVDVSEVSHSKAVEALKEAGSIVRLYVRRR

SEQ ID NO: 25 - protein, 110 aa, Homo sapiens
IPILETVVEIKLFKGPKGLGFSIAGGVGNQHIPGDNSIYVTKIIDGGAAQKDGRLQVGDRLLMVNNYSLEEVTHEEAVAILKNTSEVVYLKVGKPTTIVMTDPYGPPNSS

SEQ ID NO: 26 - protein, 105 aa, Homo sapiens
ILEGEPRKVVLHKGSTGLGFNIVGGEDGEGIFVSFILAGGPADLSGELQRGDQILSVNGIDLRGASHEQAAAALKGAGQTVTIIAQHQPEDYARFEAKIHDLNSS

SEQ ID NO: 27 - protein, 102 aa, Homo sapiens
GIPYVEEPRHVKVQKGSEPLGISIVSGEKGGIYVSKVTVGSIAHQAGLEYGDQLLEFNGINLRSATEQQARLIIGQQCDTITILAQYNPHVHQLRNSSZLTD

SEQ ID NO: 28 - protein, 103 aa, Homo sapiens
GILAGDANKKTLEPRVVFIKKSQLELGVHLCGGNLHGVFVAEVEDDSPAKGPDGLVPGDLILEYGSLDVRNKTVEEVYVEMLKPRDGVRLKVQYRPEEFIVTD

SEQ ID NO: 29 - protein, 141 aa, Homo sapiens
PTSPEIQELRQMLQAPHFKALLSAHDTIAQKDFEPLLPPLPDNIPESEEAMRIVCLVKNQQPLGATIKRHEMTGDILVARIIHGGLAERSGLLYAGDKLVEVNGVSVEGLDPEQVIHILAMSRGTIMFKVVPVSDPPVNSS

SEQ ID NO: 30 - protein, 97 aa, Homo sapiens
PTSPEIQELRQMLQAPHFKGATIKRHEMTGDILVARIIHGGLAERSGLLYAGDKLVEVNGVSVEGLDPEQVIHILAMSRGTIMFKVVPVSDPPVNSS

SEQ ID NO: 31 - protein, 93 aa, Homo sapiens
LNIVTVTLNMERHHFLGISIVGQSNDRGDGGIYIGSIMKGGAVAADGRIEPGDMLLQVNDVNFENMSNDDAVRVLREIVSQTGPISLTVAKCW

SEQ ID NO: 32 - protein, 100 aa, Homo sapiens
LNIITVTLNMEKYNFLGISIVGQSNERGDGGIYIGSIMKGGAVAADGRIEPGDMLLQVNDMNFENMSNDDAVRVLRDIVHKPGPIVLTVAKCWDPSPQNS

SEQ ID NO: 33 - protein, 95 aa, Homo sapiens
IITVTLNMEKYNFLGISIVGQSNERGDGGIYIGSIMKGGAVAADGRIEPGDMLLQVNEINFENMSNDDAVRVLREIVHKPGPITLTVAKCWDPSP

SEQ ID NO: 34 - protein, 97 aa, Homo sapiens
LTTQQIDLQGPGPWGFRLVGRKDFEQPLAISRVTPGSKAALANLCIGDVITAIDGENTSNMTHLEAQNRIKGCTDNLTLTVARSEHKVWSPLVTNSS

SEQ ID NO: 35 - protein, 89 aa, Homo sapiens
IFMDSFKVVLEGPAPWGFRLQGGKDFNVPLSISRLTPGGKAAQAGVAVGDWVLSIDGENAGSLTHIEAQNKIRACGERLSLGLSRAQPV

SEQ ID NO: 36 - protein, 100 aa, Homo sapiens
QGHELAKQEIRVRVEKDPELGFSISGGVGGRGNPFRPDDDGIFVTRVQPEGPASKLLQPGDKIIQANGYSFINIEHGQAVSLLKTFQNTVELIIVREVSS

SEQ ID NO: 37 - protein, 155 aa, Homo sapiens
QMSADAAAGAPLPRLCCLEKGPNGYGFHLHGEKGKLGQYIRLVEPGSPAEKAGLLAGDRLVEVNGENVEKETHQQVVSRIRAALNAVRLLVVDPETDEQLQKLGVQVREELLRAQEAPGQAEPPAAAEVQGAGNENEPREADKSHPEQRELRNSS

SEQ ID NO: 38 - protein, 99 aa, Homo sapiens
IQQRELRPRLCTMKKGPSGYGFNLHSDKSKPGQFIRSVDPDSPAEASGLRAQDRIVEVNGVCMEGKQHGDVVSAIRAGGDETKLLVVDRETDEFFKNSS

SEQ ID NO: 39 - protein, 107 aa, Homo sapiens
KNPSGELKTVTLSKMKQSLGISISGGIESKVQPMVKIEKIFPGGAAFLSGALQAGFELVAVDGENLEQVTHQRAVDTIRRAYRNKAREPMELVVRVPGPSPRPSPSD

SEQ ID NO: 40 - protein, 97 aa, Homo sapiens
EGHSHPRVVELPKTEEGLGFNIMGGKEQNSPIYISRIIPGGIADRHGGLKRGDQLLSVNGVSVEGEHHEKAVELLKAAQGKVKLVVRYTPKVLEEME

SEQ ID NO: 41 - protein, 88 aa, Homo sapiens
PGAPYARKTFTIVGDAVGWGFVVRGSKPCHIQAVDPSGPAAAAGMKVCQFVVSVNGLNVLHVDYRTVSNLILTGPRTIVMEVMEELEC

SEQ ID NO: 42 - protein, 97 aa, Homo sapiens
GQYGGETVKIVRIEKARDIPLGATVRNEMDSVIISRIVKGGAAEKSGLLHEGDEVLEINGIEIRGKDVNEVFDLLSDMHGTLTFVLIPSQQIKPPPA

SEQ ID NO: 43 - protein, 98 aa, Homo sapiens
ILAHVKGIEKEVNVYKSEDSLGLTITDNGVGYAFIKRIKDGGVIDSVKTICVGDHIESINGENIVGWRHYDVAKKLKELKKEELFTMKLIEPKKAFEI

SEQ ID NO: 44 - protein, 104 aa, Homo sapiens
KPSQASGHFSVELVRGYAGFGLTLGGGRDVAGDTPLAVRGLLKDGPAQRCGRLEVGDLVLHINGESTQGLTHAQAVERIRAGGPQLHLVIRRPLETHPGKPRGV

SEQ ID NO: 45 - protein, 107 aa, Homo sapiens
PVMSQCACLEEVHLPNIKPGEGLGMYIKSTYDGLHVITGTTENSPADRSQKIHAGDEVIQVNQQTVVGWQLKNLVKKLRENPTGVVLLLKKRPTGSFNFTPEFIVTD

SEQ ID NO: 46 - protein, 100 aa, Homo sapiens
LDDEEDSVKIIRLVKNREPLGATIKKDEQTGAIIVARIMRGGAADRSGLIHVGDELREVNGIPVEDKRPEEIIQILAQSQGAITFKIIPGSKEETPSNSS

SEQ ID NO: 47 - protein, 110 aa, Homo sapiens
MGLGVSAEQPAGGAEGFHLHGVQENSPAQQAGLEPYFDFIITIGHSRLNKENDTLKALLKANVEKPVKLEVFNMKTMRVREVEVVPSNMWGGQGLLGASVRFCSFRRASE

SEQ ID NO: 48 - protein, 99 aa, Homo sapiens
RASEQVWHVLDVEPSSPAALAGLRPYTDYVVGSDQILQESEDFFTLIESHEGKPLKLMVYNSKSDSCREVTVTPNAAWGGEGSLGCGIGYGYLHRIPTQ

SEQ ID NO: 49 - protein, 110 aa, Homo sapiens
MGSSQSVEIPGGGTEGYHVLRVQENSPGHRAGLEPFFDFIVSINGSRLNKDNDTLKDLLKANVEKPVKMLIYSSKTLELRETSVTPSNLWGGQGLLGVSIRFCSFDGANE

SEQ ID NO: 50 - protein, 97 aa, Homo sapiens
NENVWHVLEVESNSPAALAGLRPHSDYIIGADTVMNESEDLFSLIETHEAKPLKLYVYNTDTDNCREVIITPNSAWGGEGSLGCGIGYGYLHRIPTR

SEQ ID NO: 51 - protein, 83 aa, Homo sapiens
VVELMKKEGTTLGLTVSGGIDKDGKPRVSNLRQGGIAARSDQLDVGDYIKAVNGINLAKFRHDEIISLLKNVGERVVLEVEYE

SEQ ID NO: 52 - protein, 110 aa, Homo sapiens
RSSVIFRTVEVTLHKEGNTFGFVIRGGAHDDRNKSRPVVITCVRPGGPADREGTIKPGDRLLSVDGIRLLGTTHAEAMSILKQCGQEAALLIEYDVSVMDSVATASGNSS

SEQ ID NO: 53 - protein, 106 aa, Homo sapiens
HVATASGPLLVEVAKTPGASLGVALTTSMCCNKQVIVIDKIKSASIADRCGALHVGDHILSIDGTSMEYCTLAEATQFLANTTDQVKLEILPHHQTRLALKGPNSS

SEQ ID NO: 54 - protein, 106 aa, Homo sapiens; identical to SEQ ID NO: 53

SEQ ID NO: 55 - protein, 101 aa, Homo sapiens
AESVIPSSGTFHVKLPKKHNVELGITISSPSSRKPGDPLVISDIKKGSVAHRTGTLELGDKLLAIDNIRLDNCSMEDAVQILQQCEDLVKLKIRKDEDNSD

SEQ ID NO: 56 - protein, 90 aa, Homo sapiens
IYTVELKRYGGPLGITISGTEEPFDPIIISSLTKGGLAERTGAIHIGDRILAINSSSLKGKPLSEAIHLLQMAGETVTLKIKKQTDAQSA

SEQ ID NO: 57 - protein, 95 aa, Homo sapiens
IMSPTPVELHKVTLYKDSDMEDFGFSVADGLLEKGVYVKNIRPAGPGDLGGLKPYDRLLQVNHVRTRDFDCCLVVPLIAESGNKLDLVISRNPLA

SEQ ID NO: 58 - protein, 93 aa, Homo sapiens
LSRGCETRELALPRDGQGRLGFEVDAEGFVTHVERFTFAETAGLRPGARLLRVCGQTLPSLRPEAAAQLLRSAPKVCVTVLPPDESGRPRNSS

SEQ ID NO: 59 - protein, 114 aa, Homo sapiens
CSVMIFEVVEQAGAIILEDGQELDSWYVILNGTVEISHPDGKVENLFMGNSFGITPTLDKQYMHGIVRTKVDDCQFVCIAQQDYWRILNHVEKNTHKVEEEGEIVMVHEFIVTD

SEQ ID NO: 60 - protein, 89 aa, Homo sapiens
LENVIAKSLLIKSNEGSYGFGLEDKNKVPIIKLVEKGSNAEMAGMEVGKKIFAINGDLVFMRPFNEVDCFLKSCLNSRKPLRVLVSTKP

SEQ ID NO: 61 - protein, 85 aa, Homo sapiens
PRETVKIPDSADGLGFQIRGFGPSVVHAVGRGTVAAAAGLHPGQCIIKVNGINVSKETHASVIAHVTACRKYRRPTKQDSIQNSS

SEQ ID NO: 62 - protein, 100 aa, Homo sapiens
EDFCYVFTVELERGPSGLGMGLIDGMHTHLGAPGLYIQTLLPGSPAAADGRLSLGDRILEVNGSSLLGLGYLRAVDLIRHGGKKMRFLVAKSDVETAKKI

SEQ ID NO: 63 - protein, 100 aa, Homo sapiens
NNELTQFLPRTITLKKPPGAQLGFNIRGGKASQLGIFISKVIPDSDAHRAGLQEGDQVLAVNDVDFQDIEHSKAVEILKTAREISMRVRFFPYNYHRQKE

SEQ ID NO: 64 - protein, 109 aa, Homo sapiens
LTEFQDKQIKDWKKRFIGIRMRTITPSLVDELKASNPDFPEVSSGIYVQEVAPNSPSQRGGIQDGDIIVKVNGRPLVDSSELQEAVLTESPLLLEVRRGNDDLLFSNSS

SEQ ID NO: 65 - protein, 97 aa, Homo sapiens
HKKYLGLQMLSLTVPLSEELKMHYPDFPDVSSGVYVCKVVEGTAAQSSGLRDHDVIVNINGKPITTTTDVVKALDSDSLSMAVLRGKDNLLLTVNSS

SEQ ID NO: 66 - protein, 104 aa, Homo sapiens
IWQIEYIDIERPSTGGLGFSVVALRSQNLGKVDIFVKDVQPGSVADRDQRLKENDQILAINHTPLDQNISHQQAIALLQQTTGSLRLIVAREPVHTKSSTSSSE

SEQ ID NO: 67 - protein, 102 aa, Homo sapiens
LPETVCWGHVEEVELINDGSGLGFGIVGGKTSGVVVRTIVPGGLADRDGRLQTGDHILKIGGTNVQGMTSEQVAQVLRNCGNSVRMLVARDPAGDISVTNSS

SEQ ID NO: 68 - protein, 111 aa, Homo sapiens
PGSDSSLFETYNVELVRKDGQSLGIRIVGYVGTSHTGEASGIYVKSIIPGSAAYHNGHIQVNDKIVAVDGVNIQGFANHDVVEVLRNAGQVVHLTLVRRKTSSSTSRIHRD

SEQ ID NO: 69 - protein, 96 aa, Homo sapiens
NSDDAELQKYSKLLPIHTLRLGVEVDSFDGHHYISSIVSGGPVDTLGLLQPEDELLEVNGMQLYGKSRREAVSFLKEVPPPFTLVCCRRLFDDEAS

SEQ ID NO: 70 - protein, 102 aa, Homo sapiens
LSSPEVKIVELVKDCKGLGFSILDYQDPLDPTRSVIVIRSLVADGVAERSGGLLPGDRLVSVNEYCLDNTSLAEAVEILKAVPPGLVHLGICKPLVEFIVTD

SEQ ID NO: 71 - protein, 119 aa, Homo sapiens
PNFSHWGPPRIVEIFREPNVSLGISIVVGQTVIKRLKNGEELKGIFIKQVLEDSPAGKTNALKTGDKILEVSGVDLQNASHSEAVEAIKNAGNPVVFIVQSLSSTPRVIPNVHNKANSS

SEQ ID NO: 72 - protein, 99 aa, Homo sapiens
PGELHIIELEKDKNGLGLSLAGNKDRSRMSIFVVGINPEGPAAADGRMRIGDELLEINNQILYGRSHQNASAIIKTAPSKVKLVFIRNEDAVNQMANSS

SEQ ID NO: 73 - protein, 93 aa, Homo sapiens
PATCPIVPGQEMIIEISKGRSGLGLSIVGGKDTPLNAIVIHEVYEEGAAARDGRLWAGDQILEVNGVDLRNSSHEEAITALRQTPQKVRLVVY

SEQ ID NO: 74 - protein, 103 aa, Homo sapiens
ILTLTILRQTGGLGISIAGGKGSTPYKGDDEGIFISRVSEEGPAARAGVRVGDKLLEVNGVALQGAEHHEAVEALRGAGTAVQMRVWRERMVEPENAEFIVTD

SEQ ID NO: 75 - protein, 97 aa, Homo sapiens
PLRQRHVACLARSERGLGFSIAGGKGSTPYRAGDAGIFVSRIAEGGAAHRAGTLQVGDRVLSINGVDVTEARHDHAVSLLTAASPTIALLLEREAGG

SEQ ID NO: 76 - protein, 106 aa, Homo sapiens
ILEGPYPVEEIRLPRAGGPLGLSIVGGSDHSSHPFGVQEPGVFISKVLPRGLAARSGLRVGDRILAVNGQDVRDATHQEAVSALLRPCLELSLLVRRDPAEFIVTD

SEQ ID NO: 77 - protein, 105 aa, Homo sapiens
RELCIQKAPGERLGISIRGGARGHAGNPRDPTDEGIFISKVSPTGAAGRDGRLRVGLRLLEVNQQSLLGLTHGEAVQLLRSVGDTLTVLVCDGFEASTDAALEVS

SEQ ID NO: 78 - protein, 91 aa, Homo sapiens
PHQPIVIHSSGKNYGFTIRAIRVYVGDSDIYTVHHIVWNVEEGSPACQAGLKAGDLITHINGEPVHGLVHTEVIELLLKSGNKVSITTTPF

SEQ ID NO: 79 - protein, 121 aa, Homo sapiens
HLRLLNIACAAKAKRRLMTLTKPSREAPLPFILLGGSEKGFGIFVDSVDSGSKATEAGLKRGDQILEVNGQNFENIQLSKAMEILRNNTHLSITVKTNLFVFKELLTRLSEEKRNGAPNSS

SEQ ID NO: 80 - protein, 95 aa, Homo sapiens
IPPAPRKVEMRRDPVLGFGFVAGSEKPVVVRSVTPGGPSEGKLIPGDQIVMINDEPVSAAPRERVIDLVRSCKESILLTVIQPYPSPKSEFIVTD

SEQ ID NO: 81 - protein, 101 aa, Homo sapiens
LNKRTTMPKDSGALLGLKVVGGKMTDLGRLGAFITKVKKGSLADVVGHLRAGDEVLEWNGKPLPGATNEEVYNIILESKSEPQVEIIVSRPIGDIPRIHRD

SEQ ID NO: 82 - protein, 79 aa, Homo sapiens
QRCVIIQKDQHGFGFTVSGDRIVLVQSVRPGGAAMKAGVKEGDRIIKVNGTMVTNSSHLEVVKLIKSGAYVALTLLGSS

SEQ ID NO: 83 - protein, 87 aa, Homo sapiens
ILVQRCVIIQKDDNGFGLTVSGDNPVFVQSVKEDGAAMRAGVQTGDRIIKVNGTLVTHSNHLEVVKLIKSGSYVALTVQGRPPGNSS

SEQ ID NO: 84 - protein, 79 aa, Homo sapiens
SVEMTLRRNGLGQLGFHVNYEGIVADVEPYGYAWQAGLRQGSRLVEICKVAVATLSHEQMIDLLRTSVTVKVVIIPPHD

SEQ ID NO: 85 - protein, 96 aa, Homo sapiens
LKVMTSGWETVDMTLRRNGLGQLGFHVKYDGTVAEVEDYGFAWQAGLRQGSRLVEICKVAVVTLTHDQMIDLLRTSVTVKVVIIPPFEDGTPRRGW

SEQ ID NO: 86 - protein, 105 aa, Homo sapiens
HYIFPHARIKITRDSKDHTVSGNGLGIRIVGGKEIPGHSGEIGAYIAKILPGGSAEQTGKLMEGMQVLEWNGIPLTSKTYEEVQSIISQQSGEAEICVRLDLNML

SEQ ID NO: 87 - protein, 106 aa, Homo sapiens
LCGSLRPPIVIHSSGKKYGFSLRAIRVYMGDSDVYTVHHVVWSVEDGSPAQEAGLRAGDLITHINGESVLGLVHMDVVELLLKSGNKISLRTTALENTSIKVGNSS

SEQ ID NO: 88 - protein, 86 aa, Homo sapiens
SYSVTLTGPGPWGFRLQGGKDFNMPLTISRITPGSKAAQSQLSQGDLVVAIDGVNTDTMTHLEAQNKIKSASYNLSLTLQKSKNSS

SEQ ID NO: 89 - protein, 126 aa, Homo sapiens
TLNEEHSHSDKHPVTWQPSKDGDRLIGRILLNKRLKDGSVPRDSGAMLGLKVVGGKMTESGRLCAFITKVKKGSLADTVGHLRPGDEVLEWNGRLLQGATFEEVYNIILESKPEPQVELVVSRPIG

SEQ ID NO: 90 - protein, 101 aa, Homo sapiens
ISALGSMRPPIIIHRAGKKYGFTLRAIRVYMGDSDVYTVHHMVWHVEDGGPASEAGLRQGDLITHVNGEPVHGLVHTEVVELILKSGNKVAISTTPLENSS

SEQ ID NO: 91 - protein, 94 aa, Homo sapiens
FSDMRISINQTPGKSLDFGFTIKWDIPGIFVASVEAGSPAEFSQLQVDDEIIAINNTKFSYNDSKEWEEAMAKAQETGHLVMDVRRYGKAGSPE

SEQ ID NO: 92 - protein, 98 aa, Homo sapiens
QSAHLEVIQLANIKPSEGLGMYIKSTYDGLHVITGTTENSPADRCKKIHAGDEVIQVNHQTVVGWQLKNLVNALREDPSGVILTLKKRPQSMLTSAPA

SEQ ID NO: 93 - protein, 100 aa, Homo sapiens
ILTQTLIPVRHTVKIDKDTLLQDYGFHISESLPLTVVAVTAGGSAHGKLFPGDQILQMNNEPAEDLSWERAVDILREAEDSLSITVVRCTSGVPKSSNSS

SEQ ID NO: 94 - protein, 93 aa, Homo sapiens
GLRSPITIQRSGKKYGFTLRAIRVYMGDTDVYSVHHIVWHVEEGGPAQEAGLCAGDLITHVNGEPVHGMVHPEVVELILKSGNKVAVTTTPFE

SEQ ID NO: 95 - protein, 107 aa, Homo sapiens
QGEETKSLTLVLHRDSGSLGFNIIGGRPSVDNHDGSSSEGIFVSKIVDSGPAAKEGGLQIHDRIIEVNGRDLSRATHDQAVEAFKTAKEPIVVQVLRRTPRTKMFTP

SEQ ID NO: 96 - protein, 101 aa, Homo sapiens
QEMDREELELEEVDLYRMNSQDKLGLTVCYRTDDEDDIGIYISEIDPNSIAAKDGRIREGDRIIQINGIEVQNREEAVALLTSEENKNFSLLIARPELQLD

SEQ ID NO: 97 - protein, 91 aa, Homo sapiens
RSFQYVPVQLQGGAPWGFTLKGGLEHCEPLTVSKIEDGGKAALSQKMRTGDELVNINGTPLYGSRQEALILIKGSFRILKLIVRRRNAPVS

SEQ ID NO: 98 - protein, 102 aa, Homo sapiens
ILEKLELFPVELEKDEDGLGISIIGMGVGADAGLEKLGIFVKTVTEGGAAQRDGRIQVNDQIVEVDGISLVGVTQNFAATVLRNTKGNVRFVIGREKPGQVS

SEQ ID NO: 99 - protein, 113 aa, Homo sapiens
KDVNVYVNPKKLTVIKAKEQLKLLEVLVGIIHQTKWSWRRTGKQGDGERLVVHGLLPGGSAMKSGQVLIGDVLVAVNDVDVTTENIERVLSCIPGPMQVKLTFENAYDVKRET

SEQ ID NO: 100 - protein, 90 aa, Homo sapiens
TRGCETVEMTLRRNGLGQLGFHVNFEGIVADVEPFGFAWKAGLRQGSRLVEICKVAVATLTHEQMIDLLRTSVTVKVVIIQPHDDGSPRR

SEQ ID NO: 101 - protein, 96 aa, Homo sapiens
VENILAKRLLILPQEEDYGFDIEEKNKAVVVKSVQRGSLAEVAGLQVGRKIYSINEDLVFLRPFSEVESILNQSFCSRRPLRLLVATKAKEIIKIP

SEQ ID NO: 102 - protein, 110 aa, Homo sapiens
PDSAGPGEVRLVSLRRAKAHEGLGFSIRGGSEHGVGIYVSLVEPGSLAEKEGLRVGDQILRVNDKSLARVTHAEAVKALKGSKKLVLSVYSAGRIPGGYVTNHIEFIVTD

SEQ ID NO: 103 - protein, 106 aa, Homo sapiens
LQGGDEKKVNLVLGDGRSLGLTIRGGAEYGLGIYITGVDPGSEAEGSGLKVGDQILEVNWRSFLNILHDEAVRLLKSSRHLILTVKDVGRLPHARTTVDEEFIVTD

SEQ ID NO: 104 - protein, 87 aa, Homo sapiens
WTSGAHVHSGPCEEKCGHPGHRQPLPRIVTIQRGGSAHNCGQLKVGHVILEVNGLTLRGKEHREAARIIAEAFKTKDRDYIDFLDSL

SEQ ID NO: 105 - protein, 100 aa, Homo sapiens
ELRRAELVEIIVETEAQTGVSGINVAGGGKEGIFVRELREDSPAARSLSLQEGDQLLSARVFFENFKYEDALRLLQCAEPYKVSFCLKRTVPTGDLALRP

SEQ ID NO: 106 - protein, 102 aa, Homo sapiens
PSQLKGVLVRASLKKSTMGFGFTIIGGDRPDEFLQVKNVLKDGPAAQDGKIAPGDVIVDINGNCVLGHTHADVVQMFQLVPVNQYVNLTLCRGYPLPDDSED

SEQ ID NO: 107 - protein, 100 aa, Homo sapiens
ASSGSSQPELVTIPLIKGPKGFGFAIADSPTGQKVKMILDSQWCQGLQKGDIIKEIYHQNVQNLTHLQVVEVLKQFPVGADVPLLILRGGPPSPTKTAKM

SEQ ID NO: 108 - protein, 143 aa, Homo sapiens
LYEDKPPLTNTFLISNPRTTADPRILYEDKPPNTKDLDVFLRKQESGFGFRVLGGDGPDQSIYIGAIIPLGAAEKDGRLRAADELMCIDGIPVKGKSHKQVLDLMTTAARNGHVLLTVRRKIFYGEKQPEDDSGSPGIHRELT

SEQ ID NO: 109 - protein, 102 aa, Homo sapiens
PAPQEPYDVVLQRKENEGFGFVILTSKNKPPPGVIPHKIGRVIEGSPADRCGKLKVGDHISAVNGQSIVELSHDNIVQLIKDAGVTVTLTVIAEEEHHGPPS

SEQ ID NO: 110 - protein, 98 aa, Homo sapiens
QNLGCYPVELERGPRGFGFSLRGGKEYNMGLFILRLAEDGPAIKDGRIHVGDQIVEINGEPTQGITHTRAIELIQAGGNKVLLLLRPGTGLIPDHGLA

SEQ ID NO: 111 - protein, 84 aa, Homo sapiens
ITVVELIKKEGSTLGLTISGGTDKDGKPRVSNLRPGGLAARSDLLNIGDYIRSVNGIHLTRLRHDEIITLLKNVGERVVLEVEY

SEQ ID NO: 112 - protein, 92 aa, Homo sapiens
ILDVSLYKEGNSFGFVLRGGAHEDGHKSRPLVLTYVRPGGPADREGSLKVGDRLLSVDGIPLHGASHATALATLRQCSHEALFQVEYDVATP

SEQ ID NO: 113 - protein, 102 aa, Homo sapiens
IHTVANASGPLMVEIVKTPGSALGISLTTTSLRNKSVITIDRIKPASVVDRSGALHPGDHILSIDGTSMEHCSLLEATKLLASISEKVRLEILPVPQSQRPL

SEQ ID NO: 114 - protein, 103 aa, Homo sapiens
IQIVHTETTEVVLCGDPLSGFGLQLQGGIFATETLSSPPLVCFIEPDSPAERCGLLQVGDRVLSINGIATEDGTMEEANQLLRDAALAHKVVLEVEFDVAESV

SEQ ID NO: 115 - protein, 103 aa, Homo sapiens
IQFDVAESVIPSSGTFHVKLPKKRSVELGITISSASRKRGEPLIISDIKKGSVAHRTGTLEPGDKLLAIDNIRLDNCPMEDAVQILRQCEDLVKLKIRKDEDN

SEQ ID NO: 116 - protein, 94 aa, Homo sapiens
IQTTGAVSYTVELKRYGGPLGITISGTEEPFDPIVISGLTKRGLAERTGAIHVGDRILAINNVSLKGRPLSEAIHLLQVAGETVTLKIKKQLDR

SEQ ID NO: 117 - protein, 105 aa, Homo sapiens
ILEMEELLLPTPLEMHKVTLHKDPMRHDFGFSVSDGLLEKGVYVHTVRPDGPAHRGGLQPFDRVLQVNHVRTRDFDCCLAVPLLAEAGDVLELIISRKPHTAHSS

SEQ ID NO: 118 - protein, 91 aa, Homo sapiens
MALTVDVAGPAPWGFRITGGRDFHTPIMVTKVAERGKAKDADLRPGDIIVAINGESAEGMLHAEAQSKIRQSPSPLRLQLDRSQATSPGQT

SEQ ID NO: 119 - protein, 84 aa, Homo sapiens
SNYSVSLVGPAPWGFRLQGGKDFNMPLTISSLKDGGKAAQANVRIGDVVLSIDGINAQGMTHLEAQNKIKGCTGSLNMTLQRAS

SEQ ID NO: 120 - protein, 133 aa, Homo sapiens
TLVEHSKLYCGHCYYQTVVTPVIEQILPDSPGSHLPHTVTLVSIPASSHGKRGLSVSIDPPHGPPGCGTEHSHTVRVQGVDPGCMSPDVKNSIHVGDRILEINGTPIRNVPLDEIDLLIQETSRLLQLTLEHD

SEQ ID NO: 121 - protein, 92 aa, Homo sapiens
PYSVTLISMPATTEGRRGFSVSVESACSNYATTVQVKEVNRMHISPNNRNAIHPGDRILEINGTPVRTLRVEEVEDAISQTSQTLQLLIEHD

SEQ ID NO: 122 - protein, 82 aa, Homo sapiens
IHSVTLRGPSPWGFRLVGRDFSAPLTISRVHAGSKASLAALCPGDLIQAINGESTELMTHLEAQNRIKGCHDHLTLSVSRPE

SEQ ID NO: 123 - protein, 74 aa, Homo sapiens
VCYRTDDEEDLGIYVGEVNPNSIAAKDGRIREGDRIIQINGVDVQNREEAVAILSQEENTNISLLVARPESQLA

SEQ ID NO: 124 - protein, 106 aa, Homo sapiens
PSELKGKFIHTKLRKSSRGFGFTVVGGDEPDEFLQIKSLVLDGPAALDGKMETGDVIVSVNDTCVLGHTHAQVVKIFQSIPIGASVDLELCRGYPLPFDPDGIHRD

SEQ ID NO: 125 - protein, 79 aa, Homo sapiens
PATQPELITVHIVKGPMGFGFTIADSPGGGGQRVKQIVDSPRCRGLKEGDLIVEVNKKNVQALTHNQVVDMLVECPKGS

SEQ ID NO: 126 - protein, 93 aa, Homo sapiens
IPATQPELITVHIVKGPMGFGFTIADSPGGGGQRVKQIVDSPRCRGLKEDLIVEVNKKNVQALTHNQVVDMLVECPKGSEVTLLVQRGGNSSZ

SEQ ID NO: 127 - protein, 91 aa, Homo sapiens
QATQEQDFYTVELERGAKGFGFSLRGGREYNMDLYVLRLAEDGPAERCGKMRIGDEILEINGETTKNMKHSRAIELIKNGGRRVRLFLKRG

SEQ ID NO: 128 - protein, 119 aa, Homo sapiens
PGVVSTVVQPYDVEIRRGENEGFGFVIVSSVSRPEAGTTFAGNACVAMPHKIGRIIEGSPADRCGKLKVGDRILAVNGCSITNKSHSDIVNLIKEAGNTVTLRIIPGDESSNAEFIVTD

SEQ ID NO: 129 - protein, 102 aa, Homo sapiens
PDYQEQDIFLWRKETGFGFRILGGNEPGEPIYIGHIVPLGAADTDGRLRSGDELICVDGTPVIGKSHQLVVQLMQQAAKQGHVNLTVRRKVVFAVPKTENSS

SEQ ID NO: 130 - protein, 100 aa, Homo sapiens
PAKMEKEETTRELLLPNWQGSGSHGLTIAQRDDGVFVQEVTQNSPAARTGVVKEGDQIVGATIYFDNLQSGEVTQLLNTMGHHTVGLKLHRKGDRSPNSS

SEQ ID NO: 131 - protein, 98 aa, Homo sapiens
SENCKDVFIEKQKGEILGVVIVESGWGSILPTVIIANMMHGGPAEKSGKLNIGDQIMSINGTSLVGLPLSTCQSIIKGLKNQSRVKLNIVRCPPVNSS

SEQ ID NO: 132 - protein, 92 aa, Homo sapiens
LRCPPVTTVLIRRPDLRYQLGFSVQNGIICSLMRGGIAERGGVRVGHRIIEINGQSVVATPHEKIVHILSNAVGEIHMKTMPAAMYRLLNSS

SEQ ID NO: 133 - protein, 106 aa, Homo sapiens
HNGDLDHFSNSDNCREVHLEKRRGEGLGVALVESGWGSLLPTAVIANLLHGGPAERSGALSIGDRLTAINGTSLVGLPLAACQAAVRETKSQTSVTLSIVHCPPVT

SEQ ID NO: 134 - protein, 90 aa, Homo sapiens
PVTTAIIHRPHAREQLGFCVEDGIICSLLRGGIAERGGIRVGHRIIEINGQSVVATPHARIIELLTEAYGEVHIKTMPAATYRLLTGNSS

SEQ ID NO: 135 - protein, 86 aa, Homo sapiens
RKVRLIQFEKVTEEPMGITLKLNEKQSCTVARILHGGMIHRQGSLHVGDEILEINGTNVTNHSVDQLQKAMKETKGMISLKVIPNQ

SEQ ID NO: 136 - protein, 89 aa, Homo sapiens
PVPPDAVRMVGIRKTAGEHLGVTFRVEGGELVIARILHGGMVAQQGLLHVGDIIKEVNGQPVGSDPRALQELLRNASGSVILKILPNYQ

SEQ ID NO: 137 - protein, 99 aa, Homo sapiens
NIDEDFDEESVKIVRLVKNKEPLGATIRRDEHSGAVVVARIMRGGAADRSGLVHVGDELREVNGIAVLHKRPDEISQILAQSQGSITLKIIPATQEEDR

SEQ ID NO: 138 - protein, 99 aa, Homo sapiens
QGRHVEVFELLKPPSGGLGFSVVGLRSENRGELGIFVQEIQEGSVAHRDGRLKETDQILAINGQALDQTITHQQAISILQKAKDTVQLVIARGSLPQLV

SEQ ID NO: 139 - protein, 97 aa, Homo sapiens
PVHWQHMETIELVNDGSGLGFGIIGGKATGVIVKTILPGGVADQHGRLCSGDHILKIGDTDLAGMSSEQVAQVLRQCGNRVKLMIARGAIEERTAPT

SEQ ID NO: 140 - protein, 98 aa, Homo sapiens
QESETFDVELTKNVQGLGITIAGYIGDKKLEPSGIFVKSITKSSAVEHDGRIQIGDQIIAVDGTNLQGFTNQQAVEVLRHTGQTVLLTLMRRGMKQEA

SEQ ID NO: 141 - protein, 92 aa, Homo sapiens
LNYEIVVAHVSKFSENSGLGISLEATVGHHFIRSVLPEGPVGHSGKLFSGDELLEVNGITLLGENHQDVVNILKELPIEVTMVCCRRTVPPT

SEQ ID NO: 142 - protein, 103 aa, Homo sapiens
WEAGIQHIELEKGSKGLGFSILDYQDPIDPASTVIIIRSLVPGGIAEKDGRLLPGDRLMFVNDVNLENSSLEEAVEALKGAPSGTVRIGVAKPLPLSPEENSS

SEQ ID NO: 143 - protein, 99 aa, Homo sapiens
RNVSKESFERTINIAKGNSSLGMTVSANKDGLGMIVRSIIHGGAISRDGRIAIGDCILSINEESTISVTNAQARAMLRRHSLIGPDIKITYVPAEHLEE

SEQ ID NO: 144 - protein, 112 aa, Homo sapiens
LNWNQPRRVELWREPSKSLGISIVGGRGMGSRLSNGEVMRGIFIKHVLEDSPAGKNGTLKPGDRIVEVDGMDLRDASHEQAVEAIRKAGNPVVFMVQSIINRPRKSPLPSLL

SEQ ID NO: 145 - protein, 95 aa, Homo sapiens
LTGELHMIELEKGHSGLGLSLAGNKDRSRMSVFIVGIDPNGAAGKDGRLQIADELLEINGQILYGRSHQNASSIIKCAPSKVKIIFIRNKDAVNQ

SEQ ID NO: 146 - protein, 94 aa, Homo sapiens
LSSFKNVQHLELPKDQGGLGIAISEEDTLSGVIIKSLTEHGVAATDGRLKVGDQILAVDDEIVVGYPIEKFISLLKTAKMTVKLTIHAENPDSQ

SEQ ID NO: 147 - protein, 95 aa, Homo sapiens
LPGCETTIEISKGRTGLGLSIVGGSDTLLGAIIIHEVYEEGAACKDGRLWAGDQILEVNGIDLRKATHDEAINVLRQTPQRVRLTLYRDEAPYKE

SEQ ID NO: 148 - protein, 98 aa, Homo sapiens
KEEEVCDTLTIELQKKPGKGLGLSIVGKRNDTGVFVSDIVKGGIADADGRLMQGDQILMVNGEDVRNATQEAVAALLKCSLGTVTLEVGRIKAGPFHS

SEQ ID NO: 149 - protein, 96 aa, Homo sapiens
LQGLRTVEMKKGPTDSLGISIAGGVGSPLGDVPIFIAMMHPTGVAAQTQKLRVGDRIVTICGTSTEGMTHTQAVNLLKNASGSIEMQVVAGGDVSV

SEQ ID NO: 150 - protein, 91 aa, Homo sapiens
LGPPQCKSITLERGPDGLGFSIVGGYGSPHGDLPIYVKTVFAKGAASEDGRLKRGDQIIAVNGQSLEGVTHEEAVAILKRTKGTVTLMVLS

SEQ ID NO: 151 - protein, 93 aa, Homo sapiens
IQYEEIVLERGNSGLGFSIAGGIDNPHVPDDPGIFITKIIPGGAAAMDGRLGVNDCVLRVNEVEVSEVVHSRAVEALKEAGPVVRLVVRRRQN

SEQ ID NO: 152 - protein, 90 aa, Homo sapiens
ITLLKGPKGLGFSIAGGIGNQHIPGDNSIYITKIIEGGAAQKDGRLQIGDRLLAVNNTNLQDVRHEEAVASLKNTSDMVYLKVAKPGSLE

SEQ ID NO: 153 - protein, 119 aa, Homo sapiens
ILLHKGSTGLGFNIVGGEDGEGIFVSFILAGGPADLSGELRRGDRILSVNGVNLRNATHEQAAAALKRAGQSVTIVAQYRPEEYSRFESKIHDLREQMMNSSMSSGSGSLRTSEKRSLE

SEQ ID NO: 154 - protein, 111 aa, Homo sapiens
CVERLELFPVELEKDSEGLGISIIGMGAGADMGLEKLGIFVKTVTEGGAAHRDGRIQVNDLLVEVDGTSLVGVTQSFAASVLRNTKGRVRFMIGRERPGEQSEVAQRIHRD

SEQ ID NO: 155 - protein, 90 aa, Homo sapiens
IQPNVISVRLFKRKVGGLGFLVKERVSKPPVIISDLIRGGAAEQSGLIQAGDIILAVNGRPLVDLSYDSALEVLRGIASETHVVLILRGP

SEQ ID NO: 156 - protein, 107 aa, Homo sapiens
QANSDESDIIHSVRVEKSPAGRLGFSVRGGSEHGLGIFVSKVEEGSSAERAGLCVGDKITEVNGLSLESTTMGSAVKVLTSSSRLHMMVRRMGRVPGIKFSKEKNSS

SEQ ID NO: 157 - protein, 106 aa, Homo sapiens
PSDTSSEDGVRRIVHLYTTSDDFCLGFNIRGGKEFGLGIYVSKVDHGGLAEENGIKVGDQVLAANGVRFDDISHSQAVEVLKGQTHIMLTIKETGRYPAYKEMNSS

SEQ ID NO: 158 - protein, 115 aa, Homo sapiens
KIKKFLTESHDRQAKGKAITKKKYIGIRMMSLTSSKAKELKDRHRDFPDVISGAYIIEVIPDTPAEAGGLKENDVIISINGQSVVSANDVSDVIKRESTLNMVVRRGNEDIMITV

SEQ ID NO: 159 - protein, 100 aa, Homo sapiens
PDGEITSIKINRVDPSESLSIRLVGGSETPLVHIIIQHIYRDGVIARDGRLLPGDIILKVNGMDISNVPHNYAVRLLRQPCQVLWLTVMREQKFRSRNSS

SEQ ID NO: 160 - protein, 101 aa, Homo sapiens
HRPRDDSFHVILNKSSPEEQLGIKLVRKVDEPGVFIFNVLDGGVAYRHGQLEENDRVLAINGHDLRYGSPESAAHLIQASERRVHLVVSRQVRQRSPENSS

SEQ ID NO: 161 - protein, 104 aa, Homo sapiens
PTITCHEKVVNIQKDPGESLGMTVAGGASHREWDLPIYVISVEPGGVISRDGRIKTGDILLNVDGVELTEVSRSEAVALLKRTSSSIVLKALEVKEYEPQEFIV

SEQ ID NO: 162 - protein, 101 aa, Homo sapiens
LLTEEEINLTRGPSGLGFNIVGGTDQQYVSNDSGIYVSRIKENGAAALDGRLQEGDKILSVNGQDLKNLLHQDAVDLFRNAGYAVSLRVQHRLQVQNGIHS

SEQ ID NO: 163 - protein, 94 aa, Homo sapiens
PVDAIRILGIHKRAGEPLGVTFRVENNDLVIARILHGGMIDRQGLLHVGDIIKEVNGHEVGNNPKELQELLKNISGSVTLKILPSYRDTITPQQ

SEQ ID NO: 164 - protein, 105 aa, Homo sapiens
PNFSLDDMVKLVEVPNDGGPLGIHVVPFSARGGRTLGLLVKRLEKGGKAEHENLFRENDCIVRINDGDLRNRRFEQAQHMFRQAMRTPIIWFHVVPAANKEQYEQ

SEQ ID NO: 165 - protein, 94 aa, Homo sapiens
GKRLNIQLKKGTEGLGFSITSRDVTIGGSAPIYVKNILPRGAAIQDGRLKAGDRLIEVNGVDLVGKSQEEVVSLLRSTKMEGTVSLLVFRQEDA

SEQ ID NO: 166 - protein, 113 aa, Homo sapiens
PREFLTFEVPLNDSGSAGLGVSVKGNRSKENHADLGIFVKSIINGGAASKDGRLRVNDQLIAVNGESLLGKTNQDAMETLRRSMSTEGNKRGMIQLIVASRISKCNELKSNSS

SEQ ID NO: 167 - protein, 103 aa, Homo sapiens
PRTKDTLSDMTRTVEISGEGGPLGIHVVPFFSSLSGRILGLFIRGIEDNSRSKREGLFHENECIVKINNVDLVDKTFAQAQDVFRQAMKSPSVLLHVLPPQNR

SEQ ID NO: 168 - protein, 98 aa, Homo sapiens
SNKNAKKIKIDLKKGPEGLGFTVVTRDSSIHGPGPIFVKNILPKGAAIKDGRLQSGDRILEVNGRDVTGRTQEELVAMLRSTKQGETASLVIARQEGH

SEQ ID NO: 169 - protein, 106 aa, Homo sapiens
ITSEQLTFEIPLNDSGSAGLGVSLKGNKSRETGTDLGIFIKSIIHGGAAFKDGRLRMNDQLIAVNGESLLGKSNHEAMETLRRSMSMEGNIRGMIQLVILRRPERP

SEQ ID NO: 170 - protein, 107 aa, Homo sapiens
PETHRRVRLHKHGSDRPLGFYIRDGMSVRVAPQGLERVPGIFISRLVRGGLAESTGLLAVSDEILEVNGIEVAGKTLDQVTDMMVANSHNLIVTVKPANQRNNVNSS

SEQ ID NO: 171 - protein, 119 aa, Homo sapiens
PVSSIIDVDILPETHRRVRLYKYGTEKPLGFYIRDGSSVRVTPHGLEKVPGIFISRLVPGGLAQSTGLLAVNDEVLEVNGIEVSGKSLDQVTDMMIANSRNLIITVRPANQRNNRIHRD

SEQ ID NO: 172 - protein, 111 aa, Homo sapiens
IDVDLVPETHRRVRLHRHGCEKPLGFYIRDGASVRVTPHGLEKVPGIFISRMVPGGLAESTGLLAVNDEVLEVNGIEVAGKTLDQVTDMMIANSHNLIVTVKPANQRNNVV

SEQ ID NO: 173 - protein, 100 aa, Homo sapiens
RSRKLKEVRLDRLHPEGLGLSVRGGLEFGCGLFISHLIKGGQADSVGLQVGDEIVRINGYSISSCTHEEVINLIRTKKTVSIKVRHIGLIPVKSSPDEFH

SEQ ID NO: 174 - protein, 102 aa, Homo sapiens
IPGNRENKEKKVFISLVGSRGLGCSISSGPIQKPGIFISHVKPGSLSAEVGLEIGDQIVEVNGVDFSNLDHKEAVNVLKSSRSLTISIVAAAGRELFMTDEF

SEQ ID NO: 175 - protein, 103 aa, Homo sapiens
PEQIMGKDVRLLRIKKEGSLDLALEGGVDSPIGKVVVSAVYERGAAERHGGIVKGDEIMAINGKIVTDYTLAEADAALQKAWNQGGDWIDLVVAVCPPKEYDD

SEQ ID NO: 176 - protein, 103 aa, Homo sapiens
LTSTFNPRECKLSKQEGQNYGFFLRIEKDTEGHLVRVVEKCSPAEKAGLQDGDRVLRINGVFVDKEEHMQVVDLVRKSGNSVTLLVLDGDSYEKAGSPGIHRD

SEQ ID NO: 177 - protein, 92 aa, Homo sapiens
RLCYLVKEGGSYGFSLKTVQGKKGVYMTDITPQGVAMRAGVLADDHLIEVNGENVEDASHEEVVEKVKKSGSRVMFLLVDKETDKREFIVTD

SEQ ID NO: 178 - protein, 112 aa, Homo sapiens
QFKRETASLKLLPHQPRIVEMKKGSNGYGFYLRAGSEQKGQIIKDIDSGSPAEEAGLKNNDLVVAVNGESVETLDHDSVVEMIRKGGDQTSLLVVDKETDNMYRLAEFIVTD

SEQ ID NO: 179 - protein, 95 aa, Homo sapiens
PDTTEEVDHKPKLCRLAKGENGYGFHLNAIRGLPGSFIKEVQKGGPADLAGLEDEDVIIEVNGVNVLDEPYEKVVDRIQSSGKNVTLLVZGKNSS

SEQ ID NO: 180 - protein, 89 aa, Homo sapiens
PTVPGKVTLQKDAQNLIGISIGGGAQYCPCLYIVQVFDNTPAALDGTVAAGDEITGVNGRSIKGKTKVEVAKMIQEVKGEVTIHYNKLQ

SEQ ID NO: 181 - protein, 98 aa, Homo sapiens
SQGVGPIRKVLLLKEDHEGLGISITGGKEHGVPILISEIHPGQPADRCGGLHVGDAILAVNGVNLRDTKHKEAVTILSQQRGEIEFEVVYVAPEVDSD

SEQ ID NO: 182 - protein, 97 aa, Homo sapiens
IHVTILHKEEGAGLGFSLAGGADLENKVITVHRVFPNGLASQEGTIQKGNEVLSINGKSLKGTTHHDALAILRQAREPRQAVIVTRKLTPEEFIVTD

SEQ ID NO: 183 - protein, 98 aa, Homo sapiens
TAEATVCTVTLEKMSAGLGFSLEGGKGSLHGDKPLTINRIFKGAASEQSETVQPGDEILQLGGTAMQGLTRFEAWNIIKALPDGPVTIVIRRKSLQSK

SEQ ID NO: 184 - protein, 162 aa, Homo sapiens
IREAKYSGVLSSIGKIFKEEGLLGFFVGLIPHLLGDVVFLWGCNLLAHFINAYLVDDSVSDTPGGLGNDQNPGSQFSQALAIRSYTKFVMGIAVSMLTYPFLLVGDLMAVNNCGLQAGLPPYSPVFKSWIHCWKYLSVQGQLFRGSSLLFRRVSSGSCFALE

SEQ ID NO: 185 - protein, 99 aa, Homo sapiens
LEYEEITLERGNSGLGFSIAGGTDNPHIGDDPSIFITKIIPGGAAAQDGRLRVNDSILFVNEVDVREVTHSAAVEALKEAGSIVRLYVMRRKPPAENSS

SEQ ID NO: 186 - protein, 112 aa, Homo sapiens
HVMRRKPPAEKVMEIKLIKGPKGLGFSIAGGVGNQHIPGDNSIYVTKIIEGGAAHKDGRLQIGDKILAVNSVGLEDVMHEDAVAALKNTYDVVYLKVAKPSNAYLLEFIVTD

SEQ ID NO: 187 - protein, 75 aa, Homo sapiens [sequence omitted]

SEQ ID NO: 188 - protein, 102 aa, Homo sapiens
QNDNGDSYLVLIRITPDEDGKFGFNLKGGVDQKMPLVVSRINPESPADTCIPKLNEGDQIVLINGRDISEHTHDQVVMFIKASRESHSRELALVIRRRAVRS

SEQ ID NO: 189 - protein, 88 aa, Homo sapiens
IRMKPDENGRFGFNVKGGYDQKMPVIVSRVAPGTPADLCVPRLNEGDQVVLINGRDIAEHTHDQVVLFIKASCERHSGELMLLVRPNA

SEQ ID NO: 190 - protein, 106 aa, Homo sapiens
PEREITLVNLKKDAKYGLGFQIIGGEKMGRLDLGIFISSVAPGGPADFHGCLKPGDRLISVNSVSLEGVSHHAAIEILQNAPEDVTLVISQPKEKISKVPSTPVHL

SEQ ID NO: 191 - protein, 95 aa, Homo sapiens
GDIFEVELAKNDNSLGISVTGGVNTSVRHGGIYVKAVIPQGAAESDGRIHKGDRVLAVNGVSLEGATHKQAVETLRNTGQVVHLLLEKGQSPTSK

SEQ ID NO: 192 - protein, 104 aa, Homo sapiens
TEENTFEVKLFKNSSGLGFSFSREDNLIPEQINASIVRVKKLFAGQPAAESGKIDVGDVILKVNGASLKGLSQQEVISALRGTAPEVFLLLCRPPPGVLPEIDT

SEQ ID NO: 193 - protein, 98 aa, Homo sapiens
ELEVELLITLIKSEKASLGFTVTKGNQRIGCYVHDVIQDPAKSDGRLKPGDRLIKVNDTDVTNMTHTDAVNLLRAASKTVRLVIGRVLELPRIPMLPH

SEQ ID NO: 194 - protein, 94 aa, Homo sapiens
MLPHLLPDITLTCNKEELGFSLCGGHDSLYQVVYISDINPRSVAAIEGNLQLLDVIHYVNGVSTQGMTLEEVNRALDMSLPSLVLKATRNDLPV

SEQ ID NO: 195 - protein, 96 aa, Homo sapiens
RPSPPRVRSVEVARGRAGYGFTLSGQAPCVLSCVMRGSPADFVGLRAGDQILAVNEINVKKASHEDVVKLIGKCSGVLHMVIAEGVGRFESCSNSS

SEQ ID NO: 196 - protein, 96 aa, Homo sapiens
LCSERRYRQITIPRGKDGFGFTICCDSPVRVQAVDSGGPAERAGLQQLDTVLQLNERPVEHWKCVELAHEIRSCPSEIILLVWRMVPQVKPGIHRD

SEQ ID NO: 197 - protein, 124 aa, Homo sapiens
SEDETFSWPGPKTVTLKRTSQGFGFTLRHFIVYPPESAIQFSYKDEENGNRGGKQRNRLEPMDTIFVKQVKEGGPAFEAGLCTGDRIIKVNGESVIGKTYSQVIALIQNSDTTLELSVMPKDED

SEQ ID NO: 198 - protein, 97 aa, Homo sapiens
SAKNRWRLVGPVHLTRGEGGFGLTLRGDSPVLIAAVIPGSQAAAAGLKEGDYIVSVNGQPCRWWRHAEVVTELKAAGEAGASLQVVSLLPSSRLPSI

SEQ ID NO: 199 - protein, 112 aa, Homo sapiens
RGEKKNSSSGISGSQRRYIGVMMLTLSPSILAELQLREPSFPDVQHGVLIHKVILGSPAHRAGLRPGDVILAIGEQMVQNAEDVYEAVRTQSQLAVQIRRGRETLTLYVNSS

SEQ ID NO: 200 - protein, 109 aa, Homo sapiens
LEEKTVVLQKKDNEGFGFVLRGAKADTPIEEFTPTPAFPALQYLESVDEGGVAWQAGLRTGDFLIEVNNENVVKVGHRQVVNMIRQGGNHLVLKVVTVTRNLDPDDNSS

SEQ ID NO: 201 - protein, 110 aa, Homo sapiens
SDYVIDDKVAVLQKRDHEGFGFVLRGAKAETPIEEFTPTPAFPALQYLESVDVEGVAWRAGLRTGDFLIEVNGVNVVKVGHKQVVALIRQGGNRLVMKVVSVTRKPEEDG

SEQ ID NO: 202 - protein, 103 aa, Homo sapiens
ISNTATKGRYIYLEAFLEGGAPWGFTLKGGLEHGEPLIISKVEEGGKADTLSSKLQAGDEVVHINEVTLSSSRKEAVSLVKGSYKTLRLVVRRDVCTDPGHAD

SEQ ID NO: 203 - protein, 110 aa, Homo sapiens; identical to SEQ ID NO: 47

SEQ ID NO: 204 - protein, 109 aa, Homo sapiens
RASEQVWHVLDVEPSSPAALAGLRPYTDYVVGSDQILQESEDFFTLIESHEGKPLKLMVYNSKSDSCRESGMWHWLWVSTPDPNSAPQLPQEATWHPTTFCSTTWCPTT

SEQ ID NO: 205 - protein, 105 aa, Homo sapiens
IQPLSLPEGEITTIEIHRSNPYIQLGISIVGGNETPLINIVIQEVYRDGVIARDGRLLAGDQILQVNNYNISNVSHNYARAVLSQPCNTLHLTVLRERRFGNRAH

SEQ ID NO: 206 - protein, 98 aa, Homo sapiens
SNSPREEIFQVALHKRDSGEQLGIKLVRRTDEPGVFILDLLEGGLAAQDGRLSSNDRVLAINGHDLKYGTPELAAQIIQASGERVNLTIARPGKPQPG

SEQ ID NO: 207 - protein, 103 aa, Homo sapiens
QCVTCQEKHITVKKEPHESLGMTVAGGRGSKSGELPIFVTSVPPHGCLARDGRIKRGDVLLNINGIDLTNLSHSEAVAMLKASAASPAVALKALEVQIVEEAT

SEQ ID NO: 208 - protein, 97 aa, Homo sapiens
PSTLHSCHDIVLRRSYLGSWGFSIVGGYEENHTNQPFFIKTIVLGTPAYYDGRLKCGDMIVAVNGLSTVGMSHSALVPMLKEQRNKVTLTVICWPGS

SEQ ID NO: 209 - protein, 80 aa, Homo sapiens
SVTDGPKFEVKLKKNANGLGFSFVQMEKESCSHLKSDLVRIKRLFPGQPAEENGAIAAGDIILAVNGRSTEGLIFQEVLH

SEQ ID NO: 210 - protein, 20 aa, Homo sapiens
LLRGAPQEVTLLLCRPPPGA

SEQ ID NO: 211 - protein, 100 aa, Homo sapiens
QPEPLRPRLCRLVRGEQGYGFHLHGEKGRRGQFIRRVEPGSPAEAAALRAGDRLVEVNGVNVEGETHHQVVQRIKAVEGQTRLLVVDQETDEELRRRNSS

SEQ ID NO: 212 - protein, 99 aa, Homo sapiens
PLRELRPRLCHLRKGPQGYGFNLHSDKSRPGQYIRSVDPGSPAARSGLRAQDRLIEVNGQNVEGLRHAEVVASIKAREDEARLLVVDPETDEHFKRNSS

SEQ ID NO: 213 - protein, 92 aa, Homo sapiens
PGVREIHLCKDERGKTGLRLRKVDQGLFVQLVQANTPASLVGLRFGDQLLQIDGRDCAGWSSHKAHQVVKKASGDKIVVVVRDRPFQRTVTM

SEQ ID NO: 214 - protein, 90 aa, Homo sapiens
PFQRTVTMHKDSMGHVGFVIKKGKIVSLVKGSSAARNGLLTNHYVCEVDGQNVIGLKDKKIMEILATAGNVVTLTIIPSVIYEHIVEFIV

SEQ ID NO: 215 - protein, 96 aa, Homo sapiens
SLERPRFCLLSKEEGKSFGFHLQQELGRAGHVVCRVDPGTSAQRQGLQEGDRILAVNNDVVEHEDYAVVVRRIRASSPRVLLTVLARHAHDVARAQ

SEQ ID NO: 216 - protein, 95 aa, Homo sapiens
ISLPTKPRCLHLEKGPQGFGFLLREEKGLDGRPGQFLWEVDPGLPAKKAGMQAGDRLVAVAGESVEGLGHEETVSRIQGQGSCVSLTVVDPEADR

SEQ ID NO: 217 - protein, 96 aa, Homo sapiens
IPSVPLGSRQCFLYPGPGGSYGFRLSCVASGPRLFISQVTPGGSAARAGLQVGDVILEVNGYPVGGQNDLERLQQLPEAEPPLCLKLAARSLRGLE

SEQ ID NO: 218 - protein, 112 aa, Homo sapiens
LKEKTVLLQKKDSEGFGFVLRGAKAQTPIEEFTPTPAFPALQYLESVDEGGVAWRAGLRMGDFLIEVNGQNVVKVGHRQVVNMIRQGGNTLMVKVVMVTRHPDMDEAVQNSS

SEQ ID NO: 219 - protein, 88 aa, Homo sapiens
LEIKQGIREVILCKDQDGKIGLRLKSIDNGIFVQLVQANSPASLVGLRFGDQVLQINGENCAGWSSDKAHKVLKQAFGEKITMRIHRD

SEQ ID NO: 220 - protein, 91 aa, Homo sapiens
LRDRPFERTITMHKDSTGHVGFIFKNGKITSIVKDSSAARNGLLTEHNICEINGQNVIGLKDSQIADILSTSGTVVTITMPAFIFEHMNSS

SEQ ID NO: 221 - protein, 94 aa, Homo sapiens
QRRRVTVRKADAGGLGISIKGGRENKMPILISKIFKGLAADQTEALFVGDAILSVNGEDLSSATHDEAVQVLKKTGKEVVLEVKYMKDVSPYFK

SEQ ID NO: 222 - protein, 86 aa, Homo sapiens
PVRRVVKQEAGGLGISIKGGRENRMPILISKIFPGLAADQSRALRLGDAILSVNGTDLRQATHDQAVQALKRAGKEVLLEVKFIRE

SEQ ID NO: 223 - protein, 101 aa, Homo sapiens
EPFYSGERTVTIRRQTVGGFGLSIKGGAEHNIPVVVSKISKEQRAELSGLLFIGDAILQINGINVRKCRHEEVVQVLRNAGEEVTLTVSFLKRAPAFLKLP

SEQ ID NO: 224 - protein, 99 aa, Homo sapiens
SHQGRNRRTVTLRRQPVGGLGLSIKGGSEHNVPVVISKIFEDQAADQTGMLFVGDAVLQVNGIHVENATHEEVVHLLRNAGDEVTITVEYLREAPAFLK

SEQ ID NO: 225 - protein, 91 aa, Homo sapiens
RGETKEVEVTKTEDALGLTITDNGAGYAFIKRIKEGSIINRIEAVCVGDSIEAINDHSIVGCRHYEVAKMLRELPKSQPFTLRLVQPKRAF

SEQ ID NO: 226 - protein, 88 aa, Homo sapiens
HSIHIEKSDTAADTYGFSLSSVEEDGIRRLYVNSVKETGLASKKGLKAGDEILEINNRAADALNSSMLKDFLSQPSLGLLVRTYPELE

SEQ ID NO: 227 - protein, 97 aa, Homo sapiens
PLNVYDVQLTKTGSVCDFGFAVTAQVDERQHLSRIFISDVLPDGLAYGEGLRKGNEIMTLNGEAVSDLDLKQMEALFSEKSVGLTLIARPPDTKATL

SEQ ID NO: 228 - protein, 103 aa, Homo sapiens
QRVEIHKLRQGENLILGFSIGGGIDQDPSQNPFSEDKTDKGIYVTRVSEGGPAEIAGLQIGDKIMQVNGWDMTMVTHDQARKRLTKRSEEVVRLLVTRQSLQK

SEQ ID NO: 229 - protein, 86 aa, Homo sapiens
RKEVEVFKSEDALGLTITDNGAGYAFIKRIKEGSVIDHIHLISVGDMIEAINGQSLLGCRHYEVARLLKELPRGRTFTLKLTEPRK

SEQ ID NO: 230 - protein, 91 aa, Homo sapiens
HSHPRVVELPKTDEGLGFNVMGGKEQNSPIYISRIIPGGVAERHGGLKRGDQLLSVNGVSVEGEHHEKAVELLKAAKDSVKLVVRYTPKVL

SEQ ID NO: 231 - protein, 100 aa, Homo sapiens
LSNQKRGVKVLKQELGGLGISIKGGKENKMPILISKIFKGLAADQTQALYVGDAILSVNGADLRDATHDEAVQALKRAGKEVLLEVKYMREATPYVKNSS

SEQ ID NO: 232 - protein, 97 aa, Homo sapiens
QRSSIKTVELIKGNLQSVGLTLRLVQSTDGYAGHVIIETVAPNSPAAIADLQRGDRLIAIGGVKITSTLQVLKLIKQAGDRVLVYYERPVGQSNQGA

SEQ ID NO: 233 - protein, 110 aa, Homo sapiens
IHFSNSENCKELQLEKHKGEILGVVVVESGWGSILPTVILANMMNGGPAARSGKLSIGDQIMSINGTSLVGLPLATCQGIIKGLKNQTQVKLNIVSCPPVTTVLIKRNSS

SEQ ID NO: 234 - protein, 94 aa, Homo sapiens
IPPVTTVLIKRPDLKYQLGFSVQNGIICSLMRGGIAERGGVRVGHRIIEINGQSVVATAHEKIVQALSNSVGEIHMKTMPAAMFRLLTGQENSS

SEQ ID NO: 235 - protein, 101 aa, Homo sapiens
IWEQHTVTLHRAPGFGFGIAISGGRDNPHFQSGETSIVISDVLKGGPAEGQLQENDRVAMVNGVSMDNVEHAFAVQQLRKSGKNAKITIRRKKKVQIPNSS

SEQ ID NO: 236 - protein, 95 aa, Homo sapiens
ISSQPAKPTKVTLVKSRKNEEYGLRLASHIFVKEISQDSLAARDGNIQEGDVVLKINGTVTENMSLTDAKTLIERSKGKLKMVVQRDRATLLNSS

SEQ ID NO: 237 - protein, 90 aa, Homo sapiens
IRMKLVKFRKGDSVGLRLAGGNDVGIFVAGVLEDSPAAKEGLEEGDQILRVNNVDFTNIIREEAVLFLLDLPKGEEVTILAQKKKDVFSN

SEQ ID NO: 238 - protein, 96 aa, Homo sapiens
LIWEQYTVTLQKDSKRGFGIAVSGGRDNPHFENGETSIVISDVLPGGPADGLLQENDRVVMVNGTPMEDVLHSFAVQQLRKSGKVAAIVVKRPRKV

SEQ ID NO: 239 - protein, 79 aa, Homo sapiens
RVLLMKSRANEEYGLRLGSQIFVKEMTRTGLATKDGNLHEGDIILKINGTVTENMSLTDARKLIEKSRGKLQLVVLRDS

SEQ ID NO: 240 - protein, 90 aa, Homo sapiens
HAPNTKMVRFKKGDSVGLRLAGGNDVGIFVAGIQEGTSAEQEGLQEGDQILKVNTQDFRGLVREDAVLYLLEIPKGEMVTILAQSRADVY

SEQ ID NO: 241 - protein, 106 aa, Homo sapiens
IPGNSTIWEQHTATLSKDPRRGFGIAISGGRDRPGGSMVVSDVVPGGPAEGRLQTGDHIVMVNGVSMENATSAFAIQILKTCTKMANITVKRPRRIHLPAEFIVTD

SEQ ID NO: 242 - protein, 4 aa, Artificial
  PDZ ligand protein motif
RALL

SEQ ID NO: 243 - protein, 4 aa, Artificial
  PDZ ligand protein motif
RILL

SEQ ID NO: 244 - protein, 4 aa, Artificial
  PDZ ligand protein motif
FKNC

SEQ ID NO: 245 - protein, 4 aa, Artificial
  PDZ ligand protein motif
FKDC

SEQ ID NO: 246 - protein, 4 aa, Artificial
  PDZ ligand protein motif
YKNC

SEQ ID NO: 247 - protein, 4 aa, Artificial
  PDZ ligand protein motif
YKDC

SEQ ID NO: 248 - protein, 4 aa, Artificial
  PDZ ligand protein motif
IALL

SEQ ID NO: 249 - protein, 4 aa, Artificial
  PDZ ligand protein motif
LALL

SEQ ID NO: 250 - protein, 4 aa, Artificial
  PDZ ligand protein motif
LTLL

SEQ ID NO: 251 - protein, 4 aa, Artificial
  PDZ ligand protein motif
EILA

SEQ ID NO: 252 - protein, 4 aa, Artificial
  PDZ ligand protein motif
GILA

SEQ ID NO: 253 - protein, 4 aa, Artificial
  PDZ ligand protein motif
DILA

SEQ ID NO: 254 - protein, 4 aa, Artificial
  PDZ ligand protein motif
FTSA

SEQ ID NO: 255 - protein, 4 aa, Artificial
  PDZ ligand protein motif
WVYI

SEQ ID NO: 256 - protein, 4 aa, Artificial
  PDZ ligand protein motif
PASA

SEQ ID NO: 257 - protein, 4 aa, Artificial
  PDZ ligand protein motif
PVSA

SEQ ID NO: 258 - protein, 4 aa, Artificial
  PDZ ligand protein motif
GVDA

SEQ ID NO: 259 - protein, 4 aa, Artificial
  PDZ ligand protein motif
DVEL

SEQ ID NO: 260 - protein, 4 aa, Artificial
  PDZ ligand protein motif
QCKL

SEQ ID NO: 261 - protein, 4 aa, Artificial
  PDZ ligand protein motif
QCRL

SEQ ID NO: 262 - protein, 4 aa, Artificial
  PDZ ligand protein motif
YYRV

SEQ ID NO: 263 - protein, 4 aa, Artificial
  PDZ ligand protein motif
YYRI

SEQ ID NO: 264 - protein, 4 aa, Artificial
  PDZ ligand protein motif
QVGI

SEQ ID NO: 265 - protein, 4 aa, Artificial
  PDZ ligand protein motif
HIGI

SEQ ID NO: 266 - protein, 4 aa, Artificial
  PDZ ligand protein motif
QIGI

SEQ ID NO: 267 - protein, 4 aa, Artificial
  PDZ ligand protein motif
RIGI

SEQ ID NO: 268 - protein, 4 aa, Artificial
  PDZ ligand protein motif
IKDL

SEQ ID NO: 269 - protein, 4 aa, Artificial
  PDZ ligand protein motif
IEDL

SEQ ID NO: 270 - protein, 4 aa, Artificial
  PDZ ligand protein motif
SSWA

SEQ ID NO: 271 - protein, 4 aa, Artificial
  PDZ ligand protein motif
YTGF

SEQ ID NO: 272 - protein, 4 aa, Artificial
  PDZ ligand protein motif
GMFA

SEQ ID NO: 273 - protein, 20 aa, Artificial
  HIV peptide motif
YGRKKRRQRRRRQGFERALL

SEQ ID NO: 274 - protein, 20 aa, Artificial
  HIV peptide motif
YGRKKRRQRRRRQGLERILL

SEQ ID NO: 275 - protein, 4 aa, Artificial
  PDZ peptide motif
GLGF

(19) United States
(12) Patent Application Publication
(10) Pub. No.: US 2007/0014803 A1
(43) Pub. Date: Jan. 18, 2007

(54) METHODS AND COMPOSITIONS FOR DIAGNOSIS AND TREATMENT OF VIRAL AND BACTERIAL INFECTIONS

(75) Inventors: Peter S. Lu, Palo Alto, CA (US); Joshua D. Rabinowitz, Princeton, NJ (US); Michael P. Belmares, San Jose, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Arbor Vita Corporation, Sunnyvale, CA (US)

(21) Appl. No.: 11/428,531

(22) Filed: Jul. 3, 2006

Related U.S. Application Data
(60) Provisional application No. 60/696,221, filed on Jul. 1, 2005.

(51) Int. Cl. C12Q 1/70; G01N 33/554; A61K 39/42; A61K 39/40; C07K 16/10; C07K 16/12; A61K 48/00

(52) U.S. Cl. 424/160.1; 435/5; 435/7.32; 514/44; 424/168.1; 530/388.3; 530/388.4

(57) ABSTRACT

The invention provides method and compositions for determining the presence and amount of a virus such as HIV-1, HIV-2, Hepatitis B, Hepatitis C, RSV, Rotavirus A, and M. tuberculosis in a sample. Also provided are methods for determining whether a subject is infected with HIV, as well as, the type. The methods involve contacting a sample from the subject with a PDZ polypeptide (PDZ) or other PL binding factor and/or PDZ ligands (PL) and determining whether binding interactions occur between PDZ or other binding factor and PL. Assays for identifying anti-viral and anti-bacterial agents are also provided, as well as, methods for using the compositions to alter PDZ binding to PL in viral or bacterial infected cells.

### CROSS-REFERENCES TO RELATED APPLICATIONS

This application claims priority of U.S. Provisional application 60/696,221, filed Jul. 1, 2005, herein incorporated by reference in its entirety for all purposes.

### BACKGROUND OF THE INVENTION

Epidemic viral infections are responsible for significant worldwide loss life and income in human illnesses ranging from the common cold to life-threatening influenza, West Nile and HIV infections. Timely detection and diagnosis are key, both in determining appropriate therapy and limiting spread of disease. Rapid diagnostic methods are particularly useful in reducing patient suffering and population risks

Microbiological and immunochemical methods are known to be useful in rapid detection of viral pathogens. Traditional microbiological methods are relatively reliable, but are also slow, labor intensive and expensive. Some solutions have been offered by nucleotide probe-, PCR-and immunoassay-based methods, but these assays often are not able to discriminate the most medically important strains of pathogenic viruses and particularly those strains that are often involved in establishing chronic and life threatening illnesses and cancers.

There remains a significant need in the medical arts for improved, inexpensive, rapid, accurate and discriminatory methods capable of detecting the particular strains of pathogenic viruses most often involved in generating medically important diseases. There is also a special need for simple assay methodologies that can be routinely used by relatively untrained individuals in doctor's and veterinary offices, schools, manufacturing plants and in developing countries where resources can be limited and sophisticated lab equipment not widely available.

### BRIEF SUMMARY OF THE INVENTION

The invention provides a method for determining if a subject is infected with a virus. The method involves contacting a sample from the subject with a PDZ-containing polypeptide and determining whether the PDZ polypeptide binds to a viral PDZ ligand polypeptide. Binding between the PDZ-containing polypeptide and the viral PDZ ligand polypeptide indicates that the subject is infected with the virus. Assays for identifying anti-viral and anti-bacterial agents are also provided. The invention finds use in a variety of diagnostic and therapeutic applications.

Disclosed herein are methods for identifying whether a patient is infected with M. tuberculosis, by determining whether an M. tuberculosis PDZ ligand (PL) protein is present in a patient sample, presence indicating the patient is infected with M. tuberculosis. In one aspect the determining involves contacting a patient sample with an agent that specifically binds to the M. tuberculosis PL protein; and detecting specific binding between the agent and the PL protein, specific binding indicating presence of M. tuberculosis. In some aspects, the M. tuberculosis PL protein is ESXN, ESXS, or ESAT-6. In some aspects, the agent that specifically binds to the PL proteins binds to the PL motif. In some aspects, the method involves using the PL motif SSWA (SEQ ID NO:270) for M. tuberculosis ESXN protein, or the YTGF (SEQ ID NO:27 1) for M. tuberculosis ESXS protein, or the GMFA (SEQ ID NO:272) for M. tuberculosis ESAT-6 protein. When the agent that specifically binds to the M. tuberculosis ESXN protein PL motif is a PDZ protein it can be one of the following, TIP2, KIAA1526, and PSD95 (p2). When the agent that specifically binds to the M. tuberculosis ESXS protein PL motif is a PDZ protein, it can be one of the following, MAST2, MAST3, Shank3, APXL1, and syntenin. When the agent that specifically binds to the M. tuberculosis ESAT-6 protein PL motif is a PDZ protein, it can be one of the following, INADL (p3), RIM2, and TIP2. Disclosed herein are isolated antibodies that specifically bind to a carboxy-terminal motif in a PL protein of M. tuberculosis. Disclosed herein are methods for the treatment or prophylaxis of a patient having or at risk of tuberculosis, that involve administering to the patient an effective regime of an agent that that inhibits interaction of a PL protein of M. tuberculosis with a PDZ protein of the cell and thereby effecting treatment or prophylaxis of the infection. In some aspects, the agent is an antibody that specifically binds to the PL motif of the PL protein. In further aspects, the agent is one of the following, an antisense oligonucleotide, a small molecule, an siRNA and a zinc finger protein, and the agent inhibits expression of either the M. tuberculosis PL protein or a PDZ protein that interacts with it. When the bacteria is a member of the genus Mycobacteria, the PL protein is preferably, a member of the ESAT-6 family.

Disclosed herein are methods for identifying whether a patient is infected with HIV, by determining whether an HIV PDZ ligand (PL) protein is present in a patient sample, the presence of the HIV PL indicating the patient is infected with HIV. In some aspects, the determining involves contacting a patient sample with an agent that specifically binds to the HIV PL protein; and detecting specific binding between the agent and the PL protein, specific binding indicating presence of HIV. HIV PL proteins include Env, Nef or Vif. Preferably, the agent that specifically binds to the PL proteins binds to the PL motif. The following PL motif have been identified for HIV proteins, RALL (SEQ ID NO:242) or RILL (SEQ ID NO:243) for HIV-1 Env, FKNC (SEQ ID NO:244), FKDC (SEQ ID NO:245), YKNC (SEQ ID NO:246), or YKDC (SEQ ID NO:247) for HIV-1 Nef protein, IALL (SEQ ID NO:248), LALL (SEQ ID NO:249), or LTLL (SEQ ID NO:250) for HIV2 Env protein, and EILA(SEQ ID NO:251), GILA (SEQ ID NO:252), or DILA (SEQ ID NO:253) for HIV-2 Vif protein. In some aspects, the agent that specifically binds to the Env PL motif for HIV-1 is preferably a PDZ protein selected from the group consisting of: AIPC (p1), GORASP1 (p1), INADL (p3), KIAA0316, KIAA1284, MAGI1 (p1), MAST2, MINT1 (p1,2), NSP, NOS1, PAR3 (p3), PAR3L (p3), PAR6 beta, RIM2, Rhodophilin-like, SITAC-18 (p2), SITAC-18 (p1), KIAA1284, PICK1, Shank 1, Shank 2, Shank 3, and TIPI. In some aspects, the agent that specifically binds to the Nef PL motif for HIV-1 is a PDZ protein selected from the group consisting of: MINT1, SITAC-18, TIP1 and PICK1. In some aspects, the agent that specifically binds to the Env PL motif for HIV-2 is a PDZ protein selected from the group consisting of: EBP50 (p1), KIAA1284, MAST2, NSP, PAR3, PICK1, Shank 1, Shank 2, Shank 3, and TIP1. In some aspects, the agent that specifically binds to the Vif PL motif for HIV-2 is a PDZ protein selected from the group consisting of: INADL (p3), RIM2, and EBP50 (p1). Also disclosed herein are isolated antibodies that specifically bind to a carboxy-terminal motif in a PL protein of HIV. Also disclosed herein are methods for the treatment or prophylaxis of a patient having or at risk of HIV infection, by administering to the patient an effective regime of an agent that that inhibits interaction of a PL protein of HIV with a PDZ protein of the cell and thereby effecting treatment or prophylaxis of the infection. In some aspects, the agent is an antibody that specifically binds to the PL motif of the PL protein. In some aspects, the agent is one of the following, an antisense oligonucleotide, a small molecule, an siRNA and a zinc finger protein, and the agent inhibits expression of either the HIV-1 PL protein or a PDZ protein that interacts with it. PL proteins that have been identified for HIV include Env, Nef and Vif.

Disclosed herein are methods for identifying whether a patient is infected with Hepatitis B, by determining whether a Hepatitis B PDZ ligand (PL) protein is present in a patient sample, presence indicating the patient is infected with Hepatitis B. In some aspects, the determining involves, contacting a patient sample with an agent that specifically binds to the Hepatitis B PL protein; and detecting specific binding between the agent and the PL protein, specific binding indicating presence of Hepatitis B. Known PL proteins for Hepatitis B protein are Protein X or S antigen. Preferably, the agent that specifically binds to the PL protein binds to the PL motif. In some aspects, the PL motif is. FTSA (SEQ ID NO:254) for Hepatitis B Protein X, or WVY1 (SEQ ID NO:255) for Hepatitis B S antigen. In some aspects, when the agent that specifically binds to the Hepatitis B Protein X PL motif is a PDZ protein, the PDZ protein is one of, TIP2, KIAA1526, SITAC-18, MINT1 (p1,2), DVL3, and NOS1. In other aspects, when the agent that specifically binds to the Hepatitis B S antigen PL motif is a PDZ protein, the PDZ protein is PTPL1 (p4), HEMBA 1003117, AF6, AIPC, SYNTENIN, MUPP1 (p3,7,9,1 1), DVL2 (01), ZO-3 (p1), SIP1, AIPC (p1), GORASP1 (p1), INADL (p3), KIAA0316, KIAA1284, MAGI1 (p1), MAST2, MINT1 (p1,2), NSP, NOS1, PAR3 (p3), PAR3L (p3), PAR6 beta, RIM2, Rhodophilin-like, SITAC-18 (p2), SITAC-18 (p1), KIAA1284, PICK1, Shank 1, Shank 2, Shank 3, or TIP1. Also disclosed herein are isolated antibodies that specifically bind to a carboxy-terminal motif in a PL protein of Hepatitis B. Also disclosed herein are method for the treatment or prophylaxis of a patient having or at risk of Hepatitis B infection, involving administering to the patient an effective regime of an agent that that inhibits interaction of a PL protein of Hepatitis B with a PDZ protein of the cell and, in this way, effecting treatment or prophylaxis of the infection. Preferably at least one of the agents is an antibody that specifically binds to the PL motif of the PL protein. In one aspect the agent is an antisense oligonucleotide, a small molecule, an siRNA or a zinc finger protein, and the agent inhibits expression of either the hepatitis C PL protein or a PDZ protein that interacts with it. In some aspects, the PL protein is Protein X or S antigen.

Disclosed herein are methods for identifying whether a patient is infected with flavivirus, by determining whether a flavivirus PDZ ligand (PL) protein is present in a patient sample, presence indicating the patient is infected with flavivirus. In some aspects, the determining involves, contacting a patient sample with an agent that specifically binds to the flavivirus PL protein; and detecting specific binding between the agent and the PL protein, specific binding indicating presence of Flavivirus. Known PL proteins for the flavivirus Hepatitis C virus are PASA (SEQ ID NO:256), or PVSA(SEQ ID NO:257) for Hepatitis C Capsid C protein, or GVDA (SEQ ID NO:258) for Hepatitis C E1 protein. Preferably the agent that specifically bids to Hepatitis C Capsid C PL motif is a PDZ protein at least one of, TIP-2, and ZO-1 (p2) and the agent that specifically binds to the Hepatitis C E1 protein PL motif is at least one of, TIP2, RIM2, and INADL (p3). Also disclosed herein are isolated antibodies that specifically bind to a carboxy-terminal motif in a PL protein of flavivirus. Also disclosed herein are method for the treatment or prophylaxis of a patient having or at risk of flavivirus infection, involving administering to the patient an effective regime of an agent that that inhibits interaction of a PL protein of flavivirus with a PDZ protein of the cell and, in this way, effecting treatment or prophylaxis of the infection. Preferably at least one of the agents is an antibody that specifically binds to the PL motif of the PL protein. In one aspect the agent is an antisense oligonucleotide, a small molecule, an siRNA or a zinc finger protein, and the agent inhibits expression of either the RSV PL protein or a PDZ protein that interacts with it. In some aspects, the PL protein is Capsid C or E1 protein.

Disclosed herein are methods for identifying whether a patient is infected with RSV, by determining whether an RSV PDZ ligand (PL) protein is present in a patient sample, the presence indicating the patient is infected with RSV. In some aspects, the determining involves contacting a patient sample with an agent that specifically binds to the RSV PL protein; and detecting specific binding between the agent and the PL protein, specific binding indicating presence of RSV. A known RSV protein is Nucleoprotein. In some aspects, the agent that specifically binds to the PL protein binds to the PL motif. In other aspects, the PL motif is: DVEL (SEQ ID NO:259) for RSV Nucleoprotein and the agent that specifically binds to the RSV Nucleoprotein PL motif is one of the following PDZ proteins, ZO-1 (p2), RIM2, Novel Serine Protease, MINT1, EBP50 (p1), AIPC (p1), PAR3(p3), SIP1 (p1), PTPL1 (p4), HEMBA 1003117, AF6, AIPC, SYNTENIN, MUPP1 (p3,7,9,1 1), DVL2 (01), ZO-3 (p1), SIP1, AIPC (p1), GORASP1 (p1), INADL (p3), KIAA0316, KIAA1284, MAGI1 (p1), MAST2, MINT1 (p1,2), NSP, NOS1, PAR3 (p3), PAR3L (p3), PAR6 beta, RIM2, Rhodophilin-like, SITAC-18 (p2), SITAC-18 (p1), KIAA1284, PICK1, Shank 1, Shank 2, Shank 3, or TIP1. Also disclosed are isolated antibodies that specifically bind to a carboxy-terminal motifs in a PL protein of RSV. Also disclosed are methods for the treatment or prophylaxis of a patient having or at risk of RSV infection, involving administering to the patient an effective regime of an agent that that inhibits interaction of a PL protein of RSV with a PDZ protein of the cell and thereby effecting treatment or prophylaxis of the infection. In some aspects, the agent is an antibody that specifically binds to the PL motif of the PL protein and can be an antisense oligonucleotide, a small molecule, an siRNA or a zinc finger protein, and the agent inhibits expression of either the rotavirus A PL protein or a PDZ protein that interacts with it. In some aspects, the PL protein is a Nucleoprotein.

Disclosed herein are methods for identifying whether a patient is infected with Rotavirus A, by determining whether a Rotavirus A PDZ ligand (PL) protein is present in a patient sample, presence indicating the patient is infected with Rotavirus A. The determining can include contacting a patient sample with an agent that specifically binds to the Rotavirus A PL protein; and detecting specific binding between the agent and the PL protein, specific binding indicating presence of Rotavirus A. Known Rotavirus A proteins are VP4, VP7, NSP2, and NSP5. In some aspects, the agent that specifically binds to the PL protein binds to the PL motif. In some aspects, the PL motif is one of the following, QCKL (SEQ ID NO:260), or QCRL (SEQ ID NO:261) for Rotavirus A VP4 protein, YYRV (SEQ ID NO:262), or YYRI (SEQ ID NO:263) for Rotavirus A VP7 protein, QVGI (SEQ ID NO:264), HIGI (SEQ ID NO:265), QIGI (SEQ ID NO:266), or RIGI (SEQ ID NO:267) for Rotavirus A NSP2 protein, IKDL (SEQ ID NO:268) or IEDL (SEQ ID NO:269) for Rotavirus A NSP5 protein. In some aspects, the agent that specifically binds to the Rotavirus A VP4 protein PL motif is a PDZ protein such as, MAGI3 (p5), LIM mystique, LIM-RIL, ENIGMA, MAGI1 (p3), MAST2, MAGI2 (p5), LIM protein, and ZO-1 (p2). In some aspects, the agent that specifically binds to the Rotavirus A VP7 protein PL motif is a PDZ protein such as GRIP1 (p6), PTPL1 (p4), MAST1, MUPP1 (p3,7,9), KIAA1719 (p6), MAST2, PICK1, and ZO-1 (p2). In some methods the agent that specifically binds to the Rotavirus A NSP2 PL motif is a PDZ protein selected from the group consisting of: NOS1 (p1,2,3), MINT1 (p2), and ZO-1 (p2). Preferably, the agent that specifically binds to the Rotavirus A NSP5 PL motif is a PDZ protein selected from the group consisting of: NOS1, RIM2, and ZO-1 (p2). Disclosed herein are isolated antibodies that specifically bind to a carboxy-terminal motif in a PL protein of Rotavirus A. Disclosed herein are methods for the treatment or prophylaxis of a patient having or at risk of Rotavirus A infection, by, administering to the patient an effective regime of an agent that that inhibits interaction of a PL protein of Rotavirus A with a PDZ protein of the cell and thereby effecting treatment or prophylaxis of the infection. In some aspects, the agent is an antibody that specifically binds to the PL motif of the PL protein. In some aspects, the agent is an antisense oligonucleotide, a small molecule, an siRNA and a zinc finger protein, wherein said agent inhibits expression of either the bacterial or viral PL protein or a PDZ protein that interacts with it. In some aspects, the PL protein is VP4, VP7, NSP2, or NSP5.

### Definitions

Unless defined otherwise, all technical and scientific terms used herein have the same meaning as commonly understood by those of ordinary skill in the art to which this invention pertains. The following references provide a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); and Hale & Marham, THE HARPER COLLINS DICTIONARY OF BIOLOGY (1991). Although any methods and materials similar or equivalent to those described herein can be used in the practice or testing of the present invention, the preferred methods and materials are described. The following definitions are provided to assist the reader in the practice of the invention.

The term “modulation” as used herein refers to both upregulation, (i.e., activation or stimulation) for example by agonizing, and downregulation (i.e. inhibition or suppression) for example by antagonizing a binding activity. As used herein, the term “PDZ ligand binding modulator” refers to an agent that is able to alter binding of the PDZ-ligand (i.e., “PL”) of a PDZ ligand polypeptide with the PDZ domain of a PDZ domain-containing polypeptide that binds to the PDZ ligand polypeptide. Modulators include, but are not limited to, both activators and inhibitors. An inhibitor can cause partial or complete inhibition of binding.

A “PDZ ligand binding modulator” generally reduces binding between a PDZ ligand polypeptide and a PDZ domain-containing polypeptide by at least 20%, e.g., at least 30%, at least 40%, at least 50%,at least 60%, at least 70%, at least 80%, at least 90%, at least 95%, up to about 99% or 100%, as compared to controls that do not include the test compound. In general, agents of interest are those which exhibit IC₅₀s in a particular assay in the range of about 1 mM or less. Compounds which exhibit lower IC₅₀s, for example, in the range of about 100 μM, 10 μM, 1 μM, 100 nM, 10 nM, 1 nM, or even lower, are particularly useful for as therapeutics or prophylactics to treat or prevent binding mediated disorders.

“Non-natural” is used to mean a composition not occurring in nature. Representative examples of non-natural compositions include substantially purified compositions, as well as, those containing compounds which do not appear in the same chemical form in nature, e.g., chemically and genetically modified proteins, nucleic acids and the like.

“PDZ domain” means an amino acid sequence homologous over about 90 contiguous amino acids; preferably about 80-90; more preferably, about 70-80, more preferably about 50-70 amino acids with the brain synaptic protein PSD-95, the Drosophila septate junction protein Discs-Large (DLG) and/or the epithelial tight junction protein ZO1 (ZO1). Representative examples of PDZ domains are also known in the art as Discs-Large homology repeats (“DHRs”) and “GLGF” repeats (SEQ ID NO:275). Examples of PDZ domains are found in diverse membrane-associated proteins including members of the MAGUK family of guanylate kinase homologs, several protein phosphatases and kinases, neuronal nitric oxide synthase, tumor suppressor proteins, and several dystrophin-associated proteins, collectively known as syntrophins. The instant PDZ domains encompass both natural and non-natural amino acid sequences. Representative examples of PDZ domains include polymorphic variants of PDZ proteins, as well as, chimeric PDZ domains containing portions of two different PDZ proteins and the like. Preferably, the instant PDZ domains contain amino acid sequences which are substantially identical to those disclosed in U.S. patent application Ser. No. 10/485,788 (filed Feb. 3, 2004), International patent application PCT/US03/285/28508 (filed Sep. 9, 2003), International patent application PCT/US01/44138 (filed Nov. 9, 2001), incorporated herein by reference in their entirety. Representative non-natural PDZ domains include those in which the corresponding genetic code for the amino acid sequence has been mutated, e.g., to produce amino acid changes that alter (strengthen or weaken) either binding or specificity of binding to PL. Optionally a PDZ domain or a variant thereof has at least 50, 60, 70, 80 or 90% sequence identity with a PDZ domain from at least one of brain synaptic protein PSD-95, the Drosophila septate junction protein Discs-Large (DLG) and/or the epithelial tight junction protein ZO1 (ZO1), and animal homologs. Optionally a variant of a natural PDZ domain has at least 90% sequence identity with the natural PDZ domain. Sequence identities of PDZ domains are determined over at least 70 amino acids within the PDZ domain, preferably 80 amino acids, and more preferably 80-100 amino acids. Amino acids of analogs are assigned the same numbers as corresponding amino acids in the natural human sequence when the analog and human sequence are maximally aligned. Analogs typically differ from naturally occurring peptides at one, two or a few positions, often by virtue of conservative substitutions. The term “allelic variant” is used to refer to variations between genes of different individuals in the same species and corresponding variations in proteins encoded by the genes.

PDZ domain variants are generally at least 80% identical, at least 90% identical, at least 95% identical or, in certain embodiments at least 98% or at least 99% identical to a wild-type PDZ domain amino acid sequence. In other words, as employed in a method described herein, a PDZ domain-containing polypeptide can contain at least 1, 2, 3, 4, or 5 or more and in certain embodiments up to 10 amino acid substitutions, as compared to a wild-type sequence. A substitution can be conservative (i.e., replacing one amino acid with another within the following groups: gly, ala; val, ile, leu; asp, glu; asn, gin; ser, thr; lys, arg; and phe, tyr), or non-conservative

“PDZ protein”, used interchangeably with “PDZ-domain containing polypeptides” and “PDZ polypeptides”, means a naturally occurring or non-naturally occurring protein having a PDZ domain (supra). Representative examples of PDZ proteins have been disclosed previously (supra) and include CASK, MPP1, DLG1, DLG2, PSD95, NeDLG, TIP-33, TIP-43, LDP, LIM, LIMK1, LIMK2, MPP2, AF6, GORASP1, INADL, KIAA0316, KIAA1284, MAGI1, MAST2, MINTI, NSP, NOS1, PAR3, PAR3L, PAR6 beta, PICK1, Shank 1, Shank 2, Shank 3, SITAC-18, TIP1, and ZO-1. The instant non-natural PDZ domain polypeptides useful in screening assays can contain e.g. a PDZ domain that is smaller than a natural PDZ domain. For example a non-natural PDZ domain may optionally contain a “GLGF” motif, i.e., a motif having the GLGF amino acid sequence (SEQ ID NO:275), which typically resides proximal, e.g. usually within about 10⁻²⁰ amino acids N-terminal, to an PDZ domain. The latter GLGF motif (SEQ ID NO:275), and the 3 amino acids immediately N-terminal to the GLGF motif (SEQ ID NO:275) are often required for PDZ binding activity. Similarly, non-natural PDZ domains may be constructed that lack the β-sheet at the C-terminus of a PDZ domain, i.e., this region may often be deleted from the natural PDZ domain without affecting the binding of a PL.

“PDZ ligand”, abbreviated “PL”, means a naturally occurring protein that has an amino acid sequence which binds to and forms a molecular interaction complex with a PDZ-domain. Representative examples of PL have been provided previously in prior US and International patent applications (supra). PL motifs are provided herein in Table 1.

“PDZ agent” is used to mean a compound that interferes with the binding interaction occurring between a PDZ ligand (PL) polypeptide and a PDZ domain-containing polypeptide in a test assay by at least 20%, e.g., at least 30%, at least 40%, at least 50%,at least 60%, at least 70%, at least 80%, at least 90%, at least 95%, up to about 99% or 100%, as compared to controls that do not include the PDZ agent. While not wishing to be limited to any particular mechanism of action, the instant PDZ agent may interfere e.g. by binding to a PDZ domain that would otherwise bind to a bacterial or viral PL ligand; or alternatively, it may bind directly to the PL ligand to prevent its binding to the PDZ protein. In general, the latter PDZ agents are those which exhibit IC₅₀s in a particular assay in the range of about 1 mM or lower. Compounds which exhibit lower IC₅₀s, for example, commonly have IC₅₀s of about 100 μM, 10 μM, 1 μM, 100 nM, 10 nM, 1 nM, or even lower. The latter PDZ agents are useful in therapeutic and prophylactic medicinal compositions administered to alleviate, treat or prevent one or more symptoms of disease resulting from infection with a virus or bacteria as disclosed herein.

“PL modulator” is used in the context of a PDZ agent (supra) to mean a compound that binds to a viral or bacterial PL protein and modulates its binding to a PDZ domain.

“PDZ modulator” is used in the context of a PDZ agent (supra) to mean a compound that binds to a PDZ domain and modulates the binding of a PL protein at the subject PDZ domain site. The instant PDZ modulators and PL modulators may be peptides, peptidomimetics or small molecule mimetics designed to bind a PDZ domain or PL, respectively. Assays for determining whether a PDZ modulator binds to a PDZ domain are described in great detail in the specification including the A and G assays. Similarly, assays for determining whether a PL modulator binds to a PDZ domain are set forth, e.g., recombinant PDZ domain fusion proteins binding to recombinant PL fusion proteins.

“PDZ-mediated disorder” means one or more symptoms in a viral or bacterial infected subject that result from binding of a viral or bacterial protein PL at a host cell PDZ domain. The symptoms vary depending upon the viral or bacterial disease.

“Sick” when used herein to refer to an animal subject, includes signs and symptoms which may vary from those in the human subject. However, any signs and symptoms may be identified using a physician's desk reference or other reference material. Alternatively, for animals, the “Manual of Diagnostic Tests and Vaccines for Terrestrial Animals, 5th edition, 2004, World Organization for Animal Health” are incorporated herein by reference in their entirety.

By “PDZ ligand polypeptide-inhibitory”, as in the context of a “PDZ ligand polypeptide-inhibitory compound” (e.g., PL protein a PL-inhibitor compound) is meant having an activity that inhibits any activity of a PDZ ligand polypeptide, e.g., a binding activity.

A “PDZ ligand-mediated disorder” is any disorder that may be mediated by an activity of a protein containing a PDZ ligand. Such disorders include the symptoms caused by infection by any of the viruses or bacteria disclosed herein.

In the case of the PDZ domains described herein, a “pathogen PL-binding variant” of a particular PDZ domain is a PDZ domain variant that retains pathogen PL ligand binding activity. Assays for determining whether a PDZ domain variant binds to a pathogen PL are described in great detail below, and guidance for identifying which amino acids to change in a specific PDZ domain to make it into a variant may be found in a variety of sources. In one example, a PDZ domain may be compared to other PDZ domains described herein and amino acids at corresponding positions may be substituted, for example. In another example, the sequence a PDZ domain of a particular PDZ protein may be compared to the sequence of an equivalent PDZ domain in an equivalent PDZ protein from another species. For example, the sequence a PDZ domain from a human PDZ protein may be compared to the sequence of other known and equivalent PDZ domains from other species (e.g., mouse, rat, etc.) and any amino acids that are variant between the two sequences may be substituted into the human PDZ domain to make a variant of the PDZ domain. For example, the sequence of the human DLG1 PDZ domain 1 or 2 may be compared to equivalent DLG1 PDZ domains from other species to identify amino acids that may be substituted into the human DLG PDZ domains to make a variant thereof. Such method may be applied to any of the PDZ domains described herein. Particular variants may have 1, up to 5, up to about 10, up to about 15, up to about 20 or up to about 30 or more, usually up to about 50 amino acid changes as compared to a sequence set forth in the sequence listing.

A PDZ domain polypeptide used in screening assays, for example, may contain a PDZ domain that is smaller than the exemplary PDZ domain set forth in the sequence listing. For example, PDZ domains contain a “GLGF” motif (i.e., a motif having GLGF (SEQ ID NO:275) or a sequence similar thereto) proximal (typically within about 10⁻²⁰ amino acids of) to the N-terminus of the PDZ domain. This motif, and the 3 amino acids immediately N-terminal to the motif are required for PDZ binding activity. Similarly, the β-sheet at the C-terminus of a PDZ domain may be deleted from the PDZ domain without affecting binding.

The term “agent” includes any substance, molecule, element, compound, entity, or a combination thereof. It includes, but is not limited to, e.g., protein, oligopeptide, small organic molecule, polysaccharide, polynucleotide, and the like. It can be a natural product, a synthetic compound, or a chemical compound, or a combination of two or more substances. Unless otherwise specified, the terms “agent”, “substance”, and “compound” can be used interchangeably.

The term “analog” is used herein to refer to a molecule that structurally resembles a molecule of interest but which has been modified in a targeted and controlled manner, by replacing a specific substituent of the reference molecule with an alternate substituent. Compared to the starting molecule, an analog may exhibit the same, similar, or improved utility. Synthesis and screening of analogs, to identify variants of known compounds having improved traits (such as higher binding affinity, or higher selectivity of binding to a target and lower activity levels to non-target molecules) is is well known in pharmaceutical chemistry.

“Contacting” has its normal meaning and refers to combining two or more agents (e.g., two proteins, a polynucleotide and a cell, etc.). Contacting can occur in vitro (e.g., two or more agents, such as a test compound and a cell lysate, are combined in a test tube or other container) or in situ (e.g. two polypeptides can be contacted in a cell by coexpression in the cell, of recombinant polynucleotides encoding the two polypeptides), in the presence or absence of a cell lysate.

A “biopolymer” is a polymer of one or more types of repeating units, regardless of the source. Biopolymers may be found in biological systems and particularly include polypeptides and polynucleotides, as well as such compounds containing amino acids, nucleotides, or analogs thereof. The term “polynucleotide” refers to a polymer of nucleotides, or analogs thereof, of any length, including oligonucleotides that range from 10⁻¹⁰⁰ nucleotides in length and polynucleotides of greater than 100 nucleotides in length. The term “polypeptide” refers to a polymer of amino acids of any length, including peptides that range from 6-50 amino acids in length and polypeptides that are greater than about 50 amino acids in length.

In most embodiments, the terms “polypeptide” and “protein” are used interchangeably. The term “polypeptide” includes polypeptides in which the conventional backbone has been replaced with non-naturally occurring or synthetic backbones, and peptides in which one or more of the conventional amino acids have been replaced with one or more non-naturally occurring or synthetic amino acids. The term “fusion protein” or grammatical equivalents thereof references a protein composed of a plurality of polypeptide components, that while not attached in their native state, are joined by their respective amino and carboxyl termini through a peptide linkage to form a single continuous polypeptide. Fusion proteins may be a combination of two, three or even four or more different proteins. The term polypeptide includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, and the like.

In general, polypeptides may be of any length, e.g., greater than 2 amino acids, greater than 4 amino acids, greater than about 10 amino acids, greater than about 20 amino acids, greater than about 50 amino acids, greater than about 100 amino acids, greater than about 300 amino acids, usually up to about 500 or 1000 or more amino acids. “Peptides” are generally greater than 2 amino acids, greater than 4 amino acids, greater than about 10 amino acids, greater than about 20 amino acids, usually up to about 50 amino acids. In some embodiments, peptides are between 5 and 30 amino acids in length.

The term “capture agent” refers to an agent that binds an analyte through an interaction that is sufficient to permit the agent to bind and concentrate the analyte from a homogeneous mixture of different analytes. The binding interaction may be mediated by an affinity region of the capture agent. Representative capture agents include polypeptides and polynucleotides, for example antibodies, peptides or fragments of single stranded or double stranded DNA may employed. Capture agents usually “specifically bind” one or more analytes.

Accordingly, the term “capture agent” refers to a molecule or a multi-molecular complex which can specifically bind an analyte, e.g., specifically bind an analyte for the capture agent, with a dissociation constant (K_{D}) of less than about 10⁻⁶ M without binding to other targets.

The term “specific binding” refers to the ability of a capture agent to preferentially bind to a particular analyte that is present in a homogeneous mixture of different analytes. In certain embodiments, a specific binding interaction will discriminate between desirable and undesirable analytes in a sample, in some embodiments more than about 10 to 100-fold or more (e.g., more than about 1000- or 10,000-fold). In certain embodiments, the affinity between a capture agent and analyte when they are specifically bound in a capture agent/analyte complex is characterized by a K_{D} (dissociation constant) of less than 10⁻⁶ M, less than 10⁻⁷ M, less than 10⁻⁸ M, less than 10⁻⁹ M, usually less than about 10⁻¹⁰ M.

The term “substantial identity” means that two peptide sequences, when optimally aligned, such as by the programs GAP or BESTFIT using default gap weights, share at least 65 percent sequence identity, preferably at least 80 or 90 percent sequence identity, more preferably at least 95 percent sequence identity or more (e.g., 99 percent sequence identity or higher). Preferably, residue positions which are not identical differ by conservative amino acid substitutions.

“Binding interference”, is used in regard to the first binding interaction of a PDZ domain with a PL to form a complex in a diagnostic assay format; wherein, the subject complex is subsequently detected in a requisite second binding interaction, i.e., interference results when the first binding interaction inhibits the second binding interaction resulting in a decrease in the strength of the signal produced by a signal generating compound. The signal generated by the instant compositions in the methods of the invention are subject to less than 15% binding interference; preferably, less than 10%; and, most preferably less than about 5%.

The term “capture agent/analyte complex” is a complex that results from the specific binding of a capture agent with an analyte, i.e., a “binding partner pair”. A capture agent and an analyte for the capture agent specifically bind to each other under “conditions suitable for specific binding”, where such conditions are those conditions (in terms of salt concentration, pH, detergent, protein concentration, temperature, etc.) which allow for binding to occur between capture agents and analytes to bind in solution. Such conditions, particularly with respect to proteins, are well known in the art (see, e.g., Harlow and Lane (Antibodies: A Laboratory Manual Cold Spring Harbor Laboratory, Cold Spring Harbor, N.Y. (1989)). Conditions suitable for specific binding typically permit capture agents and target pairs that have a dissociation constant (K_{D}) of less than about 10⁻⁶ M to bind to each other, but not with other capture agents or targets.

“Binding partners” and equivalents refer to pairs of molecules that can be found in a capture agent/analyte complex, i.e., exhibit specific binding with each other.

The phrase “surface-bound capture agent” refers to a capture agent that is immobilized on a surface of a solid substrate, where the substrate can have a variety of configurations, e.g., a sheet, bead, or other structure, such as a plate with wells. In certain embodiments, the collections of capture agents employed herein are present on a surface of the same support, e.g., in the form of an array.

“Isolated” or “purified” generally refers to isolation of a substance (compound, polynucleotide, protein, polypeptide, polypeptide composition) such that the substance comprises a significant percent (e.g., greater than 2%, greater than 5%, greater than 10%, greater than 20%, greater than 50%, or more, usually up to about 90%-100%) of the sample in which it resides. In certain embodiments, a substantially purified component comprises at least 50%, 80%-85%, or 90-95% of the sample. Techniques for purifying polynucleotides and polypeptides of interest are well-known in the art and include, for example, ion-exchange chromatography, affinity chromatography and sedimentation according to density. Generally, a substance is purified when it exists in a sample in an amount, relative to other components of the sample, that is not found naturally.

The term “fusion protein” or grammatical equivalents thereof is meant a protein composed of a plurality of polypeptide components, that while typically unjoined in their native state, typically are joined by their respective amino and carboxyl termini through a peptide linkage to form a single continuous polypeptide. Fusion proteins may be a combination of two, three or even four or more different proteins. The term polypeptide includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like.

The term “assessing” includes any form of measurement, and includes determining if an element is present or not. The terms “determining”, “measuring”, “evaluating”, “assessing” and “assaying” are used interchangeably and may include quantitative and/or qualitative determinations. Assessing may be relative or absolute. “Assessing binding” includes determining the amount of binding, and/or determining whether binding has occurred (i.e., whether binding is present or absent).

The terms “treatment”, “treating”, “treat”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease and/or relieving one or more disease symptoms. “Treatment” is also meant to encompass delivery of an agent in order to provide for a pharmacologic effect, even in the absence of a disease or condition. For example, “treatment” encompasses delivery of modulator that can provide for enhanced or desirable effects in the subject (e.g., one or more symptoms that are decreased in severity, etc.).

“Subject”, “individual,” “host” and “patient” are used interchangeably herein, to refer to an animal, human or non-human, susceptible to or having a pathogen infection amenable to therapy according to the methods of the invention. Generally, the subject is a mammalian subject. Exemplary subjects include, but are not necessarily limited to, humans, non-human primates, mice, rats, cattle, chickens, ducks, birds, sheep, goats, pigs, dogs, cats, and horses, with humans being of particular interest.

“Signal generating compound”, abbreviated “SGC”, means a molecule that can be linked to a PL or a PDZ (e.g. using a chemical linking method as disclosed further below and is capable of reacting to form a chemical or physical entity (i.e., a reaction product) detectable in an assay according to the instant disclosure. Representative examples of reaction products include precipitates, fluorescent signals, compounds having a color, and the like. Representative SGC include e.g., bioluminescent compounds (e.g., luciferase), fluorophores (e.g., below), bioluminescent and chemiluminescent compounds, radioisotopes (e.g., ¹³¹I, ¹²⁵I, ¹⁴C, ³H, ³⁵S, ³²P and the like), enzymes (e.g., below), binding proteins (e.g., biotin, avidin, streptavidin and the like), magnetic particles, chemically reactive compounds (e.g., colored stains), labeled-oligonucleotides; molecular probes (e.g., CY3, Research Organics, Inc.), and the like. Representative fluorophores include fluorescein isothiocyanate, succinyl fluorescein, rhodamine B, lissamine, 9,10-diphenlyanthracene, perylene, rubrene, pyrene and fluorescent derivatives thereof such as isocyanate, isothiocyanate, acid chloride or sulfonyl chloride, umbelliferone, rare earth chelates of lanthanides such as Europium (Eu) and the like. Representative SGC's useful in a signal generating conjugate include the enzymes in: IUB Class 1, especially 1.1.1 and 1.6 (e.g., alcohol dehydrogenase, glycerol dehydrogenase, lactate dehydrogenase, malate dehydrogenase, glucose-6-phosphate dehydrogenase, glyceraldehyde-3-phosphate dehydrogenase and the like); IUB Class 1.11.1 (e.g., catalase, peroxidase, amino acid oxidase, galactose oxidase, glucose oxidase, ascorbate oxidase, diaphorase, urease and the like); IUB Class 2, especially 2.7 and 2.7.1 (e.g., hexokinase and the like); IUB Class 3, especially 3.2.1 and 3.1.3 (e.g., alpha amylase, cellulase, β-galacturonidase, amyloglucosidase, β-glucuronidase, alkaline phosphatase, acid phosphatase and the like); IUB Class 4 (e.g., lyases); IUB Class 5 especially 5.3 and 5.4 (e.g., phosphoglucose isomerase, trios phosphatase isomerase, phosphoglucose mutase and the like.) Signal generating compounds also include SGC whose products are detectable by fluorescent and chemilluminescent wavelengths, e.g., luciferase, fluorescence emitting metals such as ¹⁵²Eu, or others of the lanthanide series; compounds such as luminol, isoluminol, acridinium salts, and the like; bioluminescent compounds such as luciferin; fluorescent proteins; and the like. Fluorescent proteins include, but are not limited to the following: namely, (i) green fluorescent protein (GFP), i.e., including, but not limited to, a “humanized” versions of GFP wherein codons of the naturally-occurring nucleotide sequence are exchanged to more closely match human codon bias; (ii) GFP derived from Aequoria victoria and derivatives thereof, e.g., a “humanized” derivative such as Enhanced GFP, which are available commercially, e.g., from Clontech, Inc.; (iii) GFP from other species such as Renilla reniformis, Renilla mullei, or Ptilosarcus guernyi, as described in, e.g., WO 99/49019 and Peelle et al. (2001) J. Protein Chem. 20:507-519; (iv) “humanized” recombinant GFP (hrGFP) (Stratagene); and, (v) other fluorescent and colored proteins from Anthozoan species, such as those described in Matz et al. (1999) Nature Biotechnol. 17:969-973; and the like. The subject signal generating compounds may be coupled to a PL or PDZ domain polypeptide. Attaching certain SGC to proteins can be accomplished through metal chelating groups such as EDTA. The subject SGC share the common property of allowing detection and/or quantification of a viral or bacterial PL analyte in a test sample. The subject SGC are detectable using a visual method; preferably, an a method amenable to automation such as a spectrophotometric method, a fluorescence method, a chemilluminescent method, a electrical nanometric method involving e.g., a change in conductance, impedance, resistance and the like and a magnetic field method.

“Solid phase” means a surface to which one or more reactants may be attached electrostatically, hydrophobically, or covalently. Representative solid phases include e.g.: nylon 6; nylon 66; polystyrene; latex beads; magnetic beads; glass beads; polyethylene; polypropylene; polybutylene; butadiene-styrene copolymers; silastic rubber; polyesters; polyamides; cellulose and derivatives; acrylates; methacrylates; polyvinyl; vinyl chloride; polyvinyl chloride; polyvinyl fluoride; copolymers of polystyrene; silica gel; silica wafers glass; agarose; dextrans; liposomes; insoluble protein metals; and, nitrocellulose. Representative solid phases include those formed as beads, tubes, strips, disks, filter papers, plates and the like. Filters may serve to capture analyte e.g. as a filtrate, or act by entrapment, or act by covalently-binding PL or PDZ onto the filter (e.g., see the Examples section below). According to certain embodiments of the invention, a solid phase capture reagent for distribution to a user may consist of a solid phase (supra) coated with a “capture reagent” (below), and packaged (e.g., under a nitrogen atmosphere) to preserve and/or maximize binding of the capture reagent to a viral or bacterial PL analyte in a biological sample.

“Capture reagent” means an immobilized PDZ polypeptide (or peptide) capable of binding a viral or bacterial PL. The subject capture reagent may consist of a solution of a PDZ; or a PDZ modified so as to promote its binding to a solid phase; or a PDZ already immobilized onto the surface of a solid phase, e.g., immobilized by attaching the PDZ to a solid phase (supra) through electrostatic forces, van Der Waals forces, hydrophobic forces, covalent chemical bonds, and the like (as disclosed further below.) Representative examples of PDZ capture reagents are disclosed in the Examples section, below, and include mobile solid phase PDZ capture reagents such as PDZ immobilized on movable latex beads e.g. in a latex bead dipstick assay.

“Detect reagent” means a conjugate containing an SGC linked to a PL or PDZ polypeptide or peptide; or alternatively, an SGC linked to an antibody capable of binding specifically to a PL or a PDZ. Representative examples of the instant detect reagents include complexes of one or more PL or PDZ with one or more SGC compounds, i.e., macromolecular complexes. The subject detect reagents include mobile solid-phase detect reagents such as movable latex beads in latex bead dipstick assays.

“Biological sample” means a sample obtained from a living (or dead) organism, e.g., a mammal, fish, bird, reptile, marsupial and the like. Biological samples include tissue fluids, tissue sections, biological materials carried in the air or in water and collected there from e.g. by filtration, centrifugation and the like, e.g., for assessing bioterror threats and the like. Alternative biological samples can be taken from fetus or egg, egg yolk, and amniotic fluids. Representative biological fluids include, e.g. urine, blood, plasma, serum, cerebrospinal fluid, semen, lung lavage fluid, feces, sputum, mucus, water carrying biological materials and the like. Alternatively, biological samples include nasopharyngeal or oropharyngeal swabs, nasal lavage fluid, tissue from trachea, lungs, air sacs, intestine, spleen, kidney, brain, liver and heart, sputum, mucus, water carrying biological materials, cloacal swabs, sputum, nasal and oral mucus, and the like. Representative biological samples also include foodstuffs, e.g., samples of meats, processed foods, poultry, swine and the like. Biological samples also include contaminated solutions (e.g., food processing solutions and the like), swab samples from out-patient sites, hospitals, clinics, food preparation facilities (e.g., restaurants, slaughter-houses, cold storage facilities, supermarket packaging and the like). Biological samples may also include in-situ tissues and bodily fluids (i.e., samples not collected for testing), e.g., the instant methods may be useful in detecting the presence or severity or viral infection in the eye e.g., using eye drops, test strips applied directly to the conjunctiva; or, the presence or extent of lung infection by e.g. placing an indicator capsule in the mouth or nasopharynx of the test subject. Alternatively, a swab or test strip can be placed in the mouth. The biological sample may be derived from any tissue, organ or group of cells of the subject. In some embodiments a scrape, biopsy, or lavage is obtained from a subject. Biological samples may include bodily fluids such as blood, urine, sputum, and oral fluid; and samples such as nasal washes, swabs or aspirates, tracheal aspirates, chancre swabs, and stool samples. Methods are for the collection of biological specimens suitable for the detection of individual pathogens of interest, for example, nasopharyngeal specimens such as nasal swabs, washes or aspirates, or tracheal aspirates in the case of respiratory disease, oral swabs and the like. Thus, embodiments of the invention provide methods useful in testing a variety of different types of biological samples for the presence or amount of a viral or bacterial contamination or infection. Optionally, the biological sample may be suspended in an isotonic solution containing antibiotics such as penicillin, streptomycin, gentamycin, and mycostatin.

“Ligand” refers to a PL compound capable of binding to an PDZ binding site. Representative examples of ligands include PL-containing viral proteins and PL-containing bacterial proteins as disclosed herein. The subject ligand is capable of filling a three-dimensional space in binding site of a PDZ domain binding site so that electrostatic repulsive forces are minimized, electrostatic attractive forces are maximized, and hydrophobic and hydrogen bonding forces are maximized. Ligands bind to PDZ polypeptides in a specific and saturable manner, and binding affinities may be measured according to ligand binding assays disclosed further below.

“Specificity”, when used in the context of an assay according to an embodiment of the invention, means that the subject assay, as performed according to the steps of the invention, is capable of properly identifying an “indicated” percentage of samples from within a panel of biological samples (e.g., a panel of 100 samples). The subject panel of samples all contain one or more murein analytes (e.g., positive control samples contaminated with bacteria or fungi.) Preferably the subject “indicated” specificity is greater than 85%, (e.g., the assay is capable of indicating that more than 85 of the 100 samples contain one or more murein analyte), and most preferably, the subject assay has an indicated specificity that is greater than 90%. Optionally, the subject assay is capable of identifying “true non-viral or bacterial cases”, i.e., detecting an “indicated” percentage of negative samples from within a panel of biological samples (e.g., a panel of 100 samples). Preferably, the instant steps of the invention are capable of properly identifying “true viral or bacterial cases”; and preferably, the instant steps of the invention are capable of properly identifying “true low-pathogenic viral and bacterial cases”. In different embodiments, the subject negative control panel of samples either do not contain viral or bacterial PL analytes; or, contain other viral or bacterial PL analytes. Preferably the subject specificity is greater than 85%, (e.g., the assay is capable of indicating that more than 85 of the 100 samples and most preferably, the subject assay has specificity that is greater than 90%.

“Sensitivity”, when used in the context of an assay according to an embodiment of the invention, means that the subject assay, as performed according to the steps of the invention, is capable of identifying at an “indicated” percentage those samples which contain a viral or bacterial PL analyte from within a panel of samples containing both positive controls (supra) and negative controls (i.e., lacking PL analyte.) Preferably the subject “indicated” sensitivity is greater than 85% and most preferably greater than 90%. Optionally, the subject assay is capable of identifying “true viral or bacterial cases” at an “indicated” percentage of those samples which contain a viral or bacterial PL analyte from within a panel of samples. Preferably, the instant steps of the invention are capable of properly identifying “true viral or bacterial cases”; and, most preferably, the instant steps of the invention are capable of properly identifying “true pathogenic viral or bacterial cases”. In different embodiments, the subject positive control panel of samples either contain viral or bacterial PL analytes; or contain highly pathogenic viral or bacterial PL proteins. Preferably the subject “indicated” sensitivity is greater than about 70% and more preferably greater than about 80%. Even more preferably, the sensitivity is greater than about 85% and most preferably greater than about 90% of that of the control. Alternatively, the sensitivity can be measured with respect to the sensitivity of a PCR reaction that identifies the same protein

Nucleic acid and protein sequences that have been previously determined and electronically deposited into NCBI's Genbank database are referenced herein by Genbank accession number (GI). The sequences set forth in those Genbank entries are incorporated by reference herein in their entirety for all purposes. The Applicants expressly reserve the right to later amend the specification to specifically recite one or more of these sequences, or any indicated portion thereof.

Various biochemical and molecular biology methods referred to herein are described in, for example, Sambrook et al., Molecular Cloning: A Laboratory Manual, Cold Spring Harbor Press, N.Y. Second (1989) and Third (2000) Editions, and Current Protocols in Molecular Biology, (Ausubel, F. M. et al., eds.) John Wiley & Sons, Inc., New York (1987-1999).

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

Reference to a singular item includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

### BRIEF DESCRIPTION OF THE DRAWINGS

FIGS. 1A-D show a PDZ protein binding analysis of Peptide 1904. FIG. 1A shows the GST background for the G Assay, FIG. 1B shows a titration curve for Peptide 1904 binding to ZO-1 d2, FIG. 1C shows shows a titration curve for Peptide 1904 binding to Rim 2, FIG. 1D shows shows a titration curve for Peptide 1904 binding to NSP.

FIGS. 2A-D shows a second experiment to identify PDZ protein binding to Peptide 1904. FIG. 2A shows the GST background for the G Assay, FIG. 2B shows a titration curve for Peptide 1904 binding to ZO-1 d2, FIG. 2C shows shows a titration curve for Peptide 1904 binding to Rim 2, FIG. 2D shows shows a titration curve for Peptide 1904 binding to NSP.

FIGS. 3A-D show a PDZ protein binding analysis of Peptide 1905. FIG. 3A shows the GST background for the G Assay, FIG. 3B shows a titration curve for Peptide 1904 binding to ZO-1 d2, FIG. 3C shows shows a titration curve for Peptide 1904 binding to Rim 2, FIG. 3D shows shows a titration curve for Peptide 1904 binding to NSP.

FIGS. 4A-D show a second experiment to identify PDZ protein binding to Peptide 1905. FIG. 4A shows the GST background for the G Assay, FIG. 4B shows a titration curve for Peptide 1904 binding to ZO-1 d2, FIG. 4C shows shows a titration curve for Peptide 1904 binding to Rim 2, FIG. 4D shows shows a titration curve for Peptide 1904 binding to NSP.

FIGS. 5A-D show a third experiment to identify PDZ protein binding to Peptide 1905. FIG. 5A shows the GST background for the G Assay, FIG. 5B shows a titration curve for Peptide 1904 binding to ZO-1 d2, FIG. 5C shows shows a titration curve for Peptide 1904 binding to Rim 2, FIG. 5D shows shows a titration curve for Peptide 1904 binding to NSP.

### DETAILED DESCRIPTION OF THE INVENTION

The invention provides methods for determining if a subject is infected with a virus or bacteria. The invention is based in part on the discovery that a wide variety of pathogens, including many pathogenic viruses and bacteria, encode proteins that contain PDZ ligands (i.e., ligands to which PDZ domain-containing polypeptides specifically bind). As such, those pathogen can be detected in a sample using PDZ domains. An exemplary method involves contacting a sample from the subject with a PDZ-containing polypeptide and determining whether the PDZ polypeptide binds to a viral or bacterial PDZ ligand polypeptide. Binding between the PDZ-containing polypeptide and the viral PDZ ligand polypeptide indicates that the subject is infected with the virus or bacteria.

The use of PDZ-PL interactions for diagnostic purposes is amenable to a number of different test formats. Diagnostic tests could be formatted for ELISA assays, as a dipstick test such as is used for pregnancy tests, as a film test that can be incubated with test sample, as a slide test that sample could be placed upon, or other such media. Such formats are described in e.g. U.S. Pat. Nos. 6,180,417, 4,703,017 and 5,591,645. Assays for identifying anti-viral and anti-bacterial agents are also provided. The invention finds use in a variety of diagnostic and therapeutic applications.

I. Viral and Bacterial Pathogens

Human immunodeficiency virus (HIV) is the causative agent of AIDS (acquired immunodeficiency disease syndrome) and is found in all cases of the disease. Its primary target is the activated CD4+T4 helper lymphocyte but it can also infect several other cell types including macrophages. HIV is a lentivirus, a class of retrovirus. The name lentivirus means slow virus, so named because these viruses take a long time to cause overt disease. Most lentiviruses target cells of the immune system and thus disease is often characterized as immunodeficiency. There are five known serogroups of lentivirus that infect primates, sheep and goats, horses, cats, and cattle. There are two types of HIV, HIV-1 and HIV-2. These cause clinically indistinguishable disease although the time to disease onset is longer for HIV-2. The worldwide epidemic of HIV and AIDS is caused by HIV-1 HIV-2 is mostly restricted to west Africa. In the infected patient, HIV has previously been detected by the presence of anti-HIV antibodies or by the presence of the virus itself using polymerase chain reaction (PCR) that detects viral RNA. PCR is very sensitive and can show HIV in situations in which it is not detectable immunologically. Since the late 1970's, HIV and AIDS have spread across the United States and around the world. In sub-Saharan Africa, more than 25 million people are living with HIV infection and three million people around the world die of AIDS each year. Human immunodeficiency viruses, HIV-1 and HIV-2, are similar, by genome structure, nucleotide sequences and pathogenicity, to retroviruses existing in various primates, SIVs.

The mature HIV virus consists of a bar-shaped electron dense core containing the viral genome—two short strands of ribonucleic acid (RNA) along with the enzymes reverse transcriptase, protease, ribonuclease, and integrase, all encased in an outer lipid envelope with 72 surface projections containing an antigen, gp 120, that aids in the binding of the virus to the target cells with CD4 receptors. The genome of HIV, similar to retroviruses in general, contains three major genes—gag, pol, and env. The major structural components coded by env include the envelope glycoproteins, including the outer envelope glycoprotein gp120 and transmembrane glycoprotein gp41 derived from glycoprotein precursor gp160. Major components coded by the gag gene include core nucleocapsid proteins p55, p40, p24 (capsid, or “core” antigen), p17 (matrix), and p7 (nucleocapsid); the important proteins coded by pol are the enzyme proteins p66 and p51 (reverse transcriptase), p11 (protease), and p32 (integrase). Although most of the major HIV viral proteins, which include p24 (core antigen) and gp41 (envelope antigen), are highly immunogenic, the antibody responses vary according to the virus load and the immune competence of the host. The antigenicity of these various components provides a means for detection of antibody, the basis for most HIV testing.

HIV has the additional ability to mutate easily, in large part due to the error rate of the reverse transcriptase enzyme, which introduces a mutation approximately once per 2000 incorporated nucleotides. This high mutation rate leads to the emergence of HIV variants within the infected person's cells that can resist immune attack, are more cytotoxic, can generate syncytia more readily, or can resist drug therapy. Over time, different tissues of the body may harbor differing HIV variants.

The genetic sequences of HIV-1 and HIV-2 are only partially homologous. HIV-2, or other as yet uncharacterized members of the HIV-group of viruses, will not necessarily be detected by using the various laboratory tests for HIV-1 antibody. HIV-2 is genetically more closely related to simian immunodeficiency virus (SIV) than HIV-1.

Hepatitis B causes both acute and chronic hepatitis in some patients who are unable to eliminate the virus. Identified methods of transmission include blood , blood transfusion, now rare), tatoos (both amateur and professionally done), horizontally (sexually or through contact with blood or bodily fluids), or vertically (from mother to her unborn child). However, in about half of cases the source of infection cannot be determined. Hepatitis B is a Hepadnavirus and is partially double and single-stranded DNA genome enclosed in a icosahedral nucleocapsid (core antigen) of 27 nm in diameter which is surrounded by a glycoprotein envelope of 42 nm in diameter (surface antigen). The virus therefore, possesses two shells. Core antigen in seen in the liver but not in the blood. Anti core antibody is detected in the blood. The surface antigen is produced by the virus in large quantities and shedded in the blood stream as spherules and rods of 22 nm diameter entirely composed of glycoproteins. Among them, under electron microscopy are larger spherule of 42 nm diameter which are the intact virions, the Dane particles. HE protein is present in the blood in carriers and correlates with viremia.

Hepatitis C (originally “non-A non-B hepatitis”) can be transmitted through contact with blood (including through sexual contact). It may lead to a chronic form of hepatitis, culminating in cirrhosis. It can remain asymptomatic for 10⁻²⁰ years. No vaccine is available for hepatitis C. Patients with hepatitis C are prone to severe hepatitis if they contract either hepatitis A or B, so all hepatitis C patients should be immunized against hepatitis A and hepatitis B if they are not already immune. However, hepatitis C itself is a very lethal virus and can cause cirrhosis of the liver. The virus, if detected early on can be treated by a combination of interferon and the antiviral drug ribavirin. There are variations in the response to this treatment regimen based on the genotype of the infecting virus. The virus contains a single-stranded genome of RNA with approximately 10,000 nucleotides, a capsid, a matrix and an envelope. It encodes a single polyprotein precursor which is fragmented in 3 structural (C, E1, E2) and in 4-6 non structural proteins (NS1, NS2, NS3, NS4, NS5) forming the following antigens: c100, c22, c33, c300, 5-1-1 Other components are proteases, RNA polymerase and transcriptases, not reverse transcriptase. The structure of the HCV is not known because the virus has not been seen yet with the electron microscope due to the very scarce concentration of viral particles in the blood and tissues. Probably only 1-10 virions per ml are present in the blood. HCV appears to be similar to flaviviruses which produce only acute illnesses especially in animals, the prototype being yellow fever virus. HCV does not integrate into host DNA like Hepatitis B virus. HCV has a very high mutation rate producing many similar species. This variation accounts for resistance to antibodies.

RSV is a member of the paramyxovirus family that includes measles, parainfluenza and mumps viruses. Respiratory syncytial virus (RSV) is a negative-strand RNA virus that causes lower respiratory tract infections primarily in children. No vaccine or effective therapeutic is currently available to treat the viral infection. Several proteins are responsible for transcribing and replicating the viral RNA. The RSV-encoded phosphoprotein is required for viral replication. The phosphoprotein forms an oligomer and is constitutively phosphorylated. However, the functional significance of these events has not yet been determined. RSV does not have an HN (Hemagglutinin Neuraminidase) protein like the other members of the paramyxovirus family, but instead has a G protein. The matrix protein M lines the inner surface of the envelope. the F protein is involved in cell fusion.

Rotavirus A Rotavirus is a non-enveloped virus of the family Reoviridae with an icosahedral capsid 70 nm across. It derives its name from the wheel like appearance it has when viewed under an electron microscope (rota is latin for wheel). Its genome is made up of 11 segments of double stranded RNA held in the inner core of the three-layered virus. The genome codes for 6 virus proteins (VP 1,2,3,4,6,7) and 6 non-structural proteins (NSP1-6). Once in the small intestine, the virus undergoes a change and becomes infective to the villi. Proteins then mediate the invasion of the host cells and replication of the virus genome.

M. tuberculosis is the causative agent for tuberculosis and a member of the genus mycobacteria. Other members include M. leprae, the causative agent of leprosy. The mycobacteria tend to be very slow growing and M. tuberculosis is an obligate aerobe. Tuberculosis (TB) is the number one infectious disease killer worldwide. The World Health Organization estimates that 2 billion people have latent TB, while another 3 million people worldwide die each year due to TB. TB is treated with a number of antibiotics, but is rapidly becoming resistant to them. On average, the isoniazid (INH) resistance rate is approximately 10% and the rifampin resistance rate is approximately 1%, with lower numbers in countries with good TB programs and higher numbers in countries with poor TB programs. Humans are the only known reservoir for Mycobacterium tuberculosis. TB is transmitted by airborne droplet nuclei, which may contain fewer than 10 bacilli. Exposure to TB occurs by sharing common airspace with a patient who is infectious. When inhaled, droplet nuclei are deposited within the terminal airspaces of the lung. Upon encountering the bacilli, macrophages ingest and transport the bacteria to regional lymph nodes.

II. Viral and Bacterial PL Regions

The present inventors discovered PL proteins for viruses and bacteria by identification of one or more PL motifs and showing binding to PDZ proteins. These proteins have a variety of PL motifs. The following PL proteins were identified for the designated viruses and bacteria, the PL motifs are provided in Table 1. The PDZ binding partners that were identified by the inventors are provided in Tables 1 and 2.

A description of the activity of some of the viral and bacterial proteins follows. Related viruses and bacteria, for example, viruses from the same family and bacteria from the same genus are likely to have the same PL proteins and even the same PL motifs. However, as can be seen in Table 1A and B, the motifs identified for HIV-1 and HIV-2 Env proteins . differed. This difference can be used to differentiate between HIV-1 or HIV-2 in a diagnostic assay.

The present inventors have discovered that the HIV protein Nef is a PL protein having the motifs, FKNC (SEQ ID NO:244), FKDC (SEQ ID NO:245), YKNC (SEQ ID NO:246), and YKDC (SEQ ID NO:247) and have identified a number of PDZ protein binding partners. Although HIV-1 Nef was originally named “negative factor,” it has been shown to have a positive role in viral replication and pathogenesis. Nef is a viral protein that interacts with host cell signal transduction proteins to provide for long term survival of infected T cells and for destruction of non-infected T cells (by inducing apoptosis). Nef also advances the endocytosis and degradation of cell surface proteins, including CD4 and MHC proteins (CD4 is an integral membrane protein that functions in T-cell activation, and is the receptor for the HIV virus). This action possibly impairs cytotoxic T cell function, thereby helping the virus to evade the host immune response (e.g., Schwartz, et al., 1996). The multifunctional protein thus helps the virus maintain high viral loads and overcome host immune defenses, contributing to the progression of AIDS. Not surprisingly, persons infected with HIV-1 strains that have deletions of the Nef gene develop AIDS symptoms much more slowly than those infected with standard HIV strains. Nef may therefore be a valuable target for pharmaceutical intevention in AIDS progression. Nef is a 27 kD, N-terminal myristoylated accessory protein involved in post integration infection. Nef is found in the viral particle and is one of the first proteins to be detected after invasion of the host cell.

The present inventors have discovered that the HIV protein Vif is PL protein having the motifs, EILA (SEQ ID NO:25 1), GILA (SEQ ID NO:252), and DILA (SEQ ID NO:253) and have identified a number of PDZ binding partners. The human immunodeficiency virus type 1 (HIV-1) Vif protein is essential for virus replication in primary lymphoid and myeloid cells, but is dispensable for efficient replication in several transformed T-cell lines as well as in nonlymphoid cell lines such as HeLa and 293T. Cells that are unable to support the replication of Vif-defective HIV-1 (HIV-1ΔVif) have been termed ‘nonpermissive’, whereas cells that can sustain HIV-1ΔVif replication are termed ‘permissive’. The observation that heterokaryons formed by fusion of nonpermissive and permissive cells exhibit the nonpermissive phenotype led to the hypothesis that nonpermissive cells express an inhibitor of HIV-1 replication, lacking in permissive cells, that is blocked by the viral Vif protein. The nucleocapsid (NC) protein of HIV-2 (NCp8) contains two Cys-His arrays which function as zinc finger motifs (ZFMs).

The present inventors have discovered that Env is a PL protein in both HIV viruses. The HIV-2 protein Env was found to have the motifs, LALL (SEQ ID NO:248), LALL (SEQ ID NO:249), and LTLL (SEQ ID NO:250). HIV-1 Env had the following PL motifs, RALL (SEQ ID NO:242), and RILL (SEQ ID NO:243). It is likely that in related viruses of the lentivirus and even retrovirus families the viral proteins Nef, Vif and Env have the same function and related PL motifs.

The present inventors identified that the RSV nucleoprotein NP is a PL protein having the PL motif DVEL (SEQ ID NO:259) and identified a number of PDZ binding parters. RSV NP can be found in close relation to the RNA genome. The RSV protein NP was found herein to be a PL protein having the motif, DVEL (SEQ ID NO:259).

The present inventors discovered that Rotavirus VP4 and VP7 are PL proteins and have identified a number of PL motifs associated with them, including QCKL (SEQ ID NO:260) and QCRL (SEQ ID NO:261) for VP4 and YYRV (SEQ ID NO:262) and YYRI (SEQ ID NO:263) for VP7. VP4 and VP 7 make up the outer capsid of the Rotavirus. VP4 is an 88 kDa protein that dimerizes to create 60 spikes on virus surface. VP4 is cleaved by the pancreatic enzyme trypsin to form VP 5 and VP 8. VP4 and its cleavage products are associated with cell attachment and invasion and cleavage is necessary for infectivity. VP4 is antigenic and induces neutralizing antibodies. The specific structure of this protein is used to determine the rotavirus P serotype, as well as host specificity, virulence and protective immunity. It has also been associated with heat shock cognate protein, hsc70 during cell entry. This 37 kD glycoprtein makes up the smooth portion of the outer capsid. It can induce neutralizing antibodies and determines the G serotype. It is also a highly variable portion of the virus capable of reassortment and possible crossover with animal strains of the virus. VP7 also has associations with heat shock cognate protein (hsc 70), and some integrins, both related to viral entry of the cell.

Other Rotavirus protein PLs that were identified are NSP2 and NSP5, having the PL motifs QVGI (SEQ ID NO:264), HIGI (SEQ ID NO:265), QIGI (SEQ ID NO:266), and RIGI (SEQ ID NO:267) for NSP2 and IKDL (SEQ ID NO:268) and IEDL (SEQ ID NO:269) for NSP5. In conjunction with NSP5, NSP2 is involved in the synthesis and packaging of viral RNA and creation of viroplasms. NSP2 is a replication intermediate. The NSP5 phosphoprotein works with NSP2 in RNA synthesis and packaging, and to induce viroplasms. It is also a replication intermediate.

The inventors identified three PL proteins for Mycobacterium tuberculosis having the PL motifs SSWA (SEQ ID NO:270) for ESXN, YTGF (SEQ ID NO:271) for ESXS, and GMFA (SEQ ID NO:272) for ESAT-6. The 6-kDa early secretory antigenic target (ESAT-6) and the 10-kDa culture filtrate protein (CFP-10) from Mycobacterium tuberculosis are two dominant targets for T cells in the early phases of infection. Furthermore, ESAT-6 has recently been demonstrated to induce protective immunity when administered as either a subunitor a DNA vaccine. The genes encoding ESAT-6 and CFP-10 (esx and lhp, respectively) lie next to each other in an operon-like structure. A dual knockout of ESAT-6 and CFP-10 in M. bovis results in decreased virulence of the pathogen, indicating that the two molecules may play important roles in immunopathogenesis and virulence. ESXN and ESXS are two members of the ESAT-6 family.

Hepatitis B was found by the inventors to contain two PL proteins, Protein X was FTSA (SEQ ID NO:254) and S antigen had the PL motif WVYI (SEQ ID NO:255). Hepatitis B is a member of the hepadnavirus family.

Hepatitis C was found by the inventors to contain two PL proteins, Capsid C had a number of PL motifs, including PASA (SEQ ID NO:256) and PVSA (SEQ ID NO:257), and Ela had the PL motif GVDA (SEQ ID NO:258). HCV is designated as a flavivirus with other members of the familing including, yellow fever virus, dengue, Japanese encephalitis virus, tick-borne encephalitis, and West Nile virus.

III. PDZ Proteins

PDZ domains have recently emerged as central organizers of protein complexes at the plasma membrane. PDZ domains were originally identified as conserved sequence elements within the postsynaptic density protein PSD95/SAP90, the Drosophila tumor suppressor dlg-A, and the tight junction protein ZO-1. Although originally referred to as GLGF (SEQ ID NO:275) or DHR motifs, they are now known by an acronym representing these first three PDZ-containing proteins (PDZ: PSD95/DLG/ZO-1). These 80-90 amino acids sequences have now been identified in well over 75 proteins and are characteristically expressed in multiple copies within a single protein. PDZ domains are recognized as families by the National Center for Biotechnology Information (www.ncbi.gov) for example in Pfam. They are also found throughout phylogeny in organisms as diverse as metazoans, plants, and bacteria. Such a broad species distribution appears to be unique to this domain, but perhaps the most distinguishing feature of PDZ domains is the observation that the overwhelming majority of proteins containing them are associated with the plasma membrane. Although PDZ domains are found in many different structures, each PDZ protein is generally restricted to specific subcellular domains, such as synapses; cell-cell contacts; or the apical, basal, or lateral cell surface. This leads to the speculation that PDZ domains evolved early to provide a central role in the organization of plasma membrane domains. The most general function of PDZ domains may be to localize their ligands to the appropriate plasma membrane domain. In polarized epithelial cells, PDZ proteins clearly localize at distinct apical, basal-lateral, and junctional membrane domains and, in most cases, colocalize with their transmembrane and cytosolic binding partners. PDZ proteins also clearly have a fundamental role spatially clustering and anchoring transmembrane proteins within specific subcellular domains.

The present inventors have identified PDZ proteins that are binding partners for the virus and bacterial PL's found in Table 1, including, AF6, AIPC, AIPC (PDZ #1), GORASPI (PDZ #1), INADL (PDZ #3), KIAA0316, KIAA1284, EBP50 (PDZ #1), (Shank1; Shank2; Shank3; Syntenin; Magil (PDZ #1); Tipl; Mintl (PDZ #1,2); Novel Serine Protease; MUPP1 (PDZ#3,7,9,1 1), MAST2, NSP, NOS1, PAR3 (PDZ #3), PAR3L(PDZ #3); PAR5beta, RiM2, Rhodophilin-like, SIP-I, SITAC-18(PDZ #2), SITAC-18(PDZ #1), SIP1, ZO-1 (PDZ #1), ZO-3 (PDZ #1), DVL3, DVL2 (PDZ #1), PTPLI (PDZ#4), HEMBA 10003117, Pick1 (accession numbers are shown in Table 2).

PDZ domains contain ˜80-90 residues that fold into a structure with a beta-sandwich of 5-6 beta-strands and two alpha-helices. The peptide ligand binds in a hydrophobic cleft composed of a beta-strand (bB), an alpha-helix and a loop that binds the peptide carboxylate group. The peptide binds in an anti-parallel fashion to the bB strand, with the C-terminal residue occupying a hydrophobic pocket. PDZ heterodimers form a linear head-to-tail arrangement that involves recognition of an internal on one of the partner proteins. PDZ domain proteins are known in the art and new proteins can be identified as having PDZ domains by sequencing the protein and identifying the presence of a PDZ domain. PDZ proteins are explained in detail and a large number of examples are given in U.S. patent application Ser. No. 10/485,788, filed Aug. 2, 2004. Alternatively, a protein suspected of being a PDZ protein can be tested for binding to a variety of PL proteins or PL protein PL classes. Examples of PDZ domains can be found in Table 2.

The PDZ proteins listed in TABLE 2 are naturally occurring proteins containing a wild-type PDZ domain. Polypeptides containing functional variant PDZ domains are readily designed since the PDZ domain is well characterized at the structural level. For example, the three-dimensional structure of the PDZ domain is described and discussed in great detail in Doyle (Cell 1996 95:1067-1076) and the structure of several PDZ domains have been determined by crystallography.

When a particular PDZ domain-containing polypeptide is referenced herein, e.g., when a reference is made to a DLG1 PDZ domain-containing polypeptide, the reference is intended to encompass polypeptides containing a wild-type PDZ domain, and variants thereof that retain PDZ ligand binding activity.

TABLE 1A
Experimentally Determined Interactions

| Path- | | | C-ter- | SeqID | |
|---|---|---|---|---|---|
| ogen | Protein | GI number | minus | No | PDZ Partners |
| HIV-1 | Env | 6469525 | RALL, | 242 | AIPC (PDZ #1) |
| | | 902806 | RILL | 243 | GORASP1 (PDZ #1) |
| | | (9629363) | | | INADL (PDZ #3) |
| | | | | | KIAA0316 |
| | | | | | KIAA1284 |
| | | | | | MAGI1 (PDZ #1) |
| | | | | | MAST2 |
| | | | | | MINT1 (PDZ #1, 2) |
| | | | | | NSP |
| | | | | | NOS1 |
| | | | | | PAR3 (PDZ #3) |
| | | | | | PAR3L (PDZ #3) |
| | | | | | PAR6 beta |
| | | | | | RIM2 |
| | | | | | Rhodophilin-like |
| | | | | | SITAC-18 (PDZ #2) |
| | | | | | SITAC-18 (PDZ #1) |
| | | | | | SIP1 |
| | | | | | ZO-1 (PDZ #2) |
| | | | | | EBP50 (PDZ #1) |
| | | | | | KIAA1284 |
| | | | | | PICK1 |
| | | | | | Shank 1 |
| | | | | | Shank 2 |
| | | | | | Shank 3 |
| | | | | | TIP1 |

TABLE 1B
Predicted Interactions

| | | | | | |
|---|---|---|---|---|---|
| HIV-1 | Nef | 7416180 | FKNC, | 244 | MINT1 |
| | | 20126975 | FKDC, | 245 | SITAC-18 |
| | | 13898137 | YKNC, | 246 | TIP1 |
| | | 2992598 | YKDC | 247 | PICK1 |
| HIV-2 | Env | 119459 | IALL, | 248 | EBP50 (PDZ #1) |
| | | 221488 | LALL, | 249 | KIAA1284 |
| | | 2108172 | LTLL | 250 | MAST2 |
| | | | | | NSP |
| | | | | | PAR3 (PDZ #3) |
| | | | | | PICK1 |
| | | | | | Shank 1 |
| | | | | | Shank 2 |
| | | | | | Shank 3 |
| | | | | | TIP1 |
| | | | | | (Plus all PDZs |
| | | | | | binding HIV-1 Env) |
| | Vif | 9628883 | EILA, | 251 | INADL (PDZ # 3 |
| | | | GILA, | 252 | RIM2 |
| | | | DILA | 253 | EBP50 (PDZ #1) |
| Hepatitis B | Protein X | 20302507 | FTSA | 254 | TIP2 |
| | | (60279611) | | | KIAA1526 |
| | | | | | SITAC-18 |
| | | | | | MINT1 (PDZ 1, 2) |
| | | | | | DVL3 |
| | | | | | NOS1 |
| | S antigen | 6692561 | WVYI | 255 | PTPL1 (PDZ # 4) |
| | | (21326586) | | | MAST2 |
| | | | | | MINT1 (PDZ # 1, 2) |
| | | | | | HEMBA 1003117 |
| | | | | | PAR3 (PDZ # 3) |
| | | | | | AF6 |
| | | | | | NOS1 |
| | | | | | AIPC |
| | | | | | SYNTENIN |
| | | | | | MUPP1 (PDZ # 3, 7, 9, 11) |
| | | | | | DVL2 (PDZ #1) |
| | | | | | ZO-3 (PDZ #1) |
| | | | | | SIP1 |
| | | | | | PICK1 |
| | | | | | (plus HIV-1 Env list) |
| Hepatitis C | Capsid C | 18148510 | PASA, | 256 | TIP2 |
| | E1 | (26053621) | PVSA | 257 | ZO-1 (PDZ #2) |
| | | 402408 | GVDA | 258 | TIP2 |
| | | (26053622) | | | RIM2 |
| | | | | | INADL (PDZ # 3) |
| RSV | Nucleoprotein | 127888 | DVEL | 259 | ZO-1 (PDZ #2) |
| | | (1489820) | | | RIM2 |
| | | | | | Novel serine protease |
| | | | | | MINT1 |
| | | | | | EBP50 (PDZ#1) |
| | | | | | AIPC (PDZ#1) |
| | | | | | PAR3 (PDZ#3) |
| | | | | | SIP1 (PDZ#1) |
| | | | | | (plus HIV-1 Env list) |
| Rotavirus A | VP4 | 13111356 | QCKL, | 260 | MAGI3 (PDZ #5) |
| | | 564036 | QCRL | 261 | LIM mystique |
| | | (139264) | | | LIM-RIL |
| | | | | | ENIGMA |
| | | | | | MAGI-1 (PDZ #3) |
| | | | | | MAST2 |
| | | | | | MAGI2 (PDZ #5) |
| | | | | | TIP1 |
| | | | | | LIM protein |
| | | | | | ZO-1 (PDZ#2) |
| | VP7 | 9246985 | YYRV, | 262 | GRIP 1 (PDZ #6) |
| | | 1770358 | YYRI | 263 | PTPL1 (PDZ #4) |
| | | (139535) | | | MAST1 |
| | | | | | MUPP1 (PDZ #3, 7, 9) |
| | | | | | KIAA1719 (PDZ #6) |
| | | | | | MAST2 |
| | | | | | PICK1 |
| | | | | | ZO-1 (PDZ#2) |
| | NSP2 | 1771596 | QVGI, | 264 | NOS1 (PDZ # 1, 2, 3) |
| | (QIGI is | 33324358 | HIGI, | 265 | MINT1 (PDZ # 2) |
| | Sero2 NS35) | 549383 | QIGI, | 266 | ZO-1 (PDZ#2) |
| | | 6009568 | RIGI | 267 | |
| | | (55793488) | | | |
| | NSP5 | 540789 | IKDL, | 268 | ZO-1 (PDZ #2) |
| | | 540790 | IEDL | 269 | RIM2 |
| | | | | | NOS1 |
| Mycobacterium | ESXN | 61223753 | SSWA | 270 | TIP2 |
| Tuberculosis | | | | | KIAA1526 |
| | | | | | PSD95 (PDZ#2) |
| | ESXS | 57117047 | YTGF | 271 | MAST2 |
| | | | | | MAST3 |
| | | | | | Shank 3 |
| | | | | | APXL1 |
| | | | | | syntenin |
| | ESAT-6 | 61223745 | GMFA | 272 | INADL (PDZ #3) |
| | | | | | RIM2 |
| | | | | | TIP2 |

IV. PDZ Protein and PL Protein Interactions

TABLES 1A and 1B list PDZ domain-containing proteins (“PDZ proteins”) and pathogen PDZ ligand proteins (“PL proteins”) which have been identified as binding to one another in Example 1. Each of the pathogen PL proteins has binding affinity for at least one of the PDZ proteins. The first column of TABLES 1A and 1B lists the pathogen from which the PL protein is derived; the second column lists the name of the gene containing the PL; the third column provides the GenBank identification number (GI number) for each protein (which database entries are incorporated by reference herein, including any annotation described therein); the fourth column provides the C-terminal four amino acids of the PL protein; and the fifth column lists the SEQ ID NOs for the PLs, and the sixth column lists the PDZ proteins which have binding affinity for each PL. The proteins shown in TABLES 1A and 1B represent examples of viral and bacterial PL proteins. Table IA provides PDZ/PL interactions that were identified experimentally using the methods in Examples 1-5. Table 1B provides predicted interactions that were done based on C-terminal sequence analysis using the ARBOR VITA proprietary database based on PDZ array studies and interactions.

TABLES 1A and 1B show the C-terminal sequences of the envelope proteins from several retroviruses, flaviviruses and paramyxoviruses, including the characteristic sequences for different subgroups or strains, in the case of HIV-1, HIV-2. For example, in the case of HIV-1 and HIV-2 envelope proteins, consensus sequences for each strain are shown. Although many of the PDZ parters shown in the Table may bind to both PL motifs, some only bind to one or the other as indicated in the Examples herein with two HIV-1 PL peptides.

TABLE 2 lists the PDZ proteins that were screened for binding to the viral and bacterial PL proteins in Table 1. TABLE 2 also lists other PDZ proteins that can be tested for binding to PL proteins. TABLE 2 also lists the sequences of the PDZ domains cloned into a vector (PGEX-3X vector) for production of GST-PDZ fusion proteins (Pharmacia). More specifically, the first column (left to right) entitled “Gene Name” lists the name of the gene containing the PDZ domain. The second column labeled “GI or Acc#” is a unique Genbank identifier for the gene used to design primers for PCR amplification of the listed sequence. The next column labeled “PDZ#” indicates the Pfam-predicted PDZ domain number, as numbered from the amino-terminus of the gene to the carboxy-terminus. The last column entitled “Sequence fused to GST construct” provides the actual amino acid sequence inserted into the GST-PDZ expression vector as determined by DNA sequencing of the constructs. PDZ proteins can be produced as fusion proteins as long as they have an active PDZ domain.

Two complementary assays (the A and G assays) to detect binding between a PDZ-domain polypeptide and candidate PDZ ligand polypeptide are set out in detail in U.S. patent application Ser. Nos. 10/485,788, filed Aug. 2, 2004 and 10/714,537, filed Nov. 14, 2003. In each of the two different assays, binding is detected between a peptide having a sequence corresponding to the C-terminus of a protein anticipated to bind to one or more PDZ domains (i.e. a candidate PL peptide) and a PDZ-domain polypeptide (typically a fusion protein containing a PDZ domain).

A. Assays for Detection of Interactions Between PDZ-Domain Polypeptides and NMDA Receptor PL Proteins

Two complementary assays, termed “A” and “G,” were developed to detect binding between a PDZ-domain polypeptide and candidate PDZ ligand. In each of the two different assays, binding is detected between a peptide having a sequence corresponding to the C-terminus of a protein anticipated to bind to one or more PDZ domains (i.e. a candidate PL peptide) and a PDZ-domain polypeptide (typically a fusion protein containing a PDZ domain). In the “A” assay, the candidate PL peptide is immobilized and binding of a soluble PDZ-domain polypeptide to the immobilized peptide is detected (the “A” assay is named for the fact that in one embodiment an avidin surface is used to immobilize the peptide). In the “G” assay, the PDZ-domain polypeptide is immobilized and binding of a soluble PL peptide is detected (The “G” assay is so-named because a GST-binding surface is used to immobilize the PDZ-domain polypeptide). Exemplary assays are described below.

I. “A Assay” Detection of PDZ-Ligand Binding Using Immobilized PL Peptide.

The assay involves the following: Biotinylated candidate PL peptides are immobilized on an avidin coated surface. The binding of PDZ-domain fusion protein to this surface is then measured.

(1) Avidin is bound to a surface, e.g. a protein binding surface. Optionally, avidin is bound to a polystyrene 96 well plate (e.g., Nunc Polysorb (cat #475094) by addition of 100 μL per well of 20 μg/mL of avidin (Pierce) in phosphate buffered saline without calcium and magnesium, pH 7.4 (“PBS”, GibcoBRL) at 4° C. for 12 hours. The plate is then treated to block nonspecific interactions by addition of 200 μL per well of PBS containing 2 g per 100 mL protease-free bovine serum albumin (“PBS/BSA”) for 2 hours at 4° C. The plate is then washed 3 times with PBS by repeatedly adding 200 μL per well of PBS to each well of the, plate and then dumping the contents of the plate into a waste container and tapping the plate gently on a dry surface.

(2) Biotinylated PL peptides (or candidate PL peptides) are immobilized on the surface of wells of the plate by addition of 50 μL per well of 0.4 μM peptide in PBS/BSA for 30 minutes at 4° C. Usually, each different peptide is added to at least eight different wells so that multiple measurements (e.g. duplicates and also measurements using different (GST/PDZ-domain fusion proteins and a GST alone negative control) can be made, and also additional negative control wells are prepared in which no peptide is immobilized. Following immobilization of the PL peptide on the surface, the plate is washed 3 times with PBS.

(3) GST/PDZ-domain fusion protein is allowed to react with the surface by addition of 50 μL per well of a solution containing 5 μg/mL GST/PDZ-domain fusion protein in PBS/BSA for 2 hours at 4° C. As a negative control, GST alone (i.e. not a fusion protein) is added to specified wells, generally at least 2 wells (i.e. duplicate measurements) for each immobilized peptide. After the 2 hour reaction, the plate is washed 3 times with PBS to remove unbound fusion protein.

(4) The binding of the GST/PDZ-domain fusion protein to the avidin-biotinylated peptide surface can be detected using a variety of methods, and detectors known in the art. In one embodiment, 50 μL per well of an anti-GST antibody in PBS/BSA (e.g. 2.5 μg/mL of polyclonal goat-anti-GST antibody, Pierce) is added to the plate and allowed to react for 20 minutes at 4° C. The plate is washed 3 times with PBS and a second, detectably labeled antibody is added. In one embodiment, 50 μL per well of 2.5 μg/mL of horseradish peroxidase (HRP)-conjugated polyclonal rabbit anti-goat immunoglobulin antibody is added to the plate and allowed to react for 20 minutes at 4° C. The plate is washed 5 times with 50 mM Tris pH 8.0 containing 0.2% Tween 20, and developed by addition of 100 μL per well of HRP-substrate solution (TMB, Dako) for 20 minutes at room temperature (RT). The reaction of the HRP and its substrate is terminated by the addition of 100 μL per well of 1 M sulfuric acid and the optical density (O.D.) of each well of the plate is read at 450 nm.

(5) Specific binding of a PL peptide and a PDZ-domain polypeptide is detected by comparing the signal from the well(s) in which the PL peptide and PDZ domain polypeptide are combined with the background signal(s). The background signal is the signal found in the negative controls. Typically a specific or selective reaction will be at least twice background signal, more typically more than 5 times background, and most typically 10 or more times the background signal. In addition, a statistically significant reaction involves multiple measurements of the reaction with the signal and the background differing by at least two standard errors, more typically four standard errors, and most typically six or more standard errors. Correspondingly, a statistical test (e.g. a T-test) comparing repeated measurements of the signal with repeated measurements of the background will result in a p-value <0.05, more typically a p-value <0.01, and most typically a p-value <0.001 or less. As noted, in an embodiment of the “A” assay, the signal from binding of a GST/PDZ-domain fusion protein to an avidin surface not exposed to (i.e. not covered with) the PL peptide is one suitable negative control (sometimes referred to as “B”). The signal from binding of GST polypeptide alone (i.e. not a fusion protein) to an avidin-coated surface that has been exposed to (i.e. covered with) the PL peptide is a second suitable negative control (sometimes referred to as “B2”). Because all measurements are done in multiples (i.e. at least duplicate) the arithmetic mean (or, equivalently, average) of several measurements is used in determining the binding, and the standard error of the mean is used in determining the probable error in the measurement of the binding. The standard error of the mean of N measurements equals the square root of the following: the sum of the squares of the difference between each measurement and the mean, divided by the product of (N) and (N−1). Thus, in one embodiment, specific binding of the PDZ protein to the plate-bound PL peptide is determined by comparing the mean signal (“mean S”) and standard error of the signal (“SE”) for a particular PL-PDZ combination with the mean B1 and/or mean B2.

II. “G Assay”-Detection of PDZ-Ligand Binding Using Immobilized PDZ-Domain Fusion Polypeptide

In one aspect, the invention provides an assay in which a GST/PDZ fusion protein is immobilized on a surface (“G” assay). The binding of labeled PL peptide (for example one of those listed in Table 1) to this surface is then measured. In a preferred embodiment, the assay is carried out as follows:

(1) A PDZ-domain polypeptide is bound to a surface, e.g. a protein binding surface. In a preferred embodiment, a GST/PDZ fusion protein containing one or more PDZ domains is bound to a polystyrene 96-well plate. The GST/PDZ fusion protein can be bound to the plate by any of a variety of standard methods, although some care must be taken that the process of binding the fusion protein to the plate does not alter the ligand-binding properties of the PDZ domain. In one embodiment, the GST/PDZ fusion protein is bound via an anti-GST antibody that is coated onto the 96-well plate. Adequate binding to the plate can be achieved when:
a. 100 μL per well of 5 μg/mL goat anti-GST polyclonal antibody (Pierce) in PBS is added to a polystyrene 96-well plate (e.g., Nunc Polysorb) at 4° C. for 12 hours.
b. The plate is blocked by addition of 200 μL per well of PBS/BSA for 2 hours at 4° C.
c. The plate is washed 3 times with PBS.
d. 50 μL per well of 5 μg/mL GST/PDZ fusion protein) or, as a negative control, GST polypeptide alone (i.e. not a fusion protein) in PBS/BSA is added to the plate for 2 hours at 4° C.
e. the plate is again washed 3 times with PBS.

(2) Biotinylated PL peptides are allowed to react with the surface by addition of 50 μL per well of 20 μM solution of the biotinylated peptide in PBS/BSA for 10 minutes at 4° C., followed by an additional 20 minute incubation at 25° C. The plate is washed 3 times with ice cold PBS.

(3) The binding of the biotinylated peptide to the GST/PDZ fusion protein surface can be detected using a variety of methods and detectors. In an exemplary procedure, 100 μL per well of 0.5 μg/mL streptavidin-horse radish peroxidase (HRP) conjugate dissolved in BSA/PBS is added and allowed to react for 20 minutes at 4° C. The plate is then washed 5 times with 50 mM Tris pH 8.0 containing 0.2% Tween 20, and developed by addition of 100 μL per well of HRP-substrate solution (TMB, Dako) for 20 minutes at room temperature (RT). The reaction of the HRP and its substrate is terminated by addition of 100 μL per well of 1 M sulfuric acid, and the optical density (O.D.) of each well of the plate is read at 450 um.

(4) Specific binding of a PL peptide and a PDZ domain polypeptide is determined by comparing the signal from the well(s) in which the PL peptide and PDZ domain polypeptide are combined, with the background signal(s). The background signal is the signal found in the negative control(s). Typically a specific or selective reaction is at least twice background signal, more typically more than 5 times background, and most typically 10 or more times the background signal. In addition, a statistically significant reaction involves multiple measurements of the reaction with the signal and the background differing by at least two standard errors, more typically four standard errors, and most typically six or more standard errors. Correspondingly, a statistical test (e.g. a T-test) comparing repeated measurements of the signal with -repeated measurements of the background will result in a p-value <0.05, more typically a p-value <0.01, and most typically a p-value <0.001 or less. As noted, in an embodiment of the “G” assay, the signal from binding of a given PL peptide to immobilized (surface bound) GST polypeptide alone is one suitable negative control (sometimes referred to as “B 1”). Because all measurement are done in multiples (i.e. at least duplicate) the arithmetic mean (or, equivalently, average) of several measurements is used in determining the binding, and the standard error of the mean is used in determining the probable error in the measurement of the binding. The standard error of the mean of N measurements equals the square root of the following: the sum of the squares of the difference between each measurement and the mean, divided by the product of (N) and (N−1). Thus, in one embodiment, specific binding of the PDZ protein to the platebound peptide is determined by comparing the mean signal (“mean S”) and standard error of the signal (“SE”) for a particular PL-PDZ combination with the mean B I.

i) “G′ assay” and “G″ assay”

Two specific modifications of the specific conditions described supra for the “G assay” are particularly useful. The modified assays use lesser quantities of labeled PL peptide and have slightly different biochemical requirements for detection of PDZ-ligand binding compared to the specific assay conditions described supra.

The assay conditions described in this section are referred to as the “G′ assay” and the “G″ assay,” with the specific conditions described in the preceding section on G assays being referred to as the “G⁰ assay.” The “G′ assay” is identical to the “G⁰ assay” except at step (2) the peptide concentration is 10 uM instead of 20 uM. This results in slightly lower sensitivity for detection of interactions with low affinity and/or rapid dissociation rate. Correspondingly, it slightly increases the certainty that detected interactions are of sufficient affinity and half-life to be of biological importance and useful therapeutic targets.

The “G″ assay” is identical to the “G⁰ assay” except that at step (2) the peptide concentration is 1 μM instead of 20 μM and the incubation is performed for 60 minutes at 25° C. (rather than, e.g., 10 minutes at 4° C. followed by 20 minutes at 25° C.). This results in lower sensitivity for interactions of low affinity, rapid dissociation rate, and/or affinity that is less at 25° C. than at 4° C. Interactions will have lower affinity at 25° C. than at 4° C. if (as we have found to be generally true for PDZ-ligand binding) the reaction entropy is negative (i.e. the entropy of the products is less than the entropy of the reactants). In contrast, the PDZ-PL binding signal may be similar in the “G″ assay” and the “G⁰ assay” for interactions of slow association and dissociation rate, as the PDZ-PL complex will accumulate during the longer incubation of the “G″ assay.” Thus comparison of results of the “G″ assay” and the “G⁰ assay” can be used to estimate the relative entropies, enthalpies, and kinetics of different PDZ-PL interactions. (Entropies and enthalpies are related to binding affinity by the equations delta G=RT 1n Kd)=delta H−T delta S where delta G, H, and S are the reaction free energy, enthalpy, and entropy respectively, T is the temperature in degrees Kelvin, R is the gas constant, and Kd is the equilibrium dissociation constant). In particular, interactions that are detected only or much more strongly in the “G⁰ assay” generally have a rapid dissociation rate at 25° C. (t1/2<10 minutes) and a negative reaction entropy, while interactions that are detected similarly strongly in the “G″ assay” generally have a slower dissociation rate at 25° C. (t1/2>10 minutes). Rough estimation of the thermodynamics and kinetics of PDZ-PL interactions (as can be achieved via comparison of results of the “G⁰ assay” versus the “G″ assay” as outlined supra) can be used in the design of efficient inhibitors of the interactions. For example, a small molecule inhibitor based on the chemical structure of a PL that dissociates slowly from a given PDZ domain (as evidenced by similar binding in the “G″ assay” as in the “G⁰ assay”) may itself dissociate slowly and thus be of high affinity.

In this manner, variation of the temperature and duration of step (2) of the “G assay” can be used to provide insight into the kinetics and thermodynamics of the PDZ-ligand binding reaction and into design of inhibitors of the reaction.

The detectable labels of the invention can be any detectable compound or composition which is conjugated directly or indirectly with a molecule (such as described above). The label can be detectable by itself (e.g., radioisotope labels or fluorescent labels) or, in the case of an enzymatic label, can catalyze a chemical alteration of a substrate compound or composition which is detectable. The preferred label is an enzymatic one which catalyzes a color change of a non-radioactive color reagent.

Sometimes, the label is indirectly conjugated with the antibody. For example, the antibody can be conjugated with biotin and any of the categories of labels mentioned above can be conjugated with avidin, or vice versa (see also “A” and “G” assay above). Biotin binds selectively to avidin and thus, the label can be conjugated with the antibody in this indirect manner. See, Ausubel, supra, for a review of techniques involving biotin-avidin conjugation and similar assays. Alternatively, to achieve indirect conjugation of the label with the antibody, the antibody is conjugated with a small hapten (e.g. digoxin) and one of the different types of labels mentioned above is conjugated with an anti-hapten antibody (e.g. anti-digoxin antibody). Thus, indirect conjugation of the label with the antibody can be achieved.

Assay variations can include different washing steps. By “washing” is meant exposing the solid phase to an aqueous solution (usually a buffer or cell culture media) in such a way that unbound material (e.g., non-adhering cells, non-adhering capture agent, unbound ligand, receptor, receptor construct, cell lysate, or HRP antibody) is removed therefrom. To reduce background noise, it is convenient to include a detergent (e.g., Triton X) in the washing solution. Usually, the aqueous washing solution is decanted from the wells of the assay plate following washing. Conveniently, washing can be achieved using an automated washing device. Sometimes, several washing steps (e.g., between about 1 to 10 washing steps) can be required.

Various buffers can also be used in PDZ-PL detection assays. For example, various blocking buffers can be used to reduce assay background. The term “blocking buffer” refers to an aqueous, pH buffered solution containing at least one blocking compound which is able to bind to exposed surfaces of the substrate which are not coated with a PL or PDZ-containing protein. The blocking compound is normally a protein such as bovine serum albumin (BSA), gelatin, casein or milk powder and does not cross-react with any of the reagents in the assay. The block buffer is generally provided at a pH between about 7 to 7.5 and suitable buffering agents include phosphate and TRIS.

Various enzyme-substrate combinations can also be utilized in detecting PDZ-PL interactions. Examples of enzyme-substrate combinations include, for example:

(i) Horseradish peroxidase (HRPO) with hydrogen peroxidase as a substrate, wherein the hydrogen peroxidase oxidizes a dye precursor (e.g. orthophenylene diamine [OPD] or 3,3′,5,5′-tetramethyl benzidine hydrochloride [TMB]) (as described above).

(ii) alkaline phosphatase (AP) with para-Nitrophenyl phosphate as chromogenic substrate.

(iii) β-D-galactosidase (βD-Gal) with a chromogenic substrate (e.g. p-nitrophenyl-β-D-galactosidase) or fluorogenic substrate 4-methylumbelliferyl-β-D-galactosidase. Numerous other enzyme-substrate combinations are available. For a general review of these, see U.S. Pat. Nos. 4,275,149 and 4,318,980, both of which are herein incorporated by reference.

TABLE 2

Exemplary human PDZ domains

| | | |
|---|---|---|
| GI or | PDZ | Sequence fused to GST |

| | | | |
|---|---|---|---|
| Gene Name | Acc # | # | Construct |

| | | | |
|---|---|---|---|
| PSMD9 | 9184389 | 1 | RDMAEAHKEAMSRKLGQSESQGPPRAF |
| | | | AKVNSISPGSPSIAGLQVDDIVEFGSV |
| | | | NTQNEQSLHNIGSVVQHSEGALAPTIL |
| | | | LSVSM |
| | | | (SEQ ID NO:1) |
| AF6 | 430993 | 1 | LRKEPEIITVTLKKQNGMGLSIVAAKG |
| | | | AGQDKLGIYVKSVVKGGAADVDGRLAA |
| | | | GDQLLSVDGRSLVGLSQERAAELMTRT |
| | | | SSVVTLEVAKQG (SEQ ID NO:2) |
| AIPC | 12751451 | 1 | LIRPSVISIIGLYKEKGKGLGFSIAGG |
| | | | RDCIRGQMGIFVKTIFPNGSAAEDGRL |
| | | | KEGDEILDVNGIPIKGLTFQEAIHTFK |
| | | | QIRSGLFVLTVRTKLVSPSLTNSS |
| | | | (SEQ ID NO:3) |
| AIPC | 12751451 | 2 | GISSLGRKTPGPKDRIVMEVTLNKEPR |
| | | | VGLGIGACCLALENSPPGIYIHSLAPG |
| | | | SVAKMESNLSRGDQILEVNSVNVRHAA |
| | | | LSKVHAILSKCPPGPVRLVIGRHPNPK |
| | | | VSEQEMDEVIARSTYQESKEANSS |
| | | | (SEQ ID NO:4) |
| AIPC | 12751451 | 3 | QSENEEDVCFIVLNRKEGSGLGFSVAG |
| | | | GTDVEPKSITVHRVFSQGAASQEGTMN |
| | | | RGDFLLSVNGASLAGLAHGNVLKVLHQ |
| | | | AQLHKDALVVIKKGMDQPRPSNSS |
| | | | (SEQ ID NO:5) |
| AIPC | 12751451 | 4 | LGRSVAVHDALCVEVLKTSAGLGLSLD |
| | | | GGKSSVTGDGPLVIKRVYKGGAAEQAG |
| | | | IIEAGDEILAINGKPLVGLMHFDAWNI |
| | | | MKSVPEGPVQLLIRKHRNSS |
| | | | (SEQ ID NO:6) |
| ALP | 2773059 | 1 | REEGGMPQTVILPGPAAWGFRLSGGID |
| | | | FNQPLVITRITPGSKAAAANLCPGDVI |
| | | | LAIDGFGTESMTHADGQDRIKAAAHQL |
| | | | CLKIDRGETHLWSPSIV |
| | | | (SEQ ID NO:7) |
| APXL-1 | 13651263 | 1 | ILVEVQLSGGAPWGFTLKGGREHGEPL |
| | | | VITKIEEGSKAAAVDKLLAGDEIVGIN |
| | | | DIGLSGFRQEAICLVKGSHKTLKLVVK |
| | | | RNSS |
| | | | (SEQ ID NO:8) |
| MAGI2 | 2947231 | 1 | REKPLFTRDASQLKGTFLSTTLKKSNM |
| | | | GFGFTIIGGDEPDEFLQVKSVIPDGPA |
| | | | AQDGKMETGDVIVYINEVCVLGHTHAD |
| | | | VVKLFQSVPIGQSVNLVLCRGYP |
| | | | (SEQ ID NO:9) |
| MAGI2 | 2947231 | 2 | LSGATQAELMTLTIVKGAQGFGFTIAD |
| | | | SPTGQRVKQILDIQGCPGLCEGDLIVE |
| | | | INQQNVQNLSHTEVVDILKDCPIGSET |
| | | | SLIIHRGGFF |
| | | | (SEQ ID NO:10) |
| MAGI2 | 2947231 | 3 | HYKELDVHLRRMESGFGFRILGGDEPG |
| | | | QPILIGAVIAMGSADRDGRLHPGDELV |
| | | | YVDGIPVAGKTHRYVIDLMHHAARNGQ |
| | | | VNLTVRRKVLCG |
| | | | (SEQ ID NO:11) |
| MAGI2 | 2947231 | 4 | EGRGISSHSLQTSDAVIHRKENEGFGF |
| | | | VIISSLNRPESGSTITVPHKIGRIIDG |
| | | | SPADRCAKLKVGDRILAVNGQSIINMP |
| | | | HADIVKLIKDAGLSVTLRIIPQEEL |
| | | | (SEQ ID NO:12) |
| MAGI2 | 2947231 | 5 | LSDYRQPQDFDYFTVDMEKGAKGFGFS |
| | | | IRGGREYKMDLYVLRLAEDGPAIRNGR |
| | | | MRVGDQIIEINGESTRDMTHARAIELI |
| | | | KSGGRRVRLLLKRGTGQ |
| | | | (SEQ ID NO:13) |
| MAGI2 | 2947231 | 6 | HESVIGRNPEGQLGFELKGGAENGQFP |
| | | | YLGEVKPGKVAYESGSKLVSEELLLEV |
| | | | NETPVAGLIIRDVLAVIKHCKDPLRLK |
| | | | CVKQGGIHR |
| | | | (SEQ ID NO:14) |
| CARD11 | 12382772 | 1 | SVGHVRGPGPSVQHTTLNGDSLTSQLT |
| | | | LLGGNARGSFVHSVKPGSLAEKAGLRE |
| | | | GHQLLLLEGCIRGERQSVPLDTCTKEE |
| | | | AHWTIQRCSGPVTLHYKVNHEGYRK |
| | | | (SEQ ID NO:15) |
| CARD14 | 13129123 | 1 | RRPARRILSQVTMLAFQGDALLEQISV |
| | | | IGGNLTGIFIHRVTPGSAADQMALRPG |
| | | | TQIVMVDYEASEPLFKAVLEDTTLEEA |
| | | | VGLLRRVDGFCCLSVKVNTDGYKR |
| | | | (SEQ ID NO:16) |
| CASK | 3087815 | 1 | TRVRLVQFQKNTDEPMGITLKMNELNH |
| | | | CIVARIMHGGMIHRQGTLHVGDEIREI |
| | | | NGISVANQTVEQLQKMLREMRGSITFK |
| | | | IVPSYRTQS |
| | | | (SEQ ID NO:17) |
| CNK1 | 3930780 | 1 | LEQKAVLEQVQLDSPLGLEIHTTSNCQ |
| | | | HFVSQVDTQVPTDSRLQIQPGDEVVQI |
| | | | NEQVVVGWPRKNMVRELLREPAGLSLV |
| | | | LKKIPIP |
| | | | (SEQ ID NO:18) |
| Cytohesin | 3192908 | 1 | QRKLVTVEKQDNETFGFEIQSYRPQNQ |
| Binding | | | NACSSEMFTLICKIQEDSPAHCAGLQA |
| Protein | | | GDVLANINGVSTEGFTYKQVVDLIRSS |
| | | | GNLLTIETLNG |
| | | | (SEQ ID NO:19) |
| Densin 180 | 16755892 | 1 | RCLIQTKGQRSMDGYPEQFCVRIEKNP |
| | | | GLGFSISGGISGQGNPFKPSDKGIFVT |
| | | | RVQPDGPASNLLQPGDKILQANGHSFV |
| | | | HMEHEKAVLLLKSFQNTVDLVIQRELT |
| | | | V |
| | | | (SEQ ID NO:20) |
| DLG1 | 475816 | 1 | IQVNGTDADYEYEEITLERGNSGLGFS |
| | | | IAGGTDNPHIGDDSSIFITKIITGGAA |
| | | | AQDGRLRVNDCILQVNEVDVRDVTHSK |
| | | | AVEALKEAGSIVRLYVKRRN |
| | | | (SEQ ID NO:21) |
| DLG1 | 475816 | 2 | IQLIKGPKGLGFSIAGGVGNQHIPGDN |
| | | | SIYVTKIIEGGAAHKDGKLQIGDKLLA |
| | | | VNNVCLEEVTHEEAVTALKNTSDFVYL |
| | | | KVAKPTSMYMNDGN |
| | | | (SEQ ID NO:22) |
| DLG1 | 475816 | 3 | ILHRGSTGLGFNIVGGEDGEGIFISFI |
| | | | LAGGPADLSGELRKGDRIISVNSVDLR |
| | | | AASHEQAAAALKNAGQAVTIVAQYRPE |
| | | | EYSR |
| | | | (SEQ ID NO:23) |
| DLG2 | 12736552 | 1 | ISYVNGTEIEYEFEEITLERGNSGLGF |
| | | | SIAGGTDNPHIGDDPGIFITKIIPGGA |
| | | | AAEDGRLRVNDCILRVNEVDVSEVSHS |
| | | | KAVEALKEAGSIVRLYVRRR |
| | | | (SEQ ID NO:24) |
| DLG2 | 12736552 | 2 | IPILETVVEIKLFKGPKGLGFSIAGGV |
| | | | GNQHIPGDNSIYVTKIIDGGAAQKDGR |
| | | | LQVGDRLLMVNNYSLEEVTHEEAVAIL |
| | | | KNTSEVVYLKVGKPIVMTDPYGPPNSS |
| | | | (SEQ ID NO:25) |
| DLG2 | 12736552 | 3 | ILEGEPRKVVLHKGSTGLGFNIVGGEDG |
| | | | EGIFVSFILAGGPADLSGELQRGDQILS |
| | | | VNGIDLRGASHEQAAAALKGAGQTVTII |
| | | | AQHQPEDYARFEAKIHDLNSS |
| | | | (SEQ ID NO:26) |
| DLG5 | 3650451 | 1 | GIPYVEEPRHVKVQKGSEPLGISIVSGE |
| | | | KGGIYVSKVTVGSIAHQAGLEYGDQLLE |
| | | | FNGINLRSATEQQARLIIGQQCDTITIL |
| | | | AQYNPHVHQLRNSSZLTD |
| | | | (SEQ ID NO:27) |
| DLG5 | 3650451 | 2 | GILAGDANKKTLEPRVVFIKKSQLELGV |
| | | | HLCGGNLHGVFVAEVEDDSPAKGPDGLV |
| | | | PGDLILEYGSLDVRNKTVEEVYVEMLKP |
| | | | RDGVRLKVQYRPEEFIVTD |
| | | | (SEQ ID NO:28) |
| DLG6, | 14647140 | 1 | PTSPEIQELRQMLQAPHFKALLSAHDTI |
| splice | | | AQKDFEPLLPPLPDNIPESEEAMRIVCL |
| variant 1 | | | VKNQQPLGATIKRHEMTGDILVARIIHG |
| | | | GLAERSGLLYAGDKLVEVNGVSVEGLDP |
| | | | EQVIHILAMSRGTIMFKVVPVSDPPVNS |
| | | | S |
| | | | (SEQ ID NO:29) |
| DLG6, | AB053303 | 1 | PTSPEIQELRQMLQAPHFKGATIKRHEM |
| splice | | | TGDILVARIIHGGLAERSGLLYAGDKLV |
| variant 2 | | | EVNGVSVEGLDPEQVIHILAMSRGTIMF |
| | | | KVVPVSDPPVNSS |
| | | | (SEQ ID NO:30) |
| DVL1 | 2291005 | 1 | LNIVTVTLNMERHHFLGISIVGQSNDRG |
| | | | DGGIYIGSIMKGGAVAADGRIEPGDMLL |
| | | | QVNDVNFENMSNDDAVRVLREIVSQTGP |
| | | | ISLTVAKCW |
| | | | (SEQ ID NO:31) |
| DVL2 | 2291007 | 1 | LNIITVTLNMEKYNFLGISIVGQSNERG |
| | | | DGGIYIGSIMKGGAVAADGRIEPGDMLL |
| | | | QVNDMNFENMSNDDAVRVLRDIVHKPGP |
| | | | IVLTVAKCWDPSPQNS |
| | | | (SEQ ID NO:32) |
| DVL3 | 6806886 | 1 | IITVTLNMEKYNFLGISIVGQSNERGDG |
| | | | GIYIGSIMKGGAVAADGRIEPGDMLLQV |
| | | | NEINIFENMSNDDAVRVLREIVHKPGPI |
| | | | TLTVAKCWDPSP |
| | | | (SEQ ID NO:33) |
| ELFIN 1 | 2957144 | 1 | LTTQQIDLQGPGPWGFRLVGRKDFEQPL |
| | | | AISRVTPGSKAALANLCIGDVITAIDGE |
| | | | NTSNMTHLEAQNRIKGCTDNLTLTVARS |
| | | | EHKVWSPLVTNSS |
| | | | (SEQ ID NO:34) |
| ENIGMA | 561636 | 1 | IFMDSFKVVLEGPALPWGFRLQGGKDFN |
| | | | VPLSISRLTPGGKAAQAGVAVGDWVLSI |
| | | | DGENAGSLTHIEAQNKIRACGERLSLGL |
| | | | SRAQPV |
| | | | (SEQ ID NO:35) |
| ERBIN | 8923908 | 1 | QGHELAKQEIRVRVEKDPELGFSISGGV |
| | | | GGRGNPFRPDDDGIFVTRVQPEGPASKL |
| | | | LQPGDKIIQANGYSFINIEHGQAVSLLK |
| | | | TFQNTVELIIVREVSS |
| | | | (SEQ ID NO:36) |
| EZRIN | 3220018 | 1 | QMSADAAAGAPLPRLCCLEKGPNGYGFH |
| Binding | | | LHGEKGKLGQYIRLVEPGSPAEKAGLLA |
| Protein 50 | | | GDRLVEVNGENVEKETHQQVVSRIRAAL |
| | | | NAVRLLVVDPETDEQLQKLGVQVREELL |
| | | | RAQEAPGQAEPPAAAEVQGAGNENEPRE |
| | | | ADKSHPEQRELRNSS |
| | | | (SEQ ID NO:37) |
| EZRIN | 3220018 | 2 | IQQRELRPRLCTMKKGPSGYGFNLHSDK |
| Binding | | | SKPGQFIRSVDPDSPAEASGLRAQDRIV |
| Protein 50 | | | EVNGVCMEGKQHGDVVSALIRAGGDETK |
| | | | LLVVDRETDEFFKNSS |
| | | | (SEQ ID NO:38) |
| FLJ00011 | 10440352 | 1 | KNPSGELKTVTLSKMKQSLGISISGGIE |
| | | | SKVQPMVKIEKIFPGGAAFLSGALQAGF |
| | | | ELVAVDGENLEQVTHQRAVDTIRRAYRN |
| | | | KAREPMELVVRVPGPSPRPSPSD |
| | | | (SEQ ID NO:39) |
| FLJ11215 | 11436365 | 1 | EGHSHPRVVELPKTEEGLGFNIMGGKEQ |
| | | | NSPIYISRIIPGGIADRHGGLKRGDQLL |
| | | | SVNGVSVEGEHHEKAVELLKAAQGKVKL |
| | | | VVRYTPKVLEEME |
| | | | (SEQ ID NO:40) |
| FLJ12428 | BC012040 | 1 | PGAPYARKTFTIVGDAVGWGFVVRGSKP |
| | | | CHIQAVDPSGPAAAAGMKVCQFVVSVNG |
| | | | LNVLHVDYRTVSNLILTGPRTIVMEVME |
| | | | ELEC |
| | | | (SEQ ID NO:41) |
| FLJ12615 | 10434209 | 1 | GQYGGETVKIVRIEKARDIPLGATVRNE |
| | | | MDSVIISRIVKGGAAEKSGLLHEGDEVL |
| | | | EINGIEIRGKDVNEVFDLLSDMHGTLTF |
| | | | VLIPSQQIKPPPA |
| | | | (SEQ ID NO:42) |
| FLJ20075 | 7019938 | 1 | ILAHVKGIEKEVNVYKSEDSLGLTITDN |
| Semcap2 | | | GVGYAFIKRIKDGGVIDSVKTICVGDHI |
| | | | ESINGENIVGWRHYDVAKKLKELKKEEL |
| | | | FTMKLIEPKKAFEI |
| | | | (SEQ ID NO:43) |
| FLJ21687 | 10437836 | 1 | KPSQASGHFSVELVRGYAGFGLTLGGG |
| | | | RDVAGDTPLAVRGLLKDGPAQRCGRLE |
| | | | VGDLVLHINGESTQGLTHAQAVERIRA |
| | | | GGPQLHLVIRRPLETHPGKPRGV |
| | | | (SEQ ID NO:44) |
| FLJ31349 | AK055911 | 1 | PVMSQCACLEEVHLPNIKPGEGLGMYI |
| | | | KSTYDGLHVITGTTENSPADRSQKIHA |
| | | | GDEVIQVNQQTVVGWQLKNLVKKLREN |
| | | | PTGVVLLLKKRPTGSFNFTPEFIVTD |
| | | | (SEQ ID NO:45) |
| FLJ32798 | AK057360 | 1 | LDDEEDSVKIIRLVKNREPLGATIKKD |
| | | | EQTGAHVARIMRGGAADRSGLIHVGDE |
| | | | LREVNGIPVEDKRPEEIIQILAQSQGA |
| | | | ITFKIIPGSKEETPSNSS |
| | | | (SEQ ID NO:46) |
| GoRASP1 | NM031899 | 1 | MGLGVSAEQPAGGAEGFHLHGVQENSP |
| | | | AQQAGLEPYFDFIITIGHSRLNKENDT |
| | | | LKALLKANVEKPVKLEVFNMKTMRVRE |
| | | | VEVVPSNMWGGQGLLGASVRFCSFRRA |
| | | | SE |
| | | | (SEQ ID NO:47) |
| GoRASP1 | NM031899 | 2 | RASEQVWHVLDVEPSSPAALAGLRPYT |
| | | | DYVVGSDQILQESEDFFTLIESHEGKP |
| | | | LKLMVYNSKSDSCREVTVTPNAAWGGE |
| | | | GSLGCGIGYGYLHRIPTQ |
| | | | (SEQ ID NO:48) |
| GoRASP2 | 13994253 | 1 | MGSSQSVEIPGGGTEGYHVLRVQENSP |
| | | | GHRAGLEPFFDFIVSINGSRLNKDNDT |
| | | | LKDLLKANVEKPVKMLIYSSKTLELRE |
| | | | TSVTPSNLWGGQGLLGVSIRFCSFDGA |
| | | | NE |
| | | | (SEQ ID NO:49) |
| GoRASP2 | 13994253 | 2 | NENVWHVLEVESNSPAALAGLRPHSDY |
| | | | IIGADTVMNESEDLFSLIETHEAKPLK |
| | | | LYVYNTDTDNCREVIITPNSAWGGEGS |
| | | | LGCGIGYGYLHRIPTR |
| | | | (SEQ ID NO:50) |
| GRIP 1 | 4539083 | 1 | VVELMKKEGTTLGLTVSGGIDKDGKPR |
| | | | VSNLRQGGIAARSDQLDVGDYIKAVNG |
| | | | INLAKFRHDEIISLLKNVGERVVLEVE |
| | | | YE |
| | | | (SEQ ID NO:51) |
| GRIP 1 | 4539083 | 2 | RSSVIFRTVEVTLHKEGNTFGFVIRGG |
| | | | AHDDRNKSRPVVITCVRPGGPADREGT |
| | | | IKPGDRLLSVDGIRLLGTTHAEAMSIL |
| | | | KQCGQEAALLIEYDVSVMDSVATASGN |
| | | | SS |
| | | | (SEQ ID NO:52) |
| GRIP 1 | 4539083 | 3 | HVATASGPLLVEVAKTPGASLGVALTI |
| | | | SMCCNKQVIVIDKIKSASIADRCGALH |
| | | | VGDHILSIDGTSMEYCTLAEATQFLAN |
| | | | TTDQVKLEILPHHQTRLALKGPNSS |
| | | | (SEQ ID NO:53) |
| GRIP 1 | 4539083 | 4 | HVATASGPLLVEVAKTPGASLGVALTT |
| | | | SMCCNKQVIVIDKIKSASIADRCGALH |
| | | | VGDHILSIDGTSMEYCTLAEATQFLAN |
| | | | TTDQVKLEILPHHQTRLALKGPNSS |
| | | | (SEQ ID NO:54) |
| GRIP 1 | 4539083 | 5 | AESVIPSSGTFHVKLPKKHNVELGITI |
| | | | SSPSSRKPGDPLVISDIKKGSVAHRTG |
| | | | TLELGDKLLAIDNIRLDNCSMEDAVQI |
| | | | LQQCEDLVKLKIRKDEDNSD |
| | | | (SEQ ID NO:55) |
| GRIP 1 | 4539083 | 6 | IYTVELKRYGGPLGITISGTEEPFDPI |
| | | | IISSLTKGGLAERTGAIHIGDRILAIN |
| | | | SSSLKGKPLSEAIHLLQMAGETVILKI |
| | | | KKQTDAQSA |
| | | | (SEQ ID NO:56) |
| GRIP 1 | 4539083 | 7 | IMSPTPVELHKVTLYKDSDMEDFGFSV |
| | | | ADGLLEKGVYVKNIRPAGPGDLGGLKP |
| | | | YDRLLQVNHVRTRDFDCCLVVPLIAES |
| | | | GNKLDLVISRNPLA |
| | | | (SEQ ID NO:57) |
| GTPase | 2389008 | 1 | LSRGCETRELALPRDGQGRLGFEVDAE |
| Activating | | | GFVTHVERFTFAETAGLRPGARLLRVC |
| Enzyme | | | GQTLPSLRPEAAAQLLRSAPKVCVTVL |
| | | | PPDESGRPRNSS |
| | | | (SEQ ID NO:58) |
| Guanine | 6650765 | 1 | CSVMIFEVVEQAGAIILEDGQELDSWY |
| Exchange | | | VILNGTVEISHPDGKVENLFMGNSFGI |
| Factor | | | TPTLDKQYMHGIVRTKVDDCQFVCIAQ |
| | | | QDYWRILNHVEKNTHKVEEEGEIVMVH |
| | | | EFIVTD |
| | | | (SEQ ID NO:59) |
| HEMBA | 10436367 | 1 | LENVIAKSLLIKSNEGSYGFGLEDKNK |
| 1000505 | | | VPIIKLVEKGSNAEMAGMEVGKKIFAI |
| | | | NGDLVFMRPFNEVDCFLKSCLNSRKPL |
| | | | RVLVSTKP |
| | | | (SEQ ID NO:60) |
| HEMBA | 10436367 | 2 | PRETVKIPDSADGLGFQIRGFGPSVVH |
| 1000505 | | | AVGRGTVAAAAGLHPGQCIIKVNGINV |
| | | | SKETHASVIAHVTACRKYRRPTKQDSI |
| | | | QNSS |
| | | | (SEQ ID NO:61) |
| HEMBA | 7022001 | 1 | EDFCYVFTVELERGPSGLGMGLIDGMH |
| 1003117 | | | THLGAPGLYIQTLLPGSPAAADGRLSL |
| | | | GDRIILEVNGSSLLGLGYLRAVDLIRH |
| | | | GGKKMRFLVAKSDVETAKKI |
| | | | (SEQ ID NO:62) |
| HSPC227 | 7106843 | 1 | NNELIQFLPRTITLKKPPGAQLGFNIR |
| | | | GGKASQLGIFISKVIPDSDAHRAGLQE |
| | | | GDQVLAVNDVDFQDIEHSKAVEILKTA |
| | | | REISMRVRFFPYNYHRQKE |
| | | | (SEQ ID NO:63) |
| HTRA3 | AY040094 | 1 | LTEFQDKQIKDWKKRFIGIRMRTITPS |
| | | | LVDELKASNPDFPEVSSGIYVQEVAPN |
| | | | SPSQRGGIQDGDIIVKVNGRPLVDSSE |
| | | | LQEAVLTESPLLLEVRRGNDDLLFSNS |
| | | | S |
| | | | (SEQ ID NO:64) |
| HTRA4 | AL576444 | 1 | HKKYLGLQMLSLTVPLSEELKMHYPDF |
| | | | PDVSSGVYVCKVVEGTAAQSSGLRDHD |
| | | | VIVNINGKPITTTTDVVKALDSDSLSM |
| | | | AVLRGKDNLLLTVNSS |
| | | | (SEQ ID NO:65) |
| INADL | 2370148 | 1 | IWQIEYIDIERPSTGGLGFSVVALRSQ |
| | | | NLGKVDIFVKDVQPGSVADRDQRLKEN |
| | | | DQILAINHTPLDQNISHQQAIALLQQT |
| | | | TGSLRLIVAREPVHTKSSTSSSE |
| | | | (SEQ ID NO:66) |
| INADL | 2370148 | 2 | LPETVCWGHVEEVELINDGSGLGFGIV |
| | | | GGKTSGVVVRTIVPGGLADRDGRLQTG |
| | | | DHILKIGGTNVQGMTSEQVAQVLRNCG |
| | | | NSVRMLVARDPAGDISVTNSS |
| | | | (SEQ ID NO:67) |
| INADL | 2370148 | 3 | PGSDSSLFETYNVELVRKDGQSLGIRI |
| | | | IVGYVGTSHTGEASGIYVKSIIPGSAA |
| | | | YHNGHIQVNDKIVAVDGVNIQGFANHD |
| | | | VVEVLRNAGQVVHLTLVRRKTSSSTSR |
| | | | IHRD |
| | | | (SEQ ID NO:68) |
| INADL | 2370148 | 4 | NSDDAELQKYSKLLPIHTLRLGVEVDS |
| | | | FDGHHYISSIVSGGPVDTLGLLQPEDE |
| | | | LLEVNGMQLYGKSRREAVSFLKEVPPP |
| | | | FTLVCCRRLFDDEAS |
| | | | (SEQ ID NO:69) |
| INADL | 2370148 | 5 | LSSPEVKIVELVKDCKGLGFSILDYQD |
| | | | PLDPTRSVIVIRSLVADGVAERSGGLL |
| | | | PGDRLVSVNEYCLDNTSLAEAVEILKA |
| | | | VPPGLVHLGICKPLVEFIVTD |
| | | | (SEQ ID NO:70) |
| INADL | 2370148 | 6 | PNFSHWGPPRIVEIFREPNVSLGISIV |
| | | | VGQTVIKRLKNGEELKGIFIKQVLEDS |
| | | | PAGKTNALKTGDKILEVSGVDLQNASH |
| | | | SEAVEAIKNAGNPVVFIVQSLSSTPRV |
| | | | IPNVHNKANSS |
| | | | (SEQ ID NO:71) |
| INADL | 2370148 | 7 | PGELHIIELEKDKNGLGLSLAGNKDRS |
| | | | RMSIFVVGINPEGPAAADGRMRIGDEL |
| | | | LEINNQILYGRSHQNASAIIKTAPSKV |
| | | | KLVFIRNEDAVNQMANSS |
| | | | (SEQ ID NO:72) |
| INADL | 2370148 | 8 | PATCPIVPGQEMIIEISKGRSGLGLSI |
| | | | VGGKDIPLNAIVIHEVYEEGAAARDGR |
| | | | LWAGDQILEVNGVDLRNSSHEEAITAL |
| | | | RQTPQKVRLVVY |
| | | | (SEQ ID NO:73) |
| KIAA0147 | 1469875 | 1 | ILTLTILRQTGGLGISIAGGKGSTPYK |
| Vartul | | | GDDEGIFISRVSEEGPAARAGVRVGDK |
| | | | LLEVNGVALQGAEHHEAVEALRGAGTA |
| | | | VQMRVWRERMVEPENAEFIVTD |
| | | | (SEQ ID NO:74) |
| KIAA0147 | 1469875 | 2 | PLRQRHVACLARSERGLGFSIAGGKGS |
| Vartul | | | TPYRAGDAGIFVSRIAEGGAAHRAGTL |
| | | | QVGDRVLSINGVDVTEARHDHAVSLLT |
| | | | AASPTIALLLEREAGG |
| | | | (SEQ ID NO:75) |
| KIAA0147 | 1469875 | 3 | ILEGPYPVEEIRLPRAGGPLGLSIVGG |
| Vartul | | | SDHSSHPFGVQEPGVFISKVLPRGLAA |
| | | | RSGLRVGDRILAVNGQDVRDATHQEAV |
| | | | SALLRPCLELSLLVRRDPAEFIVTD |
| | | | (SEQ ID NO:76) |
| KIAA0147 | 1469875 | 4 | RELCIQKAPGERLGISIRGGARGHAGN |
| Vartul | | | PRDPTDEGIFISKVSPTGAAGRDGRLR |
| | | | VGLRLLEVNQQSLLGLTHGEAVQLLRS |
| | | | VGDTLTVLVCDGFEASTDAALEVS |
| | | | (SEQ ID NO:77) |
| KIAA0303 | 2224546 | 1 | PHQPIVIHSSGKNYGFTIRAIRVYVGD |
| MAST4 | | | SDIYTVHHIVWNVEEGSPACQAGLKAG |
| | | | DLITHINGEPVHGLVHTEVIELLLKSG |
| | | | NKVSITTTPF |
| | | | (SEQ ID NO:78) |
| KIAA0313 | 7657260 | 1 | HLRLLNIACAAKAKRRLMTLTKPSREA |
| | | | PLPFILLGGSEKGFGIFVDSVDSGSKA |
| | | | TEAGLKRGDQILEVNGQNFENIQLSKA |
| | | | MEILRNNTHLSITVKTNLFVFKELLTR |
| | | | LSEEKRNGALPNSS |
| | | | (SEQ ID NO:79) |
| KIAA0316 | 6683123 | 1 | IPPAPRKVEMRRDPVLGFGFVAGSEKP |
| | | | VVVRSVTPGGPSEGKLIPGDQIVMIND |
| | | | EPVSAAPRERVIDLVRSCKESILLTVI |
| | | | QPYPSPKSEFIVTD |
| | | | (SEQ ID NO:80) |
| KIAA0340 | 2224620 | 1 | LNKRTTMPKDSGALLGLKVVGGKMTDL |
| | | | GRLGAFITKVKKGSLADVVGHLRAGDE |
| | | | VLEWNGKPLPGATNEEVYNIILESKSE |
| | | | PQVEIIVSRPIGDIPRIHRD |
| | | | (SEQ ID NO:81) |
| KIAA0380 | 2224700 | 1 | QRCVIIQKDQHGFGFTVSGDRIVLVQS |
| | | | VRPGGAAMKAGVKEGDRIIKVNGTMVT |
| | | | NSSHLEVVKLIKSGAYVALTLLGSS |
| | | | (SEQ ID NO:82) |
| KIAA0382 | 7662087 | 1 | ILVQRCVIIQKDDNGFGLTVSGDNPVF |
| | | | VQSVKEDGAAMRAGVQTGDRIIKVNGT |
| | | | LVTHSNIILEVVKLIKSGSYVALTVQG |
| | | | RPPGNSS |
| | | | (SEQ ID NO:83) |
| KIAA0440 | 2662160 | 1 | SVEMTLRRNGLGQLGFHVNYEGIVADV |
| | | | EPYGYAWQAGLRQGSRLVEICKVAVAT |
| | | | LSHEQMIDLLRTSVTVKVVIIPPHD |
| | | | (SEQ ID NO:84) |
| KIAA0545 | 14762850 | 1 | LKVMTSGWETVDMTLRRNGLGQLGFHV |
| | | | KYDGTVAEVEDYGFAWQAGLRQGSRLV |
| | | | EICKVAVVTLTHDQMIDLLRTSVTVKV |
| | | | VIIPPFEDGTPRRGW |
| | | | (SEQ ID NO:85) |
| KIAA0559 | 3043641 | 1 | HYIFPHARIKITRDSKDHTVSGNGLGI |
| | | | RIVGGKEIPGHSGEIGAYIAKILPGGS |
| | | | AEQTGKLMEGMQVLEWNGIPLTSKTYE |
| | | | EVQSIISQQSGEAEICVRLDLNML |
| | | | (SEQ ID NO:86) |
| KIAA0561 | 3043645 | 1 | LCGSLRPPIVIHSSGKKYGFSLRAIRV |
| MAST3 | | | YMGDSDVYTVHHVVWSVEDGSPAQEAG |
| | | | LRAGDLITHINGESVLGLVHMDVVELL |
| | | | LKSGNKISLRTTALENTSIKVGNSS |
| | | | (SEQ ID NO:87) |
| KIAA0613 | 3327039 | 1 | SYSVTLTGPGPWGFRLQGGKDFNMPLT |
| | | | ISRITPGSKAAQSQLSQGDLVVAIDGV |
| | | | NTDTMTHLEAQNKIKSASYNLSLTLQK |
| | | | SKNSS |
| | | | (SEQ ID NO:88) |
| KIAA0751 | 12734165 | 1 | TLNEEHSHSDKHPVTWQPSKDGDRLIG |
| RIM2 | | | RILLNKRLKDGSVPRDSGAMLGLKVVG |
| | | | GKMTESGRLCAFITKVKKGSLADTVGH |
| | | | LRPGDEVLEWNGRLLQGATFEEVYNII |
| | | | LESKPEPQVELVVSRPIG |
| | | | (SEQ ID NO:89) |
| KIAA0807 | 3882334 | 1 | ISALGSMRPPIIIHRAGKKYGFTLRAI |
| MAST2 | | | RVYMGDSDVYTVHHMVWHVEDGGPASE |
| | | | AGLRQGDLIIHVNGEPVHGLVHTEVVE |
| | | | LILKSGNKVAISTTPLENSS |
| | | | (SEQ ID NO:90) |
| KIAA0858 | 4240204 | 1 | FSDMRISINQTPGKSLDFGFTIKWDIP |
| | | | GIFVASVEAGSPAEFSQLQVDDEIIAI |
| | | | NNTKFSYNDSKEWEEAMAKAQETGHLV |
| | | | MDVRRYGKAGSPE |
| | | | (SEQ ID NO:91) |
| KIAA0902 | 4240292 | 1 | QSAHLEVIQLANIKPSEGLGMYIKSTY |
| | | | DGLHVITGTTENSPADRCKKIHAGDEV |
| | | | IQVNIKIQTVVGWQLKNLVNALREDPS |
| | | | GVILTLKKRPQSMLTSAPA |
| | | | (SEQ ID NO:92) |
| KIAA0967 | 4589577 | 1 | ILTQILIPVRHTVKIDKDTLLQDYGFH |
| | | | ISESLPLTVVAVTAGGSAHGKLFPGDQ |
| | | | ILQMNNEPAEDLSWERAVDILREAEDS |
| | | | LSITVVRCTSGVPKSSNSS |
| | | | (SEQ ID NO:93) |
| KIAA0973 | 4589589 | 1 | GLRSPITIQRSGKKYGFTLRAIRVYMG |
| MAST1 | | | DTDVYSVHHIVWHVEEGGPAQEAGLCA |
| | | | GDLITHVNGEPVHGMVHPEVVELILKS |
| | | | GNKVAVTTTPFE |
| | | | (SEQ ID NO:94) |
| KIAA1095 | 5889526 | 1 | QGEETKSLTLVLHRDSGSLGFNIIGGR |
| SEMCAP3 | | | PSVDNHDGSSSEGIFVSKIVDSGPAAK |
| | | | EGGLQIHDRIIEVNGRDLSRATHDQAV |
| | | | EAFKTAKEPIVVQVLRRTPRTKMFTP |
| | | | (SEQ ID NO:95) |
| KIAA1095 | 5889526 | 2 | QEMDREELELEEVDLYRMNSQDKLGLT |
| SEMCAP3 | | | VCYRTDDEDDIGIYISEIDPNSIAAKD |
| | | | GRIREGDRIIQINGIEVQNREEAVALL |
| | | | TSEENKNFSLLIARPELQLD |
| | | | (SEQ ID NO:96) |
| KIAA1202 | 6330421 | 1 | RSFQYVPVQLQGGAPWGFTLKGGLEHC |
| | | | EPLTVSKIEDGGKAALSQKMRTGDELV |
| | | | NINGTPLYGSRQEALILIKGSFRILKL |
| | | | IVRRRNAIPVS |
| | | | (SEQ ID NO:97) |
| KIAA1222 | 6330610 | 1 | ILEKLELFPVELEKDEDGLGISIIGMG |
| | | | VGADAGLEKLGIFVKTVTEGGAAQRDG |
| | | | RIQVNDQIVEVDGISLVGVTQNFAATV |
| | | | LRNTKGNVRFVIGREKPGQVS |
| | | | (SEQ ID NO:98) |
| KIAA1284 | 6331369 | 1 | KDVNVYVNPKKLTVIKAKEQLKLLEVL |
| | | | VGIIHQTKWSWRRTGKQGDGERLVVHG |
| | | | LLPGGSAMKSGQVLIGDVLVAVNDVDV |
| | | | TTENIERVLSCIPGPMQVKLTFENAYD |
| | | | VKRET |
| | | | (SEQ ID NO:99) |
| KIAA1389 | 7243158 | 1 | TRGCETVEMTLRRNGLGQLGFHVNFEG |
| | | | IVADVEPFGFAWKAGLRQGSRLVEICK |
| | | | VAVATLTHEQMIDLLRTSVTVKVVIIQ |
| | | | PHDDGSPRR |
| | | | (SEQ ID NO:100) |
| KIAA1415 | 7243210 | 1 | VENILAKRLLILPQEEDYGFDIEEKNK |
| | | | AVVVKSVQRGSLAEVAGLQVGRKIYSI |
| | | | NEDLVFLRPFSEVESILNQSFCSRRPL |
| | | | RLLVATKAKEIIKIP |
| | | | (SEQ ID NO:101) |
| KIAA1526 | 5817166 | 1 | PDSAGPGEVRLVSLRRAKAHEGLGFSI |
| | | | RGGSEHGVGIYVSLVEPGSLAEKEGLR |
| | | | VGDQILRVNDKSLARVTHAEAVKALKG |
| | | | SKKLVLSVYSAGRIPGGYVTNHIEFIV |
| | | | TD |
| | | | (SEQ ID NO:102) |
| KIAA1526 | 5817166 | 2 | LQGGDEKKVNLVLGDGRSLGLTIRGGA |
| | | | EYGLGIYITGVDPGSEAEGSGLKVGDQ |
| | | | ILEVNWRSFLNILHDEAVRLLKSSRHL |
| | | | ILTVKDVGRLPHARTTVDEEFIVTD |
| | | | (SEQ ID NO:103) |
| KIAA1526 | 5817166 | 3 | WTSGAHVHSGPCEEKCGHPGHRQPLPR |
| | | | IVTIQRGGSAHNCGQLKVGHVILEVNG |
| | | | LILRGKEHREAARIIAEAFKTKDRDYI |
| | | | DFLDSL |
| | | | (SEQ ID NO:104) |
| KIAA1620 | 10047316 | 1 | ELRRAELVEIIVETEAQTGVSGINVAG |
| | | | GGKEGIFVRELREDSPAARSLSLQEGD |
| | | | QLLSARVFFENFKYEDALRLLQCAEPY |
| | | | KVSFCLKRTVPTGDLALRP |
| | | | (SEQ ID NO:105) |
| KIAA1634 | 10047344 | 1 | PSQLKGVLVRASLKKSTMGFGFTIIGG |
| MAGI3 | | | DRPDEFLQVKNVLKDGPAAQDGKIAPG |
| | | | DVIVDINGNCVLGHTHADVVQMFQLVP |
| | | | VNQYVNLTLCRGYPLPDDSED |
| | | | (SEQ ID NO:106) |
| KIAA1634 | 10047344 | 2 | ASSGSSQPELVTIPLIKGPKGFGFAIA |
| MAGI3 | | | DSPTGQKVKMILDSQWCQGLQKGDIIK |
| | | | EIYHQNVQNLTHLQVVEVLKQFPVGAD |
| | | | VPLLILRGGPPSPTKTAKM |
| | | | (SEQ ID NO:107) |
| KIAA1634 | 10047344 | 3 | LYEDKPPLTNTFLISNPRTTADPRILY |
| MAGI3 | | | EDKPPNTKDLDVFLRKQESGFGFRVLG |
| | | | GDGPDQSIYIGAIIPLGAAEKDGRLRA |
| | | | ADELMCIDGIPVKGKSHKQVLDLMYIT |
| | | | AARNGHVLLTVRRKIFYGEKQPEDDSG |
| | | | SPGIHRELT |
| | | | (SEQ ID NO:108) |
| KIAA1634 | 10047344 | 4 | PAPQEPYDVVLQRKENEGFGFVILTSK |
| MAGI3 | | | NKPPPGVIPHKIGRVIEGSPADRCGKL |
| | | | KVGDHISAVNGQSIVELSHDNIVQLIK |
| | | | DAGVTVTLTVIAEEEHHGPPS |
| | | | (SEQ ID NO:109) |
| KIAA1634 | 10047344 | 5 | QNLGCYPVELERGPRGFGFSLRGGKEY |
| MAGI3 | | | NMGLFILRLAEDGPAIKDGRIHVGDQI |
| | | | VEINGEPTQGITHTRAIELIQAGGNKV |
| | | | LLLLRPGTGLIPDHGLA |
| | | | (SEQ ID NO:110) |
| KIAA1719 | 1267982 | 0 | ITVVELIKKEGSTLGLTISGGTDKDGK |
| | | | PRVSNLRPGGLAARSDLLNIGDYIRSV |
| | | | NGIHLTRLRHDEIITLLKNVGERVVLE |
| | | | VEY |
| | | | (SEQ ID NO:111) |
| KIAA1719 | 1267982 | 1 | ILDVSLYKEGNSFGFVLRGGAHEDGHK |
| | | | SRIPLVLTYVRPGGPADREGSLKVGDR |
| | | | LLSVDGIPLHGASHATALATLRQCSHE |
| | | | ALFQVEYDVATP |
| | | | (SEQ ID NO:112) |
| KIAA1719 | 1267982 | 2 | IHTVANASGPLMVEIVKTPGSALGISL |
| | | | TTTSLRNKSVITIDRIKPASVVDRSGA |
| | | | LHPGDHILSIDGTSMEHCSLLEATKLL |
| | | | ASISEKVRLEILPVPQSQRPL |
| | | | (SEQ ID NO:113) |
| KIAA1719 | 1267982 | 3 | IQIVHTETTEVVLCGDPLSGFGLQLQG |
| | | | GIFATETLSSPPLVCFIEPDSPAERCG |
| | | | LLQVGDRVLSINGIATEDGTMEEANQL |
| | | | LRDAALAHKVVLEVEFDVAESV |
| | | | (SEQ ID NO:114) |
| KIAA1719 | 1267982 | 4 | IQFDVAESVIPSSGTFHVKLPKKRSVE |
| | | | LGITISSASRKRGEPLIISDIKKGSVA |
| | | | HRTGTLEPGDKLLAIDNIRLDNCPMED |
| | | | AVQILRQCEDLVKLKIRKDEDN |
| | | | (SEQ ID NO:115) |
| KIAA1719 | 1267982 | 5 | IQTTGAVSYTVELKRYGGPLGITISGT |
| | | | EEPFDPIVISGLTKRGLAERTGAIHVG |
| | | | DRILAINNVSLKGRPLSEAIHLLQVAG |
| | | | ETVTLKIKKQLDR |
| | | | (SEQ ID NO:116) |
| KIAA1719 | 1267982 | 6 | ILEMEELLLPTPLEMHKVTLHKDPMRH |
| | | | DFGFSVSDGLLEKGVYVHTVRPDGPAH |
| | | | RGGLQPFDRVLQVNIIVRTRDFDCCLA |
| | | | VPLLAEAGDVLELIISRKPHTAHSS |
| | | | (SEQ ID NO:117) |
| LIM | 12734250 | 1 | MALTVDVAGPAPWGFRITGGRDFHTPI |
| Mystique | | | MVTKVAERGKAKDADLRPGDIIVAING |
| | | | ESAEGMLHAEAQSKIRQSPSPLRLQLD |
| | | | RSQATSPGQT |
| | | | (SEQ ID NO:118) |
| LIM Protein | 3108092 | 1 | SNYSVSLVGPAPWGFRLQGGKDFNMPL |
| | | | TISSLKDGGKAAQANVRIGDVVLSIDG |
| | | | TNAQGMTHLEAQNKIKGCTGSLNMTLQ |
| | | | RAS |
| | | | (SEQ ID NO:119) |
| LIMK1 | 4587498 | 1 | TLVEHSKLYCGHCYYQTVVTPVIEQIL |
| | | | PDSPGSHLPHTVTLVSIPASSHGKRGL |
| | | | SVSIDPPHGPPGCGTEHSHTVRVQGVD |
| | | | PGCMSPDVKNSIHVGDRILEINGTPIR |
| | | | NVPLDEIDLLIQETSRLLQLTLEHD |
| | | | (SEQ ID NO:120) |
| LIMK2 | 1805593 | 1 | PYSVTLISMPATTEGRRGFSVSVESAC |
| | | | SNYATTVQVKEVNRMHISPNNRNAIHP |
| | | | GDRILEINGTPVRTLRVEEVEDAISQT |
| | | | SQTLQLLIEHD |
| | | | (SEQ ID NO:121) |
| LIM-RIL | 1085021 | 1 | IHSVTLRGPSPWGFRLVGRDFSAPLTI |
| | | | SRVHAGSKASLAALCPGDLIQAINGES |
| | | | TELMTHLEAQNRIKGCHDHLTLSVSRP |
| | | | E |
| | | | (SEQ ID NO:122) |
| LU-1 | U52111 | 1 | VCYRTDDEEDLGIYVGEVNPNSIAAKD |
| | | | GRIREGDRIIQINGVDVQNREEAVAIL |
| | | | SQEENTNISLLVARPESQLA |
| | | | (SEQ ID NO:123) |
| MAGI1 | 3370997 | 1 | PSELKGKFIHTKLRKSSRGFGFTVVGG |
| | | | DEPDEFLQIKSLVLDGPAALDGKMETG |
| | | | DVIVSVNDTCVLGHTHAQVVKIFQSIP |
| | | | IGASVDLELCRGYPLPFDPDGIHRD |
| | | | (SEQ ID NO:124) |
| MAGI1 | 3370997 | 2 | PATQPELITVHIVKGPMGFGFTIADSP |
| | | | GGGGQRVKQIVDSPRCRGLKEGDLIVE |
| | | | VNKKNVQALTHNQVVDMLVECPKGS |
| | | | (SEQ ID NO:125) |
| MAGI1 | 3370997 | 3 | IPATQPELITVHIVKGPMGFGFTIADS |
| | | | PGGGGQRVKQIVDSPRCRGLKEDLIVE |
| | | | VNKKNVQALTHNQVVDMLVECPKGSEV |
| | | | TLLVQRGGNSSZ |
| | | | (SEQ ID NO:126) |
| MAGI1 | 3370997 | 4 | QATQEQDFYTVELERGAKGFGFSLRGG |
| | | | REYNMDLYVLRLAEDGPAERCGKMRIG |
| | | | DEILEINGETTKNMKHSRAIELIKNGG |
| | | | RRVRLFLKRG |
| | | | (SEQ ID NO:127) |
| MAGI1 | 3370997 | 5 | PGVVSTVVQPYDVEIRRGENEGFGFVI |
| | | | VSSVSRPEAGTIFAGNACVAMPHKIGR |
| | | | IIEGSPADRCGKLKVGDRILAVNGCSI |
| | | | TNKSHSDIVNLIKEAGNTVTLRIIPGD |
| | | | ESSNAEFIVTD |
| | | | (SEQ ID NO:128) |
| MGC5395 | BC012477 | 1 | PDYQEQDIFLWRKETGFGFRILGGNEP |
| | | | GEPIYIGHIVPLGAADTDGRLRSGDEL |
| | | | ICVDGTPVIGKSHQLVVQLMQQAAKQG |
| | | | HVNLTVRRKVVFAVPKTENSS |
| | | | (SEQ ID NO:129) |
| MINT1 | 2625024 | 1 | PAKMEKEETTRELLLPNWQGSGSHGLT |
| | | | IAQRDDGVFVQEVTQNSPAARTGVVKE |
| | | | GDQIVGATIYFDNLQSGEVTQLLNTMG |
| | | | HHTVGLKLHRKGDRSPNSS |
| | | | (SEQ ID NO:130) |
| MINT1 | 2625024 | 2 | SENCKdVFIEKQKGEILGVVIVESGWG |
| | | | SILPTVIIANMMHGGPAEKSGKLNIGD |
| | | | QIMSINGTSLVGLPLSTCQSIIKGLKN |
| | | | QSRVKLNIVRCPPVNSS |
| | | | (SEQ ID NO:131) |
| MINT3 | 3169808 | 1 | LRCPPVTTVLIRRPDLRYQLGFSVQNG |
| | | | IICSLMRGGIAERGGVRVGHRIIEING |
| | | | QSVVATPHEKIVHILSNAVGEIHMKTM |
| | | | PAAMYRLLNSS |
| | | | (SEQ ID NO:132) |
| MINT3 | 3169808 | 2 | HNGDLDHFSNSDNCREVHLEKRRGEGL |
| | | | GVALVESGWGSLLPTAVIANLLHGGPA |
| | | | ERSGALSIGDRLTAINGTSLVGLPLAA |
| | | | CQAAVRETKSQTSVTLSIVHCPPVT |
| | | | (SEQ ID NO:133) |
| MPP1 | 189785 | 1 | PVTTAIIHRPHAREQLGFCVEDGIICS |
| | | | LLRGGIAERGGIRVGHRIIEBNGQSVV |
| | | | ATPHARIIELLTEAYGEVHIKTMPAAT |
| | | | YRLLTGNSS |
| | | | (SEQ ID NO:134) |
| MPP2 | 939884 | 1 | RKVRLIQFEKVTEEPMGITLKLNEKQS |
| | | | CTVARILHGGMIHRQGSLHVGDEILEI |
| | | | NGTNVTNHSVDQLQKAMKETKGMISLK |
| | | | VIPNQ |
| | | | (SEQ ID NO:135) |
| MPP3 | 1022812 | 1 | PVPPDAVRMVGIRKTAGEHLGVTFRVE |
| | | | GGELVIARILHGGMVAQQGLLHVGDII |
| | | | KEVNGQPVGSDPRALQELLRNASGSVI |
| | | | LKILPNYQ |
| | | | (SEQ ID NO:136) |
| MUPP1 | 2104784 | 1 | NIDEDFDEESVKIVRLVKNKEPLGATI |
| | | | RRDEHSGAVVVARIMRGGAADRSGLVH |
| | | | VGDELREVNGIAVLHKRPDEISQILAQ |
| | | | SQGSITLKIIPATQEEDR |
| | | | (SEQ ID NO:137) |
| MUPP1 | 2104784 | 2 | QGRHVEVFELLKPPSGGLGFSVVGLRS |
| | | | ENRGELGIFVQEIQEGSVAHRDGRLKE |
| | | | TDQILAINGQALDQTITHQQAISILQK |
| | | | AKDTVQLVIARGSLPQLV |
| | | | (SEQ ID NO:138) |
| MUPP1 | 2104784 | 3 | PVHWQHMETIELVNDGSGLGFGIIGGK |
| | | | ATGVIVKTILPGGVADQHGRLCSGDHI |
| | | | LKIGDTDLAGMSSEQVAQVLRQCGNRV |
| | | | KLMIARGAIEERTAPT |
| | | | (SEQ ID NO:139) |
| MUPP1 | 2104784 | 4 | QESETFDVELTKNVQGLGITIAGYIGD |
| | | | KKLEPSGIFVKSITKSSAVEHDGRIQI |
| | | | GDQIIAVDGTNLQGFTNQQAVEVLRII |
| | | | TGQTVLLTLMRRGMKQEA |
| | | | (SEQ ID NO:140) |
| MUPP1 | 2104784 | 5 | LNYEIVVAHVSKFSENSGLGISLEATV |
| | | | GHHFIRSVLPEGPVGHSGKLFSGDELL |
| | | | EVNGITLLGENHQDVVNILKELPIEVT |
| | | | MVCCRRTVPPT |
| | | | (SEQ ID NO:141) |
| MUPP1 | 2104784 | 6 | WEAGIQHIELEKGSKGLGFSILDYQDP |
| | | | IDPASTVIIIRSLVPGGIAEKDGRLLP |
| | | | GDRLMFVNDVNLENSSLEEAVEALKGA |
| | | | PSGTVRIGVAKPLPLSPEENSS |
| | | | (SEQ ID NO:142) |
| MUPP1 | 2104784 | 7 | RNVSKESFERTINIAKGNSSLGMTVSA |
| | | | NKDGLGMIVRSIIHGGAISRDGRIAIG |
| | | | DCILSINEESTISVTNAQARAMLRRHS |
| | | | LIGPDIKITYVPAEHLEE |
| | | | (SEQ ID NO:143) |
| MUPP1 | 2104784 | 8 | LNWNQPRRVELWREPSKSLGISIVGGR |
| | | | GMGSRLSNGEVMRGIFIKHVLEDSPAG |
| | | | KNGTLKPGDRIVEVDGMDLRDASHEQA |
| | | | VEAIRKAGNPVVFMVQSIINRPRKSPL |
| | | | PSLL |
| | | | (SEQ ID NO:144) |
| MUPP1 | 2104784 | 9 | LTGELHMIELEKGHSGLGLSLAGNKDR |
| | | | SRMSVFIVGIDPNGAAGKDGRLQIADE |
| | | | LLEINGQILYGRSHQNASSIIKCAPSK |
| | | | VKIIFIRNKDAVNQ |
| | | | (SEQ ID NO:145) |
| MUPP1 | 2104784 | 10 | LSSFKNVQHLELPKDQGGLGIALISEE |
| | | | DTLSGVIIKSLTEHGVAATDGRLKVGD |
| | | | QILAVDDEIVVGYPIEKFISLLKTAKM |
| | | | IVKLTIHAENPDSQ |
| | | | (SEQ ID NO:146) |
| MUPP1 | 2104784 | 11 | LPGCETTIEISKGRTGLGLSIVGGSDT |
| | | | LLGALHIHEVYEEGAACKDGRLWAGDQ |
| | | | ILEVNGIDLRKATHDEAINVLRQTPQR |
| | | | VRLTLYRDEAPYKE |
| | | | (SEQ ID NO:147) |
| MUPP1 | 2104784 | 12 | KEEEVCDTLTIELQKKPGKGLGLSIVG |
| | | | KRNDTGVFVSDIVKGGIADADGRLMQG |
| | | | DQILMVNGEDVRNATQEAVAALLKCSL |
| | | | GTVTLEVGRIKAGPFHS |
| | | | (SEQ ID NO:148) |
| MUPP1 | 2104784 | 13 | LQGLRTVEMKKGPTDSLGISIAGGVGS |
| | | | PLGDVPIFIAMMHPIGVAAQTQKLRVG |
| | | | DRIVTICGTSTEGMTHTQAVNLLKNAS |
| | | | GSIEMQVVAGGDVSV |
| | | | (SEQ ID NO:149) |
| NeDLG | 10863920 | 1 | LGPPQCKSITLERGPDGLGFSIVGGYG |
| | | | SPHGDLPIYVKTVFAKGAASEDGRLKR |
| | | | GDQIIAVNGQSLEGVTHEEAVAILKRT |
| | | | KGTVTLMVLS |
| | | | (SEQ ID NO:150) |
| NeDLG | 10863920 | 2 | IQYEEIVLERGNSGLGFSIAGGIDNPH |
| | | | VPDDPGIFITKIIPGGAAAMDGRLGVN |
| | | | DCVLRVNEVEVSEVVHSRAVEALKEAG |
| | | | PVVRLVVRRRQN |
| | | | (SEQ ID NO:151) |
| NeDLG | 10863920 | 3 | ITLLKGPKGLGFSIAGGIGNQHIPGDN |
| | | | SIYITKIIEGGAAQKDGRLQIGDRLAV |
| | | | NNTNLQDVRHEEAVASLKNISDMVYLK |
| | | | VAKPGSLE |
| | | | (SEQ ID NO:152) |
| Neurabin II | AJ401189 | 1 | ILLHKGSTGLGFNIVGGEDGEGIFVSF |
| | | | ILAGGPADLSGELRRGDRILSVNGVNL |
| | | | RNATHEQAAAALKRAGQSVTIVAQYRP |
| | | | EEYSRLFESKIHDLREQMMNSSMSSGS |
| | | | GSLRTSEKRSLE |
| | | | (SEQ ID NO:153) |
| NOS1 | 642525 | 1 | CVERLELFPVELEKDSEGLGISIIGMG |
| | | | AGADMGLEKLGIFVKTVTEGGAARDGR |
| | | | IQVNDLLVEVDGTSLVGVTQSFAASVL |
| | | | RNTKGRVREMIGRERPGEQSEVAQRIH |
| | | | RD |
| | | | (SEQ ID NO:154) |
| novel PDZ | 7228177 | 1 | IQPNVISVRLFKRKVGGLGFLVKERVS |
| gene | | | KPPVIISDLIRGGAAEQSGLIQAGDII |
| | | | LAVNGRPLVDLSYDSALEVLRGIASET |
| | | | HVVLILRGP |
| | | | (SEQ ID NO:155) |
| novel PDZ | 7228177 | 2 | QANSDESDIIHSVRVEKSPAGRLGFSV |
| gene | | | RGGSEHGLGIFVSKVEEGSSAERAGLC |
| | | | VGDKITEVNGLSLESTTMGSAVKVLTS |
| | | | SSRLHMMVRRMGRVPGIKFSKEKNSS |
| | | | (SEQ ID NO:156) |
| Novel | 1621243 | 1 | PSDTSSEDGVRRIVHLYTTSDDFCLGF |
| Serine | | | NIRGGKEFGLGIYVSKVDHGGLAEENG |
| Protease | | | IKVGDQVLAANGVRFDDISHSQAVEVL |
| | | | KGQTHIMLTIKETGRYPAYKEMNSS |
| | | | (SEQ ID NO:157) |
| Numb | AK056823 | 1 | KIKKFLTESHDRQAKGKAITKKKYIGI |
| Binding | | | RMMSLTSSKAKELKDRHRDFPDVISGA |
| Protein | | | YIIEVIPDTPAEAGGLKENDVIISING |
| | | | QSVVSANDVSDVIKRESTLNMVVRRGN |
| | | | EDIMITV |
| | | | (SEQ ID NO:158) |
| Numb | AK056823 | 2 | PDGEITSIKINRVDPSESLSIRLVGGS |
| Binding | | | ETPLVHIIIQHIYRDGVIARDGRLLPG |
| Protein | | | DIILKVNGMDISNVPHNYAVRLLRQPC |
| | | | QVLWLTVMREQKFRSRNSS |
| | | | (SEQ ID NO:159) |
| Numb | AK056823 | 3 | HRPRDDSFHVILNKSSPEEQLGIKLVR |
| Binding | | | KVDEPGVFIFNVLDGGVAYRHGQLEEN |
| Protein | | | DRVLAINGHDLRYGSPESAAHLIQASE |
| | | | RRVHLVVSRQVRQRSPENSS |
| | | | (SEQ ID NO:160) |
| Outer | 7023825 | 1 | PTITCHEKVVNIQKDPGESLGMTVAGG |
| Membrane | | | ASHREWDLPIYVISVEPGGVISRDGRI |
| | | | KTGDILLNVDGVELTEVSRSEAVALLK |
| | | | RTSSSIVLKALEVKEYEPQEFIV |
| | | | (SEQ ID NO:161) |
| p55T | 12733367 | 1 | LLTEEEINLTRGPSGLGFNIVGGTDQQ |
| | | | YVSNDSGIYVSRIKENGAAALDGRLQE |
| | | | GDKILSVNGQDLKNLLHQDAVDLFRNA |
| | | | GYAVSLRVQHRLQVQNGIHS |
| | | | (SEQ ID NO:162) |
| PAR3 | 8037914 | 1 | PVDAIRILGIHKRAGEPLGVTFRVENN |
| | | | DLVIARILHGGMIDRQGLLHVGDIIKE |
| | | | VNGHEVGNNPKELQELLKNISGSVTLK |
| | | | ILPSYRDTITPQQ |
| | | | (SEQ ID NO:163) |
| PAR3 | 8037914 | 2 | PNFSLDDMVKLVEVPNDGGPLGIHVVP |
| | | | FSARGGRTLGLLVKRLEKGGKAENLFR |
| | | | ENDCIVRINDGDLRNRRFEQAQHMFRQ |
| | | | AMRTPHWFHVVPAANKEQYEQ |
| | | | (SEQ ID NO:164) |
| PAR3 | 8037914 | 3 | GKRLNIQLKKGTEGLGFSITSRDVTIG |
| | | | GSAPIYVKNILPRGAAIQDGRLKAGDR |
| | | | LIEVNGVDLVGKSQEEVVSLLRSTKME |
| | | | GTVSLLVFRQEDA |
| | | | (SEQ ID NO:165) |
| PAR3-like | AF428250 | 1 | PREFLTFEVPLNDSGSAGLGVSVKGNR |
| | | | SKENHADLGIFVKSIINGGAASKDGRL |
| | | | RVNDQLIAVNGESLLGKTNQDAMETLR |
| | | | RSMSTEGNKRGMIQLIVASRISKCNEL |
| | | | KSNSS |
| | | | (SEQ ID NO:166) |
| PAR3-like | AF428250 | 2 | PRTKDTLSDMTRTVEISGEGGPLGIHV |
| | | | VPFFSSLSGRILGLFIRGIEDNSRSKR |
| | | | EGLFHENECIVKINNVDLVDKTFAQAQ |
| | | | DVFRQAMKSPSVLLHVLPPQNR |
| | | | (SEQ ID NO:167) |
| PAR3-like | AF428250 | 3 | SNKINAKKIKIDLKKGPEGLGFTVVTR |
| | | | DSSIHGPGPIFVKNILPKGAAIKDGRL |
| | | | QSGDRILEVNGRDVTGRTQEELVAMLR |
| | | | STKQGETASLVIARQEGH |
| | | | (SEQ ID NO:168) |
| PAR6 | 2613011 | 1 | ITSEQLTFEIPLNDSGSAGLGVSLKGN |
| | | | KSRETGTDLGIFIKSIIHGGAAFKDGR |
| | | | LRMNDQLIAVNGESLLGKSNIIEAMET |
| | | | LRRSMSMEGNIRGMIQLVILRRPERP |
| | | | (SEQ ID NO:169) |
| PAR6 | 13537116 | 1 | PETHRRVRLHKHGSDRPLGFYIRDGMS |
| BETA | | | VRVAPQGLERVPGIFISRLVRGGLAES |
| | | | TGLLAVSDEILEVNGIEVAGKTLDQVT |
| | | | DMMVANSHNLIVTVKPANQRNNVNSS |
| | | | (SEQ ID NO:170) |
| PAR6 | 13537118 | 1 | PVSSIIDVDILPETHRRVRLYKYGTEK |
| GAMMA | | | PLGFYIRDGSSVRVTPHGLEKVPGIFI |
| | | | SRLVPGGLAQSTGLLAVNDEVLEVNGI |
| | | | EVSGKSLDQVTDMMIANSRNLIITVRP |
| | | | ANQRNNRIHRD |
| | | | (SEQ ID NO:171) |
| PDZ-73 | 5031978 | 1 | IDVDLVPETHRRVRLHRHGCEKPLGFY |
| | | | IRDGASVRVTPHGLEKVPGIFISRMVP |
| | | | GGLAESTGLLAVNDEVLEVNGIEVAGK |
| | | | TLDQVTDMMIANSHLIVTVKPANQRNN |
| | | | VV |
| | | | (SEQ ID NO:172) |
| PDZ-73 | 5031978 | 2 | RSRKLKEVRLDRLHPEGLGLSVRGGLE |
| | | | FGCGLFISHLIKGGQADSVGLQVGDEI |
| | | | VRINGYSISSCTHEEVINLIRTKKTVS |
| | | | IKVRHIGLIPVKSSPDEFH |
| | | | (SEQ ID NO:173) |
| PDZ-73 | 5031978 | 3 | IPGNRENKEKKVFISLVGSRGLGCSIS |
| | | | SGPIQKPGIFISHVKPGSLSAEVGLIG |
| | | | DQIVEVNGVDFSNLDHKEAVNVLKSSR |
| | | | SLTISIVAAAGRELFMTDEF |
| | | | (SEQ ID NO:174) |
| PDZK1 | 2944188 | 1 | PEQIMGKDVRLLRIKKEGSLDLALEGG |
| | | | VDSPIGKVVVSAVYERGAAERHGGIVK |
| | | | GDEIMAIINGKIVTDYTLAEADAALQK |
| | | | AWNQGGDWIDLVVAVCPPKEYDD |
| | | | (SEQ ID NO:175) |
| PDZK1 | 2944188 | 2 | LTSTFNPRECKLSKQEGQNYGFFLRIE |
| | | | Q KDTEGHLVRVVEKCSPAEKAGLQDGDR |
| | | | VLRINGVFVDKEEHMQVVDLVRKSGNS |
| | | | VTLLVLDGDSYEKQGSPGIHRD |
| | | | (SEQ ID NO:176) |
| PDZK1 | 2944188 | 3 | RLCYLVKEGGSYGFSLKTVQGKKGVYM |
| | | | TDITPQGVAMRAGVLADDHLIEVNGEN |
| | | | VEDASHEEVVEKVKKSGSRVMFLLVDK |
| | | | ETDKREFIVTD |
| | | | (SEQ ID NO:177) |
| PDZK1 | 2944188 | 4 | QFKRETASLKLLPHQPRIVEMKKGSNG |
| | | | YGFYLRAGSEQKGQIIKDIDSGSPAEE |
| | | | AGLKNNDLVVAVNGESVETLDHDSVVE |
| | | | MIRKGGDQTSLLVVDKETDNMYRLAEF |
| | | | IVTD |
| | | | (SEQ ID NO:178) |
| PICK1 | 4678411 | 1 | PDTTEEVDHKPKLCRLAKGENGYGFHL |
| | | | NAIRGLPGSFIKEVQKGGPADLAGLED |
| | | | EDVIIEVNGVNVLDEPYEKVVDRIQSS |
| | | | GKNVTLLVZGKNSS |
| | | | (SEQ ID NO:179) |
| PIST | 98374330 | 1 | PTVPGKVTLQKDAQNLIGISIGGGAQY |
| | | | CPCLYIVQVFDNTPAALDGTVAAGDEI |
| | | | TGVNGRSIKGKTKVEVAKMIQEVKGEV |
| | | | TIHYNKLQ |
| | | | (SEQ ID NO:180) |
| prIL16 | 1478492 | 1 | SQGVGPIRKVLLLKEDHEGLGISITGG |
| | | | KEHGVPILISEIHPGQPADRCGGLHVG |
| | | | DAILAVNGVNLRDTKHKEAVTILSQQR |
| | | | GEIEFEVVYVAPEVDSD |
| | | | (SEQ ID NO:181) |
| prIL16 | 1478492 | 2 | IHVTILHKEEGAGLGFSLAGGADLENK |
| | | | VITVHRVFPNGLASQEGTIQKGNEVLS |
| | | | INGKSLKGTTHHDALAILRQAREPRQA |
| | | | VIVTRKLTPEEFIVTD |
| | | | (SEQ ID NO:182) |
| PSAP | 6409315 | | TAEATVCTVTLEKMSAGLGFSLEGGKG |
| | | | SLHGDKPLTINRIFKGAASEQSETVQP |
| | | | GDEILQLGGTAMQGLTRFEAWNIIKAL |
| | | | PDGPVTIVIRRKSLQSK |
| | | | (SEQ ID NO:183) |
| PSD95 | 3318652 | 1 | IREAKYSGVLSSIGKIFKEEGLLGFFV |
| | | | GLIPHLLGDVVFLWGCNLLAHFINAYL |
| | | | VDDSVSDTPGGLGNDQNPGSQFSQALA |
| | | | IRSYTKFVMGIAVSMLTYPFLLVGDLM |
| | | | AVNNCGLQAGLPPYSPVFKSWIHCWKY |
| | | | LSVQGQLFRGSSLLFRRVSSGSCFALE |
| | | | (SEQ ID NO:184) |
| PSD95 | 3318652 | 2 | LEYEeITLERGNSGLGFSIAGGTDNPH |
| | | | IGDDPSIFITKIIPGGAAAQDGRLRVN |
| | | | DSILFVNEVDVREVTHSAAVEALKEAG |
| | | | SIVRLYVMRRKPPAENSS |
| | | | (SEQ ID NO:185) |
| PSD95 | 3318652 | 3 | HVMRRKPPAEKVMEIKLIKGPKGLGFS |
| | | | IAGGVGNQHIPGDNSIYVTKIIEGGAA |
| | | | HKDGRLQIGDKILAVNSVGLEDVMHED |
| | | | AVAALKNTYDVVYLKVAKPSNAYLLEF |
| | | | IVTD |
| | | | (SEQ ID NO:186) |
| PTN-3 | 179912 | 1 | RERHTPRTEANCDHRGSTGLGFNIVGG |
| | | | EDGEGILSPLSWPGALQTSVGSCGRGT |
| | | | RSCRSTVWTSEMPAMSRLPLP |
| | | | (SEQ ID NO:187) |
| PTN-4 | 190747 | 1 | QNDNGDSYLVLIRITPDEDGKFGFNLK |
| | | | GGVDQKMPLVVSRINPESPADTCIPKL |
| | | | NEGDQIVLINGRDISEHTHDQVVMFIK |
| | | | ASRESHSRELALVIRRRAVRS |
| | | | (SEQ ID NO:188) |
| PTPL1 | 515030 | 1 | IRMKPDENGRFGFNVKGGYDQKMPVIV |
| | | | SRVAPGTPADLCVPRLNEGDQVVLIIS |
| | | | IGRDIAEHTHDQVVLFIKASCERHSGE |
| | | | LMLLVRPNA |
| | | | (SEQ ID N:189) |
| PTPL1 | 515030 | 2 | PEREITLVNLKKDAKYGLGFQIIGGEK |
| | | | MGRLDLGIFISSVAPGGPADFHGCLKP |
| | | | GDRLISVNSVSLEGVSHHAAIEILQNA |
| | | | PEDVTLVISQPKEKISKVPSTPVHL |
| | | | (SEQ ID NO:190) |
| PTPL1 | 515030 | 3 | GDIFEVELAKNDNSLGISVTGGVNTSV |
| | | | RHGGIYVKAVIPQGAAESDGRIHKGDR |
| | | | VLAVNGVSLEGATHKQAVETLRNTGQV |
| | | | VHLLLEKGQSPTSK |
| | | | (SEQ ID NO:191) |
| PTPL1 | 515030 | 4 | TEENTFEVKLFKNSSGLGFSFSREDNL |
| | | | IPEQINASIVRVKKLFAGQPAAESGKI |
| | | | DVGDVILKVNGASLKGLSQQEVISALR |
| | | | GTAPEVFLLLCRPPPGVLPEIDT |
| | | | (SEQ ID NO:192) |
| PTPL1 | 515030 | 5 | ELEVELLITLIKSEKASLGFTVTKGNQ |
| | | | RIGCYVHDVIQDPAKSDGRLKPGDRLI |
| | | | KVNDTDVTNMTHTDAVNLLRAASKTVR |
| | | | LVIGRVLELPRIPMLPH |
| | | | (SEQ ID NO:193) |
| RGS12 | 3290015 | 1 | MLPHLLPDITLTCNKEELGFSLCGGHD |
| | | | SLYQVVYISDINPRSVAAIEGNLQLLD |
| | | | VIHYVNGVSTQGMTLEEVNRALDMSLP |
| | | | SLVLKATRINDLPV |
| | | | (SEQ ID NO:194) |
| RGS3 | 18644735 | 1 | RPSPPRVRSVEVARGRAGYGFTLSGQA |
| | | | PCVLSCVMRGSPADFVGLRAGDQILAV |
| | | | NEINVKKASHEDVVKLIGKCSGVLHMV |
| | | | IAEGVGRFESCSNSS |
| | | | (SEQ ID NO:195) |
| Rho-GAP | NM020824 | 1 | LCSERRYRQITIPRGKDGFGFTICCDS |
| 10 | | | PVRVQAVDSGGPAERAGLQQLDTVLQL |
| | | | NERPVEHWKCVELAHEIRSCPSEIILL |
| | | | VWRMVPQVKPGIHRD |
| | | | (SEQ ID NO:196) |
| Rhophilin- | 14279408 | 1 | SEDETFSWPGPKTVTLKRTSQGFGFTL |
| like | | | RHFIVYPPESAIQFSYKDEENGNRGGK |
| | | | QRNRLEPMDTIFVKQVKEGGPAFEAGL |
| | | | CTGDRIIKVNGESVIGKTYSQVIALIQ |
| | | | NSDTTLELSMPKDED |
| | | | (SEQ ID NO:197) |
| Serine | 2738914 | 1 | SAKNRWRLVGPVHLTRGEGGFGLTLRG |
| Protease | | | DSPVLIAAVIPGSQAAAAGLKEGDYIV |
| | | | SVNGQPCRWWRHAEVVTELKAAGEAGA |
| | | | SLQVVSLLPSSRLPSI |
| | | | (SEQ ID NO:198) |
| Shank 2 | 6049185 | 1 | RGEKKNSSSGISGSQRRYIGVMMLTLS |
| | | | PSILAELQLREPSFPDVQHGVLIHKVI |
| | | | LGSPARAGLRPGDVILAIGEQMVQNAE |
| | | | DVYEAVRTQSQLAVQIRRGRETLTLYV |
| | | | NSS |
| | | | (SEQ ID NO:199) |
| Shank 3 | * | 1 | LEEKTVVLQKKDNEGFGFVLRGAKADT |
| | | | PIEEFTPTPAFPALQYLESVDEGGVAW |
| | | | QAGLRTGDFLIEVNNENVVKVGHRQVV |
| | | | NMIRQGGNHLVLKVVTVTRNLDPDDNS |
| | | | S |
| | | | (SEQ ID NO:200) |
| Shroom | 18652858 | 1 | SDYVIDDKVAVLQKRDHEGFGFVLRGA |
| | | | KAETPIEEFTPTPAFPALQYLESVDVE |
| | | | GVAWRAGLRTGDFLIEVNGVNVVKVGH |
| | | | KQVVALIRQGGNRLVMKVVSVTRKPEE |
| | | | DG |
| | | | (SEQ ID NO:201) |
| Similar to | 14286261 | 1 | ISNTATKGRYIYLEAFLEGGAPWGFTL |
| GRASP65 | | | KGGLEHGEPLIISKVEEGGKADTLSSK |
| | | | LQAGDEVVHINEVTLSSSRKEAVSLVK |
| | | | GSYKTLRLVVRRDVCTDPGHAD |
| | | | (SEQ ID NO:202) |
| Similar to | 14286261 | 2 | MGLGVSAEQPAGGAEGFHLHGVQENSP |
| GRASP65 | | | AQQAGLEPYFDFIITIGHSRLNKENDT |
| | | | LKALLKANVEKPVKLEVFNMKTMRVRE |
| | | | VEVVPSNMWGGQGLLGASVRFCSFRRA |
| | | | SE |
| | | | (SEQ ID NO:203) |
| Similar to | BC036755 | 1 | RASEQVWHVLDVEPSSPAALAGLRPYT |
| Ligand of | | | DYVVGSDQILQESEDFFTLIESHEGKP |
| Numb px2 | | | LKLMVYNSKSDSCRESGMWHWLWVSTP |
| | | | DPNSAPQLPQEATWHPTTFCSTTWCPT |
| | | | T |
| | | | (SEQ ID NO:204) |
| Similar to | BC036755 | 2 | IQPLSLPEGEITTIEIHRSNPYIQLGI |
| Ligand of | | | SIVGGNETPLINIVIQEVYRDGVIARD |
| Numb px2 | | | GRLLAGDQILQVNNYNISNVSHNYARA |
| | | | VLSQPCNTLHLTVLRERRFGNRAH |
| | | | (SEQ ID NO:205) |
| Similar to | BC036755 | 3 | SNSPREEIFQVALHKRDSGEQLGIKLV |
| Ligand of | | | RRTDEPGVFILDLLEGGLAAQDGRLSS |
| Numb px2 | | | NDRVLAINGHDLKYGTPELAAQIIQAS |
| | | | GERVNLTIARPGKPQ |
| | | | (SEQ ID NO:206) |
| Similar to | BC036755 | 4 | QCVTCQEKHITVKKEPHESLGMTVAGG |
| Ligand of | | | RGSKSGELPIFVTSVPPHGCLARDGRI |
| Numb px2 | | | KRGDVLLNINGIDLTNLSHSEAVAMLK |
| | | | ASAASPAVALKALEVQIVEEAT |
| | | | (SEQ ID NO:207) |
| Similar to | 21595065 | 1 | PSTLHSCHDIVLRRSYLGSWGFSIVGG |
| PTP | | | YEENHTNQPFFIKTIVLGTPAYYDGRL |
| Homolog | | | KCGDMIVAVNGLSTVGMSHSALVPMLK |
| | | | EQRNKVTLTVICWPGS |
| | | | (SEQ ID NO:208) |
| SIP1 | 2047327 | 1 | SVTDGPKFEVKLKKNANGLGFSFVQME |
| | | | KESCSHLKSDLVRIKRLFPGQPAEENG |
| | | | AIAAGDIILAVNGRSTEGLIFQEVLH |
| | | | (SEQ ID NO:209) |
| SIP1 | 2047327 | 2 | LLRGAPQEVTLLLCRPPPGA |
| | | | (SEQ ID NO:210) |
| SITAC-18 | 8886071 | 1 | QPEPLRPRLCRLVRGEQGYGFHLHGEK |
| | | | GRRGQFIRRVEPGSPAEAAALRAGDRL |
| | | | VEVNGVNVEGETHHQVVQRIKAVEGQT |
| | | | RLLVVDQETDEELRRRNSS |
| | | | (SEQ ID NO:211) |
| SITAC-18 | 8886071 | 2 | PLRELRPRLCHLRKGPQGYGFNLHSDK |
| | | | SRPGQYIRSVDPGSPAARSGLRAQDRL |
| | | | IEVNGQNVEGLRHAEVVASIKAREDEA |
| | | | RLLVVDPETDEHFKRNSS |
| | | | (SEQ ID NO:212) |
| SNPCIIA | 20809633 | 1 | PGVREIHLCKDERGKTGLRLRKVDQGL |
| | | | FVQLVQANTPASLVGLRFGDQLLQIDG |
| | | | RDCAGWSSHKAHQVVKKASGDKIVVVV |
| | | | RDRPFQRTVTM |
| | | | (SEQ ID NO:213) |
| SNPCIIA | 20809633 | 3 | PFQRTVTMHKDSMGHVGFVIKKGKIVS |
| | | | LVKGSSAARNGLLTNHYVCEVDGQNVI |
| | | | GLKDKKIMEILATAGNVVTLTIIPSVI |
| | | | YEHIVEFIV |
| | | | (SEQ ID NO:214) |
| SNPCIIA | 20809633 | 4 | SLERPRFCLLSKEEGKSFGFHLQQELG |
| | | | RAGHVVCRVDPGTSAQRQGLQEGDRIL |
| | | | AVNNDVVEHEDYAVVVRRIRASSPRVL |
| | | | LTVLARHAHDVARAQ |
| | | | (SEQ ID NO:215) |
| Shank1 | 7025450 | 1 | ISLPTKPRCLHLEKGPQGFGFLLREEK |
| | | | GLDGRPGQFLWEVDPGLPAKKAGMQAG |
| | | | DRLVAVAGESVEGLGHEETVSRIQGQG |
| | | | SCVSLTVVDPEADR |
| | | | (SEQ ID NO:216) |
| SYNTENIN | 2795862 | 1 | IPSVPLGSRQCFLYPGPGGSYGFRLSC |
| | | | VASGPRLFISQVTPGGSAARAGLQVGD |
| | | | VILEVNGYPVGGQNDLERLQQLPEAEP |
| | | | PLCLKLAARSLRGLE |
| | | | (SEQ ID NO:217) |
| SYNTENIN | 2795862 | 2 | LKEKTVLLQKKDSEGFGFVLRGAKAQT |
| | | | PIEEFTPTPAFPALQYLESVDEGGVAW |
| | | | RAGLRMGDFLIEVNGQNVVKVGHRQVV |
| | | | NMIRQGGNTLMVKVVMVTRHPDMDEAV |
| | | | QNSS |
| | | | (SEQ ID NO:218) |
| Syntrophin | 1145727 | 1 | LEIKQGIREVILCKDQDGKIGLRLKSI |
| 1 alpha | | | DNGIFVQLVQANSPASLVGLRFGDQVL |
| | | | QINGENCAGWSSDKAHKVLKQAFGEKI |
| | | | TMRIHRD |
| | | | (SEQ ID NO:219) |
| Syntrophin | 476700 | 1 | LRDRPFERTITMHKDSTGHVGFIFKNG |
| beta 2 | | | KITSIVKDSSAARNGLLTEHNICEING |
| | | | QNVIGLKDSQIADILSTSGTVVTITMP |
| | | | AFIFEHMNSS |
| | | | (SEQ ID NO:220) |
| Syntrophin | 9507162 | 1 | QRRRVTVRKADAGGLGISIKGGRENKM |
| gamma 1 | | | PILISKIFKGLAADQTEALFVGDAIIL |
| | | | SVNGEDLSSATHDEAVQVLKKTGKEVV |
| | | | LEVKYMKDVSPYFK |
| | | | (SEQ ID NO:221) |
| Syntrophin | 9507164 | 1 | PVRRVVKQEAGGLGISIKGGRENRMPI |
| gamma 2 | | | LISKIFPGLAADQSRALRLGDAILSVN |
| | | | GTDLRQATHDQAVQALKRAGKEVLLEV |
| | | | KFIRE |
| | | | (SEQ ID NO:222) |
| TAX2-like | 3253116 | 1 | EPFYSGERTVTIRRQTVGGFGLSIKGG |
| protein | | | AEHNIPVVVSKISKEQRAELSGLLFIG |
| | | | DAILQINGINVRKCRHEEVVQVLRNAG |
| | | | EEVTLTVSFLKRAPAFLKLP |
| | | | (SEQ ID NO:223) |
| TIAM 1 | 4507500 | 1 | SHQGRNRRTVTLRRQPVGGLGLSIKGG |
| | | | SEHNVPVVIISKIFEDQAADQTGMLFV |
| | | | GDAVLQVNGIHVENATHEEVVHLLRNA |
| | | | GDEVTITVEYLREAPAFLK |
| | | | (SEQ ID NO:224) |
| TIAM 2 | 6912703 | 1 | RGETKEVEVTKTEDALGLTITDNGAGY |
| | | | AFIKRIKEGSIINRIEAVCVGDSIEAI |
| | | | NDHSIVGCRHYEVAKMLRELPKSQPFT |
| | | | LRLVQPKRAF |
| | | | (SEQ ID NO:225) |
| TIP1 | 2613001 | 1 | HSIHIEKSDTAADTYGFSLSSVEEDGI |
| | | | RRLYVNSVKETGLASKKGLKAGDEILE |
| | | | IINNRAADALNSSMLKDFLSQPSLGLL |
| | | | VRTYPELE |
| | | | (SEQ ID NO:226) |
| TIP2 | 2613003 | 1 | PLNVYDVQLTKTGSVCDFGFAVTAQVD |
| | | | ERQHLSRIFISDVLPDGLAYGEGLRKG |
| | | | NEIMTLNGEAVSDLDLKQMEALFSEKS |
| | | | VGLTLIARPPDTKATL |
| | | | (SEQ ID NO:227) |
| TIP33 | 2613007 | 1 | QRVEIHKLRQGENLILGFSIGGGIDQD |
| | | | PSQNPFSEDKTDKGIYVTRVSEGGPAE |
| | | | IAGLQIGDKIMQVNGWDMTMVTHDQAR |
| | | | KRLTKRSEEVVRLLVTRQSLQK |
| | | | (SEQ ID NO:228) |
| TIP43 | 2613011 | 1 | RKEVEVFKSEDALGLTITDNGAGYAFI |
| | | | KRIKEGSVIDHIHLISVGDMIEAINGQ |
| | | | SLLGCRHYEVARLLKELPRGRTFTLKL |
| | | | TEPRK |
| | | | (SEQ ID NO:229) |
| Unknown | | 1 | HSHPRVVELPKTDEGLGFNVMGGKEQN |
| PDZ gene | | | RGDQLLSVNGVSVEGEHHEKAVELLKA |
| | | | AKDSVKLVVRYTPKVL |
| | | | (SEQ ID NO:230) |
| X-11 beta | 3005559 | 1 | LSNQKRGVKVLKQELGGLGISIKGGKE |
| | | | NKMPILISKIFKGLAADQTQALYVGDA |
| | | | ILSVNGADLRDATHDEAVQALKRAGKE |
| | | | VLLEVKYMREATPYVKNSS |
| | | | (SEQ ID NO:231) |
| X-11 beta | 3005559 | 2 | QRSSIKTVELIKGNLQSVGLTLRLVQS |
| | | | TDGYAGHVIIETVAPNSPAAIADLQRG |
| | | | DRLIAIGGVKITSTLQVLKLIKQAGDR |
| | | | VLVYYERIPVGQSNQGA |
| | | | (SEQ ID NO:232) |
| ZO-1 | 292937 | 1 | IHFSNSENCKELQLEKHKGEILGVVVV |
| | | | ESGWGSILPTVILANMMNGGPAARSGK |
| | | | LSIGDQIMSINGTSLVGLPLATCQGII |
| | | | KGLKNQTQVKLNIVSCPPVTTVLIKRN |
| | | | SS |
| | | | (SEQ ID NO:233) |
| ZO-1 | 292937 | 2 | IPPVTTVLIKRPDLKYQLGFSVQNGII |
| | | | CSLMRGGIAERGGVRVGHRIIEINGQS |
| | | | VVATAHEKIVQALSNSVGEIHMKTMPA |
| | | | AMFRLLTGQENSS |
| | | | (SEQ ID NO:234) |
| ZO-1 | 292937 | 3 | IWEQHTVTLHRAPGFGFGIAISGGRDN |
| | | | PHFQSGETSIVISDVLKGGPAEGQLQE |
| | | | NDRVAMVNGVSMDNVEHAFAVQQLRKS |
| | | | GKNAKITIRRKKKVQIPNSS |
| | | | (SEQ ID NO:235) |
| ZO-2 | 12734763 | 1 | ISSQPAKPTKVTLVKSRKNEEYGLRLA |
| | | | SHIFVKEISQDSLAARDGNIQEGDVVL |
| | | | KINGTVTENMSLTDAKTLIERSKGKLK |
| | | | MVVQRDRATLLNSS |
| | | | (SEQ ID NO:236) |
| ZO-2 | 12734763 | 2 | IRMKLVKFRKGDSVGLRLAGGNDVGIF |
| | | | VAGVLEDSPAAKEGLEEGDQILRVNNV |
| | | | DFTNIIREEAVLFLLDLPKGEEVTILA |
| | | | QKKKDVFSN |
| | | | (SEQ ID NO:237) |
| ZO-2 | 12734763 | 3 | LIWEQYTVTLQKDSKRGFGIAVSGGRD |
| | | | NPHFENGETSIVISDVLPGGPADGLLQ |
| | | | ENDRVVMVNGTPMEDVLHSFAVQQLRK |
| | | | SGKVAAIVVKRPRKV |
| | | | (SEQ ID NO:238) |
| ZO-3 | 10092690 | 1 | RVLLMKSRANEEYGLRLGSQIFVKEMT |
| | | | RTGLATKDGNLHEGDIILKINGTVTEN |
| | | | MSLTDARKLEKSRGKLQLVVLRDS |
| | | | (SEQ ID NO:239) |
| ZO-3 | 10092690 | 2 | HAPNTKMVRFKKGDSVGLRLAGGNDVG |
| | | | IFVAGIQEGTSAEQEGLQEGDQILKVN |
| | | | TQDFRGLVREDAVLYLLEIPKGEMVTI |
| | | | LAQSRADVY |
| | | | (SEQ ID NO:240) |
| ZO-3 | 10092690 | 3 | IPGNSTIWEQHTATLSKDPRRGFGIAI |
| | | | SGGRDRPGGSMVVSDVVPGGPSAEGRL |
| | | | QTGDHIVMVNGVSMENATSAFAIQILK |
| | | | TCTKMANITVKRPRRIHLPAEFIVTD |
| | | | (SEQ ID NO:241) |

*No GI number for this PDZ domain containing protein as it was computer cloned using rat Shank3 sequence against human genomic clone AC000036 and in silico spliced together nucleotides 6400-6496, 6985-7109, 7211-7400 to create hypothetical human Shank3.

VI. Screening for Other PL-Binding Agents

PL binding agents suitable for use in a diagnostic assay include any agent that specifically binds to one or more PL motifs. Such agents can be identified using the same methods as disclosed in methods of screening for anti-viral and anti-bacterial agents. For example, agents can be identified using a protein containing a PL motif. Test compounds can be identified using any type of library, including expression libraries and small molecule libraries for example. A preferred source of test compounds for use in screening for therapeutics or therapeutic leads is a phage display library. See, e.g., Devlin, WO 91/18980; Key, B. K., et al., eds., Phage Display of Peptides and Proteins, A Laboratory Manual, Academic Press, San Diego, CA, 1996. Phage display is a powerful technology that allows one to use phage genetics to select and amplify peptides or proteins of desired characteristics from libraries containing 10⁸-10⁹ different sequences. Libraries can be designed for selected variegation of an amino acid sequence at desired positions, allowing bias of the library toward desired characteristics. Libraries are designed so that peptides are expressed fused to proteins that are displayed on the surface of the bacteriophage. The phage displaying peptides of the desired characteristics are selected and can be regrown for expansion. Since the peptides are amplified by propagation of the phage, the DNA from the selected phage can be readily sequenced facilitating rapid analyses of the selected peptides.

Phage encoding peptide inhibitors can be selected by selecting for phage that bind specifically to a PDZ domain protein and/or to a PL protein PL. Libraries are generated fused to proteins such as gene III that are expressed on the surface of the phage. The libraries can be composed of peptides of various lengths, linear or constrained by the inclusion of two Cys amino acids, fused to the phage protein or can also be fused to additional proteins as a scaffold. One can also design libraries biased toward the PL regions disclosed herein or biased toward peptide sequences obtained from the selection of binding phage from the initial libraries provide additional test inhibitor compound.

VII. Antibodies for Diagnostic and Therapeutic Uses

The PL protein, PL protein PL, PDZ and PDZ PL binding domain polypeptides of the invention are useful for generating antibodies for use in diagnostics and therapeutics. The antibodies can be polyclonal antibodies, distinct monoclonal antibodies or pooled monoclonal antibodies with different epitopic specificities. Monoclonal antibodies are made from antigen-containing fragments of the protein by standard procedures according to the type of antibody (see, e.g., Kohler, et al., Nature, 256:495, (1975); and Harlow & Lane, Antibodies, A Laboratory Manual (C.S.H.P., NY, 1988) Queen et al., Proc. Natl. Acad. Sci. USA 86:10029-10033 (1989) and WO 90/07861; Dower et al., WO 91/17271 and McCafferty et al., WO 92/01047 (each of which is incorporated by reference for all purposes). Phage display technology can also be used to mutagenize CDR regions of antibodies previously shown to have affinity for the peptides of the present invention. Some antibodies bind to an epitope present in one form of PL protein or PDZ protein but not others. For example, some antibodies bind to an epitope within the C-terminus PL site of PL protein. Those antibodies that bind to specific PL protein PL motifs can be classified as PL protein PL class-specific antibodies. Further, some antibodies bind to an epitope within the PDZ domain of a PDZ protein. Some antibodies specifically bind to a PDZ polypeptide such as that shown in Table 2 without binding to others. Some antibodies specifically bind to a PL motif on a PL protein such as that shown in Table 1. The antibodies can be purified, for example, by binding to and elution from a support to which the polypeptide or a peptide to which the antibodies were raised is bound.

The term “antibody” or “immunoglobulin” is used to include intact antibodies and binding fragments thereof. Typically, fragments compete with the intact antibody from which they were derived for specific binding to an antigen fragment including separate heavy chains, light chains Fab, Fab′ F(ab′)2, Fabc, and Fv. Fragments are produced by recombinant DNA techniques, or by enzymatic or chemical separation of intact immunoglobulins. The term “antibody” also includes one or more immunoglobulin chains that are chemically conjugated to, or expressed as, fusion proteins with other proteins. The term “antibody” also includes bispecific antibody. A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

The antibodies may be utilized as reagents (e.g., in pre-packaged kits) for prognosis and diagnosis of viral and bacterial infection and subtypes thereof. A variety of methods can be used to prognosticate and diagnose viral and bacterial infection.

B. Monoclonal Antibody Surrogates of PDZ Proteins

As shown above and in the examples, there are a wide variety of PDZ proteins that recognize and bind to the PL motif on the viral and bacterial PL proteins. Antibodies that recognize the same motif can also be used as surrogates of these PDZ proteins. Preferably, the PDZ protein is one of the following: AF6, AIPC, AIPC (PDZ #1), GORASP1 (PDZ #1), INADL (PDZ #3), KIAA0316, KIAA1284, EBP50 (PDZ #1), (Shank1; Shank2; Shank3; Syntenin; Magil (PDZ #1); Tip1; Mint1 (PDZ #1,2); Novel Serine Protease; MUPP1 (PDZ#3,7,9,1 1), MAST2, NSP, NOS1, PAR3 (PDZ #3), PAR3L(PDZ #3); PAR5beta, RiM2, Rhodophilin-like, SIP-1, SITAC-18(PDZ #2), SITAC-18(PDZ #1), SIP1, ZO-1 (PDZ #1), ZO-3 (PDZ #1), DVL3, DVL2 (PDZ #1), PTPL1 (PDZ#4), HEMBA 10003117, Pick1, or an analog or fragment. More preferably the antibodies mimic any PDZ protein that specifically recognizes the PL motifs for the following viruses and bacteria: RALL or RILL (SEQ ID NOS:242 and 243) for HIV-1 Env, FKNC, FKDC, YKNC, or YKDC (SEQ ID NOS:244, 245, 246 and 247) for HIV-1 Nef protein, IALL, LALL, or LTLL (SEQ ID NOS:248, 249 and 250) for HIV2 Env protein, EILA, GILA, or DILA (SEQ ID NOS:251, 252 and 253) for HIV-2 Vif protein, FTSA (SEQ ID NO:254) for Hepatitis B Protein X, WVYI (SEQ ID NO:255) for Hepatitis B S antigen, PASA, or PVSA (SEQ ID NOS:256 and 257) for Hepatitis C Capsid C protein, GVDA (SEQ ID NO:258) for Hepatitis C E1 protein, DVEL (SEQ ID NO:259) for RSV Nucleoprotein, QCKL, or QCRL (SEQ ID NOS:260 and 261) for Rotavirus A VP4 protein, YYRV, or YYRI (SEQ ID NOS:262 and 263) for Rotavirus A VP7 protein, QVGI, HIGI, QIGI, or RIGI (SEQ ID NOS:264, 265, 268 and 267) for Rotavirus A NSP2 protein, IKDL or IEDL (SEQ ID NOS:268 and 269) for Rotavirus A NSP5 protein, SSWA (SEQ ID NO:270) for M. tuberculosis ESXN protein, YTGF (SEQ ID N0271) for M. tuberculosis ESXS protein, and GMFA (SEQ ID NO:272) for M. tuberculosis ESAT-6 protein. The antibody surrogates that recognize specific PL protein PL motifs can be designated PL class-specific. For example an antibody that recognizes the PL motif, YTGF (SEQ ID NO:271) for M. tuberculosis ESXS protein can be designated ESXS PL class specific.

C. Mixture of Antibodies and Other Binding Agents

A mixture of antibodies and PDZ proteins (and/or aptamers) can be used in any of the assays. The PDZ proteins and antibodies can be used for identification of different sub-types of PL proteins, identification of virus and bacteria taught herein, and identification of pathogenic forms as compared to those that are less pathogenic. In some assays, the antibody(s) and PDZ protein(s) are mixed and administered together to a sample. In other assays, the antibody(s) and PDZ protein(s) are separated and allowed to bind to different samples for identification of two different subtypes or for confirmation of the identification of a subtype.

VIII. Aptamers

Aptamers are RNA or DNA molecules selected in vitro from vast populations of random sequence that recognize specific ligands by forming binding pockets. Allosteric ribozymes are RNA enzymes whose activity is modulated by the binding of an effector molecule to an aptamer domain, which is located apart from the active site. These RNAs act as precision molecular switches that are controlled by the presence or absence of a specific effector. Aptamers can bind to nucleic acids, proteins, and even entire organisms. Aptamers are different from antibodies, yet they mimic properties of antibodies in a variety of diagnostic formats. Thus, aptamers can be used instead of or in combination with antibodies and/or PDZ proteins to identify the presence of general and specific PL protein PL regions.

X. Diagnostic Tests

Diagnostic capture and detect reagents useful in assay methods for identifying bacteria and viruses are provided herein, including HIV, Hepatitis B, Hepatitis C, RSV, Rotavirus A, M. tuberculosis and their products in a variety of different types of biological samples. Representative assay formats useful for detecting these viruses and bacteria include enzyme-linked solid-phase absorbent assays, radiolabeled binding assays, fluorescence PDZ- and PL-binding assays, time-resolved PDZ and PL fluorescence assays, as well as, sandwich- and enzyme-cascade assay formats. Illustrative methods adaptable from the immunoassay art for use in the subject assays include homogeneous and heterogeneous assay formats; competitive and non-competitive assay formats; enzyme-linked solid phase assay formats, fluorescence assay formats, time resolved fluorescence assay formats, bioluminescent assay formats, cascade enzyme assays and the like.

In certain exemplary procedures, one or more PDZ proteins are used as capture agents to isolate one or more PL analytes from a biological sample. In other exemplary procedures, one or more PDZ proteins are conjugated with one or more signal generating compounds and used as detect reagents for identifying the presence or amount of one or more PL analytes in a biological sample. In yet other exemplary procedures, PL proteins and PL peptides are conjugated with signal generating compounds (PL-SGC) and used in competitive ligand inhibition assays, i.e., where the presence of a viral PL competes the binding of one or more PL-SGC to a PDZ. Preferably, the PDZ proteins are at least one of the PDZ proteins found to bind to viral and bacterial PL's in Table 1. Table 1 is subdivided into several boxes to illustrate which PDZ's bind to which PL protein. PDZ's shown in the same box as one or more PL motifs specifically bind to at least one of the PL motifs. For example, for either HIV-1 Env PL shown in column 4 of the table, a PDZ protein is chosen from column six of the same box containing HIV-1 Env. The PDZ proteins listed bind to one or both PL motifs in Env. For detecting the Env protein of HIV-1 any of the PDZ proteins in column 6 of the same box can be used because it is sufficient that the PDZ proteins bind to one PL motif in Env. For detecting the Nef protein of HIV-1 any of the PDZ proteins in column 6 of the same box as HIV-1 Nef can be used for one or both PL motifs in Table 1. For detecting the Env protein of HIV-2, any of the PDZ proteins in column 6 of the same box can be used for one or both PL motifs in column 4 of Table 1. For detecting the Vif protein of HIV-2, any of the PDZ proteins in column 6 of the same box can be used for the PL motifs in column 4 of Table 1. For detecting Protein X of Hepatitis B, any of the PDZ proteins in column 6 of the same box can be used for the PL motif in Table 1. For detecting the S antigen of Hepatitis B, any of the PDZ proteins in column 6 of the same box can be used for one or both PL motifs in Table 1. For detecting Capsid C of Hepatitis C any of the PDZ proteins in column 6 of the same box can be used for one or both PL motifs in column 4 of Table 1. For detecting the E1 protein of Hepatitis C, any of the PDZ proteins in column 6 of the same box can be used for one or both PL motifs in column 4 of Table 1. For detecting the Nucleoprotein of RSV, any of the PDZ proteins in column 6 of the same box can be used for the PL motif in column 4 of Table 1. For detecting VP4 of Rotavirus A, any of the PDZ proteins in column 6 of the same box can be used for one or both PL motifs in column 4 of Table 1. For detecting VP7 of Rotavirus A, any of the PDZ proteins in column 6 of the same box can be used for one or both PL motifs in column 4 of Table 1. For detecting NSP2 of Rotavirus A, any of the PDZ proteins in column 6 of the same box can be used for one or both PL motifs in column 4 of Table 1. For detecting NSP5 of Rotavirus A, any of the PDZ proteins in column 6 of the same box can be used for one or both PL motifs in column 4 of Table 1. For detecting the ESXN protein of M. tuberculosis, any of the PDZ proteins in column 6 of the same box can be used for the PL motif in column 4 of Table 1. For detecting the ESXS protein of M. tuberculosis, any of the PDZ proteins in column 6 of the same box can be used for the PL motif in column 4 of Table 1. For detecting the ESAT-6 protein of M. tuberculosis, any of the PDZ proteins in column 6 of the same box as contains the ESAT-6 protein can be used for the PL motif in column 4 of Table 1. For detecting other Flaviviruses, the PL proteins corresponding to the PL proteins identified in Hepatitis C (Capsid C and E1) can be used and tested for binding to PDZ proteins, particularly those identified in column 6 of Table 1. For testing other lentiviruses, the PL proteins corresponding to the PL proteins identified in HIV-I and HIV-2 (Env, Nef, and Vif) can be used and tested for binding to PDZ proteins, particularly those identified in column 6 of Table 1. For testing other Mycobacteria species, the PL proteins corresponding to the PL proteins identified in M. tuberculosis (ESAT-6 family and related proteins) can be used and tested for binding to PDZ proteins, particularly those identified in column 6 of Table 1. The mixtures of PDZ proteins, antibodies and other binding agents can be used for tests that identify all PLs associated with a protein, all strains or subtypes, and/or all members of a family or species. For example, mixtures can be used to identify all HIV-1 Env proteins (mixtures that would identify both PL's), all HIV-1 strains or subtypes (this could include agents that bind to mixtures of PL proteins), all HIV (including HIV-2), all lentiviruses, all retroviruses. For tests that generally identify HIV-1 Env, for example, a mixture of PDZ proteins and antibodies can be used to identify both PL's. For these and other tests, the PDZ protein can include one of the above in admixture with others that recognize other pathogen-specific or HIV specific PL motifs.

The present invention provides methods of detecting pathogen PL proteins in a sample and finds utility in diagnosing viral infection in a subject. In many exemplary procedures, a biological sample is obtained from a subject, and, the presence of a pathogen PL protein in the sample is determined. The presence of a detectable amount of pathogen PL protein in a sample indicates that the individual is infected with a particular virus. In other exemplary procedures, the level of pathogen PL protein in a biological sample is determined, and compared to the amount of a control in the sample. The relative amount of pathogen PL protein in a sample indicates the severity of the infection by the pathogen. Preferably the PL protein and the PL motif is at least one of the following, RALL (SEQ ID NO:242) or RILL (SEQ ID NO:243) for HIV-1 Env, FKNC (SEQ ID NO:244), FKDC (SEQ ID NO:245), YKNC (SEQ ID NO:246), or YKDC (SEQ ID NO:247) for HIV-1 Nef protein, IALL (SEQ ID NO:248), LALL (SEQ ID NO:249), or LTLL (SEQ ID NO:250) for HIV2 Env protein, EILA(SEQ ID NO:251), GILA (SEQ ID NO:252), or DILA (SEQ ID NO:253) for HIV-2 Vif protein, FTSA (SEQ ID NO:254) for Hepatitis B Protein X, WVYI (SEQ ID NO:255) for Hepatitis B S antigen, PASA (SEQ ID NO:256), or PVSA(SEQ ID NO:257) for Hepatitis C Capsid C protein, GVDA (SEQ ID NO:258) for Hepatitis C El protein, DVEL (SEQ ID NO:259) for RSV Nucleoprotein, QCKL (SEQ ID NO:260), or QCRL (SEQ ID NO:261) for Rotavirus A VP4 protein, YYRV (SEQ ID NO:262), or YYRI (SEQ ID NO:263) for Rotavirus A VP7 protein, QVGI (SEQ ID NO:264), HIGI (SEQ ID NO:265), QIGI (SEQ ID NO:266), or RIGI (SEQ ID NO:267) for Rotavirus A NSP2 protein, IKDL (SEQ ID NO:268) or IEDL (SEQ ID NO:269) for Rotavirus A NSP5 protein, SSWA (SEQ ID NO:270) for M. tuberculosis ESXN protein, YTGF (SEQ ID NO:271) for M. tuberculosis ESXS protein, GMFA (SEQ ID NO:272) for M. tuberculosis ESAT-6 protein.

The methods can employ two binding partners specific for viral and/or bacterial PL proteins, one of which is a PDZ domain polypeptide, as described above. In general, the methods involve a) isolating the pathogen PL from a sample using one of the binding partners, and b) detecting the pathogen PL protein with the other binding partner.

A. ELISA Sandwich Heterogeneous Assay Format

Using the instant PDZ capture and monoclonal anti-PL proteins, a sandwich assay format is constructed to detect viral and bacterial strains in biological samples. The instant assays have a sensitivity in the range of 1-1,000 ng/ml, i.e., sufficiently sensitive for commercial use in detecting the type or amount of a virus or bacterium in a biological sample, with the following caveats: namely,
a) Immunoassays are capable of distinguishing between the PL proteins in the HIV-1, HIV-2, Hepatitis B, Hepatitis C, Rotavirus A, RSV, and M. tuberculosis microorganisms.
b) The cross-reactivity profiles of different assay formats vary and also depend upon the particular virus or bacteria being detected, as well as, the absolute sensitivity in biological samples that contain cell lysates; and,
c) It is now relatively routine in the art of diagnostic devices to determine the detection limits for different assay formats.

Although a variety of competitive and non-competitive assay formats are identifiable for possible use in the instant methods, a sandwich assay format is presently preferred because these assays have proven performance characteristics and a variety of well established signal amplification strategies. In a presently preferred sandwich immunoassay procedure, a specific high affinity non-natural PDZ protein is employed to capture a natural viral PL protein antigen from within a biological sample; an anti-PL protein mouse monoclonal antibody is used to detect the bound PL protein antigen; and, the presence of the bound anti-PL protein antibody is detected using a signal generating compound, e.g. with either an enzyme-conjugated second antibody (e.g., horse radish peroxidase-conjugated antibody; HRP) or a biotinylated second antibody and streptavidin-enzyme conjugate (e.g., HRP).

In general, methods of the invention involve the steps of (i) separating (i.e., isolating) native viral PL protein analyte from within a complex biological sample using a first binding agent, i.e., a capture agent; and, (ii) detecting the isolated PL analyte using a second binding agent, i.e., a detect agent. The separating and detecting steps can be achieved using binding partners that have different levels of specificity for the PL analyte, e.g., if the capture agent is highly specific then lesser specificity may be used in the detect reagent and vice versa. In certain exemplary procedures, the capture agent is preferably a PDZ domain polypeptide. More preferably, the capture agent is one of those listed in Table 1 and/or Table 2. In alternative exemplary procedures, the first binding partner is an anti-pathogen PL protein antibody or mixture of antibodies, with the proviso that in these exemplary procedures at least one component of the detect reagent is a PDZ polypeptide, e.g., a PDZ protein detect agent that binds to the captured/isolated PL analyte and whose presence in the complex is then detected using an anti-PDZ antibody conjugated with a signal generating compound. In certain exemplary procedures, a PDZ capture agent is bound, directly or via a linker, to a solid phase. For example, in one non-limiting example the PDZ domain polypeptide is bound to a magnetic bead. In the latter example, when brought into contact with a biological sample the PDZ capture agent immobilized on the magnetic bead is effective in forming a PDZ-PL interaction complex with a bacterial or viral PL protein in the sample. Next, a magnetic field is applied and the interaction complex, with the bound bacterial or viral PL protein, is isolated from the sample. In another non-limiting example, a PDZ domain polypeptide capture agent is immobilized on the surface of a microtiter plate; a biological sample containing a bacterial or viral PL protein is brought into contact with the immobilized capture reagent resulting in binding of the PL to the surface of the plate; the plate is washed with buffer removing non-PL viral or bacterial analytes from the plate; and, the immobilized PL analyte is, thus, isolated from the biological sample. Different separation/isolation means are known, e.g., applying a magnetic field, washing and the like. The particular means employed is dependent upon the particular assay format. For example, separation can be accomplished by a number of different methods including but not limited to washing; magnetic means; centrifugation; filtration; chromatography including molecular sieve, ion exchange and affinity; separation in an electrical field; capillary action as e.g. in lateral flow test strips; immunoprecipitation; and, the like as disclosed further below.

In certain exemplary procedures, a bacterial or viral PL protein is separated from other viral or bacterial and cellular proteins in a biological sample by bringing an aliquot of the biological sample into contact with one end of a test strip, and then allowing the proteins to migrate on the test strip, e.g., by capillary action such as lateral flow. The instant methods are distinguished from prior immunoassay methods by the presence in the assay of one or more PDZ polypeptide agents, antibodies, and/or aptamers, e.g., as capture and/or detect reagents, conferring upon the assay the ability to specifically identify the presence or amount of a viral or bacterial strain. The instant methods are distinguished from prior immunoassay methods by the fact that they identify a bacterial or viral protein that is present in the patient sample, rather than an antibody. Methods and devices for lateral flow separation, detection, and quantification are known in the art, e.g., U.S. Pat. Nos. 6,942,981, 5,569,608; 6,297,020; and 6,403,383 incorporated herein by reference in their entirety. In one non-limiting example, a test strip comprises a proximal region for loading the sample (the sample-loading region) and a distal test region containing a PDZ polypeptide capture agent and buffer reagents and additives suitable for establishing binding interactions between the PDZ polypeptide and any PL protein in the migrating biological sample. In exemplary procedures, the test strip comprises two test regions that contain different PDZ domain polypeptides, i.e., each capable of specifically interacting with a different viral and/or bacterial PL protein analyte.

According to the methods disclosed above, viral or bacterial PL protein analytes are separated from other proteins in a biological sample, i.e., in such a manner that the analyte in the sample is suitable for detection and/or quantification. Novel methods are provided for detection of isolated PL proteins using PDZ polypeptides, PDZ polypeptides conjugated with signal generating compounds, antibodies, aptamers and the like. According to some exemplary procedures, viral or bacterial PL analytes bound to a PDZ capture agent, antibody and/or aptamer is detected using an antibody or antibodies specific for the pathogen PL protein, e.g., an antibody conjugated with a signal generating compound. A variety of detection methods are, of course, known in the diagnostic arts and it is not the intention of the present (non-limiting) disclosure to set forth all alternative well-known methods. Rather, the instant disclosure is intended to satisfy the requirement for setting forth the best mode of practicing the invention and to act as a general reference guide to alternative methods.

In some exemplary procedures, a PDZ domain conjugated with an SGC (signal generating compound) is used to detect the presence of a pathogen PL protein analyte in a sample in a homogeneous assay format, i.e., without need for a separation step. In this assay method the binding of a PL to the PDZ domain induces a change in the signal produced by the SGC, e.g., a change in fluorescent anisotropy.

In some exemplary procedures, heterogeneous solid phase assay formats (disclosed supra) are useful for detecting bacterial or viral PL analytes in biological samples. As noted in the Background section above, PDZ proteins bind cellular proteins containing PL. Similarly, in infected cells viral or bacterial proteins containing PL bind host cell PDZ proteins. While these interactions would normally be expected to compete with binding in a diagnostic assay format, further guidance is provided hereby that, unexpectedly, the affinities and mass balance of these latter natural interactions are sufficiently weak, or are sufficiently disrupted in detergent extracted cell lysates, that viral or bacterial PL analytes are detectable in the instant diagnostic assay formats. Accordingly, lysates can be prepared and assays conducted in the presence of less than about 0.5% of a detergent such as Tween-20 or Triton X100; preferably, less than about 0.2%; and, most preferably, less than about 0.1%.

In some exemplary procedures, the level of viral PL protein in a sample can be quantified and/or compared to controls. Suitable negative control samples are e.g. obtained from individuals known to be healthy, e.g., individuals known not to have the specific bacterial or viral infection being assayed. Specificity controls can be collected from individuals having known related virus or bacterial infection (if said virus or bacteria does not have the specific PL protein), or individuals infected with lower virulence strains. Control samples can be from individuals genetically related to the subject being tested, but can also be from genetically unrelated individuals. A suitable negative control sample can also be a sample collected from an individual at an earlier stage of infection, i.e., a time point earlier than the time point at which the test sample is taken. Some exemplary procedures also include non-infectious positive controls, i.e., recombinant proteins having amino acid sequences of bacterial or viral PLs.

Initial Western blots can be used to show that PL protein levels in biological samples are sufficient to allow detection of these antigens in a variety of different possible immunoassay formats. However, should the levels of PL protein in a particular biological sample prove to be limiting for detection in a particular immunoassay format, then, as one other exemplary procedure, the live virus in a biological sample can be-amplified by infecting cells in vitro, or growing bacteria on or in an appropriate growth media i.e., the PL protein in the virus-amplified sample should be detectable in about 6 hrs to about 12 hr. In other other exemplary procedures, methods for improving the yield of PL protein antigen in biological samples and virus-amplified samples include uses of protease inhibitors and proteasome inhibitors, e.g. MG132.

B. Preparation of Reagents

PL peptides, PDZ domain polypeptides, and aptamers can be made synthetically (i.e., using a machine) or using recombinant means, as is known in the art. For example, methods and conditions for expression of recombinant proteins are described in e.g., see Sambrook, supra, and Ausubel, supra. The use of mammalian tissue cell culture to express polypeptides is discussed generally in Winnacker, “From Genes to Clones, VCH Publishers, N.Y., N.Y., 1987; and, in Ausubel, supra.

Details of the binding assays are also disclosed in U.S. patent application Ser. No. 10/630,590, filed Jul. 29, 2003 and published as US20040018487 and in U.S. Pat. No. 6,942,981.

Cell-based assays generally involve co-producing (i.e., producing in the same cell, regardless of the time at which they are produced), the subject PDZ domain polypeptides and viral or bacterial PL using recombinant expression systems. Suitable cells for producing the subject polypeptides in eukaryotic cells are disclosed in the Examples section, below. Cell types that are potentially suitable for expression of subject PDZ domain polypeptide and viral or bacterial PL include the following: e.g., monkey kidney cells (COS cells), monkey kidney CVI cells transformed by SV40 (COS-7, ATCC CRL 165 1); human embryonic kidney cells (HEK-293, Graham et al. J. Gen Virol. 36:59 (1977)); HEK-293T cells; baby hamster kidney cells (BHK, ATCC CCL 10); Chinese hamster ovary-cells (CHO, Urlaub and Chasin, Proc. Natl. Acad. Sci. (USA) 77:4216, (1980); mouse sertoli cells (TM4, Mather, Biol. Reprod. 23:243-251 (1980)); monkey kidney cells (CVI ATCC CCL 70); african green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (hep G2, HB 8065); mouse mammary tumor (MMT 060562, ATCC CCL 51); TRI cells (Mather et al., Annals N.Y. Acad. Sci 383:44-68 (1982)); NIH/3T3 cells (ATCC CRL-1658); and mouse L cells (ATCC CCL-1). Additional cell lines will be apparent. A wide variety of cell lines are available from the American Type Culture Collection, 10801 University Boulevard, Manassas, Va. 20110-2209.

C. Sample Preparation

Any sample can be used that contains a detectable concentration of PL proteins and preferably of the viral or bacterial PL proteins disclosed herein. Examples of samples that can be used are lung exudates, cell extracts (respiratory, epithelial lining nose), blood, mucous, and nasal swabs, for example.

Binding of the PL protein to the PDZ protein and/or to an antibody was shown in the Examples to occur in the presence of up to 0.05% SDS, including 0.03% and 0.01%. Therefore, when the nasal or other bodily secretion is not likely to easily be used in a lateral flow format, it can be treated with SDS. Preferably, the amount of SDS added is up to a final concentration of 0.01%, more preferably 0.03% and even more preferably, 0.05%. Other detergents and the like can be used that do not interfere with binding of the PDZ protein, antibody, or aptamer or other agent to the PL protein. Other methods of sample treatment that do not interfere with protein/protein interactions can be used, including dilution with a buffer or water.

H. Test for Serum Antibodies

Tests to identify the presence of serum antibodies that bind to specific PL protein PL motifs can be used in any of the diagnostic methods for formats. The specific PL protein PL peptide can be used as capture reagents in lateral flow or other formats.

I. Use of the Assay in an Epidemic Setting

Assay sensitivity and specificity can be changed to achieve different absolute levels of detection of viral or bacterial PL proteins in a biological sample, e.g., by decreasing the levels of a competitive ligand in a competition assay format, changing the amounts of capture and detect reagent in sandwich assays and the like. Thus, the instant test methods encompass a variety of assays having different performance attributes to meet different needs encountered in different uses as illustrated in the Examples section, below. For instance, in a typical epidemic setting the highest positive predictive value (PPV) is commonly recorded and positive test results are more likely to be true, i.e., with the lowest negative predictive value (NPV) and false negative results tending to be more likely. Also in monitoring epidemics of viral or bacterial disease in human, animal, or bird subjects, it is presently common practice to submit all samples to reference laboratories for testing. By identifying the true positive samples in the instant screening assay, e.g., in the field or at the point of care, the instant test assays find uses in reducing the number of samples that must ultimately be submitted to a reference laboratory for testing, i.e., a particular value when the burden of testing is high during an epidemic. Because it is current practice to slaughter all animals, irrespective of whether they are infected, a relatively high false positive rate may be acceptable, but it is preferably accompanied by a relatively low false negative rate. In certain exemplary procedures, the invention provides test kits having different specificity, sensitivity, PPV and NPV for use during epidemics, referred to herein as “epidemic test methods”. Preferably to suit current needs, the instant epidemic test methods have assay performance as follows: namely, specificity greater than about 65%; sensitivity greater than about 90%; PPV greater than about 85%; NPV greater than about 65%; false positive values of less than about 25% and false negative values of less than about 5%.

In contrast, in times of low bacterial or viral disease incidence in animals, reservoirs or human subjects, the lowest PPV is commonly recorded with false positive test results more likely and with the highest NPV and negative tests results tending to be more likely and true. During these times of low incidence the aim in screening may be to rapidly identify potentially infected animals and isolate them until confirmatory testing is completed e.g. in a reference laboratory. Thus, in certain exemplary procedures test methods are provided having increased sensitivity and NPV for use during times of low bacterial or viral disease incidence where monitoring is essential, i.e., referred to herein as “monitoring test methods”. Preferably, the instant monitoring test methods have assay performance as follows: namely, specificity greater than about 65%; sensitivity greater than about 90%; PPV greater than about 85%; NPV greater than about 65%; false positive values of less than about 20% and false negative values of less than about 5%. When the instant monitoring test methods are used to screen more than 100 members of a group, the PPV for the group as a whole is significantly higher than the predictive values achieved in any one particular assay. Thus, when a positive test result is obtained in a monitoring test method it may prove beneficial to retest the members of the group using an epidemic test assay, supra.

In human, rather than animal, testing the aims are of course different. Timely evidence of a bacterial or viral infection can have important case management implications, e.g., prompting early administration of anti-bacterial or anti-viral agents in children or aged subjects. Generally with human diagnostic products a high degree of specificity and sensitivity are needed, e.g., greater than 90% specificity and sensitivity with greater than 90% PPV. However, in a defined epidemic setting, e.g., a cruise ship infection, where PPV is high; the likelihood of false positives is low and likelihood of false negatives is high; and, when samples are submitted for confirmatory testing, it can be desirable to have a lesser specificity such as 65% in order to yet further lower the number of false negative test results e.g. to a value less than about 5%.

J. Diagnostic and Therapeutic Kits

Kits are provided for carrying out the instant methods. In certain exemplary procedures, a PDZ protein is used to detect the presence of a PL protein in a sample, e.g., a sample of a cell infected by a pathogenic agent. Examples of pathogenic agents encoding PL sequences include, but are not limited to, viruses and bacteria, e.g., retrovirus such as HIV-1, HIV-2, HTLV-1, and HTLV-2;, a hepadnovirus such as hepatitis B, a flavivirus such as hepatitis C, dengue, Japanese encephalitis, tick-borne encephalitis, West Nile, and Yellow Fever; a reovirus such as rotavirus, a paramyxovirus such respiratory syncytial virus (RSV) and a bacterium such as Mycobacterium tuberculosis, Helicobacterpylori, Treponema pallidum, and Streptococcus pyogenes.

The present invention provides methods of detecting pathogen PL proteins in a sample and finds utility in diagnosing viral or bacterial infection in a subject. In exemplary procedures, a biological sample is obtained from a subject, and, the presence of a pathogen PL protein in the sample is determined. The presence of a detectable amount of pathogen PL protein in a sample indicates that the individual is infected with a particular virus or bacterium. In some exemplary procedures, the level of pathogen PL protein in a biological sample is determined, and compared to the amount of a control in the sample. The relative amount of pathogen PL protein in a sample indicates the severity of the infection by the pathogen.

The methods generally involve two binding partners of a pathogen PL protein, one of which is a PDZ domain polypeptide, as described above. In general, the methods involve a) isolating the pathogen PL from a sample using one of the binding partners, and b) detecting the pathogen PL protein with the other binding partner.

The instant kit is distinguished from immunoassay kits by at least the presence of one or more of: (i) a PDZ domain polypeptide and (ii) printed instructions for conducting an assay to identify a bacterial or viral strain in a biological sample using the PDZ domain polypeptide. The kit allows for the identification of a bacterial or viral protein in the patient sample rather than an antibody, making it more specific to an infected individual. The instant kit optionally contains one or more of the reagents, buffers or additive compositions or reagents disclosed supra; and, in certain exemplary procedures the kit can also contain antibodies specific for the bacteria or viral PL, preferably PL protein. In yet other exemplary procedures, the instant kit can further comprise a means, such as a device or a system, for removing the bacterial or viral PL from other potential interfering substances in the biological sample. The instant kit can further include, if desired, one or more of various components useful in conducting an assay: e.g., one or more assay containers; one or more control or calibration reagents; one or more solid phase surfaces on which to conduct the assay; or, one or more buffers, additives or detection reagents or antibodies; one or more printed instructions, e.g. as package inserts and/or container labels, for indicating the quantities of the respective components that are to be used in performing the assay, as well as, guidelines for assessing the results of the assay. The instant kit can contain components useful for conducting a variety of different types of assay formats, including e.g. test strips, sandwich ELISA, Western blot assays, latex agglutination and the like. The subject reference, control and calibrators within the instant kits can contain e.g. one or more natural and non-natural viral or bacterial PL proteins, recombinant PL polypeptides, synthetic PL peptides, PDZ domain polypeptides, PDZ domain peptides and/or appropriate colorimetric and enzyme standards for assessing the performance and accuracy of the instant methods.

The instructions for practicing the subject methods are commonly recorded on a suitable recording medium and included with the kit, e.g., as a package insert. For example, the instructions can be printed on a substrate such as paper or plastic. In other embodiments, the instructions can be digitally recorded on an electronic computer-readable storage medium, e.g. CD-ROM, diskette and the like. In yet other embodiments, instructions for conducting the instant methods can be obtained by a user from a remote digital source, e.g. at an internet website in the form of a downloadable document file.

Optionally, the kits can include reagents for performing a general test for HIV as well as specific tests. For example a lateral flow test can have a lane for identifying the presence of a general HIV virus and a lane for identifying whether that virus is HIV-1 or HIV-2. The general test can be any test that identified the presence of an HIV virus, including the test for the presence of PL protein. Other types of general HIV tests that can be included can identify any HIV-specific protein. Alternatively the presence of HIV or other viruses or bacteria can be identified by the presence of antibodies in the blood of the patient. Finally, PCR tests can be used to generally identify the presence of HIV or other viruses or bacteria.

K. Arrays

In yet other exemplary procedures PDZ, antibody, and/or aptamer arrays are provided consisting of different PDZ polypeptides, antibodies, and/or aptamers or comparable binding agents immobilized at identifiable selected locations on a solid phase. Each of the immobilized PDZ polypeptides, antibodies and/or aptamers in the array has a defined binding affinity and specificity for PL ligands, i.e., including identified binding interactions with PL in bacterial or viral proteins. The discriminatory activity of the array is contributed by (i) the binding affinity of the respective different PDZ polypeptides, antibodies, and/or aptamers; (ii) the binding specificities of the respective different PDZ polypeptides, antibodies, and/or aptamers for PL; and, (iii) the assay conditions, e.g., ionic strength, time, pH and the like. PDZ domains are highly specific, e.g., the PDZ domain in MAST205 is capable of distinguishing between C-terminal PL sequences containing TDV and SDV. Similarly, within the same PDZ protein the different respective domains can have different binding specificities and affinities, i.e., PSD-95 domains-1, -2 and -3 have different binding specificities and affinities. Applicants have cloned, expressed and disclosed in prior US Patent Applications, the sequences of more than 255 different human PDZ domains comprising greater than 90% of all the PDZ domains in the human genome. Mapped interactions of the PDZ domain fusion proteins with different PL peptides constitute the basis for selecting particular members of the instant viral and/or bacterial PL array. Unexpectedly, the selectivity of the array is based in the findings of: (i) distinguishingly different PL amino acid sequence motifs in different bacteria and viruses; and, combined with (ii) the different PL sequence motifs in different bacterial and viral proteins, i.e, Env, Vif, Nef, NA, M1, Nucleoprotein, Protein X, S antigen, Capsid C, E1, VP4, VP7, NSP2, NSP5, ESXN, ESXS, ESAT-6 and the like.

Exemplary procedures and methods are provided for distinguishing between the different strains of an HIV virus, or other retroviruses, in a test sample based on the constituent binding properties of the PL in the viral proteins, e.g., Env, Nef, Vif and the like, in which the different strains and/or subtypes of HIV or retroviruses produce a distinctive pattern of binding on the array. The methods involve the steps of: (a) bringing into contact aliquots of a test sample at different predefined positions in the array; (b) detecting the presence or absence of binding at a particular position in the array; (c) determining from the pattern of binding in the array that (i) HIV PL are present in test sample and (ii) that the pattern of PL binding in the array constitutes a distinguishing signature for a particular strain of HIV virus. Representative examples of the HIV viruses that are distinguishable based in arrays include e.g. HIV-1 and HIV-2. Preferably, the array is at least partly based on the binding to PL protein PLs. More preferably, the PDZ, antibody, and/or aptamer arrays specifically identify the presence of at least one PL protein PL from Table 1. Preferably, the PDZ protein is at least one of those selected from Tables 1 or 2, fragments or analogs. More preferably, the array includes at least one PDZ protein, antibody or aptamer mimic of any PDZ protein listed in Tables 1 and 2, analogs and active fragments. Preferably, the PL protein is selected from column 2 having the PL motif shown in column 4 and the PDZ protein is selected from those listed in column 6 for each virus or bacteria. The array can be configured to specifically identify different strains or species of virus or bacteria or to include PL proteins for a variety of viruses and bacteria, for a more general diagnostic.

L. Lateral Flow Designs

Similar to a home pregnancy test, lateral flow devices work by applying fluid to a test strip that has been treated with specific biologicals. Carried by the liquid sample, phosphors labeled with corresponding biologicals flow through the strip and can be captured as they pass into specific zones. The amount of phosphor signal found on the strip is proportional to the amount of the target analyte.

A sample suspected of containing one of the viruses, bacteria or related microbes disclosed herein is added to a lateral flow device by some means, the sample is allowed to move by diffusion and a line or colored zone indicates the presence of the virus or bacteria. The lateral flow typically contains a solid support (for example nitrocellulose membrane) that contains three specific areas: a sample addition area, a capture area containing one or more PDZ proteins and antibodies immobilized, and a read-out area that contains one or more zones, each zone containing one or more labels. The lateral flow can also include positive and negative controls. Thus, for example a lateral flow device in certain exemplary procedures would perform as follows: a viral or bacterial PL protein is separated from other bacterial, viral and cellular proteins in a biological sample by bringing an aliquot of the biological sample into contact with one end of a test strip, and then allowing the proteins to migrate on the test strip, e.g., by capillary action such as lateral flow. One or more PL binding agents such as PDZ polypeptide agents, antibodies, and/or aptamers are included as capture and/or detect reagents. Methods and devices for lateral flow separation, detection, and quantification are known in the art, e.g., U.S. Pat. Nos. 5,569,608; 6,297,020; and 6,403,383 incorporated herein by reference in their entirety. In one non-limiting example, a test strip comprises a proximal region for loading the sample (the sample-loading region) and a distal test region containing a PDZ polypeptide capture agent and buffer reagents and additives suitable for establishing binding interactions between the PDZ polypeptide and any viral or bacterial PL protein in the migrating biological sample. In alternative exemplary procedures, the test strip comprises two test regions that contain different PDZ domain polypeptides, i.e., each capable of specifically interacting with a different viral or bacterial PL protein analyte. Optionally, the lateral flow can include tests for a variety of different viruse and bacteria, for example those identified in Table 1.

A. Separating Pathogen PL Proteins

In general, methods are provided that involve separating (i.e., isolating) native pathogen PL protein from a sample. This separation is usually achieved using a first binding partner for the pathogen PL protein. The first binding partner can be a PDZ domain polypeptide, or, an anti-pathogen PL protein antibody or mixture of antibodies.

In some exemplary procedures, one of the binding partners is bound, directly or via a linker, to an insoluble support. Insoluble supports are known in the art and include, but are not limited to, a bead (e.g, magnetic beads, polystyrene beads, latex beads, and the like); a membrane; and the like. In one non-limiting example, a PDZ domain polypeptide is bound to a magnetic bead. The PDZ domain polypeptide bound to the magnetic bead is contacted with the sample, and, after a complex is formed between the antibody and any pathogen PL protein in the sample, a magnetic field is applied, such that the complex is removed from the sample. Where the PDZ domain polypeptide is bound to an insoluble support, such as a membrane, the pathogen PL protein bound to the PDZ domain polypeptide is removed from the sample by removing the membrane, or by transferring the sample to a separate container. Where the PDZ domain polypeptide is bound to a bead, the pathogen PL protein bound to the bead is removed from the sample by centrifugation or filtration. Such embodiments are envisioned using a different pathogen-PL binding partner, e.g., an antibody.

In general, a suitable separation means is used with a suitable platform for performing the separation. For example, where a pathogen PL protein is separated by binding to PDZ domain polypeptides, the separation is performed using any of a variety of platforms, including, but not limited to, affinity column chromatography, capillary action or lateral flow test strips, immunoprecipitation, etc.

In certain exemplary procedures, pathogen PL protein is separated from other proteins in the sample by applying the sample to one end of a test strip, and allowing the proteins to migrate by capillary action or lateral flow. Methods and devices for lateral flow separation, detection, and quantitation are known in the art. See, e.g., U.S. Pat. Nos. 5,569,608; 6,297,020; and 6,403,383. In these embodiments, a test strip comprises, in order from proximal end to distal end, a region for loading the sample (the sample-loading region) and a test region containing a pathogen PL protein binding partner, e.g., a region containing an PDZ domain polypeptide or, in other embodiments, a region containing a pathogen PL protein antibody. The sample is loaded on to the sample-loading region, and the proximal end of the test strip is placed in a buffer. Pathogen PL protein is captured by the bound antibody in the first test region. In alternative exemplary procedures, the test strip comprises two test regions that contain different pathogen PL binding partners, e.g, PDZ domain polypeptides that specifically recognize, e.g., HIV-1, HIV-2, Hepatits B, Hepatitis C, RSV, Rotavirus A, M. tuberculosis. Detection of the captured protein is carried out as described below. For example, detection of captured pathogen PL protein is carried out using detectably labeled antibody specific for an epitope of the pathogen PL protein. In alternative exemplary procedures, an anti-pathogen PL protein antibody can be present in the test region and detection of pathogen PL protein bound to the antibody uses a labeled PDZ domain polypeptide.

B. Detecting and Quantitating Viral and Bacterial PL Proteins

Once, pathogen PL protein is separated from other proteins in the sample, the protein is detected and/or the level or amount of protein is determined (e.g., measured). As discussed above, pathogen PL protein is generally detected using a binding partner, e.g. an antibody or antibodies specific to the pathogen PL protein, or a PDZ domain polypeptide.

Detection with a specific antibody is carried out using well-known methods. In general, the binding partner is detectably labeled, either directly or indirectly. Direct labels include radioisotopes (e.g., ¹²⁵I, ³⁵S, and the like); enzymes whose products are detectable (e.g., luciferase, beta-galactosidase, horseradish peroxidase, and the like); fluorescent labels (e.g., fluorescein isothiocyanate, rhodamine, phycoerythrin, and the like); fluorescence emitting metals, e.g., ¹⁵²Eu, or others of the lanthanide series, attached to the antibody through metal chelating groups such as EDTA; chemiluminescent compounds, e.g., luminol, isoluminol, acridinium salts, and the like; bioluminescent compounds, e.g., luciferin; fluorescent proteins; and the like. Fluorescent proteins include, but are not limited to, a green fluorescent protein (GFP), including, but not limited to, a “humanized” version of a GFP, e.g., wherein codons of the naturally-occurring nucleotide sequence are changed to more closely match human codon bias; a GFP derived from Aequoria victoria or a derivative thereof, e.g., a “humanized” derivative such as Enhanced GFP, which are available commercially, e.g., from Clontech, Inc.; a GFP from another species such as Renilla reniformis, Renilla mulleri, or Ptilosarcus guernyi, as described in, e.g., WO 99/49019 and Peelle et al. (2001) J. Protein Chem. 20:507-519; “humanized” recombinant GFP (hrGFP) (Stratagene); any of a variety of fluorescent and colored proteins from Anthozoan species, as described in, e.g., Matz et al. (1999) Nature Biotechnol. 17:969-973; and the like.

Indirect labels include second antibodies specific for anti-pathogen PL protein antibodies, wherein the second antibody is labeled as described above; and members of specific binding pairs, e.g., biotin-avidin, and the like.

In some exemplary procedures, a level of pathogen PL protein is quantitated. Quantitation can be carried out using any known method, including, but not limited to, enzyme-linked immunosorbent assay (ELISA); radioimmunoassay (RIA); and the like. In general, quantitation is accomplished by comparing the level of expression product detected in the sample with a standard curve.

In some exemplary procedures, pathogen PL protein is separated on a test strip, as described above. In these exemplary procedures, pathogen PL protein is detected using a detectably labeled binding partner that binds to the pathogen PL protein. Pathogen PL protein can be quantitated using a reflectance spectrophotometer, or by eye, for example.

Pathogen PL proteins can be detected by their ability to bind to PDZ domains. This could be a developed into a single detection stage approach or as a two-stage or ‘sandwich’ approach for increased sensitivity and specificity.

For single stage approaches, a ‘tagged’ version of a PDZ domain that specifically recognizes pathogen PL proteins, such as those disclosed in TABLES 1A, 1B and 2, can be used to directly probe for the presence of a pathogen PL protein in a sample. As noted supra, an example of this would be to attach the test sample to a solid support (for example, host cells or tissue could be coated on a slide and ‘fixed’ to permeablize the cell membranes), incubate the sample with a tagged ‘PL detector’ protein (a PDZ domain fusion) under appropriate conditions, wash away unbound PL detector, and assay for the presence of the ‘tag’ in the sample. One should note, however, that PDZ domains can also bind endogenous cellular proteins. Thus, frequency of binding must be compared to control cells that do not contain pathogen PL protein or the ‘PL detector’ should be modified such that it is significantly more specific for the pathogen PL protein than for any endogenous host cell PL proteins.

For two-stage or sandwich approaches, use of the PL detector is coupled with a second method of either capturing or detecting captured proteins. The second method could be using an antibody that binds to the pathogen PL protein or a second compound or protein that can bind to the pathogen PL protein at a location on the pathogen PL protein that does not reduce the availability of the pathogen PL sequence. Such proteins can include, but not be limited to, antibodies, other viral or bacterial proteins, or engineered compounds that bind pathogen PL proteins.

Biological samples to be analyzed using the methods of the invention are obtained from vertebrates, including but not limited to humans, cattle, horses, sheep, goats, pigs, chickens, ducks, and geese. In particular exemplary procedures biological samples are obtained from, e.g., a human or a non-human animal model, or cultured cells thereof. In many exemplary procedures, the biological sample is obtained from a living subject, human or animal.

In some exemplary procedures, the subject from whom the sample is obtained is apparently healthy, where the analysis is performed as a part of routine screening. In other exemplary procedures, the subject is one who is susceptible to a viral or bacterial infection, (e.g., as determined by family history; exposure to certain environmental factors; etc.). In other exemplary procedures, the subject has symptoms of a viral or bacterial infection (e.g., a cough, or the like). In other embodiments, the subject has been provisionally diagnosed as (or at risk of, e.g., having been exposed to infected animals) having a viral or bacterial infection (e.g. as determined by other tests based on e.g., PCR).

The biological sample can be derived from any tissue, organ or group of cells of the subject. In some exemplary procedures a scrape, biopsy, or lavage is obtained from a subject. Biological samples can include bodily fluids such as blood, urine, sputum, and oral fluid; and samples such as nasal washes, swabs or aspirates, tracheal aspirates, chancre swabs, and stool samples. Methods are known for the collection of biological specimens suitable for the detection of individual pathogens of interest, for example, nasopharyngeal specimens such as nasal swabs, washes or aspirates, or tracheal aspirates in the case of viruses or bacteria involved in respiratory disease (e.g., RSV, M. tuberculosis); blood samples (for detection of, e.g., HIV, hepatitis B, hepatitis C), stool samples for the detection of viruses or bacteria involved in gastric diseases (e.g., rotavirus, H. pylori); oral swabs for the detection of HIV, and chancre swabs for the detection of syphilis.

In some exemplary procedures, the biological sample is processed, e.g., to remove certain components that can interfere with an assay method of the invention, using methods that are standard in the art. In some exemplary procedures, the biological sample is processed to enrich for proteins, e.g., by salt precipitation, and the like. In certain exemplary procedures the sample is treated so as to break open the host cells and release viral or bacterial proteins from the host cells (e.g., by lysis or sonication). In certain exemplary procedures, the sample is processed in the presence of a proteasome inhibitor to inhibit degradation of the bacterial or viral proteins.

In some exemplary assay methods, the level of viral or bacterial PL protein in a sample can be quantified and/or compared to controls. Suitable control samples are from individuals known to be healthy, e.g., individuals known not to have a viral or bacterial infection. Control samples can be from individuals genetically related to the subject being tested, but can also be from genetically unrelated individuals. A suitable control sample also includes a sample from an individual taken at a time point earlier than the time point at which the test sample is taken, e.g., a biological sample taken from the individual prior to exhibiting possible symptoms of a viral or bacterial infection.

XI. Pharmaceutical Compositions

The above screening processes can identify one or more types of compounds that can be incorporated into pharmaceutical compositions. These compounds include agents that are inhibitors of transcription, translation and post-translational processing of either at least one PL protein, at least one PDZ protein. The agents also can also inhibit or block binding of a PL protein and a PDZ protein, or mixtures thereof. These compounds also include agents that are inhibitors of either one or more PL proteins, one or more PDZ proteins or the interaction between a PL protein and a PDZ protein and have an inherent respiratory and/or digestive or epithelial cell-specific activity or imaging activity. The compounds also include conjugates in which a pharmaceutical agent or imaging component is linked to an inhibitor of either a PL protein, a PDZ protein or the interaction between PL proteins and PDZ proteins. Conjugates comprising an agent with a pharmacological activity and a conjugate moiety having decreased substrate capacity for a PDZ protein relative to the agent alone are also provided for the purpose of reducing transport of the agent into non-infected cells, where the agent would confer undesired side effects. Preferably, the compound or agent inhibits or blocks the binding of at least one of the following PLs to a PDZ protein: RALL (SEQ ID NO:242) or RILL (SEQ ID NO:243) for HIV-1 Env, FKNC (SEQ ID NO:244), FKDC (SEQ ID NO:245), YKNC (SEQ ID NO:246), or YKDC (SEQ ID NO:247) for HIV-1 Nef protein, IALL (SEQ ID NO:248), LALL (SEQ ID NO:249), or LTLL (SEQ ID NO:250) for HIV2 Env protein, EILA(SEQ ID NO:251), GILA (SEQ ID NO:252), or DILA (SEQ ID NO:253) for HIV-2 Vif protein, FTSA (SEQ ID NO:254) for Hepatitis B Protein X, WVYI (SEQ ID NO:255) for Hepatitis B S antigen, PASA (SEQ ID NO:256), or PVSA(SEQ ID NO:257) for Hepatitis C Capsid C protein, GVDA (SEQ ID NO:258) for Hepatitis C E1 protein, DVEL (SEQ ID NO:259) for RSV Nucleoprotein, QCKL (SEQ ID NO:260), or QCRL (SEQ ID NO:261) for Rotavirus A VP4 protein, YYRV (SEQ ID NO:262), or YYRI (SEQ ID NO:263) for Rotavirus A VP7 protein, QVGI (SEQ ID NO:264), HIGI (SEQ ID NO:265), QIGI (SEQ ID NO:266), or RIGI (SEQ ID NO:267) for Rotavirus A NSP2 protein, IKDL (SEQ ID NO:268) or IEDL (SEQ ID NO:269) for Rotavirus A NSP5 protein, SSWA (SEQ ID NO:270) for M. tuberculosis ESXN protein, YTGF (SEQ ID NO:271) for M. tuberculosis ESXS protein, GMFA (SEQ ID NO:272) for M. tuberculosis ESAT-6 protein. More preferably, the PL protein PL is RALL (SEQ ID NO:242). More preferably, the PL protein is RALL (SEQ ID NO:242) or RILL (SEQ ID NO:243) for HIV-1 Env, FKNC (SEQ ID NO:244), FKDC (SEQ ID NO:245), YKNC (SEQ ID NO:246), or YKDC (SEQ ID NO:247) for HIV-1 Nef protein, IALL (SEQ ID NO:248), LALL (SEQ ID NO:249), or LTLL (SEQ ID NO:250) for HIV2 Env protein, EILA(SEQ ID NO:251), GILA (SEQ ID NO:252), or DILA (SEQ ID NO:253) for HIV-2 Vif protein. Preferably, the compound or agent inhibits the binding to at least one of the PDZ proteins from Tables 1 or 2. More preferably, the PDZ protein or interaction that is inhibited is at least one of the PDZ/PL interactions identified in Table 1 with the specific viral or bacterial PL in column 4 and the PDZ protein selected from one of those listed in column 6 of the same box or an analog or fragment and/or antibodies (or aptamers) that mimic any PDZ protein.

One or more of the above entities can be combined with pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, buffered water, physiological saline, phosphate buffered saline (PBS), Ringer's solution, dextrose solution, and Hank's solution. In addition, the pharmaceutical composition or formulation can also include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents, detergents and the like (see, e.g., Remington's Pharmaceutical Sciences, Mace Publishing Company, Philadelphia, Pa., 17th ed. (1985); for a brief review of methods for drug delivery, see, Langer, Science 249:1527-1533 (1990); each of these references is incorporated by reference in its entirety).

Pharmaceutical compositions for oral administration can be in the form of e.g., tablets, pills, powders, lozenges, sachets, cachets, elixirs, suspensions, emulsions, solutions, or syrups. Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methylcellulose. Preserving agents such as methyl- and propylhydroxy-benzoates; sweetening agents; and flavoring agents can also be included. Depending on the formulation, compositions can provide quick, sustained or delayed release of the active ingredient after administration to the patient. Polymeric materials can be used for oral sustained release delivery (see “Medical Applications of Controlled Release,” Langer and Wise (eds.), CRC Pres., Boca Raton, Fla. (1974); “Controlled Drug Bioavailability,” Drug Product Design and Performance, Smolen and Ball (eds.), Wiley, New York (1984); Ranger and Peppas, 1983, J. Macromol. Sci. Rev. Macromol Chem. 23:61; see also Levy et al., 1985, Science 228: 190; During et al., 1989, Ann. Neurol. 25:351; Howard et al., 1989, J. Neurosurg. 71:105). Sustained release can be achieved by encapsulating conjugates within a capsule, or within slow-dissolving polymers. Preferred polymers include sodium carboxymethylcellulose, hydroxypropylcellulose, hydroxypropylmethylcellulose and hydroxyethylcellulose (most preferred, hydroxypropyl methylcellulose). Other preferred cellulose ethers have been described (Alderman, Int. J. Pharm. Tech. & Prod. Mfr., 1984, 5(3) 1-9). Factors affecting drug release have been described in the art (Bamba et al., Int. J. Pharm., 1979, 2, 307). For administration by inhalation, the compounds for use according to the disclosures herein are conveniently delivered in the form of an aerosol spray preparation from pressurized packs or a nebulizer, with the use of a suitable propellant, e.g., dichlorodifluoromethane, trichlorofluoromethane, dichlorotetrafluoroethane, carbon dioxide or other suitable gas, or from propellant-free, dry-powder inhalers. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

Effective dosage amounts and regimes (amount and frequency of administration) of the pharmaceutical compositions are readily determined according to any one of several well-established protocols. For example, animal studies (e.g., mice, rats) are commonly used to determine the maximal tolerable dose of the bioactive agent per kilogram of weight. In general, at least one of the animal species tested is mammalian. The results from the animal studies can be extrapolated to determine doses for use in other species, such as humans for example.

A compound can be administered to a patient for prophylactic and/or therapeutic treatments. A therapeutic amount is an amount sufficient to remedy a disease state or symptoms, or otherwise prevent, hinder, retard, or reverse the progression of disease or any other undesirable symptoms in any way whatsoever. In prophylactic applications, a compound is administered to a patient susceptible to or otherwise at risk of a particular disease or infection. Hence, a “prophylactically effective” amount is an amount sufficient to prevent, hinder or retard a disease state or its symptoms. In either instance, the precise amount of compound contained in the composition depends on the patient's state of health and weight.

An appropriate dosage of the pharmaceutical composition is determined, for example, using animal studies (e.g., mice, rats) are commonly used to determine the maximal tolerable dose of the bioactive agent per kilogram of weight. In general, at least one of the animal species tested is mammalian. The results from the animal studies can be extrapolated to determine doses for use in other species, such as humans for example.

The components of pharmaceutical compositions are preferably of high purity and are substantially free of potentially harmful contaminants (e.g., at least National Food (NF) grade, generally at least analytical grade, and more typically at least pharmaceutical grade).

To the extent that a given compound must be synthesized prior to use, the resulting product is typically substantially free of any potentially toxic agents, particularly any endotoxins, which can be present during the synthesis or purification process. Compositions are usually made under GMP conditions. Compositions for parenteral administration are usually sterile and substantially isotonic.

A. Antiviral and Anti-Bacterial Agents

Pharmaceutical compositions suitable for use in the present invention include compositions wherein the active agents are contained in an effective dosage. Anti-viral and anti-bacterial agents include inhibitors of PL protein, PDZ, and/or PL protein/PDZ interactions that preferably show at least 30, 50, 75, 95, or 99% inhibition of levels of PL protein or PDZ mRNA or protein. Protein expression can be quantified by forming immunological analyses using an antibody that specifically binds to the protein followed by detection of complex formed between the antibody and protein. mRNA levels can be quantified by, for example, dot blot analysis, in-situ hybridization, RT-PCR, quantitative reverse-transcription PCR (i.e., the so-called “TaqMan” methods), Northern blots and nucleic acid probe array methods. Preferably, the PL protein PL used to identify inhibitors is one of: RALL (SEQ ID NO:242) or RILL (SEQ ID NO:243) for HIV-1 Env, FKNC (SEQ ID NO:244), FKDC (SEQ ID NO:245), YKNC (SEQ ID NO:246), or YKDC (SEQ ID NO:247) for HIV-1 Nef protein, IALL (SEQ ID NO:248), LALL (SEQ ID NO:249), or LTLL (SEQ ID NO:250) for HIV2 Env protein, EILA(SEQ ID NO:251), GILA (SEQ ID NO:252), or DILA (SEQ ID NO:253) for HIV-2 Vif protein, FTSA (SEQ ID NO:254) for Hepatitis B Protein X, WVYI (SEQ ID NO:255) for Hepatitis B S antigen, PASA (SEQ ID NO:256), or PVSA(SEQ ID NO:257) for Hepatitis C Capsid C protein, GVDA (SEQ ID NO:258) for Hepatitis C E1 protein, DVEL (SEQ ID NO:259) for RSV Nucleoprotein, QCKL (SEQ ID NO:260), or QCRL (SEQ ID NO:261) for Rotavirus A VP4 protein, YYRV (SEQ ID NO:262), or YYRI (SEQ ID NO:263) for Rotavirus A VP7 protein, QVGI (SEQ ID NO:264), HIGI (SEQ ID NO:265), QIGI (SEQ ID NO:266), or RIGI (SEQ ID NO:267) for Rotavirus A NSP2 protein, IKDL (SEQ ID NO:268) or IEDL (SEQ ID NO:269) for Rotavirus A NSP5 protein, SSWA (SEQ ID NO:270) for M. tuberculosis ESXN protein, YTGF (SEQ ID NO:271) for M. tuberculosis ESXS protein, GMFA (SEQ ID NO:272) for M. tuberculosis ESAT-6 protein. More preferably, the PL protein PL is RALL (SEQ ID NO:242). More preferably, the PL protein is RALL (SEQ ID NO:242) or RILL (SEQ ID NO:243) for HIV-1 Env, FKNC (SEQ ID NO:244), FKDC (SEQ ID NO:245), YKNC (SEQ ID NO:246), or YKDC (SEQ ID NO:247) for HIV-1 Nef protein, IALL (SEQ ID NO:248), LALL (SEQ ID NO:249), or LTLL (SEQ ID NO:250) for HIV2 Env protein, EILA(SEQ ID NO:251), GILA (SEQ ID NO:252), or DILA (SEQ ID NO:253) for HIV-2 Vif protein. Preferably, the PDZ protein used to identify inhibitors is at least one of those selected from Tables 1 or 2, fragments or analogs. More preferably, the PDZ protein used to identify inhibitors is at least one of the PDZ proteins in column 6 of Table 1 that is paired with one of the PL motifs in column 4, for the viral protein in column 2 of each virus or bacteria. For identifying agents that inhibit PL/PDZ binding to the Env protein of HIV-2, any of the PDZ proteins in column 6 can be used for one or both PL motifs in column 4 of Table 1. For identifying inhibitors of PDZ/PL binding for the Vif protein of HIV-2, any of the PDZ proteins in column 6 can be used for the PL motifs in column 4 of Table 1. For identifying inhibitors of PDZ/PL binding for Protein X of Hepatitis B, any of the PDZ proteins in column 6 can be used for the PL motif in Table 1. For identifying inhibitors of PDZ/PL binding for the S antigen of Hepatitis B, any of the PDZ proteins in column 6 can be used for one or both PL motifs in Table 1. For identifying inhibitors of PDZ/PL binding for Capsid C of Hepatitis C any of the PDZ proteins in column 6 can be used for one or both PL motifs in column 4 of Table 1. For identifying inhibitors of PDZ/PL binding for the E1 protein of Hepatitis C, any of the PDZ proteins in column 6 can be used for one or both PL motifs in column 4 of Table 1. For identifying inhibitors of PDZ/PL binding for the Nucleoprotein of RSV, any of the PDZ proteins in column 6 can be used for the PL motif in column 4 of Table 1. For identifying inhibitors of PDZ/PL binding for the VP4 of Rotavirus A, any of the PDZ proteins in column 6 can be used for one or both PL motifs in column 4 of Table 1. For identifying inhibitors of PDZ/PL binding for the VP7 of Rotavirus A, any of the PDZ proteins in column 6 can be used for one or both PL motifs in column 4 of Table 1. For identifying inhibitors of PDZ/PL binding for the NSP2 of Rotavirus A, any of the PDZ proteins in column 6 can be used for one or both PL motifs in column 4 of Table 1. For identifying inhibitors of PDZ/PL binding for the NSP5 of Rotavirus A, any of the PDZ proteins in column 6 can be used for one or both PL motifs in column 4 of Table 1. For identifying inhibitors of PDZ/PL binding for the ESXN protein of M. tuberculosis, any of the PDZ proteins in column 6 can be used for the PL motif in column 4 of Table 1. For identifying inhibitors of PDZ/PL binding for the ESXS protein of M. tuberculosis, any of the PDZ proteins in column 6 can be used for the PL motif in column 4 of Table 1. For identifying inhibitors of PDZ/PL binding for the ESAT-6 protein of M. tuberculosis, any of the PDZ proteins in column 6 can be used for the PL motif in column 4 of Table 1. For identifying inhibitors of PDZ/PL binding for other Flaviviruses, the PL proteins corresponding to the PL proteins identified in Hepatitis C (Capsid C and E1) can be used and tested for binding to PDZ proteins, particularly those identified in column 6 of Table 1. For identifying inhibitors of PDZ/PL binding for other lentiviruses, the PL proteins corresponding to the PL proteins identified in HIV-1 and HIV-2 (Env, Nef, and Vif) can be used and tested for binding to PDZ proteins, particularly those identified in column 6 of Table 1. For identifying inhibitors of PDZ/PL binding for other Mycobacteria species, the PL proteins corresponding to the PL proteins identified in M. tuberculosis (ESAT-6 family and related proteins) can be used and tested for binding to PDZ proteins, particularly those identified in column 6 of Table 1.

Anti-viral agents can include PL peptide therapeutics identified as binding to the PDZ protein that interacts with the viral or bacterial PL protein. The peptides, conservative substitutions or truncations thereof that leave the C-terminal PL are agents suitable for treating viral or bacterial diseases. For example, the C-terminal sequences (3 to 20 amino acids long or more preferably, 5-10 amino acids long) of a PL peptide can be converted into a therapeutic by attaching a transporter peptide (protein transduction domain) to the N-terminus of the peptide sequence. Subfragments of these peptides of at least 5 amino acids long with the C-terminal 3 amino acids conserved are used as therapeutic inhibitors of the viral PL/PDZ interaction, preferably, at least 6 amino acids long, 7 amino acids long, 8 amino acids long, 9 amino acids long, and 10 amino acids long. Preferably at least the C-terminal 4 amino acids are conserved, more preferably, the C-terminal 5 amino acids are conserved, the C-terminal 6 amino acids, or the C-terminal 7 amino acids. The peptide therapeutics also include peptides containing conservative substitutions of the amino acids in the peptide mimetics. However, preferably the conservative substitution is in a region other than the last 3 or 4 amino acids. Several transporter peptide sequences can be used, including Tat and antennapedia. Similarly, small molecule and COX2 inhibitors are identified as inhibiting a viral or bacterial PL protein interaction with a PDZ can be used as anti-viral therapeutics. The PL peptide therapeutic inhibitors are then tested in vitro and in vivo for inhibitory effects.

B. Methods of Screening for Anti-Viral and Anti-Bacterial Agents

Methods of screening for agents that bind to PL proteins and/or PDZ proteins are disclosed herein. The agents are initially screened for binding to the PL protein PL or the PDZ domain of the PDZ protein. Then they are tested for the ability to inhibit the PDZ/PL interaction. These methods are also provided below in “B. assay for anti-viral and anti-bacterial agents.” The binding assay can be performed in vitro using natural or synthetic PL proteins. Alternatively, natural or synthetic PDZ domain containing proteins can be used to identify agents capable of binding to a particular PDZ protein.

Methods of screening for anti-viral and anti-bacterial agents disclosed herein identify agents that block or inhibit the interaction between the viral PL and any PDZ protein that it interacts with. Inhibitors and DNA encoding them can also be screened for capacity to inhibit expression of PL protein and/or PDZ, and thus indirectly inhibit their interaction. An initial screen can be performed to select a subset of agents capable of inhibiting or stopping the PDZ/PL interaction. Such an assay can be performed in vitro using an isolated PDZ protein and PL protein or fragments thereof capable of binding to each other. Agents identified by such a screen can then be assayed functionally. Agents can also be screened in cells expressing PL proteins and either expressing the PDZ protein naturally or transformed to express the PDZ protein.

In addition to the diagnostic assays disclosed and illustrated above, assays are provided for identifying candidate anti-viral or anti-bacterial agents capable of modulating one or more binding interactions occurring between a bacterial or viral PL and a host cell PDZ polypeptide in a bacterial or viral infected cell. The instant methods involve testing the binding of a control PL, e.g., a synthetic PL peptide, to a PDZ domain polypeptide, e.g., a recombinant PDZ fusion protein, in the presence of an anti-viral or anti-bacterial test agent. A candidate anti-viral or anti-bacterial agent modulates the binding between the control PL and the PDZ domain polypeptide. Applicant has previously disclosed assays for measuring binding interactions between control PL and PDZ domain polypeptides in US and International patent applications, e.g., U.S. Pat. Nos. 5,569,608; 6,297,020; and 6,403,383 incorporated herein by reference in their entirety.

Particularly useful screening assays employ cells which express both one or more viral or bacterial PL protein PLs and one or more PDZ domain proteins. Such cells can be made recombinantly by co-transfection of the cells with polynucleotides encoding the proteins, or can be made by transfecting a cell which naturally contains one of the proteins with the second protein. The cells can be grown up in multi-well culture dishes and are exposed to varying concentrations of a test compound or compounds for a pre-determined period of time, which can be determined empirically. Whole cell lysates, cultured media or cell membranes are assayed for inhibition of the PL/PDZ interaction. Test compounds that significantly inhibit activity compared to control (as discussed below) are considered therapeutic candidates. Agents can also be tested for capacity to reduce viral or bacterial load in an animal model infected with the virus or bacteria against which the agent is directed. A reduction in load relative to a control untreated animal indicates the agent has antibacterial or antivirus activity.

Isolated PDZ domain proteins or PL-binding fragments thereof, can be used for screening therapeutic compounds in any of a variety of drug screening techniques. Alternatively, isolated PL protein PL proteins or fragments containing the PL motif can be used The protein employed in such a test can be membrane-bound, free in solution, affixed to a solid support, borne on a cell surface, or located intracellularly. The formation of binding complexes between the PDZ domain or PL protein PL and the agent being tested can be measured. More specifically, a test compound is considered as an inhibitor of the PDZ/PL interaction if the interaction is significantly lower than the interaction measured in the absence of test compound. In this context, the term “significantly lower” means that in the presence of the test compound the PDZ/PL interaction, when compared to that measured in the absence of test compound, is measurably lower, within the confidence limits of the assay method.

Random libraries of peptides or other compounds can also be screened for suitability as inhibitors of the PDZ/PL binding, or for simply binding to either the PDZ domain protein or the PL protein PL protein. Combinatorial libraries can be produced for many types of compounds that can be synthesized in a step-by-step fashion. Such compounds include polypeptides, beta-turn mimetics, polysaccharides, phospholipids, hormones, prostaglandins, steroids, aromatic compounds, heterocyclic compounds, benzodiazepines, oligomeric N-substituted glycines and oligocarbamates. Large combinatorial libraries of the compounds can be constructed by the encoded synthetic libraries (ESL) method described in Affymax, WO 95/12608, Affymax, WO 93/06121, Columbia University, WO 94/08051, Pharmacopeia, WO 95/35503 and Scripps, WO 95/30642 (each of which is incorporated by reference for all purposes).

A preferred source of test compounds for use in screening for therapeutics or therapeutic leads is a phage display library. See, e.g., Devlin, WO 91/18980; Key, B. K., et al., eds., Phage Display of Peptides and Proteins, A Laboratory Manual, Academic Press, San Diego,CA, 1996. Phage display is a powerful technology that allows one to use phage genetics to select and amplify peptides or proteins of desired characteristics from libraries containing 10⁸-10⁹ different sequences. Libraries can be designed for selected variegation of an amino acid sequence at desired positions, allowing bias of the library toward desired characteristics. Libraries are designed so that peptides are expressed fused to proteins that are displayed on the surface of the bacteriophage. The phage displaying peptides of the desired characteristics are selected and can be regrown for expansion. Since the peptides are amplified by propagation of the phage, the DNA from the selected phage can be readily sequenced facilitating rapid analyses of the selected peptides.

Phage encoding peptide inhibitors can be selected by selecting for phage that bind specifically to a PDZ domain protein and/or to a viral or bacterial PL protein PL. Libraries are generated fused to proteins such as gene II that are expressed on the surface of the phage. The libraries can be composed of peptides of various lengths, linear or constrained by the inclusion of two Cys amino acids, fused to the phage protein or can also be fused to additional proteins as a scaffold. One can also design libraries biased toward the PL regions disclosed herein or biased toward peptide sequences obtained from the selection of binding phage from the initial libraries provide additional test inhibitor compound.

In addition to the detection assays illustrated above, the invention also provides a variety of assays for identifying anti-viral and anti-bacterial agents that inhibit binding between the pathogen PL and a host cell PDZ polypeptide. In general, the methods involve testing binding of a PDZ ligand polypeptide to a polypeptide having a PDZ domain in the presence of a test agent. A test agent that modulates binding between the PDZ ligand polypeptide and a polypeptide having a PDZ domain modulates (i.e., increases or decreases, including abolishes) binding between the two proteins. As will be described below, binding between the two polypeptides can be assessed using a variety of means. Also as will be described in greater detail below, the assay can be performed in a cell-free environment (i.e., “in vitro”) using isolated polypeptides. The assay can be a cellular assay in which binding of the polypeptides within a cell, in the presence of a test agent, is evaluated. A wide variety of assay platforms are therefore available.

Binding of the polypeptides can be assayed using methods that are well known in the art. For example, binding can be assayed biochemically, or, in other exemplary procedures, the two proteins can be assayed by detecting a signal that is only produced when the proteins are bound together. In testing candidate agents, such a signal can be evaluated in order to assess binding between the two proteins. For example, as used in the subject assays, the polypeptides can form a fluorescence resonance energy transfer (FRET) system, bioluminescence resonance energy transfer (BRET) system, or colorimetric signal producing system that can be assayed.

The assay, whether it is performing in vitro or in a cellular environment, generally requires a) a polypeptide containing the PDZ ligand and b) a polypeptide containing the PDZ domain. At least one of the polypeptides can be a fusion protein that facilitates detection of binding between the polypeptides. Accordingly one of the polypeptides can contain, for example, an affinity tag domain or an optically detectable reporter domain.

Suitable affinity tags include any amino acid sequence that can be specifically bound to another moiety, usually another polypeptide, most usually an antibody. Suitable affinity tags include epitope tags, for example, the V5 tag, the FLAG tag, the HA tag (from hemagglutinin influenza virus), the myc tag, etc. Suitable affinity tags also include domains for which, binding substrates are known, e.g., HIS, GST and MBP tags, etc., and domains from other proteins for which specific binding partners, e.g., antibodies, particularly monoclonal antibodies, are available. Suitable affinity tags also include any protein-protein interaction domain, such as a IgG Fc region, which can be specifically bound and detected using a suitable binding partner, e.g. the IgG Fc receptor.

Suitable reporter domains include any domain that can optically report the presence of a polypeptide, e.g., by emitting light or generating a color. Suitable light emitting reporter domains include luciferase (from, e.g., firefly, Vargula, Renilla reniformis or Renilla muelleri), or light emitting variants thereof. Other suitable reporter domains include fluorescent proteins, (from e.g., jellyfish, corals and other coelenterates as such those from Aequoria, Renilla, Ptilosarcus, Stylatula species), or light emitting variants thereof. Light emitting variants of these reporter proteins can be brighter, dimmer, or have different excitation and/or emission spectra, as compared to a native reporter protein. For example, some variants are altered such that they no longer appear green, and can appear blue, cyan, yellow, enhanced yellow red (termed BFP, CFP, YFP eYFP and RFP, respectively) or have other emission spectra, as is known in the art. Other suitable reporter domains include domains that can report the presence of a polypeptide through a biochemical or color change, such as β-galactosidase, β-glucuronidase, chloramphenicol acetyl transferase, and secreted embryonic alkaline phosphatase. The reporter domain can be Renilla luciferase (e.g., pRLCMV; Promega, catalog number E2661).

An affinity tag or a reporter domain can be present at any position in a polypeptide of interest. However, in certain cases, they are present at the C- or N-terminal end of a polypeptide.

In particular exemplary procedures, one or both of the polypeptides can contain a tag or reporter. For example, if FRET or BRET methods are employed, the polypeptides can both be tagged using different autofluorescent polypeptides.

The PDZ domain-containing polypeptide contains at least the PDZ domain one of the polypeptides as binding a pathogen PL. The PDZ domain can contain the PDZ domain of a “wild-type” PDZ containing polypeptide, or a variant thereof that retains ability to bind to the PDZ ligand of interest. The sequence of the PDZ domains for wild-type PDZ domains are illustrated in TABLE 2. Any length of PDZ domain can be employed herein.

The pathogen PDZ ligand-containing polypeptide contains at least the PDZ ligand a pathogen protein, e.g., the PL protein. The PDZ ligand can contain the PDZ ligand of a “wild-type” polypeptide, or a variant thereof that retains ability to bind to the PDZ domains employed. Any length of PDZ domain can be employed herein.

Such polypeptides can be made synthetically (i.e., using a machine) or using recombinant means, as is known in the art. Methods and conditions for expression of recombinant proteins are described by e.g., Sambrook, supra, and Ausubel, supra. Typically, polynucleotides encoding the polypeptides used in the invention are expressed using expression vectors. Expression vectors typically include transcriptional and/or translational control signals (e.g., the promoter, ribosome-binding site, and ATG initiation codon). In addition, the efficiency of expression can be enhanced by the inclusion of enhancers appropriate to the cell system in use. For example, the SV40 enhancer or CMV enhancer can be used to increase expression in mammalian host cells. Typically, DNA encoding a polypeptide of the invention is inserted into DNA constructs capable of introduction into and expression in an in vitro host cell, such as a bacterial (e.g., E. coli, Bacillus subtilus), yeast (e.g., Saccharomyces), insect (e.g., Spodoptera frugiperda), or mammalian cell culture systems. Mammalian cell systems are preferred for many applications. Examples of mammalian cell culture systems useful for expression and production of the polypeptides of the present invention include human embryonic kidney line (293; Graham et al., 1977, J. Gen. Virol. 36:59); CHO (ATCC CCL 61 and CRL 9618); human cervical carcinoma cells (HeLa, ATCC CCL 2); and others known in the art. The use of mammalian tissue cell culture to express polypeptides is discussed generally in Winnacker, FROM GENES TO CLONES (VCH Publishers, N.Y., N.Y., 1987) and Ausubel, supra. In some exemplary procedures, promoters from mammalian genes or from mammalian viruses are used, e.g., for expression in mammalian cell lines. Suitable promoters can be constitutive, cell type-specific, stage-specific, and/or modulatable or regulatable (e.g.; by hormones such as glucocorticoids). Useful promoters include, but are not limited to, the metallothionein promoter, the constitutive adenovirus major late promoter, the dexamethasone-inducible MMTV promoter, the SV40 promoter, and promoter-enhancer combinations known in the art.

As noted above, the subject assay can be performed in vitro (i.e., in which the polypeptides are present in a solution a not in a cell) or in a cellular environment (in which the polypeptides are present in a cell).

In Vitro Assays

In vitro assays can be performed using a wide variety of. Certain methods involve linking, either covalently or non-covalently, a first polypeptide (either the PDZ domain polypeptide or the PDZ ligand polypeptide) to a substrate, contacting the substrate-bound polypeptide with the second polypeptide, and detecting the presence of the second polypeptide. The method can he performed in the presence of a test agent. In the cases in which the second polypeptide is detectably labeled (e.g., as an optically-detectable fusion protein), the presence of the second polypeptide is detected by detecting the label.

A substrate contains a solid, semi-solid, or insoluble support and is made from any material appropriate for linkage to a polypeptide, and does not interfere with the detection method used. As will be appreciated by those in the art, the number of possible affinity substrates is very large. Possible substrates include, but are not limited to, glass and modified or functionalized glass, plastics (including acrylics, polystyrene and copolymers of styrene and other materials, polypropylene, polyethylene, polybutylene, polyurethanes, Teflon, etc.), polysaccharides, nylon or nitrocellulose, resins, silica or silica-based materials including silicon and modified silicon, carbon, metals, inorganic glasses, plastics, ceramics, and a variety of other polymers. In one exemplary procedure, the substrates allow optical detection and do not themselves appreciably fluoresce or emit light. In addition, as is known the art, the substrate can be coated with any number of materials, including polymers, such as dextrans, acrylamides, gelatins, agarose, biocompatible substances such as proteins including bovine and other mammalian serum albumin.

In certain exemplary procedures, the substrate is coated in an agent that facilitates the specific binding (either directly or indirectly) of a polypeptide to the substrate. For example, the substrate is coated in streptavidin, and can bind a biotinylated polypeptide with affinity to the polypeptide of interest. In another example, the substrate is directly or indirectly (e.g., through protein A) coated with an antibody specific for the polypeptide.

As mentioned above, after the first polypeptide is linked to the substrate, the second polypeptide is contacted with the substrate and maintained under conditions suitable for specific binding of the second polypeptide to the first polypeptide, typically in the presence of a test agent. The second polypeptide is only detectable on the substrate only if the first and second polypeptides form a complex. Detection of the second polypeptide indicates that the first and second polypeptides form a complex. Detection of the second polypeptide that is bound to the affinity substrate is carried out directly (while the second polypeptide is bound to the substrate), or indirectly (e.g., after elution of the polypeptide from the substrate).

In cases where the second polypeptide contains a reporter domain, the second polypeptide can be detected by detecting reporter activity. Methods of determining reporter activity, e.g. luciferase and GFP activity, are described by e.g. Ramsay et al., Br. J. Pharmacology, 2001, 133:315-323. Detection of the second polypeptide can also be accomplished using an antibody, e.g., a labeled antibody. Methods for detecting polypeptides using antibodies are described by e.g., Ausubel et al, Short Protocols in Molecular Biology, 3rd ed., Wiley & Sons, 1995; and Harlow et al., Antibodies: A Laboratory Manual, First Edition 1988 Cold Spring Harbor, N.Y.).

To determine whether a test agent modulates binding between the subject polypeptides, the above assay can be performed in the presence or absence of a test agent.

Two complementary assays, termed “A” and “G”, were developed to detect binding between a PDZ-domain polypeptide and candidate PDZ ligand. In each of the two different assays, binding is detected between a peptide having a sequence corresponding to the C-terminus of a protein anticipated to bind to one or more PDZ domains (i.e. a candidate PL peptide) and a PDZ-domain polypeptide (typically a fusion protein containing a PDZ domain). In the “A” assay, the candidate PL peptide is immobilized and binding of a soluble PDZ-domain polypeptide to the immobilized peptide is detected (the “A” assay is named for the fact that an avidin surface is typically used to immobilize the peptide). In the “G” assay, the PDZ-domain polypeptide is immobilized and binding of a soluble PL peptide is detected (The “G” assay is named for the fact that a GST-binding surface is typically used to immobilize the PDZ-domain polypeptide).

Details of the A and G assays are set forth in U.S. patent application Ser. No. 10/630,590, filed Jul. 29, 2003 and published as US20040018487 and in the following description

Cellular Assays

Cellular assays generally involve co-producing (i.e., producing in the same cell, regardless of the time at which they are produced), the subject polypeptides using recombinant DNA. Suitable cells for producing the subject polypeptides include prokaryotic, e.g., bacterial cells, as well as eukaryotic cells e.g. an animal cell (for example an insect, mammal, fish, amphibian, bird or reptile cell), a plant cell (for example a maize or Arabidopsis cell), or a fungal cell (for example a S. cerevisiae cell). Any cell suitable for expression of subject polypeptide-encoding nucleic acid can be used as a host cell. Usually, an animal host cell line is used, examples of which are as follows: monkey kidney cells (COS cells), monkey kidney CVI cells transformed by SV40 (COS-7, ATCC CRL 165 1); human embryonic kidney cells (HEK-293, Graham et al. J. Gen Virol. 36:59 (1977)); HEK-293T cells; baby hamster kidney cells (BHK, ATCC CCL 10); chinese hamster ovary-cells (CHO, Urlaub and Chasin, Proc. Natl. Acad. Sci. (USA) 77:4216, (1980); mouse sertoli cells (TM4, Mather, Biol. Reprod. 23:243-251 (1980)); monkey kidney cells (CVI ATCC CCL 70); african green monkey kidney cells (VERO-76, ATCC CRL-1 587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (hep G2, HB 8065); mouse mammary tumor (MMT 060562, ATCC CCL 51); TRI cells (Mather et al., Annals N.Y. Acad. Sci 383:44-68 (1982)); NIH/3T3 cells (ATCC CRL-1658); and mouse L cells (ATCC CCL-1). A wide variety of cell lines are available from the American Type Culture Collection, 10801 University Boulevard, Manassas, Va. 20110-2209.

Again, a wide variety of platforms can be employed to detect binding between the subject polypeptides in a cell. For example, so-called “two-hybrid” methods can be employed, or a wide variety of fluorescence-based methods, e.g., FRET or BRET-based methods. In general, these methods involve contacting a cell that produces the subject polypeptides with a test agent, and determining if the test agent has any effect on binding between the subject polypeptides.

The GAL4 system can be used to screen agents that modulate binding between the subject polypeptides. Such methods can employ a vector (or vector system) encoding two polypeptides: a DNA binding domain polypeptide that contains either a PDZ domain or a PDZ ligand and DNA activation domain polypeptide containing the region not in the DNA binding domain polypeptide. The interaction between the PDZ domain and the PDZ ligand activates the expression of a reporter gene or selectable marker. The levels of α- or β-galactosidase, β-lactamase are measured by quantifying their enzymatic activity using colorimetric substrates, such as orthomethylphenylthiogalactoside (OMTP) or X-gal; the levels of light, e.g., fluorescence, can be assessed photometrically, e.g., fluorometrically. Pools of agents or individual agents are added to cultures in wells and the levels of inhibition or facilitation of the interaction by the agents are determined from the levels of the reporter gene activity.

Alternatively, Fluorescence Resonance Energy Transfer (FRET) can be used to detect binding between two polypeptides in a cell. Fluorescent molecules having the proper emission and excitation spectra that are brought into close proximity with one another can exhibit FRET. The fluorescent molecules are chosen such that the emission spectrum of one of the molecules (the donor molecule) overlaps with the excitation spectrum of the other molecule (the acceptor molecule). The donor molecule is excited by light of appropriate intensity within the donor's excitation spectrum. The donor then emits the absorbed energy as fluorescent light. The fluorescent energy it produces is quenched by the acceptor molecule. FRET can be manifested as a reduction in the intensity of the fluorescent signal from the donor, reduction in the lifetime of its excited state, and/or re-emission of fluorescent light at the longer wavelengths (lower energies) characteristic of the acceptor. When the fluorescent proteins physically separate, FRET effects are diminished or eliminated. (See, U.S. Pat. No. 5,981,200, the disclosure of which is hereby incorporated by reference in its entirety.)

For example, a cyan fluorescent protein is excited by light at roughly 425-450 nm wavelength and emits light in the range of 450-500 nm. Yellow fluorescent protein is excited by light at roughly 500-525 nm and emits light at 525-500nm. If these two proteins are present in a cell but not in close proximity, the cyan and yellow fluorescence can be separately visualized. However, if these two proteins are forced into close proximity with each other, the fluorescent properties will be altered by FRET. The bluish light emitted by CFP will be absorbed by YFP and re-emitted as yellow light. FRET is typically monitored by measuring the spectrum of emitted light in response to stimulation with light in the excitation range of the donor and calculating a ratio between the donor-emitted light and the acceptor-emitted light. When the donor:acceptor emission ratio is high, FRET is not occurring and the two fluorescent proteins are not in close proximity. When the donor: acceptor emission ratio is low, FRET is occurring and the two fluorescent proteins are in close proximity. In this manner, the interaction between a first and second polypeptide fused to a first and second reactive module, wherein the first and second reactive modules are donor and acceptor fluorescent molecules, respectively, can be measured. As such, the two polypeptides can contain a system that provides for FRET, e.g., one polypeptide contains GFP whereas the other contains YFP.

In a further embodiment, the first and seconds provide a Bioluminescence Resonance Energy Transfer (BRET) system. In such a system, one polypeptide of interest produces (or destroys) a fluorescent product (or substrate) and the other polypeptide of interest is a fluorescent protein that undergoes resonant energy transfer with the fluorescent product (or substrate). In one exemplary procedure, a BRET system comprises a luciferase from Renilla and a GFP. Exemplary BRET methodologies are described in Kroeger et al., J Biol Chem. 2001 Apr. 20;276(16):12736-43 and Xu et al., Proc Natl Acad Sci USA. 1999 January 5;96(1):151-6. A variety of calorimetric signal producing systems can also be employed.

The test agents employed in the subject methods can be any type of compound. The candidate agents or test compounds can be any of a large variety of compounds, both naturally occurring and synthetic, organic and inorganic, and including polymers (e.g., oligopeptides, polypeptides, oligonucleotides, and polynucleotides), small molecules (i.e., under about 500 Da in weight), antibodies, sugars, fatty acids, nucleotides and nucleotide analogs, analogs of naturally occurring structures (e.g., peptide mimetics, nucleic acid analogs, and the like), and numerous other compounds. Test agents can be prepared from diversity libraries, such as random or combinatorial peptide or non-peptide libraries. Many libraries are known in the art that can be used, e.g., chemically synthesized libraries, recombinant (e.g., phage display libraries), and in vitro translation-based libraries. Examples of chemically synthesized libraries are described in Fodor et al., 1991, Science 251:767-773; Houghten et al., 1991, Nature 354:84-86; Lam et al., 1991, Nature 354:82-84; Medynski, 1994, Bio/Technology 12:709-710; Gallop et al., 1994, J. Medicinal Chemistry 37(9):1233-1251; Ohlmeyer et al., 1993, Proc. Natl. Acad. Sci. USA 90:10922-10926; Erb et al., 1994, Proc. NatL. Acad. Sci. USA 91:11422-11426; Houghten et al., 1992, Biotechniques 13:412; Jayawickreme et al., 1994, Proc. Natl. Acad. Sci. USA 91:1614-1618; Salmon et al., 1993, Proc. Natl. Acad. Sci. USA 90:11708-11712; PCT Publication No. WO 93/20242; and Brenner and Lerner, 1992, Proc. Natl. Acad. Sci. USA 89:5381-5383. Examples of phage display libraries are described in Scott and Smith, 1990, Science 249:386-390; Devlin et al., 1990, Science, 249:404-406; Christian, R. B., et al., 1992, J. Mol Biol. 227:711-718); Lenstra, 1992, J. Immunol. Meth. 152:149-157; Kay et al., 1993, Gene 128:59-65; and PCT Publication No. WO 94/18318 dated Aug. 18, 1994. In vitro translation-based libraries include but are not limited to those described in PCT Publication No. WO 91/05058 dated Apr. 18, 1991; and Mattheakis et al., 1994, Proc. Natl. Acad. Sci. USA 91:9022-9026. By way of examples of nonpeptide libraries, a benzodiazepine library (see e.g., Bunin et al., 1994, Proc. Natl. Acad. Sci. USA 91:4708-4712) can be adapted for use. Peptoid libraries (Simon et al., 1992, Proc. Natl. Acad. Sci. USA 89:9367-9371) can also be used. Another example of a library that can be used, in which the amide functionalities in peptides have been permethylated to generate a chemically transformed combinatorial library, is described by Ostresh et al. (1994, Proc. Natl. Acad. Sci. USA 91:11138-11142).

A test agent can be a PDZ domain that binds to the PL being tested, or an analog thereof.

Once identified as an agent that modulates binding of a subject PL polypeptide to a subject PDZ polypeptide, i.e., a binding-modulatory agent, the agent can be tested in a variety of different assays, including cell-free assays, cellular assays and assays that employ animals or isolated organs thereof. For example, the binding-modulatory agent can be tested to determine if the agent viral virulence or pathogencity in cells in tissue culture, or in test animals, e.g., chickens or another bird, in any appropriate system.

C. Types of Anti-Viral and Anti-Bacterial Agents

Any of the agents set out below can be used as pharmaceuticals as well as those identified in screening methods. Inhibitors can be identified from any type of library, including RNA expression libraries, bacteriophage expression libraries, small molecule libraries, peptide libraries. Inhibitors can also be produced using the known sequence of the nucleic acid and/or polypeptide. The compounds also include several categories of molecules known to regulate gene expression, such as zinc finger proteins, ribozymes, siRNAs and antisense RNAs.

(a) siRNA Inhibitors

siRNAs are relatively short, at least partly double stranded, RNA molecules that serve to inhibit expression of a complementary mRNA transcript. Although an understanding of mechanism is not required for practice of the invention, it is believed that siRNAs act by inducing degradation of a complementary mRNA transcript. Principles for design and use of siRNAs generally are described by WO 99/32619, Elbashir, EMBO J. 20, 6877-6888 (2001) and Nykanen et a/, Cell 107, 309-321 (2001); WO 01/29058.

siRNAs of the invention are formed from two strands of at least partly complementary RNA, each strand preferably of 10-30, 15-25, or 17-23 or 19-21 nucleotides long. The strands can be perfectly complementary to each other throughout their length or can have single stranded 3′-overhangs at one or both ends of an otherwise double stranded molecule. Single stranded overhangs, if present, are usually of 1-6 bases with 1 or 2 bases being preferred. The antisense strand of an siRNA is selected to be substantially complementary (e.g., at least 80, 90, 95% and preferably 100%) complementary to a segment of a PL protein or PDZ transcript. Any mismatched based preferably occur at or near the ends of the strands of the siRNA. Mismatched bases at the ends can be deoxyribonucleotides. The sense strand of an siRNA shows an analogous relationship with the complement of the segment of the PL protein or PDZ transcript. siRNAs having two strands, each having 19 bases of perfect complementarity, and having two unmatched bases at the 3′ end of the sense strand and one at the 3′ end of the antisense strand are particularly suitable.

If an siRNA is to be administered as such, as distinct from the form of DNA encoding the siRNA, then the strands of an siRNA can contain one or more nucleotide analogs. The nucleotide analogs are located at positions at which inhibitor activity is not substantially effected, e.g. in a region at the 5′-end and/or the 3′-end, particularly single stranded overhang regions. Preferred nucleotide analogues are sugar- or backbone-modified ribonucleotides. Nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8 position, e.g. 8-bromo guanosine; deaza nucleotides, e.g. 7-deaza-adenosine; O- and N-alkylated nucleotides, e.g. N6-methyl adenosine are also suitable. In preferred sugar-modified ribonucleotides, the 2′ OH-group is replaced by a group selected from H, OR, R, halo, SH, SR, NH2, NHR, NR2 or CN, wherein R is C1-C6 alkyl, alkenyl or alkynyl and halo is F, Cl, Br or I. In preferred backbone-modified ribonucleotides the phosphoester group connecting to adjacent ribonucleotides is replaced by a modified group, e.g. of phosphothioate group. A further preferred modification is to introduce a phosphate group on the 5′ hydroxide residue of an siRNA. Such a group can be introduced by treatment of an siRNA with ATP and T4 kinase. The phosphodiester linkages of natural RNA can also be modified to include at least one of a nitrogen or sulfur heteroatom. Modifications in RNA structure can be tailored to allow specific genetic inhibition while avoiding a general panic response in some organisms which is generated by dsRNA. Likewise, bases can be modified to block the activity of adenosine deaminase.

A number of segments within the PL protein or PDZ transcript are suitable targets for design of siRNAs. When a selected segment of PL protein PL is used to selectively target a subtype, the segment preferably shows a lack of perfect sequence identity with other PL protein PL regions of the transcript. Preferably, the selected segment of a PL protein or PDZ protein shows at least at least 1, 2, 3, 4 or more nucleotide differences from a corresponding segment (if any) of a PL protein PL. Target sites can be chosen from the coding region, 5′UTR and 3′UTR of PL protein or PDZ, in some cases, the PL site of PL protein is preferred. A preferred target site is that of the siRNA termed PL protein PL (see Examples). This site is at the C-terminus and is specific for subtypes of the bacteria or virus. Other preferred sites include the PL binding site of the PDZ protein.

siRNA can be synthesized recombinantly by inserting a segment of DNA encoding the siRNA between a pair of promoters that are oriented to drive transcription of the inserted segment in opposite orientations. Transcription from such promoters produces two complementary RNA strands that can subsequently anneal to form the desired dsRNA. Exemplary plasmids for use in such systems include the plasmid (PCR 4.0 TOPO) (available from Invitrogen). Another example is the vector pGEM-T (Promega, Madison, Wis.) in which the oppositely oriented promoters are T7 and SP6; the T3 promoter can also be used. Alternatively, DNA segments encoding the strands of the siRNA are inserted downstream of a single promoter. In this system, the sense and antisense strands of the siRNA are co-transcribed to generate a single RNA strand that is self-complementary and thus can form dsRNA. Vectors encoding siRNAs can be transcribed in vitro, or in cell culture or can be introduced into transgenic animals or patients for expression in situ. Suitable vectors are described below. The selection of promoters and optionally other regulatory sequences for recombinant expression can determine the tissue specificity of expression. For example, PDGF, prion, neural enolase, or thy-1 promoters are suitable for expression in the central nervous system.

The strands of an siRNAs can also be synthesized by organic chemical synthesis and annealed in vitro. If synthesized chemically or by in vitro enzymatic synthesis, the RNA can be purified prior to introduction into the cell. For example, RNA can be purified from a mixture by extraction with a solvent or resin precipitation, electrophoresis, chromatography; or a combination thereof. The RNA can be dried for storage or dissolve in an aqueous solution. The solution can contain buffers or salts to promote annealing, and/or stabilization of the duplex stands. siRNAs can be introduced into cells or organisms either as RNA or in the form of DNA encoding the RNA by a variety of approaches, as described below.

(b) Antisense Polynucleotides

Antisense polynucleotides can cause suppression by binding to, and interfering with, the translation of sense mRNA, interfering with transcription, interfering with processing or localization of RNA precursors, repressing transcription of mRNA or acting through some other mechanism. The particular mechanism by which the antisense molecule reduces expression is not critical.

Typically antisense polynucleotides comprise a single-stranded antisense sequence of at least 7 to 10 to typically 20 or more nucleotides that specifically hybridize to a sequence from mRNA of a gene. Some antisense polynucleotides are from about 10 to about 50 nucleotides in length or from about 14 to about 35 nucleotides in length. Some antisense polynucleotides are polynucleotides of less than about 100 nucleotides or less than about 200 nucleotides. In general, the antisense polynucleotide should be long enough to form a stable duplex but short enough, depending on the mode of delivery, to administer in vivo, if desired. The minimum length of a polynucleotide required for specific hybridization to a target sequence depends on several factors, such as G/C content, positioning of mismatched bases (if any), degree of uniqueness of the sequence as compared to the population of target polynucleotides, and chemical nature of the polynucleotide (e.g., methylphosphonate backbone, peptide nucleic acid, phosphorothioate), among other factors.

To ensure specific hybridization, the antisense sequence is at least substantially complementary to a segment of target mRNA or gene encoding the same. Some antisense sequences are exactly complementary to their intended target sequence. The antisense polynucleotides can also include, however, nucleotide substitutions, additions, deletions, transitions, transpositions, or modifications, or other nucleic acid sequences or non-nucleic acid moieties so long as specific binding to the relevant target sequence corresponding to RNA or its gene is retained as a functional property of the polynucleotide. Antisense polynucleotides intended to inhibit PL protein or PDZ protein expression are designed to show perfect or a substantial degree of sequence identity to a specific PL protein or PDZ gene or transcript and imperfect and a lower degree of sequence identity to different PDZ gene.

Some antisense sequences are complementary to relatively accessible sequences of mRNA (e.g., relatively devoid of secondary structure). This can be determined by analyzing predicted RNA secondary structures using, for example, the MFOLD program (Genetics Computer Group, Madison Wis.) and testing in vitro or in vivo as is known in the art. Another useful method for identifying effective antisense compositions uses combinatorial arrays of oligonucleotides (see, e.g., Milner et al., 1997, Nature Biotechnology 15:537).

Antisense nucleic acids (DNA, RNA, modified, analogues, and the like) can be made using any suitable method for producing a nucleic acid, such as the chemical synthesis and recombinant methods disclosed herein. Antisense RNA can be delivered as is or in the form of DNA encoding the antisense RNA. DNA encoding antisense RNA can be delivered as a component of a vector, or in nonreplicable form, such as described below.

(c) Zinc Finger Proteins

Zinc finger proteins can also be used to suppress expression of the PL protein or PDZ protein or nucleic acid or a specific PL protein subtype. Zinc finger proteins can be engineered or selected to bind to any desired target site within a target gene. In some methods, the target site is within a promoter or enhancer. In other methods, the target site is within the structural gene. In some methods, the zinc finger protein is linked to a transcriptional repressor, such as the KRAB repression domain from the human KOX-I protein (Thiesen et al., New Biologist 2, 363-374 (1990); Margolin et al., Proc. Natl. Acad. Sci. USA 91, 4509-4513 (1994); Pengue et al., Nucl. Acids Res. 22:2908-2914 (1994); Witzgall et al., Proc. Natl. Acad. Sci. USA 91, 4514-4518 (1994). Methods for selecting target sites suitable for targeting by zinc finger proteins, and methods for design zinc finger proteins to bind to selected target sites are described in WO 00/00388. Methods for selecting zinc finger proteins to bind to a target using phage display are described by EP.95908614.1. The target site used for design of a zinc finger protein is typically of the order of 9-19 nucleotides. For inhibition of PL protein or PDZ protein or polynucleotide, a target site is chosen within the PL protein or PDZ protein or polynucleotide that shows imperfect or lack of substantial sequence identity to a different PDZ gene or transcript as discussed above. Methods for using zinc finger proteins to regulate endogenous genes are described in WO 00/00409. Zinc finger proteins can be administered either as proteins or in the form of nucleic acids encoding zinc fingers. In the latter situation, the nucleic acids can be delivered using vectors or in nonreplicable form as described below.

(d) Ribozymes

Ribozymes are RNA molecules that act as enzymes and can be engineered to cleave other RNA molecules at specific sites. The ribozyme itself is not consumed in this process, and can act catalytically to cleave multiple copies of mRNA target molecules. General rules for the design of ribozymes that cleave target RNA in trans are described in Haseloff & Gerlach, (1988) Nature 334:585-591 and Uhlenbeck, (1987) Nature 328:596-603 and U.S. Pat. No. 5,496,698.

Ribozymes typically include two flanking segments that show complementarity to and bind to two sites on a transcript (target subsites) and a catalytic region between the flanking segments. The flanking segments are typically 5-9 nucleotides long and optimally 6 to 8 nucleotides long. The catalytic region of the ribozyme is generally about 22 nucleotides in length. The mRNA target contains a consensus cleavage site between the target subsites having the general formula NUN, and preferably GUC. (Kashani-Sabet and Scanlon, (1995) Cancer Gene Therapy 2:213-223; Perriman, et al., (1992) Gene (Amst.) 113:157-163; Ruffner, et al., (1990) Biochemistry 29: 10695-10702); Birikh, et al., (1997) Eur. J.Biochem. 245:1-16; Perrealt, et al., (1991) Biochemistry 30:4020-4025).

The specificity of a ribozyme can be controlled by selection of the target subsites and thus the flanking segments of the ribozyme that are complementary to such subsites. For an inhibitor of PL protein or PDZ proteins, the target subsites are preferably chosen so that there are no exact corresponding subsites in other PDZ proteins and preferably no corresponding subsites with substantial sequence identity. Ribozymes can be delivered either as RNA molecules or in the form of DNA encoding the ribozyme as a component of a replicable vector or in nonreplicable form as described below.

(e). Antibodies

The compounds include antibodies, both intact and binding fragments thereof, such as Fabs, Fvs, which specifically bind to a protein encoded by a gene of the invention. Usually the antibody is a monoclonal antibody although polyclonal antibodies can also be expressed recombinantly (see, e.g., U.S. Pat. No. 6,555,310). Examples of antibodies that can be expressed include mouse antibodies, chimeric antibodies, humanized antibodies, veneered antibodies and human antibodies. Chimeric antibodies are antibodies whose light and heavy chain genes have been constructed, typically by genetic engineering, from immunoglobulin gene segments belonging to different species (see, e.g., Boyce et al., Annals of Oncology 14:520-535 (2003)). For example, the variable (V) segments of the genes from a mouse monoclonal antibody can be joined to human constant (C) segments. A typical chimeric antibody is thus a hybrid protein consisting of the V or antigen-binding domain from a mouse antibody and the C or effector domain from a human antibody. Humanized antibodies have variable region framework residues substantially from a human antibody (termed an acceptor antibody) and complementarity determining regions substantially from a mouse-antibody, (referred to as the donor immunoglobulin). See Queen et al., Proc. Natl. Acad. Sci. USA 86:10029-10033 (1989) and WO 90/07861, U.S. Pat. No. 5,693,762, U.S. Pat. No. 5,693,761, U.S. Pat. No. 5,585,089, U.S. Pat. No. 5,530,101 and Winter, U.S. Pat. No. 5,225,539. The constant region(s), if present, are also substantially or entirely from a human immunoglobulin. Antibodies can be obtained by conventional hybridoma approaches, phage display (see, e.g., Dower et al., WO 91/17271 and McCafferty et al., WO 92/01047), use of transgenic mice with human immune systems (Lonberg et al., WO93/12227 (1993)), among other sources. Nucleic acids encoding immunoglobulin chains can be obtained from hybridomas or cell lines producing antibodies, or based on immunoglobulin nucleic acid or amino acid sequences in the published literature.

(f). Mimetic Compounds

The inhibitor compound can be a mimetic of a subject PDZ domain or PDZ ligand, i.e., a synthetic chemical compound that has substantially the same structural and/or functional characteristics as a subject PDZ domain or PDZ ligand. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or inhibitory or binding activity. As with polypeptides of the invention which are conservative variants, routine experimentation will determine whether a mimetic is within the scope of the invention, i.e., that its structure and/or function is not substantially altered. Thus, a mimetic composition is within the scope of the invention if it is capable of inhibiting binding between the subject polypeptides.

Mimetics can contain any combination of normatural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like.

A polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N═-dicyclohexylcarbodiimide (DCC) or N,N═-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, A Peptide Backbone Modifications, Marcell Dekker, NY).

A polypeptide can also be characterized as a mimetic by containing all or some non-natural residues in place of naturally occurring amino acid residues. Normatural residues are well described in the scientific and patent literature; a few exemplary normatural compositions useful as mimetics of natural amino acid residues and guidelines are described below.

Mimetics of aromatic amino acids can be generated by replacing by, e.g., D- or L-naphylalanine; D- or L- phenylglycine; D- or L-2 thieneylalanine; D- or L-1, -2, 3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluorophenylalanine; D- or L-p-biphenylphenylalanine; K- or L-p-methoxybiphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a normatural amino acid include, e.g., thiazolyl, thiophenyl, pyrazolyl, benzimidazolyl, naphthyl, furanyl, pyrrolyl, and pyridyl aromatic rings.

Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R═—N—C—N—R═) such as, e.g., 1-cyclohexyl-3(2-morpholinyl-(4-ethyl) carbodiimide or 1-ethyl-3(4-azonia-4,4- dimetholpentyl) carbodiimide. Aspartyl or glutamyl can also be converted to asparaginyl and glutaminyl residues by reaction with ammonium ions.

Mimetics of basic amino acids can be generated by substitution with, e.g., (in addition to lysine and arginine) the amino acids ornithine, citrulline, or (guanidino)-acetic acid, or (guanidino)alkyl-acetic acid, where alkyl is defined above. Nitrile derivative (e.g., containing the CN-moiety in place of COOH) can be substituted for asparagine or glutamine. Asparaginyl and glutaminyl residues can be deaminated to the corresponding aspartyl or glutamyl residues.

Arginine residue mimetics can be generated by reacting arginyl with, e.g., one or more conventional reagents, including, e.g., phenylglyoxal, 2,3-butanedione, 1,2-cyclohexanedione, or ninhydrin, preferably under alkaline conditions.

Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively.

Cysteine residue mimetics can be generated by reacting cysteinyl residues with, e.g., alpha-haloacetates such as 2-chloroacetic acid or chloroacetamide and corresponding amines, to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl) propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole.

Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitrobenzenesulfonic acid, O-methylisourea, 2,4, pentanedione, and transamidase-catalyzed reactions with glyoxylate.

Mimetics of methionine can be generated by reaction with, e.g., methionine sulfoxide. Mimetics of proline include, e.g., pipecolic acid, thiazolidine carboxylic acid, 3- or 4-hydroxy proline, dehydroproline, 3- or 4-methylproline, or 3,3,-dimethylproline. Histidine residue mimetics can be generated by reacting histidyl with, e.g., diethylprocarbonate or para-bromophenacyl bromide.

Other mimetics include, e.g., those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

An amino acid of a subject polypeptide can also be replaced by an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, generally referred to as the D- amino acid, but which can additionally be referred to as the R- or S- form.

The mimetics of the invention can also include compositions that contain a structural mimetic residue, particularly a residue that induces or mimics secondary structures, such as a beta turn, beta sheet, alpha helix structures, gamma turns, and the like. For example, substitution of natural amino acid residues with D-amino acids; N-alpha-methyl amino acids; C-alpha-methyl amino acids; or dehydroamino acids within a peptide can induce or stabilize beta turns, gamma turns, beta sheets or alpha helix conformations. Beta turn mimetic structures have been described, e.g., by Nagai (1985) Tet. Lett. 26:647-650; Feigl (1986) J. Amer. Chem. Soc. 108:181-182; Kahn (1988) J. Amer. Chem. Soc. 110:1638-1639; Kemp (1988) Tet. Lett. 29:5057-5060; Kahn (1988) J. Molec. Recognition 1:75-79. Beta sheet mimetic structures have been described, e.g., by Smith (1 992) J. Amer. Chem. Soc. 114:10672-10674. For example, a type VI beta turn induced by a cis amide surrogate, 1,5-disubstituted tetrazol, is described by Beusen (1995) Biopolymers 36:181-200. Incorporation of achiral omega-amino acid residues to generate polymethylene units as a substitution for amide bonds is described by Baneijee (1996) Biopolymers 39:769-777. Secondary structures of polypeptides can be analyzed by, e.g., high-field 1H NMR or 2D NMR spectroscopy, see, e.g., Higgins (1997) J. Pept. Res. 50:421-435. See also, Hruby (1997) Biopolymers 43:219-266, Balaji, et al., U.S. Pat. No. 5,612,895.

The subject compounds can be further modified to make the compound more soluble or to facilitate its entry into a cell. For example, the compound can be PEGylated at any position, or the compound can contain a transmembrane transporter region.

A number of peptide sequences have been described in the art as capable of facilitating the entry of a peptide linked to these sequences into a cell through the plasma membrane (Derossi et al., 1998, Trends in Cell Biol. 8:84). For the purpose of this invention, such peptides are collectively referred to as transmembrane transporter peptides. Examples of these peptide include, but are not limited to, tat derived from HIV (Vives et al., 1997, J. Biol. Chem. 272:16010; Nagahara et al., 1998, Nat. Med. 4:1449), antennapedia from Drosophila (Derossi et al., 1994, J. Biol. Chem. 261:10444), VP22 from herpes simplex virus (Elliot and D'Hare, 1997, Cell 88:223-233), complementarity-determining regions (CDR) 2 and 3 of anti-DNA antibodies (Avrameas et al., 1998, Proc. Natl Acad. Sci. U.S.A., 95:5601-5606), 70 KDa heat shock protein (Fujihara, 1999, EMBO J. 18:411-419) and transportan (Pooga et al., 1998, FASEB J. 12:67-77). A truncated HIV tat peptide can be employed.

D. Improving Anti-Viral and Anti-Bacterial Agents

To improve acceptance and introduction of the anti-viral or anti-bacterial agent into a cell of choice, there are a number of known methods. For example, PEGylation of proteins can be used to make them more resistant to the immune system. Alternatively, intracellular signals or moieties can be added to proteins and vectors to allow them to more easily enter the cell of choice. Moieties that make the protein or vector specifically acceptable to uptake by infected cells can be added, in this case a ligand that is specific for a receptor expressed by respiratory cells. The moiety can be specific for a virally or bacterially infected cell, such as a receptor or cell-type specific receptor.

The instant therapeutic compounds can be further modified to make the compound more soluble or to facilitate its entry into a cell. For example, the compound can be PEGylated at any position, or the compound can be conjugated to a membrane translocating peptide such as a tat, Antennapedia or signal sequence membrane translocation peptide such as described by U. Langel, “Cell Penetrating Peptides”, CRC Press, Boca Rotan, 2002, i.e., incorporated herein by reference in its entirety.

A number of peptide sequences have been described in the art as capable of facilitating the entry of a peptide linked to these sequences into a cell through the plasma membrane (Derossi et al., 1998, Trends in Cell Biol. 8:84). For the purpose of this invention, such peptides are collectively referred to as “transmembrane transporter peptides”, which is used interchangeably with “cell penetrating peptides”. Examples of the latter cell penetrating peptides include, but are not limited to the following: namely, tat derived from HIV (Vives et al., 1997, J. Biol. Chem. 272:16010; Nagahara et al., 1998, Nat. Med. 4:1449), antennapedia from Drosophila (Derossi et al., 1994, J. Biol. Chem. 261:10444), VP22 from herpes simplex virus (Elliot and D'Hare, 1997, Cell 88:223-233), complementarity-determining regions (CDR) 2 and 3 of anti-DNA antibodies (Avrameas et al., 1998, Proc. Natl Acad. Sci. U.S.A., 95:5601-5606), 70 KDa heat shock protein (Fujihara, 1999, EMBO J. 18:411-419) and transportan (Pooga et al., 1998, FASEB J. 12:67-77). A truncated HIV tat peptide can be employed.

F. Methods of Treatment

Pharmaceutical compositions disclosed herein are used in methods of treatment or prophylaxis of bacterial or viral diseases.

As can be appreciated from the disclosure above, the present invention has a wide variety of applications. For example, the inhibitors of either PL protein, PDZ protein or the interaction between a PL and PDZ protein, can be used to identify an agent or conjugate that interacts with the transporter and that can cross into the infected cell. The inhibitors of either PL protein, PDZ protein or the interaction between PL protein and PDZ protein also can be used to increase the capacity of an agent to bind to an infected cell by identifying a conjugate moiety that binds to the infected cell and linking the conjugate moiety to the agent.

In prophylactic application, pharmaceutical compositions or medicants are administered to a patient susceptible to, or otherwise at risk for developing bacterial or viral infections in an amount sufficient to prevent, reduce, or arrest the development of the infections. In therapeutic applications, compositions or medicants are administered to a patient suspected to develop, or already suffering from a viral or bacterial disease in an amount sufficient to reverse, arrest, or at least partially arrest, the symptoms of the bacterial or viral infections. In both prophylactic and therapeutic regimes, active agents in the form of inhibitors of PL proteins, PDZ, and/or the PL -PDZ interaction, of the present invention are usually administered in several dosages until a sufficient response has been achieved. However, in both prophylactic and therapeutic regimes, the active agents can be administered in a single dosages until a sufficient response has been achieved. Typically, the treatment is monitored and repeated dosages can be given. Furthermore, the treatment regimes can employ similar dosages; routes of administration and frequency of administration to those used in treating bacteria or viral infection or progression of a bacterial or viral infection.

The amount of the inhibitors of PL protein, PDZ protein and/or the PL /PDZ interaction and other active agents that can be combined with a carrier material to produce a single dosage form vary depending upon the disease treated, the mammalian species, and the particular mode of administration. The “effective dosage”, “pharnacologically acceptable dose” or “pharmacologically acceptable amount” for any particular patient can depend on a variety of factors including the activity of the specific compound employed, the species, age, body weight, general health, sex and diet of the patient being treated; the time and route of administration; the rate of metabolism or excretion; other drugs which are concurrently or have previously been administered; the type and severity of the disease; severity of side-effects, whether the patient is animal or human, and the like. Usually the patient is human, but nonhuman mammals, including transgenic mammals, can also be treated. Full length or active fragments of the active agents can be administered in effective dosages.

For any inhibitors of PL protein, PDZ protein and/or the PL protein/PDZ interaction and other active agents used in the methods of the present invention, an effective dose for humans can be estimated initially from non-human animal models. An effective dose can be determined by a clinician using parameters known in the art. Generally, dosing begins with an amount somewhat less than the optimal effective dose. Dosing is then increased by small increments thereafter until an effective dosage is achieved. (See The Merck Manual of Diagnosis and Therapy, 16^{th} Edition, § 22, 1992, Berkow, Merck Research Laboratories, Rahway, N.J., which is incorporated herein by reference).

Dosages need to be titrated to optimize safety and efficacy. Toxicity and therapeutic efficacy of the compounds described herein can be determined by standard pharmaceutical procedures in experimental animals, e.g., by determining the LD₅₀, (the dose lethal to 50% of the population tested) and the ED₅₀ (the dose therapeutically effective in 50% of the population tested). The dose ratio between toxic and therapeutic effect is the therapeutic index and can be expressed as the ratio between LD₅₀ and ED₅₀. Compounds that exhibit high therapeutic indices are preferred. The data obtained from these nonhuman animal studies can be used in formulating a dosage range that is not toxic for use in humans. The dosage of such compounds lies preferably within a range of circulating concentrations that include the ED₅₀ with little or no toxicity. The exact formulation, route of administration and dosage can be chosen by the individual physician in view of the patient's condition. (See, e.g., Fingl et al (1975) In: The Pharmacological Basis of Therapeutics, Chapter 1, which is incorporated herein by reference).

G. Methods of Administration

Inhibitors of PL protein, PDZ protein and/or the PL protein/PDZ interaction and other active agents can be delivered or administered to a mammal, e.g., a human patient or subject, alone, in the form of a pharmaceutically acceptable salt or hydrolyzable precursor thereof, or in the form of a pharmaceutical composition wherein the compound is mixed with suitable carriers or excipient(s) in an effective dosage. An effective regime means that a drug or combination of drugs is administered in sufficient amount and frequency and by an appropriate route to at least detectably prevent, delay, inhibit or reverse development of at least one symptom of bacterial or viral infection. An “effective dosage”, “pharmacologically acceptable dose”, “pharmacologically acceptable amount” means that a sufficient amount of an inhibitors of PL proteins or expression, PDZ proteins or expression and/or the PL /PDZ protein interaction, an active agent or inhibitors of PL, PDZ protein and/or the PL /PDZ protein interaction in combination with other active agents is present to achieve a desired result, e.g., preventing, delaying, inhibiting or reversing a symptom of bacterial or viral infections or the progression of bacterial or viral infections when administered in an appropriate regime.

Inhibitors of PL proteins from bacteria or virus, one or more PDZ proteins and/or the PL /PDZ protein interaction and other active agents that are used in the methods of the present invention can be administered as pharmaceutical compositions comprising the inhibitors of PL protein, PDZ protein and/or the PL /PDZ protein interaction or active agent, together with a variety of other pharmaceutically acceptable components. Pharmaceutical compositions can be in the form of solids (such as powders, granules, dragees, tablets or pills), semi-solids (such as gels, slurries, or ointments), liquids, or gases (such as aerosols or inhalants).

Suitable formulations for use in the present invention are found in Remington's Pharmaceutical Sciences (Mack Publishing Company 1985) Philadelphia, Pa., 17^{th} edition) and Langer, Science (1990) 249:1527-1533, which are incorporated herein by reference. The pharmaceutical compositions described herein can be manufactured in a conventional manner, i.e., mixing, dissolving, granulating, dragee-making, levigating, emulsifying, encapsulating, entrapping or lyophilizing processes.

Inhibitors of PL proteins, PDZ protein and/or the PL/PDZ protein interaction and other active agents can be formulated with common excipients, diluents or carriers, and compressed into tablets, or formulated as elixirs or solutions for convenient oral administration. Inhibitors of PL proteins, PDZ proteins and/or PL/PDZ protein interactions and other active agents can also be formulated as sustained release dosage forms and the like.

Administration of the compounds can be achieved in various ways, including oral, buccal, rectal, parenteral, intraperitoneal, intradermal, transdermal, intratracheal, intravenous, and intramuscular administration. The compound can be administered in a local rather than systemic manner, in a depot or sustained release formulation. In addition, the compounds can be administered in a liposome. Moreover, the compound can be administered by gene therapy.

For buccal administration, the compositions can take the form of tablets or lozenges formulated in a conventional manner.

For administration by inhalation, the compounds for use according to the present invention are conveniently delivered in the form of an aerosol spray preparation from pressurized packs, a nebulizer or a syringe sprayer, with the use of a suitable propellant, e.g., dichlorodifluoromethane, trichlorofluoromethane, dichlorotetrafluoroethane, carbon dioxide or other suitable gas, or from propellant-free, dry-powder inhalers. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The compounds can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions or emulsions in oil-based or aqueous vehicles, and can contain formulator agents such as suspending, stabilizing and/or dispersing agents. The compositions are formulated as sterile, substantially isotonic and in full compliance with all Good Manufacturing Practice (GMP) regulations of the U.S. Food and Drug Administration.

Inhibitors of PL protein, PDZ protein and/or the PL/PDZ protein interaction and other active agents can also be formulated in rectal compositions such as suppositories or retention enemas, e.g., containing conventional suppository bases such as cocoa butter, carbowaxes, polyethylene glycols or other glycerides, all of which melt at body temperature, yet are solidified at room temperature.

In addition to the formulations described previously, the compounds can also be formulated as a depot preparation. Such long acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, for example, as a sparingly soluble salt. (See, e.g., Urquhart et al., (1984), Ann Rev. Pharmacol. Toxicol. 24:199; Lewis, ed., 1981, Controlled Release of Pesticides and Pharmaceuticals, Plenum Press, New York, N.Y., U.S. Pat. Nos. 3,773,919, and 3,270,960, which are incorporated herein by reference).

Alternatively, other delivery systems for hydrophobic pharmaceutical compounds can be employed. Liposomes and emulsions are examples of delivery vehicles or carriers for hydrophobic drugs. In some methods, long-circulating, i.e., stealth, liposomes can be employed. Such liposomes are generally described in Woodle, et al., U.S. Pat. No. 5,013,556, the teaching of which is hereby incorporated by reference. The compounds of the present invention can also be administered by controlled release means and/or delivery devices such as those described in U.S. Pat. Nos. 3,845,770; 3,916,899; 3,536,809; 3,598,123; and 4,008,719; the disclosures of which are hereby incorporated by reference.

For administration by gene therapy, genetic material (e.g., DNA or RNA) of interest is transferred into a host to treat or prevent bacterial or viral infections. In the present invention, the genetic material of interest encodes an inhibitor of PL protein, PDZ and/or the PL /PDZ interaction, an active agent or a fragment thereof. According to one aspect of the invention, the genetic material should be therapeutically effective. Many such proteins, vectors, DNA are known per se. (See Culver, K. W., “Gene Therapy”, 1994, p. xii, Mary Ann Liebert, Inc., Publishers, New York, N.Y., incorporated herein by reference in its entirety). For the purposes of example only, vectors can be selected from the group consisting of Moloney murine leukemia virus vectors, adenovirus vectors with tissue specific promoters, herpes simplex vectors, vaccinia vectors, artificial chromosomes, receptor mediated gene delivery, and mixtures of the above vectors. Gene therapy vectors are commercially available from different laboratories such as Chiron, Inc., Emeryville, Calif.; Genetic Therapy, Inc., Gaithersburg, Md.; Genzyme, Cambridge, Mass.; Somtax, Alameda, Calif.; Targeted Genetics, Seattle, Wash.; Viagene and Vical, San Diego, Calif.

Adenoviruses are promising gene therapy vectors for genetic material encoding inhibitors of PL protein, PDZ and/or PL/PDZ interaction, active agent or a fragment thereof. Adenovirus can be manipulated such that it encodes and expresses the desired gene product (e.g., inhibitors of PL, PDZ and/or PL/PDZ interaction or a fragment thereof) and at the same time is inactivated in terms of its ability to replicate in a normal lytic viral life cycle. Adenovirus expression is achieved without integration of the viral DNA into the host cell chromosome, thereby alleviating concerns about insertional mutagenesis. Furthermore, adenoviruses have been used as live enteric vaccines for many years with an excellent safety profile (Schwartz, A. R. et al. (1974) Am. Rev. Respir. Dis. 109:233-238). Finally, adenovirus mediated gene transfer has been demonstrated in a number of instances including transfer of alpha-1-antitrypsin and CFTR to the lungs of cotton rats (Rosenfeld, M. A. et al. (1991) Science 252:431-434; Rosenfeld et al., (1992) Cell 68:143-155). Furthermore, extensive studies to attempt to establish adenovirus as a causative agent in human cancer were uniformly negative (Green, M. et al. (1979) PNAS USA 76:6606).

The pharmaceutical compositions also can comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

### EXAMPLES

The following examples are put forth so as to provide a complete disclosure and description of how to make and use the present invention, and are not intended to limit the scope of the invention. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### PDZ Analysis

This example describes the binding of PDZ proteins to various bacterial and viral PL motifs. The PDZ proteins were assessed using a modified ELISA. Briefly, a GST-PDZ fusion was produced that contained the entire PDZ domain of the PDZ proteins. See Tables 1 and 2 for specific PDZ/PL pairs (Table 1) and PDZ proteins sequences (Table 2) that were used in the analysis. In addition, biotinylated peptides corresponding to the C-terminal 20 amino acids of various virus and bacterial strain PL proteins were synthesized and purified by HPLC. Binding between these entities was detected through the “G” Assay, a colorimetric assay using avidin-HRP to bind the biotin and a peroxidase substrate. The sequences of the PL proteins from the specific virus and bacteria are shown Table 1.

Binding of PL protein PLs (or C terminus) to human PDZ proteins was determined using both (i) biotinylated synthetic 20-mer peptides selected to mimic certain of the PL protein PL (or C terminus) sequences; and, (ii) recombinant PL proteins encoded by synthetic genes in recombinant systems, i.e., PL protein DNA snythesized and fused to sequences encoding a MBP immunochemical tag in an expression system (maltose binding protein; NEB; produced according to manufacturer's instructions).

Matrix graphPeptides and proteins were tested in an array format constituting a near complete set (255) of all the different PDZ domains in the human genome. Each PDZ domain polypeptide was expressed as a recombinant GST-PDZ polypeptide in a commercial glutathione S-transferase tagged expression system. Specific binding of biotinylated-PL peptides to PDZ domain polypeptides was detected using streptavidin-HRP and TMB substrate. Similarly, specific binding of PL protein-MBP fusion proteins to PDZ domain polypeptides was visualized using biotinylated anti-MBP, streptavidin-HRP and TMB substrate. The relative strength of binding was analyzed and the strong and weak binders are shown for each PL. A PDZ protein that binds more strongly is preferable when used for capturing or identifying PL proteins.

### Example 2

### HIV Peptide Testing Protocols

Two different types of ELISA assays were used to test the HIV Peptides 1904 and 1905. The primary MATRIX screen against all PDZ proteins in the library was performed under pre-incubation conditions, which are a modification of the G assay. A pre-incubation assay incubates the peptide with the HRP-streptavidin before addition of the mixture to the PDZ-coated plate. The subsequent titrations of the peptide against the PDZs of interest were performed under normal ELISA G assay conditions. In normal conditions the peptide is incubated on the PDZ coated plate before the later addition of the HRP-streptavidin. The reagents and supplies for both assays are listed below, as well as the two different protocols.

The PRISM Matrix ELISA G Assay was used for titrations. The reagents and supplies used were: Nunc Maxisorp 96 well Immuno-plate, Nunc cat#62409-002,PBS pH 7.4 (phosphate buffered saline, 8g NaCl, 0.29g KCl, 1.44g Na₂HPO₄, 0.24g KH₂PO₄, add H20 to 1 L and pH 7.4; 0.2μ filter) Assay Buffer: 2% BSA in PBS (20g of bovine serum albumin per liter PBS, fraction V, ICN Biomedicals, cat#IC15142983, Goat anti-GST polyclonal Ab, stock 5 mg/ml, stored at 4° C., Amersham Pharmacia cat#27-4577-01. Dilute 1:1000 in PBS, final concentration 5 μg/ml. HRP-Streptavidin, 2.5 mg/2 ml stock stored @ 4° C., Zymed cat#43-4323, dilute 1:2000 into Assay buffer, final [0.5 μg/ml]. Biotinylated peptides (from Anaspec, stored in −20° C. freezer), GST-PRISM proteins (stock stored (−80° C., after 1^{st} thaw store in −101C freezer), TMB (3,3′,5,5′, teramethylbensidine), Sigma cat#T5525-1 OOAB, 0.18M H₂SO₄, Sigma cat.#S 1526, 12-w multichannel pipettor, Rainin cat#L12-200, 200 μL LTS tips, VWR cat#37001-602, 50 ml reagent reservoirs, Costar#4870, 50 polypropylene conical tubes, SARSTEDT cat#62.547.004, 15 mL polypropylene round-bottom tubes, Falcon cat#352059, 1.5 mL microtubes, SARSTEDT cat#72.690, Costar Transtar 96 Costar#7605, Transtar 96 Cartridge Costar#7610, Molecular Devices microplate reader (450 nm filters), SoftMax Pro software, *When using reagents stored at or 4° C. or -20° C., reagents are removed & kept on ice.

The protocol was as follows:

1. Coat plate 50ul/well directly with anti-GST, incubate O/N (4° C.

2. Dump liquid out of plate and tap plate on paper towels

3. Block plate with 200ul/well with 1×PBS/2% BSA at RT for 1-2 hours

4. Prepare proteins in 1×PBS at 5 μg/ml

5. Add proteins at 50 μl per well, incubate 1-2 hours at 4° C.

6. Prepare peptides in Assay Buffer

7. Wash plate 3× with cold PBS*

8. Add peptides at 50 μl per well (write time on plate)

9. Incubate at 4° C. for 10 min then at room temp. for 2 minutes

10. Prepare Streptavidin-HRP at 1:2000

11. Wash plate 3× with cold PBS*

12. Add HRP at 100 μl per well (write time on plate)

13. Incubate at 4° C. for 20 minutes.

14. Turn on plate reader and prepare files

15. Promptly wash plate 5× with RT 1×PBS

16. Add 100 μl/well TMB substrate (write time on plate)

17. Incubate in dark at room temp for a maximum of 30 minutes

18. Stop reaction with 100 μl of 0.18M H₂SO₄, 30 min. after adding TMB

19. Take last reading at 450 nm soon after stopping reaction

* do not let plates dry out

The PRISM Matrix ELISA modified G Assay was also used for pre-incubation and for the MATRIX assay. The protocol was as follows.

1. Coat plate with 100 μl of 5 μg/ml anti-GST, O/N @ 4° C.

2. Dump contents out of plate and tap dry on paper towels

3. Block with 200 μl Assay Buffer for 1 to 2 hrs at room temperature

4. Prepare proteins in Assay Buffer

5. Wash plate 3× with cold PBS*

6. Add proteins at 50 μl per well, incubate 1 to 2 hrs at 4° C.

7. Prepare peptides in Assay Buffer:

Prepare peptides in half of volume need with double of desired concentration.
Dilute HRP (1:1000) in another half of volume (same volume with peptide) then mix with peptides.
Incubate the peptide-HRP mixtures for 20 minutes at RT
   8. Wash plate 3× with cold PBS*
   9. Add peptide-HRP mixtures at 50 μl per well (write time on plate)
   10. Incubate at RT after last peptide has been added for exactly 30 minutes
   11. Turn on plate reader and prepare files
   12. Promptly wash plate 5× with room temperature PBS
   13. Add 100 μl/well TMB substrate (write time on plate)
   14. Incubate in dark at room temp for a maximum of 30 minutes
   15. Stop reaction with 100 μl of 0.18M H₂SO₄, 30 min. after adding TMB
   16. Take last reading at 450 nm soon after stopping reaction

* do not let plates dry out

### Example 3

### HIV-1 Peptide 1904 Binding to PDZ Proteins

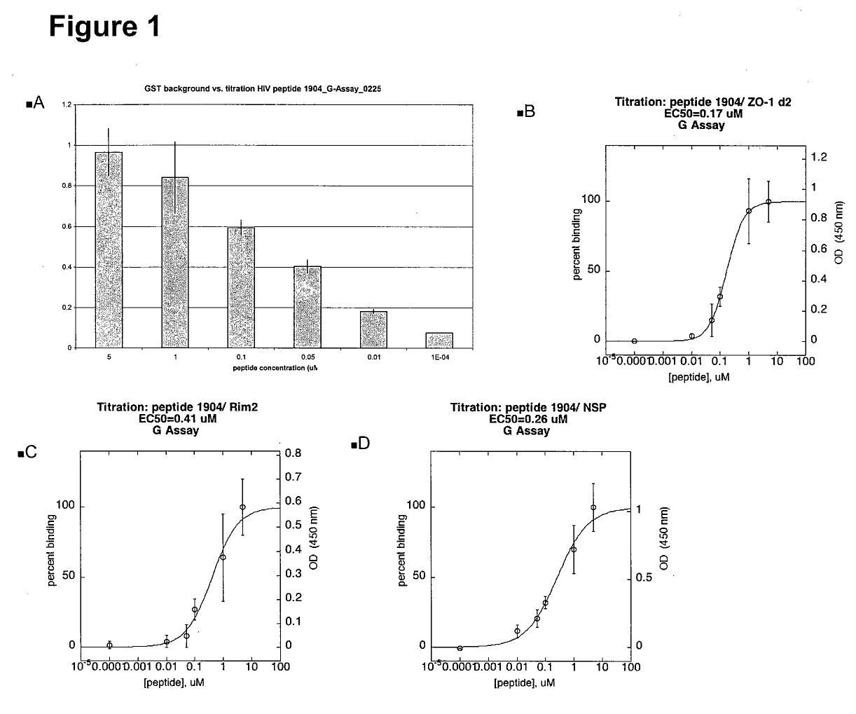

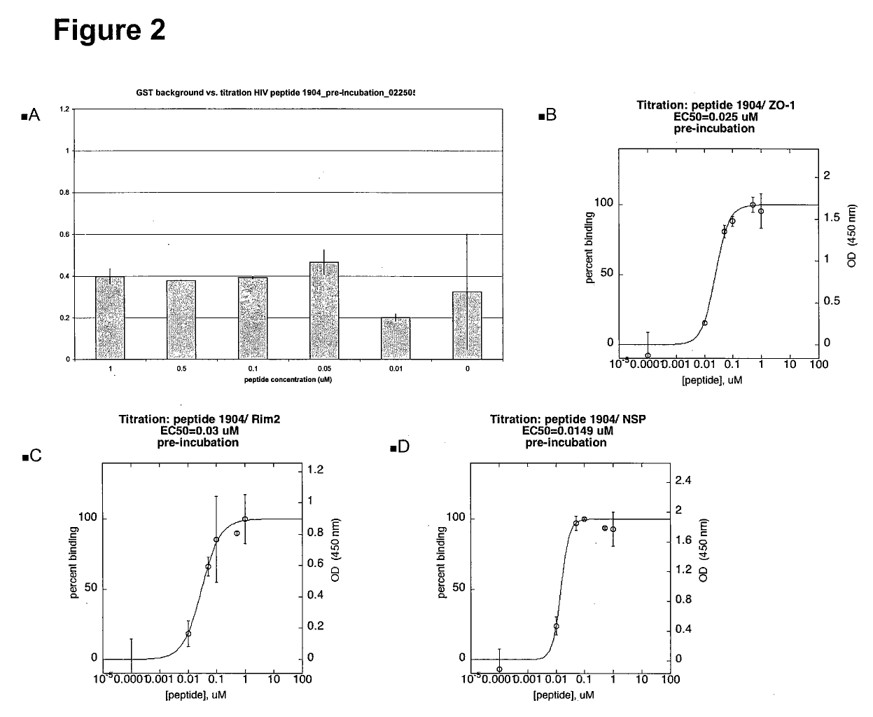

FIGS. 1 and 2 show the binding analysis for Peptide 1904 from HIV-1 with the sequence YGRKKRRQRRRRQGLERILL (SEQ ID NO:274). Peptide 1904 has a PL corresponding to RILL (SEQ ID NO:243). The analysis was done using a number of GST PDZ fusions. After a PDZ proteins was identified to bind to the peptide, a titration was performed to determine the EC50. FIGS. 1 and 2 are experiments, performed using two different assays, the regular G assay and modified G assay for the same 3 GST/PDZ fusions. FIG. 1A shows the GST background versus titration of HIV peptide 1904 in the G-Assay. The x-axis shows the background level. The y-axis shows the peptide concentration in μM. FIG. 1B shows the titration analysis for binding to the PDZ protein ZO-1 d2 (domain 2) to have an EC50 of 0.17 μM. FIG. 1 C shows the titration analysis for binding to the PDZ protein Rim2 to have an EC50 of 0.41 μM. FIG. 1D shows the titration analysis for binding to the PDZ protein NSP to have an EC50 of 0.26 1 μM. Thus, Peptide 1904 shows strong binding to the PDZ proteins ZO-1 d2, Rim2, and NSP. FIG. 2A shows the GST background versus titration of HIV peptide 1904 in the modified G-Assay. FIG. 2B shows the titration analysis for binding to the PDZ protein ZO-1 d2 (domain 2) to have an EC50 of 0.025 μM. FIG. 2C shows the titration analysis for binding to the PDZ protein Rim2 to have an EC50 of 0.03 μM. FIG. 2D shows the titration analysis for binding to the PDZ protein NSP to have an EC50 of 0.0149 μM. Analysis of other PDZ proteins can be seen in Table 3.

TABLE 3
Peptide 1904 (YGRKKRR1RRRRQGLERILL -
SEQ ID NO: 274)

| PDZ | Intensity of Hit |
|---|---|
| SIP1 d1 | WEAK |
| INADL d3 | STRONG |
| MUPP1d3 | WEAK |
| AIPC d1 | STRONG |
| KIAA0316 | STRONG |
| SITAC-18 d1 | STRONG |
| SITAC-18 d2 | STRONG |
| Magi1 d1 | STRONG |
| MINT1 d1, 2 | STRONG |
| RIM2 | STRONG |
| ZO-1 d4 | STRONG |
| PAR6 beta | STRONG |
| NSP | STRONG |
| GORASP1 d1 | STRONG |
| MUPP1d11 | STRONG |
| MAST2 | STRONG |
| PAR3L d3 | STRONG |
| NOS1 | STRONG |
| PAR3 d3 | STRONG |
| RhophilinLike | STRONG |
| KIAA1284 | STRONG |

### Example 5

### HIV-1 Peptide 1905 Binding to PDZ Proteins

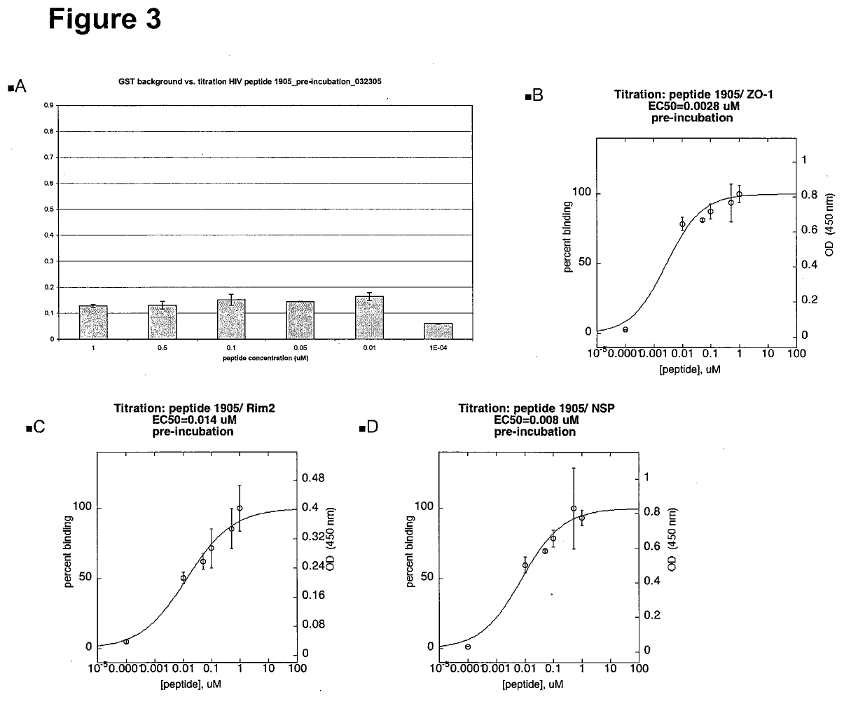

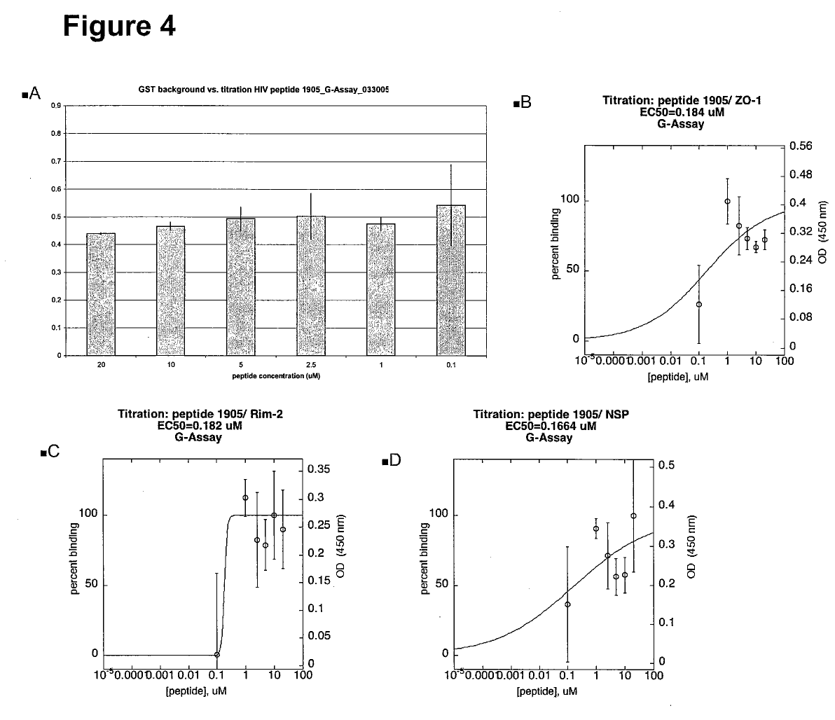

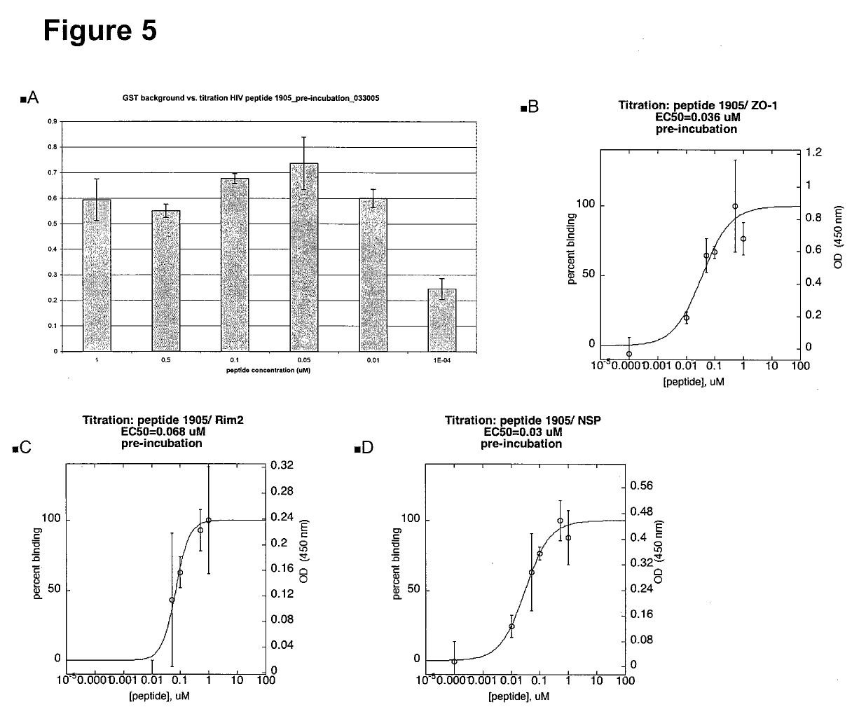

FIGS. 3-5 show the binding analysis for Peptide 1905 from HIV-1 with the sequence YGRKKRRQRRRRQGFERALL (SEQ ID NO:273). Peptide 1905 has a PL corresponding to RALL (SEQ ID NO:242). The analysis was done using a number of GST PDZ fusions. After a PDZ proteins was identified to bind to the peptide, a titration was performed to determine the EC50. FIGS. 3-5 are triplicates of the analysis of three PDZ proteins. FIG. 3A shows the GST background versus titration of HIV peptide 1905 in the modified G-Assay. The x-axis shows the background level. The y-axis shows the peptide concentration in 1M. FIG. 3B shows the titration analysis for binding to the PDZ protein ZO-1 d2 (domain 2) to have an EC50 of 0.0028 μM. FIG. 3C shows the titration analysis for binding to the PDZ protein Rim2 to have an EC50 of 0.014 μM. FIG. 3D shows the titration analysis for binding to the PDZ protein NSP to have an EC50 of 0.008 μM. FIG. 4A shows the GST background versus titration of HIV peptide 1905 in the G-Assay. FIG. 4B shows the titration analysis for binding to the PDZ protein ZO-1 d2 (domain 2) to have an EC50 of 0.184 μM. FIG. 4C shows the titration analysis for binding to the PDZ protein Rim2 to have an EC50 of 0.182 μM. FIG. 4D shows the titration analysis for binding to the PDZ protein NSP to have an EC50 of 0.1664 μM. FIG. 5A shows the GST background versus titration of HIV peptide 1905 in the modified G-Assay. FIG. 5B shows the titration analysis for binding to the PDZ protein ZO-1 d2 (domain 2) to have an EC50 of 0.036 μM. FIG. 3C shows the titration analysis for binding to the PDZ protein Rim2 to have an EC50 of 0.068 μM. FIG. 3D shows the titration analysis for binding to the PDZ protein NSP to have an EC50 of 0.03 μM. Thus, Peptide 1905 shows strong binding to the PDZ proteins ZO-1 d2, Rim2, and NSP. Analysis of other PDZ proteins can be seen in Table 4.

### Example 6

### Lateral Flow

Examples of lateral flow formats for detection of PL proteins are provided using PDZ capture followed by monoclonal antibody detection or monoclonal antibody capture followed by PDZ detection. For all cases, recombinant PDZ domain proteins or antibodies are deposited on RF120 Millipore membrane using a striper, typically at a concentration of about 1 mg/ml. A control band is also deposited composed of goat anti-mouse antibody (GAM) also at 0.5 mg/ml. PL proteins are combined with gold conjugated monoclonal anti-PL antibody in 100 ul volume in TBS-T buffer. Some recombinant PL proteins are used as positive controls, and a control lane does not contain the PL protein. In all cases, the samples are diluted and stored in saline or M4, as indicated. The samples are directly mixed with gold conjugated antibody (or PDZ proteins).

When using the PDZ as a capture agent, the PDZ is striped onto the membrane and the membrane is inserted into the sample and flow initiated by capillary action and a wicking pad. Optionally a number of different monoclonal antibodies are deposited on the lateral flow device alone or in combination as capture agents. The sample is mixed with gold conjugated PDZ proteins, and applied to the lateral flow device. If the virus or bacteria is present a line is formed on the strip.The binding strength is quantified by using the following symbols: (−) for no binding, (+) for weak binding, (+++) for strong binding and (++) for moderate binding.

The materials that are used include strips previously striped with goat anti-mouse/PDZ proteins; TBST/2% BSA/0.25% Tween 20 buffer; Stocks of viral or bacterial PL proteins HIV-1 Env, HIV-1 Nef, HIV-2 Env, and HIV-2 Vif for the HIV test, a gold-conjugated monoclonal antibody; and Maxisorp ELISA plates. The procedure is performed as follows:
1) Stock PL proteins are diluted down in TBST/2% BSA/0.25% Tween 20 to 100 ng/uL (using no less than 5uL of proteins to perform the dilutions)
2.) The 100 ng/uL dilution is diluted down to 50 ng/uL by adding 10 uL of the protein (100 ng/μl) to 10uL of TBST/2% BSA/0.25% Tween 20
3.) A stock solution of gold-conjugated antibody in TBST/2% BSA/0.25% Tween 20 buffer is prepared. Four uL of the antibody is added to every 100 uL of the buffer, and enough buffer is prepared for 6 of 100 uL reactions (which provides extra dead volume).
4.) 98uL of the antibody/buffer mix is added to separate wells in the ELISA plate
5.) 2 uL of the PL protein dilutions are added to the buffer-containing wells (one PL per well)
6.) One well is left with just antibody and buffer to serve as a negative “no PL” control
7.) The ELISA plate is tapped several times to mix the contents of the wells
8.) The pre-striped strips are added to the wells and observed during development. After approximately 30 minutes the strips are removed from the wells and scanned into the computer.

A mixed viral test is prepared as follows: a GST-TIP2 protein is striped onto a membrane at 3 mg/mL for a Hepatitis B, or alternatively a mixture of two monoclonal antibodies can be used (about 2 mg/mL monoclonal antibody). A second line of 1 mg/mL polyclonal goat anti-mouse antibody is used for the test capture line. The steps are set out below.

1. Prepare cards with a membrane and sink pad.

2. Stripe membrane with the PDZ protein and/or antibodies (see above for conc.)

3. Dry the membrane overnight at 56 degrees, then cut the cards into strips 4.26 mm wide.

4. Attach a glass fiber sample pad to the bottom of the strip and place the entire strip inside a cassette for testing.

5. Thaw sample to be tested and add 80 μl of sample to 20 μl of buffer. Pipette up and down several times to mix.

6. Spike 8 μl of the gold-conjugated (Au-) detector mix into the sample/buffer solution. This detector mix is about 8 μl of Au-monoclonal antibodies for the PL protein. Pipette up and down several times to mix.

7. Add 100 μl of the prepared sample to the sample well on the cassette.

8. Read the test and control lines on the cassette at 15 minutes post-addition of sample. The control line is clearly visible for any test results to be read reliably. The top arrow is pointing to the control and the bottom arrow is pointing to the test.

TABLE 4
Peptide 1905 (YGRKKRR1RRRRQGFERALL -
SEQ ID NO: 273)

| PDZ | Intensity of Hit |
|---|---|
| SIP1 d1 | STRONG |
| EBP50 | STRONG |
| Shank 2 | STRONG |
| Tip | STRONG |
| NSP | STRONG |
| Shank 1 | STRONG |
| Shank 3 | STRONG |
| EBP50 d1 | STRONG |
| MAST2 | STRONG |
| PAR3 d3 | STRONG |
| PICK1 F.L. | STRONG |
| KIAA1284 | STRONG |

All publications and patents cited in this specification are herein incorporated by reference as if each individual publication or patent were specifically and individually indicated to be incorporated by reference. Genbank records referenced by GID or accession number, particularly any polypeptide sequence, polynucleotide sequences or annotation thereof, are incorporated by reference herein. The citation of any publication is for its disclosure prior to the filing date and should not be construed as an admission that the present invention is not entitled to antedate such publication by virtue of prior invention.

While the present invention has been described with reference to the specific embodiments thereof, it various changes can be made and equivalents can be substituted without departing from the true spirit and scope of the invention. In addition, many modifications can be made to adapt a particular situation, material, composition of matter, process, process step or steps, to the objective, spirit and scope of the present invention. All such modifications are intended to be within the scope of the claims appended hereto.

1. A method for identifying whether a patient is infected with M. tuberculosis, comprising:
determining whether an M. tuberculosis PDZ ligand (PL) protein is present in a patient sample, presence indicating the patient is infected with M. tuberculosis.

2. The method of claim 1, wherein the determining comprises contacting a patient sample with an agent that specifically binds to the M. tuberculosis PL protein; and
detecting specific binding between the agent and the PL protein, specific binding indicating presence of M. tuberculosis.

3. The method of claim 1, wherein the M. tuberculosis PL protein is ESXN, ESXS, or ESAT-6.

4. The method of claim 2 wherein the agent that specifically binds to the PL protein binds to a PL motif.

5. The method of claim 4, wherein the PL motif is SSWA (SEQ ID NO: 270) for M. tuberculosis ESXN protein, YTGF (SEQ ID NO: 271) for M. tuberculosis ESXS protein, GMFA (SEQ ID NO: 272) for M. tuberculosis ESAT-6 protein.

6. The method of claim 5, wherein the agent that specifically binds to the M. tuberculosis ESXN protein PL motif is a PDZ protein selected from the group consisting of: TIP2, KIAA1526, and PSD95 (p2).

7. The method of claim 5, wherein the agent that specifically binds to the M. tuberculosis ESXS protein PL motif is a PDZ protein selected from the group consisting of: MAST2, MAST3, Shank3, APXL1, and syntenin.

8. The method of claim 5, wherein the agent that specifically binds to the M. tuberculosis ESAT-6 protein PL motif is a PDZ protein selected from the group consisting of: INADL (p3), RIM2, and TIP2.

9. An isolated antibody that specifically binds to a carboxy-terminal PL motif in a PL protein of M. tuberculosis.

10. A method for the treatment or prophylaxis of a patient having or at risk of tuberculosis, comprising:
administering to the patient an effective regime of an agent that that inhibits interaction of a PL protein of M. tuberculosis with a PDZ protein of the patient and thereby effecting treatment or prophylaxis of the infection.

11. The method of claim 10, wherein said agent is an antibody that specifically binds to the PL motif of a PL protein.

12. The method of claim 10, wherein the agent is selected from the group consisting of: an antisense oligonucleotide, a small molecule, an siRNA and a zinc finger protein.

13. The method of claim 10, wherein the PL protein is a member of the ESAT-6 family.

14. A method for identifying whether a patient is infected with HIV, comprising:
determining whether an HIV PDZ ligand (PL) protein is present in a patient sample, presence indicating the patient is infected with HIV.

15. The method of claim 14, wherein the determining comprises contacting a patient sample with an agent that specifically binds to the HIV PL protein; and
detecting specific binding between the agent and the PL protein, specific binding indicating presence of HIV.

16. The method of claim 14, wherein the HIV protein is Env, Nef or Vif.

17. The method of claim 15 wherein the agent that specifically binds to the PL protein binds to a PL motif.

18. The method of claim 17, wherein the PL motif is RALL (SEQ ID NO:242) or RILL (SEQ ID NO:243) for HIV-1 Env, FKNC (SEQ ID NO:244), FKDC (SEQ ID NO:245), YKNC (SEQ ID NO:246), or YKDC (SEQ ID NO:247) for HIV-1 Nef protein, IALL (SEQ ID NO:248), LALL (SEQ ID NO:249), or LTLL (SEQ ID NO:250) for HIV2 Env protein, and EILA(SEQ ID NO:251), GILA (SEQ ID NO:252), or DILA (SEQ ID NO:253) for HIV-2 Vif protein.

19. The method of claim 18, wherein the agent that specifically binds to the Env PL motif for HIV-1 is a PDZ protein selected from the group consisting of: AIPC (p1), GORASP1 (p1), INADL (p3), KIAA0316, KIAA1284, MAGI1 (p1), MAST2, MINT1 (p1,2), NSP, NOSI, PAR3 (p3), PAR3L (p3), PAR6 beta, RIM2, Rhodophilin-like, SITAC-18 (p2), SITAC-18 (p1), KIAA1284, PICK1, Shank 1, Shank 2, Shank 3, and TIP1.

20. The method of claim 18, wherein the agent that specifically binds to the Nef PL motif for HIV-1 is a PDZ protein selected from the group consisting of: MINT1, SITAC-18, TIP1 and PICK1.

21. The method of claim 18, wherein the agent that specifically binds to the Env PL motif for HIV-2 is a PDZ protein selected from the group consisting of: EBP50 (p1), KIAA1284, MAST2, NSP, PAR3, PICK1, Shank 1, Shank 2, Shank 3, and TIP1.

22. The method of claim 18, wherein the agent that specifically binds to the Vif PL motif for HIV-2 is a PDZ protein selected from the group consisting of: INADL (p3), RIM2, and EBP50 (p1).

23. An isolated antibody that specifically binds to a carboxy-terminal PL motif in a PL protein of HIV.

24. A method for the treatment or prophylaxis of a patient having or at risk of HIV infection, comprising:
administering to the patient an effective regime of an agent that that inhibits interaction of a PL protein of HIV with a PDZ protein of the patient and thereby effecting treatment or prophylaxis of the infection.

25. The method of claim 24, wherein said agent is an antibody that specifically binds to the PL motif of the PL protein.

26. The method of claim 24, wherein the agent is selected from the group consisting of: an antisense oligonucleotide, a small molecule, an siRNA and a zinc finger protein.

27. The method of claim 24, wherein the PL protein is Env, Nef or Vif.

28. A method for identifying whether a patient is infected with Hepatitis B, comprising:
determining whether a Hepatitis B PDZ ligand (PL) protein is present in a patient sample, presence indicating the patient is infected with Hepatitis B.

29. The method of claim 28, wherein the determining comprises contacting a patient sample with an agent that specifically binds to the Hepatitis B PL protein; and
detecting specific binding between the agent and the PL protein, specific binding indicating presence of Hepatitis B.

30. The method of claim 28, wherein the Hepatitis B protein is Protein X or S antigen.

31. The method of claim 30 wherein the agent that specifically binds to the PL protein binds to a PL motif.

32. The method of claim 31, wherein the PL motif is. FTSA (SEQ ID NO:254) for Hepatitis B Protein X, or WVYI (SEQ ID NO:255) for Hepatitis B S antigen.

33. The method of claim 30, wherein the agent that specifically binds to the Hepatitis B Protein X PL motif is a PDZ protein selected from the group consisting of:
TIP2, KIAA1526, SITAC-18, MINT1 (p1,2), DVL3, and NOS1.

34. The method of claim 30, wherein the agent that specifically binds to the Hepatitis B S antigen PL motif is a PDZ protein selected from the group consisting of:
PTPL1 (p4), HEMBA 1003117, AF6, AIPC, SYNTENIN, MUPP1 (p3,7,9,11), DVL2 (01), ZO-3 (p1), SIP1, AIPC (p1), GORASP1 (p1), INADL (p3), KIAA0316, KIAA1284, MAGI1 (p1), MAST2, MINT1 (p1,2), NSP, NOS1, PAR3 (p3), PAR3L (p3), PAR6 beta, RIM2, Rhodophilin-like, SITAC-18 (p2), SITAC-18 (p1), KIAA1284, PICK1, Shank 1, Shank 2, Shank 3, and TIP1.

35. An isolated antibody that specifically binds to a carboxy-terminal PL motif in a PL protein of Hepatitis B.

36. A method for the treatment or prophylaxis of a patient having or at risk of Hepatitis B infection, comprising:
administering to the patient an effective regime of an agent that that inhibits interaction of a PL protein of Hepatitis B with a PDZ protein of the patient and thereby effecting treatment or prophylaxis of the infection.

37. The method of claim 36, wherein said agent is an antibody that specifically binds to the PL motif of the PL protein.

38. The method of claim 36, wherein the agent is selected from the group consisting of: an antisense oligonucleotide, a small molecule, an siRNA and a zinc finger protein,.

39. The method of claim 36, wherein the PL protein is Protein X or S antigen.

40. A method for identifying whether a patient is infected with a flavivirus, comprising:
determining whether a flavivirus PDZ ligand (PL) protein is present in a patient sample, presence indicating the patient is infected with a flavivirus.

41. The method of claim 40, wherein the determining comprises contacting a patient sample with an agent that specifically binds to the flavivirus PL protein; and
detecting specific binding between the agent and the PL protein, specific binding indicating presence of flavivirus.

42. The method of claim 40, wherein the flavivirus protein is Capsid C or E1.

43. The method of claim 41 wherein the agent that specifically binds to the PL protein binds to a PL motif.

44. The method of claim 41, wherein the flavivirus is Hepatitis C virus and the PL motif is: PASA (SEQ ID NO:256), or PVSA(SEQ ID NO:257) for Hepatitis C Capsid C protein, or GVDA (SEQ ID NO:258) for Hepatitis C E1 protein,.

45. The method of claim 44, wherein the agent that specifically binds to the Hepatitis C Capsid C PL motif is a PDZ protein selected from the group consisting of:
TIP-2, and ZO-1 (p2).

46. The method of claim 44, wherein the agent that specifically binds to the Hepatitis C E1 protein PL motif is a PDZ protein selected from the group consisting of:
TIP2, RIM2, and INADL (p3).

47. An isolated antibody that specifically binds to a carboxy-terminal motif in a PL protein of a flavivirus, said flavivirus selected from the group consisting of:
Hepatitis C virus, West Nile virus, Dengue, Japanese encephalitis virus, Yellow fever virus, tich borne encephalitis virus.

48. A method for the treatment or prophylaxis of a patient having or at risk of flavivirus infection, comprising:
administering to the patient an effective regime of an agent that that inhibits interaction of a PL protein of flavivirus with a PDZ protein of the patient and thereby effecting treatment or prophylaxis of the infection.

49. The method of claim 48, wherein said agent is an antibody that specifically binds to the PL motif of a PL protein.

50. The method of claim 48, wherein the agent is selected from the group consisting of: an antisense oligonucleotide, a small molecule, an siRNA and a zinc finger protein.

51. The method of claim 48, wherein the PL protein is Capsid C or E1.

52. A method for identifying whether a patient is infected with RSV, comprising:
determining whether an RSV PDZ ligand (PL) protein is present in a patient sample, presence indicating the patient is infected with RSV.

53. The method of claim 52, wherein the determining comprises contacting a patient sample with an agent that specifically binds to the RSV PL protein; and
detecting specific binding between the agent and the PL protein, specific binding indicating presence of RSV.

54. The method of claim 52, wherein the RSV protein is Nucleoprotein.

55. The method of claim 53 wherein the agent that specifically binds to the PL protein binds to a PL motif.

56. The method of claim 55, wherein the PL motif is: DVEL (SEQ ID NO:259) for RSV Nucleoprotein.

57. The method of claim 56, wherein the agent that specifically binds to the RSV Nucleoprotein PL motif is a PDZ protein selected from the group consisting of: ZO-1 (p2), RIM2, Novel Serine Protease, MINT1, EBP50 (p1), AIPC (p1), PAR3(p3), SIP1 (p1), PTPL1 (p4), HEMBA 1003117, AF6, AIPC, SYNTENIN, MUPP1 (p3,7,9,11), DVL2 (01), ZO-3 (p1), SIP1, AIPC (p1), GORASP1 (p1), INADL (p3), KIAA0316, KIAA1284, MAGI1 (p1), MAST2, MINT1 (p1,2), NSP, NOS1, PAR3 (p3), PAR3L (p3), PAR6 beta, RIM2, Rhodophilin-like, SITAC-18 (p2), SITAC-18 (p1), KIAA1284, PICK1, Shank 1, Shank 2, Shank 3, and TIPL.

58. An isolated antibody that specifically binds to a carboxy-terminal PL motif in a PL protein of RSV.

59. A method for the treatment or prophylaxis of a patient having or at risk of RSV infection, comprising:
administering to the patient an effective regime of an agent that that inhibits interaction of a PL protein of RSV with a PDZ protein of the patient and thereby effecting treatment or prophylaxis of the infection.

60. The method of claim 59, wherein said agent is an antibody that specifically binds to a PL motif of the PL protein.

61. The method of claim 59, wherein the agent is selected from the group consisting of: an antisense oligonucleotide, a small molecule, an siRNA and a zinc finger protein.

62. The method of claim 59, wherein the PL protein is Nucleoprotein.

63. A method for identifying whether a patient is infected with Rotavirus A, comprising:
determining whether a Rotavirus A PDZ ligand (PL) protein is present in a patient sample, presence indicating the patient is infected with Rotavirus A.

64. The method of claim 63, wherein the determining comprises contacting a patient sample with an agent that specifically binds to the Rotavirus A PL protein; and
detecting specific binding between the agent and the PL protein, specific binding indicating presence of Rotavirus A.

65. The method of claim 63, wherein the Rotavirus A protein is VP4, VP7, NSP2, or NSP5.

66. The method of claim 63 wherein the agent that specifically binds to the PL protein binds to the PL motif.

67. The method of claim 63, wherein the PL motif is: QCKL (SEQ ID NO:260), or QCRL (SEQ ID NO:261) for Rotavirus A VP4 protein, YYRV (SEQ ID NO:262), or YYRI (SEQ ID NO:263) for Rotavirus A VP7 protein, QVGI (SEQ ID NO:264), HIGI (SEQ ID NO:265), QIGI (SEQ ID NO:266), or RIGI (SEQ ID NO:267) for Rotavirus A NSP2 protein, IKDL (SEQ ID NO:268) or IEDL (SEQ ID NO:269) for Rotavirus A NSP5 protein.

68. The method of claim 67, wherein the agent that specifically binds to the Rotavirus A VP4 protein PL motif is a PDZ protein selected from the group consisting of:
MAGI3 (p5), LIM mystique, LIM-RIL, ENIGMA, MAGI1 (p3), MAST2, MAGI2 (p5), LIM protein, and ZO-1 (p2).

69. The method of claim 67, wherein the agent that specifically binds to the Rotavirus A VP7 protein PL motif is a PDZ protein selected from the group consisting of:
GRIP1 (p6), PTPL1 (p4), MAST1, MUPP1 (p3,7,9), KIAA1719 (p6), MAST2, PICK1, and ZO-1 (p2).

70. The method of claim 67, wherein the agent that specifically binds to the Rotavirus A NSP2 PL motif is a PDZ protein selected from the group consisting of: NOS 1 (p1,2,3), MINT1 (p2), and ZO-1 (p2).

71. The method of claim 67, wherein the agent that specifically binds to the Rotavirus A NSP5 PL motif is a PDZ protein selected from the group consisting of: NOS1, RIM2, and ZO-1 (p2).

72. An isolated antibody that specifically binds to a carboxy-terminal PL motif in a PL protein of Rotavirus A.

73. A method for the treatment or prophylaxis of a patient having or at risk of Rotavirus A infection, comprising:
administering to the patient an effective regime of an agent that that inhibits interaction of a PL protein of Rotavirus A with a PDZ protein of the patient and thereby effecting treatment or prophylaxis of the infection.

74. The method of claim 73, wherein said agent is an antibody that specifically binds to the PL motif of the PL protein.

75. The method of claim 73, wherein the agent is selected from the group consisting of: an antisense oligonucleotide, a small molecule, an siRNA and a zinc finger protein.

76. The method of claim 73, wherein the PL protein is VP4, VP7, NSP2, or NSP5.
